(19) United States
(12) Patent Application Publication
(10) Pub. No.: US 2009/0162315 A1
(43) Pub. Date: Jun. 25, 2009

(54) Enterotoxin gene cluster (egc) superantigens to treat malignant disease

(76) Inventors: David S. Terman, Pebble Beach, CA (US); Jerome Etienne, Caluire (FR); Francois Vaudensch, Lyon (FR); Gerard Lina, Lyon (FR); Gregory A. Bohach, Moscow, ID (US)

Correspondence Address: CENTRAL COAST PATENT AGENCY, INC, 3 HANGAR WAY SUITE D, WATSONVILLE, CA, 95076, US

(21) Appl. No.: 11/571,188

(22) PCT Filed: Jun. 27, 2005
(86) PCT No.: PCT/US05/22638

§ 371 Date: Dec. 10, 2007

Related U.S. Application Data
(60) Provisional application No. 60/583,692, filed on Jun. 29, 2004.
(60) Provisional application No. 60/626,159, filed on Nov. 6, 2004.
(60) Provisional application No. 60/665,654, filed on Mar. 23, 2005.

(51) Int. Cl. A61K 38/20; A61K 39/085; A61P 37/04

(52) U.S. Cl. 424/85.2; 424/184.1; 424/237.1

(57) ABSTRACT

The use of classical superantigens for treatment of cancer has resulted in a low response rates and serious toxicity in humans which is attributable, in part, to the presence of preformed superantigen specific antibodies in the plasma of treated patients. The present invention addresses this problem by providing a method for treating tumors comprising the administration of one or a plurality of egc (enterotoxin gene cluster) staphylococcal enterotoxins comprising staphylococcal enterotoxins G, I, M, N, O. These superantigens in native unmodified form can be administered intrathecally, intratumorally, intravenously to humans with advanced lung cancer while resolving pleural effusions and prolonging survival to 300% above control patients treated with talc pleurodesis. Intratumoral egc superantigens induces a significant and sustained reduction of the tumor size. In contrast to classic Sags, the egc superantigens induced minimal toxicity, are rarely associated with the presence of preformed antibodies and are used as a plurality with a broad T cell Vβ profile. Useful egc superantigen compositions for parenteral administration native egc enterotoxins, homologues, fragments and fusion proteins of native egc enterotoxins capable of activating a broad spectrum of T cells expressing T cell receptor/α motifs. T cell survival-enhancing cytokines IL-7, Il-15, Il-23 are used. together with parenteral egc SE therapy. Also disclosed is combined therapy that includes parenteral, intratumoral or intrathecal superantigen compositions in combination with (i) intratumoral low, non-toxic doses of one or more chemotherapeutic drugs or (ii) systemic chemotherapy at reduced and non-toxic doses of chemotherapeutic drugs or (iii) radiation therapy or (iv) anti-angiogenic and tyrosine kinase inhibitors.

### CROSS REFERENCE TO RELATED DOCUMENTS

The present application claims priority to U.S. provisional application Ser. No. 60/583,692 filed on Jun. 29, 2004 and U.S. provisional application Ser. No. 60/626,159 filed on Nov. 6, 2004 and U.S. provisional application Ser. No. 60/665,654 filed on Mar. 23, 2005

### BACKGROUND OF THE INVENTION

1. Field of the Invention

The invention in the fields of immunology and medicine is directed to a method for treating a category of neoplastic diseases that are manifest in sheaths surrounding organs (intrathecal) by administering tumoricidal superantigens such as bacterial enterotoxins and various biologically active derivatives thereof.

2. Description of the Background Art

Staphylococcal enterotoxins (“SE's”) are representative of a family of proteins known as “superantigens” (SAgs)—the most powerful T lymphocyte mitogens known. They can activate between about 5 and about 30% or the total T cell population compared to the activation of 0.01% or fewer T cells by conventional antigens. Moreover, these enterotoxins elicit strong polyclonal proliferative responses at concentrations about 10³-fold lower than other T cell mitogens. The most potent SE on a per weight basis, Staphylococcal enterotoxin A (SEA), stimulates human T cell proliferation (measured as DNA synthesis) at concentrations of as low as 10⁻¹³-10⁻¹⁶M.

Mycoplasmal, viral, and other bacterial proteins are SAgs. In addition to SEs and SpEs, examples include Yersinia pseudotuberculosis mitogenic protein (“YPM”), and Clostridium perfringens toxin A. All SAgs activate T cells without a requirement for conventional antigen processing, and the responding T cells do not respond in a conventional MHC restricted manner. As noted, SAgs bind to and evoke responses from all T cells expressing certain TCR Vβ gene products independently of other TCR structures. CD4− CD8− TCR α/β T cells and γ/δ T cells all respond to SAgs by proliferation, production of TH1 cytokines and generation of cytotoxic activity.

SAg-activated T cells produce a variety of cytokines, including interferon-γ (IFNγ), various interleukins and tumor necrosis factor-α (TNFα) (Dohlsten et al., Int. J. Cancer 54:482-488 (1993)).

SAg also stimulate other cell populations involved in innate and adaptive immunity and contribute to anti-tumor immunity. For example, SE's engage the variable (V) region of the T cell receptor (TCR) chain on the exposed face of the pleated sheet and the sides of the MHC class II molecule (Kotzin B L et al., Adv Immunol. 1993; 54:99-166). SAgs augment TH1 cytokine response by CD4+ cells while also activating cells of the NK, NKT and γ/δ T cell lineages. Cytotoxic action of NK cells is augmented by the IFNγ produced by SAg activated T cells (Morita et al., Immunity 14:331-44. (2001); D'Orazio et al., J Immunol. 154:1014-23 (1995).

In addition to these biological activities, the SE's share common physicochemical properties. They are heat stable, trypsin-resistant, and soluble in water and salt solutions, have similar sedimentation coefficients, diffusion constants, partial specific volumes, isoelectric points, and extinction coefficients. Prior to more recent discoveries of additional SE's, earlier-described SEs were divided into five serological types designated SEA, Staphylococcal enterotoxin B (SEB), Staphylococcal enterotoxin C (SEC), Staphylococcal enterotoxin D (SED) and Staphylococcal enterotoxin E (SEE), which exhibit striking structural similarities.

An SE is a single polypeptide chain of about 30 kDa. All SEs have a characteristic disulfide loop near the middle of the chain. SEA is a flat monomer consisting or 233 amino acids divided into two domains: domain I comprising residues 31-116 and domain II comprising residues 117-233 together with the amino tail of residues 1-30. The biologically active regions of the proteins are evolutionarily conserved and show a relatively higher degree of sequence homology/similarity. One region of striking amino acid sequence homology between SEA, SEB, SEC, SED, and SEE is located immediately on the C-terminal side of Cys-106 (in SEA). This conserved region is thought to be responsible for T cell activation. A second conserved homology region, at about residue 147, is believed to be responsible for emetic activity. This emesis-inducing region can be deleted from SE's through genetic engineering; such modified SE's are also useful therapeutics in accordance with this invention.

Sequence analysis of SEs and comparison with other bacterial toxins revealed SEA, SEB, SEC, SED, Staphylococcal toxic shock-associated toxin (TSST-1, also known as SEF), and the Streptococcal pyrogenic exotoxins (SpE's) share considerable nucleic acid and amino acid sequence similarity (Betley et al., J. Bacteriol. 170: 34-41 (1988)). Thus, the SEs belong to a family of evolutionarily related proteins.

SEs bind to MHC class II molecules and TCRs in a manner quite distinct from conventional antigens. SEs engage the V region of the TCR β chain (Vβ region) on an exposed face of the β pleated sheet. SEs engage the “sides” of MHC class II molecule rather than engaging the groove as do conventional antigens. In contrast to SEB and the SEC, which bind only to the MHC class II α chain, SEA, as well as SEE and SED, also interact with the MHC class II α chain in a zinc-dependent manner (Fraser J D et al., Proc. Natl. Acad. Sci. 89:5507-11 (1992)).

T cell recognition of SAgs, such as SEs, via the TCR Vβ region is independent of other TCR components and diversity elements. Single amino acid positions and regions important for SAg-TCR interactions have been defined. These residues are located in the vicinity of the shallow cavity formed between the two SE domains. (Lavoie P M et al., Immunol. Rev. 168: 257-269 (1999). Substitution of amino acid residue Asn23 in SEB by Ala has demonstrated the importance of this position in SEB/TCR interactions. This particular residue is conserved among all of the SE's and may constitute a common anchor position for SE interaction with TCR Vβ structures. Amino acid residues in positions 60-64 of SEA contribute to the TCR interaction as do the Cys residues forming the intramolecular disulfide bridge Kappler J et al., J. Exp. Med. 175 387-96 (1992)). For SEC2 and SEC3, the key points of interaction in the TCR Vβ region are located in the CDR1, CDR2 and HRV4 regions of the TCR Vβ3 chain (Deringer J R et al., Mol. Microbiol. 22: 523-534 (1996)). Hence, multiple and highly variable parts of the Vβ region contribute to the formation of the TCRs SE binding site.

Thus far, no single, linear consensus motif in the TCR Vβ displaying a high affinity interaction with particular enterotoxins has been identified. A significant contribution of the TCRα chain in SE-TCR recognition is acknowledged (Smith et al., J. Immunol. 149: 887-896 (1992)). It is apparently the distinctive binding characteristics of SEs which bypass the highly variable parts of the MHC class II and TCR molecules that endows SEs with their ability to activate such a high frequency of T cells and cause massive proliferation, cytokine induction and cytotoxic T cell generation. These properties are shared by other proteins produced by various infectious agents. Together, these proteins form a well recognized family of molecules, SAgs, because of their aforementioned biological effects.

### The egc Staphylococcal Enterotoxins

Staphylococcal enterotoxins (SE) G and 1 were originally identified in two separate strains of Staphylococcus aureus. It was subsequently shown that the corresponding genes seg and sei are present in S. aureus in tandem orientation, on a 3.2-kb DNA fragment (Jarraud, S. et al. J. Clin. Microbiol. 37:2446-2449 (1999)). Sequence analysis of seg-sei intergenic DNA and flanking regions revealed three enterotoxin-like open reading frames related to seg and sei, designated sem, sen, and seo, and two pseudogenes, ψent1 and ψent2. RT-PCR analysis showed that all these genes, including seg and sei, belong to an operon, designated the enterotoxin gene cluster (egc). Recombinant SEG, SEI, SEM, SEN, and SEQ showed superantigen activity, each with a specific Vβ pattern. Distribution studies of genes encoding superantigens in clinical S. aureus isolates showed that most strains harbored such genes and, in particular, the enterotoxin gene cluster, whatever the disease they caused. Phylogenetic analysis of enterotoxin genes indicated that they all potentially derived from this cluster, identifying egc as a putative nursery of enterotoxin genes (Jarraud et al., J. Immunol, 166: 669-677. (2001)).

While most SE-producing strains of S. Aureus express genes encoding several superantigens. Becker and others found that the egc SEs were expressed about 75% of all SE-producing S. aureus strains usually in association with one or more classical superantigen. Only a rare strain produced egc SEs alone.

Bavari and Lina and others have shown that showed that up to 80% of all human sera contain factors (presumably neutralizing antibodies) that inhibit stimulation of human T cells by classical superantigen (SEs A-E and TSST-1). When classical SEs (as SE-antibody fusion proteins) were used in the treatment of cancer, the neutralizing antibodies present in patient sera inhibited SE-induced T cell proliferation and abrogated any significant anti-tumor effects. Indeed, the presence of the SE specific antibodies correlated with significant host toxicity and each successive SE treatment resulted in a progressively increased titer of SE associated antibodies and significant toxicity (Giantonio et al., J. Clin. Oncol. 15:1994-2007 (1997); Alpaugh et al., Clin. Cancer Res. 4:1903-14 (1998); Persson et al., Adv. Drug Del. Res. 31: 143-152 (1998)). Investigators attempted to reduce the toxicity and improve the efficacy by reducing the MHC class II binding sites and neutralizing antibody binding epitopes in the molecule (Hansson et al., Proc. Natl. Acad. Sci. 94:2489 (1997); Erlandsson et al., J. Mol. Biol. 333:893-905 (2003)). Reduction of MHC class II toxicity was accomplished at the expense of reducing the number of activated Vβ T cell clones. However, even these extensively modified SEs still retained binding to neutralizing antibodies while toxicity was only modestly improved.

In contrast to the classic SEs (alone or part of a fusion protein with tumor specific antibodies) which require genetic modification of antibody binding epitopes and MHC class II binding sites to improve their efficacy and reduce their toxicity in humans (Giantonio et al., J. Clin. Oncol. 15:1994-2007 (1997); Alpaugh et al., Clin. Cancer Res. 4:1903-14 (1998), Persson et al., Adv. Drug Del. Res. 31: 143-152 (1998); Erlandsson et al., J. Mol. Biol. 333:893-905 (2003)), egc SAgs in native form given intravenously, intrathecally or intratumorally (as described in the instant specification) induce significant tumoricidal effects with minimal toxicity in humans. Whereas neutralizing antibodies against the classic SEs that interfere with their T cell proliferative function are commonly present in human sera, antibodies against the egc SEs which inhibit their T cell stimulating ability are rarely found in human sera. As a result, toxicity of treatment with the egc SEs has been negligible. Third, since neutralizing antibodies against egc SEs are absent, the egc SEs induced a greater therapeutic effect than the classical SEs in humans. Moreover, with the egc SEs, it was not necessary to measure antibodies in patient's sera before each treatment to determine an effective dose, whereas it was required for non-egc SAgs to avert toxic effects (Cheng J D et al., J Clin Oncol. 22:602-9 (2004)). Forth, because they are less toxic the non-egc superantigens, the egc SAgs may be used as a plurality to activate a larger number of Vβ-tumor specific T cell clones thus increasing their anti-tumor potency compared to the non-egc SAgs which because of their toxicity can only be used safely as a single agents.

Most importantly, the efficacy of the egc superantigens was far superior against human lung cancer than the classical SEs used alone or fused SEs to a tumor targeting device. Whereas the egc SEs induced a 400% increase in survival in patients with advanced NSCLC, SE monoclonal antibody preparations produced a negligible response rate against human colon and pancreatic carcinoma and only a modest prolongation of survival in patients with advanced non-small cell lung cancer (NSCLC). Moreover, the egc SEs could be used in native form without requiring extensive genetic mutation in order to provide safety and efficacy.

Fusion polypeptides comprising SEA fused to a tumor specific monoclonal antibody (mAb), designated “SEA-mAb,” induced tumoricidal responses in the murine B16 melanoma model (Dohlsten M et al., Proc Natl Acad Sci 91:8945-9 (1994); Dohlsten M et al., Proc. Natl. Acad. Sci. 88:9287-91 (1991). Because native SEA alone was found to be ineffective in such models, Dohlsten and colleagues (U.S. Pat. No. 5,858,363) stated that native superantigen would be of “low value” particularly against MHC class II-negative carcinomas which represent the vast majority of clinically significant human tumors. It is therefore evident that those working in this field, led by the investigators cited above, focussed on extensively mutated SE and clearly, they did not envision that native, non-mutated SEs such as the egc SEs, much less a plurality of them, could be used parenterally (intravenously, intrathecally, intrapleurally, intravesicularly, intrapericardially and intratumorally) to induce significant anti-tumor effects in humans with minimal toxicity. Indeed, the egc SEs do not require mutation of their SE antibody binding epitopes or reduction of MHC class II binding sites. Unlike the classical native SEs and SE fusion proteins, neutralizing antibodies against egc SEs are rarely found in patient sera. Indeed, measurement of SE-neutralizing antibodies before each treatment to determine an effective dose is not necessary when using the egc SEs. In contrast to the classical native or mutated SEs or SE-antibody fusion proteins, the present inventors have found that unmutated, native egc SEs may be administered intravenously, intrathecally and intratumorally to humans with minimal toxicity and induce potent tumoricidal effects.

Native SEs are known to induce anti-tumor effects in animals which are generally less sensitive to the toxic effects of these molecules than humans. Administration of SEB produced antitumor effects against established tumors in two animal species, rabbits and mice, with tumors of five different histologic types: rabbit VX-2 carcinoma (Terman et al., U.S. Pat. No. 6,126,945; Terman, U.S. Pat. No. 6,340,461), murine CL62 melanomas (Penna C. et al., Cancer Res. 54: 2738-2743 (1994)), murine A/20 lymphoma (Kalland T. Declaration in U.S. Ser. No. 07/689/799 (1992)), murine PRO4L fibrosarcoma (Newell et al., Proc Natl. Acad. Sci. 88: 1074-1079 (1991)) and human SW620 colon carcinoma (Dohlsten et al., Eur. J. Immunol. 21: 1229-1233 (1991)). In these studies, parenterally-administered SEB induced objective anti-tumor effects at primary and metastatic sites. SEB was used ex vivo to stimulate a population of T cells pre-exposed to tumor, which, upon re-infusion into host animals with established pulmonary metastases, induced a substantial reduction of metastases. (Shu S and Terman et al., J. Immunol. 152: 1277-88 (1994)). SEB transfected murine mammary carcinoma cells which expressed and secreted SEB were effective in reducing pulmonary metastases in a post-surgical metastatic model (Pulaski and Terman et al., Cancer Res. 60: 2710-5 (2000).

However when native classical SEB and SEA were used systemically to treat humans with metastatic breast or colon cancer significant toxicity was observed (Terman et al., et al., N. Engl. J. Med. 305:1195-2000 (1981); Young et al., Am. J. Med. 75:278-88 (1983)). Native SEB (together with protein A) administration to patients with metastatic breast cancer resulted in severe pulmonary toxicity which manifested as objectively confirmed acute respiratory distress syndrome (ARDS) with hypoxemia (due to non-cardiogenic pulmonary edema). The hypoxemia was worse in a patient with preexisting metastatic lung tumor who also developed severe bronchospasm and a large pleural effusion requiring repeated thoracenteses. This strong reaction prompted the above authors to warn that SEB treatment should not be carried out in patients with pulmonary metastases (Terman, D S CRC Crit. Rev. Oncol. Hematol. 4:103-24 (1985)). Moreover, pathology studies of primates infused with SEB showed a tendency for the protein to localize in the pulmonary vasculature injuring endothelial cells and causing pulmonary edema (Finegold M J, Lab. Invest. 16:912-924 (1967)). In the case of native SEA, neutralizing antibodies were noted frequently whose presence correlated with severe toxicity (Young et al. Am. J. Med. (1983); Giantonio et al., J. Clin. Oncol. (1998). Hence, classical native SEs were not considered to be useful for treatment of human cancer

In view of toxicity noted with the classical native SEs in human, those skilled in the art were not inclined to consider administering classical native SAgs systemically much less intrathecally into the pleural space in patients with metastatic cancer of the lung with or without MPE (or intratumorally into patients with malignant lung or brain nodules.) Indeed, based on the foregoing, a person of ordinary skill in the art would have concluded that administration of a native SAg directly into the pleural space to treat an MPE was contraindicated because it was liable to exacerbate the effusion and induce life threatening hypoxemia and bronchospasm.

Surprisingly, as presented below, the present inventors discovered that, notwithstanding the earlier results cited above that taught away from intrapleural and intratumoral administration of SAgs, intrathecal administration and intratumoral administration of native egc SEs directly into the pleural space resulted in successful treatment of 14 consecutive and unselected patients with MPE (intrathecal=intrapleural) from non-small cell lung cancer and disappearance of a large lung carcinoma (intratumoral). Indeed, all fourteen of the first patients with MPE treated in this manner showed partial or complete resolutions of their pleural effusions with minimal toxicity and a significant survival benefit above palliative-treated controls. In addition, a patient with a large lung adenocarcinoma treated with intratumoral SAg and low dose cisplatinum (5 mg q 7 days×3 dose) showed a complete regression of his tumor. Importantly, Examples 1 and 2 prove that a plurality of native egc SEs, is an effective antitumor therapeutic agent. It would be expected that since the native egc SEs induced an anti-tumor effect with minimal toxicity, that homologues of the native egc SEs (as defined herein) including but not limited to mutants, variants, fusion proteins would also exert tumoricidal effects with limited toxicity when given intravenously, intrathecally or intratumorally.

### Intrathecal Administration of egc SEs

The appearance of tumors in sheaths (“theca”) encasing organs often results in production and accumulation of large volumes of fluid in the organs' sheath. Examples include (1) pleural effusion due to fluid in the pleural sheath surrounding the lung, (2) ascites originating from fluid accumulating in the peritoneal membrane and (3) cerebral edema due to metastatic carcinomatosis of the meninges. Such effusions and fluid accumulations generally develop at an advanced stage of the disease. Malignant pleural effusion (“MPE”) is the prototype of this condition. In the United States and Western Europe, 300,000 new cases of malignant pleural effusion are diagnosed annually (Antony V B et al., Eur. Respir. J. 18: 402-419 (2001)). This condition is caused by different types of tumors: lung cancer (35%), breast cancer (25%), lymphoma (10%), unknown primary malignancy (30%). It is the presenting manifestation in 10-50% of all cancers. When first evaluated, about 15% of lung cancer patients exhibit a pleural effusion. Fifty percent of cancer patients develop MPE at some point in their disease process. In 40-60 percent of patients with MPE from non-small cell lung cancer (NSCLC), MPE will be the initial presenting manifestation. Since the majority of these patients are dyspneic from their MPE, prompt treatment is required. In contrast to MPE from small cell carcinoma of the lung, breast carcinoma or lymphoma, chemotherapy is not the first option in patients with symptomatic MPE from NSCLC. Most of these patients are symptomatic and/or disabled from their effusions and they are not candidates for chemotherapy or surgery. They are usually offered palliative local therapy to control their MPE using chemical pleurodesis or indwelling catheter drainage. Even after successful pleurodesis or drainage, the majority of these patients exhibit poor performance (ECOG ≦2 or KPS ≧70) and are still not eligible for systemic chemotherapy. (Chernow B et al., Am J. Med. 63: 695-702 (1977); Sahn S A. Ann Intern Med. 108: 345-349 (1988); Walker-Renard P B et al., Ann Intern Med. 120:56-64 (1994); Sahn S A Clin Chest Med. 19: 351-361 (1998); Antony V et al., Eur Respir J. 18:402-19 (2001); Light R W. Pleural Diseases. Fourth Edition. Lippincott Williams & Wilkins, Philadelphia, Pa., pp. 87-184, 2001). The appearance of a pleural effusion in non small cell lung cancer (NSCLC) signifies Stage IIIb or Stage 1V disease and a poor prognosis with a median survival on the order to 2-3 months (Putnam J B Jr et al., Ann Thorac Surg 69: 369-375 (2000); Putnam J B Jr Cancer. 86:1992-1999 (1999); Heffner J E et al., Chest 117: 79-86 (2000); Swanson K et al., Am J Respir Crit Care Med. 165: A149 (2002); Burrows C M et al., Chest. 117: 73-781 (2000)).

Malignant ascites is associated with 30-50% of ovarian tumors. Endometrial, breast, colonic, gastric and pancreatic carcinomas make up more than 80% or the tumors associated with intra-abdominal seeding of tumor cells and ascites formation. Ascites may be the presenting manifestation in 4-69% of cases.

The major therapies for MPE include talc poudrage, talc slurry, doxycycline and bleomycin instillation (Veena et al. Am J. Crit. Care Med. 162: 1987-2001 (2000)). These therapies require 3-12 days of hospitalization with EKG and oximetry monitoring. A chest tube is inserted, and the therapeutic agent is infused and allowed to distribute over the pleural membranes. The chest tube is then connected to closed negative-pressure water seal drainage until pleural fluid volume drops below 100 ml/24 hours. Respiratory therapy is usually given at least once daily.

Talc poudrage requires the use of operating room and general anesthesia for thoracostomy and talc insufflation, followed by recovery room observation. Talc induces respiratory complications in up to 33% of patients and acute respiratory distress and hypoxemia in 10% of patients. Response rates to bleomycin and doxycycline range between 50% and 70%, respectively and both require continuous chest tube drainage until the output is below 100 ml/24 hours (Walker-Renard P B et al., supra (1994); Light R W supra (2001); Sahn S A supra (1998)). Indwelling pleural catheters for drainage and/or injection of a pleurodesis agent are an additional option; however, the catheter requires surgical placement followed by intermittent drainage of effusion fluid at home by the patient or a caregiver.

Intrapleurally administered agents or modalities that include (a) chemotherapeutic agents such as cisplatinum, cytarabine, doxorubicin, fluorouracil, etoposide, and mitomycin C, (b) radiation and (c) biotherapeutic agents such as IL-2, IFN-α, α, and γ, TNFα and bacterially derived immunostimulatory agents such as Corynebacterium parvum have been ineffective against MPEs. Thoracentesis or chest tube drainage alone results in recurrence rates of 98% and 85% respectively within 30 days (Walker-Renard P B et al., supra (1994); Light R W supra (2001); Sahn S A supra (1998), Belani C P et al., Chest 113: 78S-85S (1998)). Intraperitoneal cisplatinum and etoposide has produced a complete response rate of 30% in malignant ascites. However the only randomized study has failed to show any benefit for intraperitoneal therapy over conventional intravenous chemotherapy in the initial management of stage IIC to IV ovarian cancer.

The present invention overcomes these deficiencies in the treatment of MPE and malignant ascites by providing a new therapeutic approach to these manifestations of cancer. Unlike existing therapies, the present invention is more effective in controlling MPEs and malignant ascites and offers a significant survival benefit. The therapy is particularly effective in patients with poor performance status (KPS 30-60 or ECOG 3) who are not candidates for systemic chemotherapy. In contrast to existing palliative treatments for MPEs and malignant ascites, the present invention is carried out entirely in an outpatient setting and requires no chest tube insertion or hospitalization. Cost of treatment is several hundred percent below that of palliative measures. Major costs of the other therapies originating from hospitalization, chest tube insertion, operating and recovery room expense, respiratory therapy and in-hospital chest tube drainage, are eliminated.

### Intratumoral SAg Therapy

Prior to the present invention, therapeutic uses of classical native SAgs in humans have been limited to systemic administration which was associated with significant toxicity. In addition, researchers expressly asserted (U.S. Pat. No. 5,858,363) that native superantigens would be of “low value” for in vivo antitumor therapy of the most clinically important tumors (e.g., MHC class II negative carcinomas) because the non-neoplastic MHC class II+ cells (e.g., macrophages and lymphocytes) would outcompete the MHC class II_{neg} carcinoma cells for binding of the native SE. To improve the ability native SEs to localize to a tumor, they conjugated the SAg to a tumor specific antibody (Dohlsten M et al., Proc Natl Acad Sci USA 91:8945-9 (1994); Dohlsten M et al., Proc Natl Acad Sci USA 88:9287-91 (1991)). Secondly, in order to reduce cytokine-mediated toxicity, they a produced mutant SAg molecules with lower binding affinity to MHC class II molecules (Hansson J et al., Proc. Natl. Acad. Acad Sci USA 94: 2489-94 (1997)). However, as these investigators noted, (Persson B et al., supra (1998)) because SE-specific antibodies are found in all humans, their SE-engineered molecules, rather than localizing to tumors, are more likely to be re-directed to reticuloendothelial tissues where they are degraded and eliminated. Attempts to overcome this problem by delivering amounts of SE conjugate that exceed the SE-antibody neutralizing capacity only induced greater toxicity and higher levels of SE-specific antibodies while deimmunizing the SE molecule by mutation of their SE binding epitopes have met with limited success.

It is clear that these workers did not envision the use of a native egc SE much less a plurality of native egc SAgs for intratumoral use and in fact, by their focus on genetically altering the MHC class II and antibody binding properties of the native SE molecule for systemic use appear to have taught away from this approach. Indeed, they have carried out no studies in humans using a native SE, much less a plurality of native SEs via the intravenous, intratumoral or intrathecal mode of administration. In contrast, the present inventors have recognized that a plurality of native egc SEs are capable of inducing an anti-tumor effect in humans with minimal toxicity when they are administered intratumorally as well as intravenously and intrathecally.

Use of T Cell Survival Enhancing Cyokines with SAgs to Prevent Activation-Induced T Cell Death In Vitro and In Vivo.

The present invention contemplates the in vivo administration of T cell survival-enhancing cytokines IL-7, IL-15, IL-23 together with SAg in order to prevent SAg-induced activation-driven T cell death and to promote the longevity of a population of long-lived effector CD4+ and CD8+ tumor killing T cells. One or more of the above cytokines may be used in vitro with SAg to activate a population of tumor sensitized T cells to be used for adoptive immunotherapy of various tumors. The same cytokines are administered to the tumor bearing host together with the activated and expanded T cells in order to promote their persistence as T effector cells in vivo.

OKT3 is commonly used in protocols during the in vitro production of T cells for adoptive immunotherapy of cancer. However, SAg is superior to OKT3 (anti-CD3) as a T cell activator for in vitro. For instance, unlike SAgs, OKT3 does not produce significant T cell proliferation or differentiation of T cells to cytotoxic effector cells in vitro. in contrast to SAg, OKT3 is incapable of selectively expanding clones of tumor specific T cells with Vβ specificity and is also a much weaker stimulant of T cell cytokine production in vitro than SAgs.

The Use of Chemotherapy and Radiation with SAg In Vivo

The present invention contemplates the synergy of chemotherapy and radiation in enhancing the tumor killing effects of SAg in vivo. It has been observed by one of the inventors that SAg induces both histologic and physiologic alterations in tumor cells in vitro and in vivo that enhance the uptake of chemotherapy and the markedly promote the tumor killing effects of both radiation and chemotherapy. As a result of the physiologic changes induced by SAg, tumor cells are killed in vivo by chemotherapy in doses that are up to 90% lower than the FDA-recommended doses with little or no toxicity to the patient. Because the chemotherapy induces remission in such small doses, the patient is spared the drug resistance and dose-limiting effects of the chemotherapeutic agents.

### SUMMARY OF THE INVENTION

The present invention provides a method for treating malignant tumors including those presenting with pleural effusion, ascites, pericardial effusion and meningeal carcinomatosis by intravenous, intrathecal (defined below) or intratumoral administration of an effective amount of native egc SEs and/or their homologues. The present invention contemplates the use of one or preferably a plurality of staphylococcal enterotoxins (“SE”) G, I, M, N, O which are gene products encoded by the enterotoxin gene complex (egc). The invention includes all natural or man-made recombinations comprising native egc SEs, or homologues to include variants, mutants, fusion proteins with a fusion partner encoded by the egc SE and another species of molecule including but not limited to an antibody, antibody fragment, receptor ligand, bacterial virulence factor, costimulant, cytokine, chemokine or coaguligand. These agents activate/recognize a broad human T cell TCR Vβ/α repertoire and are administered repeatedly in picrogram quantities by injection, infusion, instillation or implantation intravenously, intratumorally or intrathecally into a cavity or space (thecum) surrounding an organ or body region in which a tumor is present or is causing fluid accumulation.

Such spaces include the pleural space, peritoneum, subarachnoid space or dural space, or pericardial space. The generic term for administration into a sheath encasing an organ is termed “intrathecal,” defined in Dorland's Medical Dictionary 29th Edition, WB Saunders (2000) and Stedman's Medical Dictionary, 27th Edition, Lippincott, Williams & Wilkins (2000) as meaning “within a sheath.” As used herein, this term is intended to be broader than a more commonly used definition which is limited to intracranial spaces.

Previous publications disclose administration of a single classical native SE humans with cancer via intravenous intravenous injection or infusion (See, for example, U.S. Pat. No. 6,126,945) showing limited effectiveness and significant toxicity (Young Am. J. Med. Giantonio et al. J. Clin. Oncol.). Other document disclose the administration of SAg “locally or systemically” (U.S. Pat. No. 6,197,299; U.S. Pat. No. 5,858,363) or in adjuvants with slow release (U.S. Pat. No. 6,126,945, by one of the present inventors). The prior art does not disclose the use of native egc SEs as a plurality administered intravenously, intratumorally or intrathecally to induce an anti-tumor effect against human carcinoma with minimal toxicity.

In addition, when administered intravenously classical native SEs alone or mutated classical SEs conjugated to tumor specific antibodies (SAg-mAb fusion proteins) do not reach their targets in effective concentrations for two reasons. First, the SAgs are neutralized rapidly by “natural” neutralizing SAg-specific antibodies. (Giantonio et al., supra; Alpaugh et al supra; Persson et al., supra). Second, SAg-mAb fusion proteins bind to cells present in the circulation that express MHC class II proteins. One approach to overcoming these obstacles was to mutate the SE to reduce its affinity for MHC class II molecules (Hansson et al., Proc. Natl. Acad. Sci. 94:2489 (1997)) and to reduce the number of SE epitopes which bind neutralizing antibodies (Erlandson et al. J. Mol. Biol 2003). These agents have met with only modest success when used in humans with advanced lung, breast, colon and pancreatic cancer.

The present invention obviates this obstacle to a large extent by using native egc SEs to which humans only rarely make natural antibodies intravenously, intrathecally, or intratumorally. The present invention also covers compositions of one or more native egc SAg or egc SAg homologues consisting of amino acid substitution and deletion variants (mutants), additions (e.g., fusion proteins) and fragments with Z values ≧10 when the sequence is compared to a native superantigen using the FASTA/FASTP programs and Monte Carlo analysis. The present invention contemplates the use of one or preferably a plurality of native egc SAgs or egc superantigen homologues or mixtures of native egc superantigens and egc superantigen homologues which preferably exhibit a Vβ/Vα profile with a minimum recognition of 5 different Vβ/α-expressing T cell clones or T cell populations expressing at least 5 different TCR Vβ/Vα (using well described in vitro RNA/DNA-PCR or surface expression assays) after stimulation with individual egc SECs.

The preferred SE composition includes a mixture of SEG, SEI, SEM, SEQ and SEN or any one or plurality of egc superantigens or egc superantigen homologues or mixtures of native egc superantigens and egc superantigen homologues. The egc SAgs have unique properties compared to the non-egc SAgs in that they do not induce toxicity in humans when administered either intrathecally, intratumorally or intravenously and patients rarely display neutralizing antibodies against them. Thus, unlike the SE molecule in the SE-antibody fusion proteins which is genetically modified to eliminate antibody binding epitopes and reduce MHC class II binding affinity, the native egc SEs do not require any structural alterations of the native egc SE molecules to be useful as anti-tumor agents. Nor do they require determination of neutralizing antibodies in the patient before each treatment in order to provide an effective dose of SE. The egc SEs or homologues are injected or infused systemically or intrathecally into patients with malignant tumors and/or pleural effusions and/or ascites respectively or intratumorally into tumor site(s) and induce a tumoricidal response with minimal toxicity. The egc SAg composition is preferably administered after partial or complete drainage of the fluid from the sheath as for example in pleural effusions via thoracentesis and ascites via paracentesis. However, the egc SAg composition may also be administered directly into an undrained space containing the effusion, ascites and/or carcinomatosis. The invention also contemplates the use of the nucleic acid counterparts of the native egc superantigens and homologues as useful for the same indications as the polypeptide forms of the molecule.

To enhance the effectiveness and specificity of the egc SAg, it or a biologically active fragment or homologue may be fused to another protein such as (1) a tumor specific antibody, or an antigen binding fragment of such an antibody, such as an F(ab′)2, Fv or Fd fragment, which antibody is specific for an epitope expressed on the tumor or (b) a receptor ligand specific for any receptors selectively or preferentially expressed on tumor cells. The fusion partner can also be a powerful costimulant such as OX-40 or 4-1BB1 which enhances the T cells proliferative response to the SAg or a “Coaguligand” which promotes coagulation in the tumor vasculature.

The egc SAg composition is administered once every 3 to 10 days, preferably once weekly, and this schedule is continued until there is no re-accumulation of the effusion or ascites or reduction in the size of the tumor mass being injected. Three such treatments may suffice for intrathecal administration although this is an average; the number of treatments may varying from 1-6 or even higher. For intrathecal administration, the egc SAg composition is preferentially administered immediately after removal of pleural fluid via thoracentesis. Unlike the other therapies for malignant pleural effusions, the present method is carried out entirely in an outpatient setting and requires no hospitalization, chest tube insertion, use of the operating room or recovery room, respiratory therapy or in-hospital chest tube drainage. In contrast to the conventional treatment for MPE noted above, instillation of the egc SAg composition into the pleural space has a response rate of nearly 100%. Unlike talc therapy in which up to 10% of cases may experience hypotension or acute respiratory distress syndrome, the present egc SAg therapeutic method has not induced any significant morbidity. Hence, this invention offers decided advantages of effectiveness, safety, convenience and cost/effectiveness over the prior art.

The present invention contemplates the use of SAg therapy to enhance the effects of chemotherapy and radiation on tumors. For this to occur, the chemotherapy should be administered together with or 1-48 hours after the SAg treatment. Tumors treated with SAg undergo morphologic and functional alterations as described herein that make them unusually susceptible to the effects of various chemotherapeutics and radiation. These effects may be induced by any SAg although egc SAgs are preferred. The SAg may be administered by injection, infusion or instillation via any route such as parenterally, intravenously, intrathecally, intratumorally, intraperitoneally, intramuscularly, subcutaneously, intralymphatically, intrapleurally, intravesicularly, intrapericardially and the chemotherapy can be of any type to which is indicated for a specific tumor. The chemotherapy can be administered alone or in combination with other chemo or biological therapies. The chemo- or biological therapy can be administered parenterally, intravenously, intrathecally, intratumorally, intraperitoneally, intramuscularly, subcutaneously, intralymphatically, intrapleurally, intravesicularly, intrapericardially in conventional doses. Moreover, because of the physiologic changes induced in the tumor cells by SAg therapy, the chemo- and biologic therapies can be administered in dosages that are significantly lower than conventionally recommended and in a range considered to be subtherapeutic by themselves. Since the chemo and biological therapies are administered in significantly lower doses, they avoid the toxic effects and morbidity commonly seen with these agents when used in conventional dosages.

Moreover the present invention contemplates the in vivo administration of native SAgs and egc SEs in particular, as well as SAg homologues, derivatives, conjugates and fusion proteins together with one or more cytokines IL-7, IL-15, IL-23 in order to ensure the survival and prevent activation-driven death of the tumor killing T cell populations induced by the SAgs or SAg homologues, derivatives, conjugates and fusion proteins.

The egc SAgs may be used for adoptive immunotherapy of cancer. In vitro, one or a plurality of SAgs are used to induce a population of CD4+ and CD8+ tumor sensitized T cells to become effector T cells. One or a plurality of cytokines are coincubated in vitro with the SAgs to ensure the survival of SAg-induced long-lived memory CD4+ and CD8+ effector T cells by preventing SAg-induced activation-driven T cell death. Because SAgs can activate a broad Vβ profile, they are used in vitro as a plurality to maximally activate tumor sensitized T cells. The latter can be derived from tumor infiltrating T cells (TIL), lymph nodes, spleen, bone marrow or peripheral blood of the tumor bearing subject. In some instances, a clone of TIL showing tumor specificity has an identifiable Vβ profile that can be specifically activated and expanded by a SAg with same Vβ specificity. In the case of multiple T cell clones exhibiting tumor and Vβ specificity, a plurality of SAgs can be used to specifically activate those Vβ expressing T cells as well. egc SEs and other SAgs can be used for these purposes. Likewise, mutant and variant SEs with narrower Vβ specificities than their native counterparts may be used for activation of single clones of T cells in vitro. One or more of the same cytokines IL-7, IL-15, IL-23 are administered with the activated and expanded T cell population and for several days thereafter to ensure the survival of the adoptively transferred T cells

### BRIEF DESCRIPTION OF FIGURES

FIG. 1. T cell proliferative activity of 200 μL of agent B36873 containing egc SEs compared to a conventional superantigen SEC1. B36873 induces significant T cell proliferation at doses of SEC ranging from 1-100 picrograms and ED50 of B36873 (8 pg) exceeded that of SEC1 (64 pg).

FIG. 2. Kaplan Meir survival curve of 14 patients who received egc SSAg intrapleurally for treatment of MPE from NSCLC showing a median survival of 7.9 months (range 2-32 months) (95% CI, 5.9-11.4 months). Solid line represents survival of egc SSAg-treated patients and dotted lines are 95% CIs.

FIG. 3. Kaplan-Meir survival curve comparing 14 patients who received egc SSAg intrapleurally with 13 patients who received talc poudrage for treatment of MPE from NSCLC who had similar pre-treatment KPS [range (10-60) and median (40 and 30), respectively] and distribution (See text). The patients who received egc SSAg had a significantly increased median survival of 7.9 months compared to 2.0 months for the patients who received talc pleruodesis (p=0.0023).

### DESCRIPTION OF THE PREFERRED EMBODIMENT

### Production and Isolation of Superantigens

The superantigens disclosed herein are prepared by either biochemical isolation, or, preferably by recombinant methods. The following SAgs, including their sequences and biological activities have been known for a number of years. Studies of these SAgs are found throughout the biomedical literature. For, biochemical and recombinant preparation of these SAgs, see the following references: Borst, D W et al., Infect. Immun. 61: 5421-5425 (1993); Couch, J L et al., J. Bacteriol. 170: 2954-2960 (1988); Jones, C L et al., J. Bacteriol. 166: 29-33 (1986); Bayles K W et al., J. Bacteriol. 171: 4799-4806 (1989); Blomster-Hautamaa, D A et al., J. Biol. Chem. 261:15783-15786 (1986); Johnson, L P et al., Mol. Gen. Genet. 203, 354-356 (1986); Bohach G A et al., Infect. Immun. 55: 428-433 (1987); Iandolo J J et al., Methods Enzymol 165:43-52 (1988); Spero L et al., Methods Enzymol 78(Pt A):331-6 (1981); Blomster-Hautamaa D A, Methods Enzymol 165: 37-43 (1988); Iandolo J J Ann. Rev. Microbiol. 43: 375-402 (1989); U.S. Pat. No. 6,126,945 and U.S. provisional patent application 60/389,366 filed Jun. 15, 2002. These references and the references cited therein are hereby incorporated by reference in their entirety.

These SAgs are Staphylococcal enterotoxin A (SEA), Staphylococcal enterotoxin B (SEB), Staphylococcal enterotoxin C (SEC—actually three different proteins, SEC1, SEC2 and SEC3)), Staphylococcal enterotoxin D (SED), Staphylococcal enterotoxin E (SEE) and toxic shock syndrome toxin-1 (TSST-1) (U.S. Pat. No. 6,126,945 and U.S. provisional patent application 60/389,366 filed Jun. 15, 2002, and the references cited therein). The amino acids sequences of the above group of native (wild-type) SAgs is provided below:

| SEA (Huang, I. Y. et al., J. Biol. Chem. 262: 7006-7013 (1987)) | |
|---|---|
| [SEQ ID NO: 1] | |
| 1 | SEKSEEINEK DLRKKSELQG TAGNKQIY YYNEKAKTEN KESHDQFLQH TTLFKGFFTD |
| 61 | HSWYNDLLVD FDSKDIVDKY KGKKVDLYGA YYGYQCAGGT PNKTACMYGG VTLHDNNRLT |
| 121 | EEKKVPINLW LDGKQNTVPL ETVKTNKKNV TVQELDLQAR RYLQEKYNLY NSDVFDGKVQ |
| 181 | RGLIVFHTST EPSVNYDLFG AQGQYSNTLL RTYRDNKSIN SENMHIDIYL YTS |
| SEB (Papageorgiou, A. C. et al. J. Mol. Biol. 277: 61-79 (1998)) | |
| [SEQ ID NO: 2] | |
| 1 | ESQPDPKPDE LHKSSKFTGL MENMKVLYDD NHVSAINVKS IDQFLYFDLI YSIKDTKLGN |
| 61 | YDNVRVEFKN KDLADKYKDK YVDVFGANYY YQCYFSKKTN DTNSHQTDKR KTCMYGGVTE |
| 121 | HNGNQLDKYR SITVRVFEDG KNLLSFDVQT NKKKVTAQEL DYLTRHYLVK NKKLYEFNNS |
| 181 | PYETGYIKFI ENENSFWYDM MPAPGDKFDQ SKYLMMYNDN KMVDSKDVKI EVYLTTKK |
| SEC1 (Bohach, G. A. et al., MOL. Gen. Genet. 209: 15-20 (1987)) | |
| [SEQ ID NO: 3] | |
| 1 | MNKSRFISCV ILIFALILVL FTPNVLAESQ PDPTPDELHK ASKFTGLMEN MKVLYDDHYV |
| 61 | SATKVKSVDK FTAHDLIYNI SDKKLKNYDK VKTELLNEGL AKKYKDEVVD VYGSNYYVNC |
| 121 | YFSSKDNVGK VTGGKTCMYG GTTKHEGNHF DNGNLQNVLI RVYENKRNTI SFEVQTDKKS |
| 181 | VTAQELDIKA RNFLINKKNL YEFNSSPYET GYIKFIENNG NTFWYDMMPA PGDKFDQSKY |
| SEC2 (Papageorgiou, A. C., et al., Structure 3: 769-779 (1995)) | |
| [SEQ ID NO: 4] | |
| 1 | ESQPDPTPDE LHKSSEFTGT MGNMKYLYDD HYVSATKVMS VDKFLAHDLI YNISDKKLKN |
| 61 | YDKVKTELLN EDLAKKYKDE VVDVYGSNYY VNCYFSSKDN VGKVTGGKTC MYGGITKHEG |
| 121 | NHFDNGNLQN VLIRVYENKR NTISFEVQTD KKSVTAQELD IKARNFLINK KNLYEFNSSP |
| 181 | YETGYIKFIE NNGNTFWYDM MPAPGDKFDQ SKYLMMYNDN KTVDSKSVKI EVHLTTKNG |
| SEC3 (Hovde ,C. J. et al., Mol. Gen. Genet. 220: 329-333 (1990)) | |
| [SEQ ID NO: 5] | |
| 1 | MYKRLFISRV ILIFALILVI STPNVLAESQ PDPMPDDLHK SSEFTGTMGN MKYLYDDHYV |
| 61 | SATKVKSVDK FLAHDLIYNI SDKKLKNYDK VKTELLNEDL AKKYKDEVVD VYGSNYYVNC |
| 121 | YFSSKDNVGK VTGGKTCMYG GITKHEGNHF DNGNLQNVLV RVYENKRNTI SFEVQTDKKS |
| 181 | VTAQELDIKA RNFLINKKNL YEFNSSPYET GYIKFIENNG NTFWYDMMPA PGDKFDQSKY |
| 241 | LMMYNDNKTV DSKSVKIEVH LTTKNG |
| SED (Bayles, K. W. et al., J. Bacteriol. 171: 4799-4806 (1989)) | |
| [SEQ ID NO: 6] | |
| 1 | MKKFNILIAL LFFTSLVISP LNVKANENID SVKEKELHKK SELSSTALNN MKHSYADKNP |
| 61 | IIGENKSTGD QFLENTLLYK KFFTDLINFE DLLINFNSKE MAQHFKSKNV DVYPIRYSIN |
| 121 | CYGGEIDRTA CTYGGVTPHE GNKLKERKKI PINLWINGVQ KEVSLDKVQT DKKNVTVQEL |
| 181 | DAQARRYLQK DLKLYNNDTL GGKIQRGKIE FDSSDGSKVS YDLFDVKGDF PEKQLRIYSD |
| 241 | NKTLSTEHLH IDIYLYEK |
| SEE (Couch, J. L. et al., J. Bacteriol. 170: 2954-2960 (1988)) | |
| [SEQ ID NO: 7] | |
| 1 | MKKTAFILLL FIALTLTTSP LVNGSEKSEE INEKDLRKKS ELQRNALSNL RQIYYYNEKA |
| 61 | ITENKESDDQ FLENTLLFKG FFTGHPWYND LLVDLGSKDA TNKYKGKKVD LYGAYYGYQC |
| 121 | AGGTPNKTAC MYGGVTLHDN NRLTEEKKVP INLWIDGKQT TVPIDKVKTS KKEVTVQELD |
| 181 | LQARHYLHGK FGLYNSDSFG GKVQRGLIVF HSSEGSTVSY DLFDAQGQYP DTLLRIYRDN |
| 241 | KTINSENLHI DLYLYTT |
| TSST-1 (Prasad, G. S. et al., Protein Sci. 6: 1220-1227 (1997)) | |
| [SEQ ID NO: 8] | |
| 1 | MNKKLLMNFF IVSPLLLATT ATDFTPVPLS SNQIIKTAKA STNDNIKDLL DWYSSGSDTF |
| 61 | TNSEVLDNSL GSMRIKNTDG SISLIIFPSP YYSPAFTKGE KVDLNTKRTK KSQHTSEGTY |
| 121 | IHFQISGVTN TEKLPTPIEL PLKVKVHGKD SPLKYGPKFD KKQLAISTLD FEIRHQLTQI |
| 181 | HGLYRSSDKT GGYWKITMND GSTYQSDLSK KFEYNTEKPP INIDEIKTIE AEIN |

The sections which follow discuss SAgs which have been discovered and characterized more recently.

### Staphylococcal Enterotoxins SEG, SEH, SEI, SEJ, SEK, SEL, SEM, SEN, SEO, SEP, SEQ, SER, SEU

New Staphylococcal enterotoxins G, H, I, J, K, L and M (SEG, SEH, SEI, SEJ, SEK, SEL, SEM, SEN, SEQ, SEP, SEQ, SER, SEU; abbreviated below as “SEG-SEU”) were described in Jarraud, S. et al., J. Immunol. 166: 669-677 (2001); Jarraud S et al., J. Clin. Microbiol. 37: 2446-2449 (1999) and Munson, S H et al., Infect. Immun. 66:3337-3345 (1998). SEG-SEU show superantigenic activity and are capable of inducing tumoricidal effects. The homology of these SE's to the better known SE's in the family ranges from 27-64%. Each induces selective expansion of TCR Vβ subsets. Thus, these SEs retain the characteristics of T cell activation and Vβ usage common to all the other SE's. RT-PCR was used to show that SEH stimulates human T cells via the Vα domain of TCR, in particular Vα (TRAV27), while no TCR Vβ-specific expansion was seen. This is in sharp contrast to all other studied bacterial superantigens, which are highly specific for TCR Vβ. Vβ binding superantigens form one group, whereas SEH has different properties that fit well with Vα reactivity. It is suggested that SEH directly interacts with the TCR Vα domain. (Petersson K et al., J Immunol. 170:4148-54 (2003)).

SEG and SEH of this group and other enterotoxins including SPEA, SPEC, SPEG, SPEH, SME-Z, SME-Z2, (see below) utilize zinc as part of high affinity MHC class II receptor. Amino acid substitution(s) at the high-affinity, zinc-dependent class II binding site are created to reduce their affinity for MHC class II molecules.

### Egc Staphylococcal Enterotoxins

Jarraud S et al., 2001, supra, discloses methods used to identify and characterize egc SEs SEG-SEM, and for cloning and recombinant expression of these proteins. The egc comprises SEG, SEI, SEM, SEN, SEQ and pseudogene products designated ψent 1 and ψent 2. Purified recombinant SEN, SEM, SEI, SEQ, and SEGL29P (a mutant of SEN) were expressed in E. coli. Recombinant SEG, SEN, SEM, SEI, and SEQ consistently induced selective expansion of distinct subpopulations of T cells expressing particular Vβ genes.

The yeast expression system is the preferred recombinant method for production of clinically useful egc SEs. Yeast is recognized as non pathogenic for human. By providing a secretion signal sequence, the egc SEs allows for secretion of substantial quantities of egc SEs into the culture media. This method allows the production of the superantigen in the yeast supernatant without the addition of any N- or C-terminus marker. The most prominent examples of yeast that can be used are S. cerevisiae, Hansenula polymorpha, Pichia pastoris, Kluyveromyces lactis, Yarrowia lipolytica, Pichia methanolica, Pichia stipitis, Zygosaccharomyces rouxii and Z. bailii, Candida boidinii, and Schwanniomyces (Debaryomyces) occidentalis. The methylotrophic yeast of the Pichia genus are used and methanol is employed as inducer of the alcohol oxidase (AOX 1) promoter in the expression systems. The enterotoxin-coding DNA sequence is cloned within an expression cassette containing a yeast promoter and transcriptional termination sequences.

cDNA of each egc SE is amplified by PCR using gene specific primers with overhangs generating NotI/EcoRI restriction sites at the 5′ and 3′ ends, respectively. A yeast secretion signal sequence is added to ensure full secretion of the enterotoxins into the culture supernatant. The primers are designed to ensure in-frame cloning of the cDNA of interest into the expression cassette. Therefore, sequences providing the restriction sites for cloning (NotI/EcoRI) are fused to gene specific sequences. Digested PCR products are inserted in-frame into the NotI/EcoRI restriction sites of the multiple cloning site. The expression vector pICZ A (Invitrogen) is prepared by sequential cutting with NotI and EcoRI, respectively. Ligation reactions and transformation into E. coli JM109 cells are carried out using standard methods.

Plasmid DNA of E. coli clones carrying an insert of the expected size is isolated linearized and transfected into via electroporation using a Bio-Rad GenePulser II. Settings are 1500 V, 50° F., and 200. Routinely, the alcohol oxidase 1 (AOX 1) promoter is employed for the expression of recombinant proteins. This promoter is tightly regulated and highly inducible by methanol, which also serves as the main carbon source during the expression. Using defined minimal media, P. pastoris can easily be grown to high cell densities. Thus, the cells are cultivated in WM9 medium without carbon source with 1% (v/v) methanol and 0.1% (w/v) glucose and incubated at 28° C. for 24 h. The supernatant from the cells is harvested. The egc SEs are then purified by at least two steps of High Pressure Liquid Chromatography. Each toxin purified separately will then be combined (likely in equimolar amounts) in order to produce the final preparation. Using the optimized feeding and induction protocol, we are now able to screen for and identify expression clones that produce heterologous protein with a yield of 2 mg per L culture volume or higher.

Egc SEs have been produced in E. Coli as follows: Primers were designed following identification of suitable hybridization sites in seg, sei, sem, sen, and seo as given in Jarraud et al., (2001) supra. The 5′ primers were chosen within the coding sequence of each gene, omitting the region predicted to encode the signal peptide, as determined by hydrophobicity analysis with GeneJockey software and SignalIP V1.11 World Wide Web Prediction Server (http://www.cbs.dtu.dk/services/SignalP/); the 3′ primers were chosen to overlap the stop codon of each gene. A restriction site was included in each primer. DNA was extracted from A900322 or MJB1316 and used as a template for PCR amplification. PCR products and plasmid DNA were prepared using the Qiagen plasmid kit. PCR fragments were digested with £coRI and Pst1 (Boehringer Mannheim) and ligated (T4 DNA ligase; Boehringer Mannheim) with the pMAL-c2 expression vector from New England Biolabs (Ozyme) digested with the same restriction enzymes. The resulting plasmids were transformed into E. coli TG1. The integrity of the ORF of each construct was verified by DNA sequencing of the junction between pMAL-c2 and the different inserts. The fusion proteins were purified from cell lysates of transfected E. coli by affinity chromatography on an amylose column according to the supplier's instructions (New England Biolabs).

Additional Methods for recombinant production of egc SE proteins, hosts, vectors and promoters and are given in Recombinant Gene Expression Reviews and Protocols, Second Edition, Eds: P Balbás, A. Lorence, Humana Press Inc. Totowa, N.J. (2004) which is herein incorporated by reference in its entirety.

Jarraud S et al., 2001, supra, indicates that the seven genes and pseudogenes composing the egc (enterotoxin gene cluster) operon are co-transcribed. The association of related co-transcribed genes suggested that the resulting peptides might have complementary effects on the host's immune response. One hypothesis is that gene recombination created new SE variants differing by their superantigen activity profiles. By contrast. SEGL29P failed to trigger expansion of any of 23 Vβ subsets, and the L29P mutation accounted for the complete loss of superantigen activity (although this mutation did not induce a major conformational change). It is believed that this substitution mutation located at a position crucial for proper superantigen/MHC II interaction.

Overall, TCR repertoire analysis confirm the superantigenic nature of SEG, SEI, SEM, SEN, SEQ. These investigators used a number of TCR-specific mAbs (Vβ specificity indicated in brackets) for flow cytometric analysis: E2.2E7.2 (Vβ2), LE89 (Vβ3), IMMU157 (Vβ5.1), 3D11 (Vβ5.3), CRI304.3 (Vβ6.2), 3G5D15 (Vβ7), 56C5.2 (Vβ8.1/8.2), FIN9 (Vβ9), C21 (Vβ [symbol] 1), S511 (Vβ12), IMMU1222 (Vβ13.1), JIJ74 (Vβ13.6), CAS1.1.13 (Vβ14), Tamaya1.2 (Vβ16), E17.5F3 (Vβ17), PA62.6 (Vβ18), ELL1.4 (Vβ20), IG125 (Vβ21.3), IMMU546 (Vβ22), and HUT78.1 (Vβ23). Flow cytometry also revealed preferential expansion of CD4+ T cells in SEI and SEM cultures. By contrast, the CD4/CD8 ratios in SEQ-, SEN-, and SEG-stimulated T cell lines were close to those in fresh PBL.

A preferred method of producing recombinant egc SE's is to use the pET43 vector (Novagen) and the E. Coli BL21DE3 strain (Invitrogen). Primers for each egc SE were prepared according to Jarraud et al., (J. Immunol. (2000) supra). To increase soluble expression of the egc SE's, each of them was inserted into the pET43.1a vector (Novagen) to produce a fusion protein with a NusA-tag (NusA protein) which facilitates protein folding, a His-tag for protein selection and isolation and an enterokinase and a thrombin cleavage sites for removal of the NusA-His-tag polypeptide. Each egc SE DNA was cloned into the SmaI and HindIII or XbaI/avrII sites of pET43.1 (Novagen) which encodes Nus and 6×His tags at its NH₂ terminus and transformed in Escherichia coli BL21DE3 (Novagen) bacteria as 6His-NusA-fusion proteins. Cells are grown at 37° C. to A600 0.5-0.6, induced with 1 mM isopropyl-D-thiogalactoside for 4 h at 37° C. and in some cases is continued overnight at 15° C. Cells were lysed by lysozyme/sonication in lysis buffer (50 mM NaH₂PO₄, 300 mM NaCl, 10 mM imidazole, pH 8.0 and protease inhibitor cocktail tablets (ROCHE)), and insoluble cellular debris is cleared by centrifugation.

The cleared solutions are incubated with Ni₂+-nitrilotriacetic acid agarose beads (QIAGEN) at 4° C. for 2 h. After several washes (wash buffer 50 mM NaH₂PO₄, 300 mM NaCl, 20 mM Imidazole, pH 8.0), the recombinant proteins are eluted from the beads with elution buffer (50 mM NaH₂PO₄, 300 mM NaCl, 250 mM imidazole, pH 8.0). Fraction of elution are analyzed by SDS-PAGE, and fractions containing the NusA-Egc fusion proteins are pooled, and concentrated and dialysed against PBS using Amicon Ultra-PL30 or PL-50 centrifugal filter devices (Millipore).

The NusA-tag is removed from the fusion protein by digestion with Thrombin protease (Amersham) in cleavage buffer (50 mM Tris HCl, 0.1 M NaCl, 0.25 mM CaCl₂, pH 8.5) for 18 h at 22° C. or for 18 h at 37° C., with or without previous heating at 95° C. for 10 minutes to improve access to cleavage site. The ratio of fusion protein to protease is optimized and set to 0.2 unit/mg protein. The thrombin-treated solution is loaded directly onto an anion exchange chromatography on HiTrap Q HP column (Amersham) equilibrated with buffer A (50 mM Tris HCl, pH8.5). The protein were eluted through a 0-50% gradient of buffer B (50 mM Tris HCl, 1 M NaCl, pH8.5. Fraction of elution were analyzed by SDS-PAGE, and fractions containing cleaved egc SE's are pooled and further purified by gel filtration through a HiLoad 16/60 Superdex 200 prep grade column (Amersham). The final protein concentrations was measured by UV spectrophotometry.

With this method, each egc SE showed mitogenicity in a T cell proliferation assay using a CD69-specific cytofluorimetric assay measuring T-cell activation (Lina G et al., J. Clin. Micro. 36:1042-1045 (1998)). The Vβ profile of the egc SEs prepared in this fashion matched that of purified recombinant egc SE's using the plasmid pMAL-c2 vector in E. Coli strain TG1 (See Example 1).

pET (T7 promoter system) vectors without tags and with the kanamycin resistance marker (either pET9 or 28) or others are feasible for use in this system as well as are vectors with pelB leading sequence. The E. coli BL21(DE3)AI is also a feasible host for expressions.

A particularly preferred method of production of EGC SE's is given below:

Generation of rEGC SE Clones
1. Synthetic primers or complete oligonucleotides derived from known EGC SEs as described above (Jarraud et al., 2000 supra) are cloned into pET24b for scale-up production by first introducing a NdeI site into the 5′ end of the gene by PCR.
2. An original seed stock of E. coli containing each recombinant SEG, SEI, SEM, SEN, SEQ gene on plasmid pET24b is used to prepare a master cell bank (MCB).
3. The cells are aliquoted at 1 ml in a 2-ml cryo-vial and subjected to a slow-rate freeze. 6. Vials are tested for purity, Gram stain, biological type, and recombinant SE gene-insert sequence integrity. The average cell count is 3.98×10⁸ CFU/ml.

### Fermentation

1. For seed buildup, three 500-ml, triple-baffled, shake flasks are batched with 100 ml of sterile seed medium (24 g/L yeast extract, 12 g/L soytone, 4 g/L glycerol, 2.31 g/L K₂HPO₄, 12.5 g/L K₂HPO₄, and 50/1 g/ml kanamycin).
2. Each first-stage seed flask is inoculated aseptically with 1 ml (1% v/v) of pooled material from four thawed vials of the MCB.
3. First-stage seed flasks are placed on a rotary shaker at 250 rpm and incubated at 37° C. One flask is selected to obtain time-course samples at 1-h intervals. The time-course samples are processed for OD₆₀₀, pH, and TSAG plate and observed by wet mount.
4. Based on the growth curve (OD₆₀₀ values vs time), the first-stage seed culture is considered ready to scale to the second-stage seed when late log phase growth is observed.
5. In the second stage, three 2000-ml, triple-baffled, shake flasks are batched with 480 ml of the sterile seed medium formulation previously described. Each second-stage seed flask is inoculated aseptically with 9.6 ml (2% v/v) of the first-stage seed culture and grown under identical conditions.
6. Criteria to scale the second-stage seed to the production fermentor are based on the resulting growth curve (late log phase or early stationary phase) and a requirement to reach a specified OD₆₀₀ (8.0±2.0).
9. The production stage is conducted in an 80-L fermentor with a working volume of 48 L. The production fermentor is batched with the medium formulation described above.
10. P2000 antifoam is added to 0.1% v/v and kanamycin (Sigma, St. Louis, Mo.) to 50 yttg/ml. The fermentor is maintained at a temperature of 37° C., an agitation rate of 177 to 461 rpm, an aeration rate of 16 to 58 slpm, and a vessel pressure of 3 to 7 psig. The % DO within the fermentor is maintained at >20% by first adjusting the sparging and then the agitation rate incrementally.
11. The culture is aseptically fed 2.4 L of a sterile 10× nutrient feed (24 g/L yeast extract, 12 g/L soytone, 4 g/L glycerol, 9.6 L purified water) at a rate of 50 ml/min when the OD₆₀₀ reaches 8±2, or the amount of glutamate in the production culture is less than 50% of the original glutamate concentration at inoculation. This 10× nutrient feed is continued as long as the DO levels are greater than 20%.
12. The culture is induced with filter-sterilized, dioxane-free IPTG at a 1 mM final concentration when the OD₆₀₀ reached 12.5±2.5. Immediately after IPTG induction, the culture is fed with 2.4 L of a filter-sterilized 20× yeast nitrogen base solution (2 g/L yeast nitrogen base, 2.4 L purified water). The culture is prepared for harvest 4 h after IPTG induction.

### Cell Recovery, Disruption, and Precolumn Treatment

1. Each step of the recovery process is performed using equipment cooled with chilled water (<55° F.).
2. The fermented broth is transferred to a chilled tank and concentrated using a Microgon 0.2-μm 3-m² surface area, hollow-fiber module (Spectrum Laboratories, Rancho Dominguez, Calif.). The broth is recirculated through the module using a peristaltic pump.
3. The broth is concentrated from a starting volume of 55 L to a calculated cell density of approximately 250 to 300 g/L. Residual soluble medium components are removed from the retentate by diafiltration with 6 vol of 50 mM phosphate, pH 7.4, and 100 mM sodium chloride.
4. At the completion of the diafiltration, the filter is rinsed with a calculated volume of 50 mM phosphate, pH 7.4, 100 mM sodium chloride, and 140 mM EDTA to bring the final EDTA concentration up to 20 mM.
5. Cells are disrupted at 55 MPa (8000±500 psig) by passing the cell suspension twice through a Model 15MR8TA Gaulin high-pressure homogenizer equipped with a heat exchanger for cooling (APV Gaulin, Inc., Everett, Mass.). Disruption efficiency after two passes is >90% as determined by A₆₀₀.
6. The cell lysate is clarified by diafiltration with 6 vol of 50 mM phosphate, pH 7.4, 100 mM sodium chloride, and 20 mM EDTA using a 0.1-μm, 1-m² Septoport filter module (NC-SRT, Inc., Carey, N.C.). The retentate is recirculated by means of a rotary lobe pump.
7. The lysate filtrate is then subjected to tangential flow filtration using an Amicon S10Y100,100-kDa spiral membrane module with a 1-m² surface area (Millipore, Bedford, Mass.). The retentate is concentrated to 1/30-1/50 of the starting volume.
8. The 100-kDa filtrate is then concentrated using two Amicon SI0Y30 30-kDa spiral membrane modules, 2 m² surface area, in tandem (Millipore). The retentate is first concentrated to 1/10-1/20 of the starting volume, then diafiltered with 10 vol of 50 mM phosphate, pH 7.4, 100 mM sodium chloride, and 20 mM EDTA.
9. An optional step is as follows: The 30-kDa retentate is subjected to differential ammonium sulfate precipitation by adding solid ammonium sulfate to 45% saturation. The pH is monitored during this procedure and maintained between 7.0 and 7.4 by adding 0.5 M ammonium hydroxide. 10. The suspension is then stored for 4 h minimum at 2 to 8° C., after which the precipitate is recovered by means of bottle centrifugation at 13,700# for 15 min in a 2 to 8° C. refrigerated centrifuge (Beckman, Palo Alto, Calif.). The supernatant is decanted into a sterile, depyrogenated container, and the pellets are discarded. Solid ammonium sulfate is then added to the 45% precipitation supernatant to a final concentration of 75% saturation. The suspension is stored for 4 h minimum at 2 to 8° C., after which the precipitate is recovered as previously described by means of bottle centrifugation. The supernatant is decanted as waste, and the recovered pellets are stored at or below −60° C. in 250-ml polycarbonate screw-cap containers.
10. One hundred six grams of frozen ammonium sulfate pellets are combined into a single, sterile, pyrogen-free container and solubilized by the addition of 200 ml (2 ml/g) of cold 25 mM sodium phosphate buffer (pH 7.0). Pellets are completely solubilized by gentle stirring. Samples are removed for determination of protein content by BCA assay.
11. Based on the protein concentration, solubilized pellets are diluted by the addition of 1.0 M ammonium sulfate, 25 mM sodium phosphate buffer, pH 7.0, to obtain a final protein concentration of 1.5 mg/ml. Solubilized pellets are then filtered into a sterile, chilled, pyrogen-free sterile vessel using a 0.22-/ml Millipack filter (Millipore). In-process testing consisted of SDS-PAGE, protein concentration, endotoxin, and bioburden determination.

### Column Chromatography

Column 1

1. A 5.0-cm (i.d.) XK chromatography column [Amersham Pharmacia Biotech (APB), Uppsala, Sweden] is packed with 650 ml of depyrogenated phenyl-Sepharose, fast-flow, high-substitution HIC resin (APB). In development studies, the resin load limit is determined to be approximately 20 mg protein per milliliter of resin at 2 to 8° C.
2. The separation is performed by first equilibrating the resin with 10 column volumes of buffer (25 mM sodium phosphate, pH 7.0, 1.0 M ammonium sulfate) at 60 cm/h.
3. Resuspended ammonium sulfate pellets are loaded onto the column at 60 cm/h.
4. The column, buffers, and load material are maintained at 2 to 8° C. for the duration of the separation. The column is washed with 2.0 column volumes of equilibration buffer at 60 cm/h. The FT with a UV absorbency (OD₂₈₀ nm)>0.1 AU above baseline is collected into a sterile, pyrogen-free container until the UV absorbancy returned to baseline.

### Column 2

1. The second chromatography step is a buffer exchange using Sephadex G25 fine resin (APB). A 20.0-cm (i.d.) BPG column (APB) containing 12.2 L of resin is equilibrated with 5 column volumes of buffer (24 mM sodium phosphate, pH 6.0) at 30 cm/h prior to loading.
2. Product is loaded at 25% column volume at a linear flow rate of 30 cm/h. The load is ished using 1 column volume of 25 mM sodium phosphate, pH 6.0. Three column runs are required to desalt all the material in preparation for the cation-exchange separation.

### Column 3

1. The third chromatography step is a cation exchange on Poros 50HS medium (PerSeptive Biosystems, Framingham, Mass.). In development studies, the resin load limit is determined to be approximately 10.0 mg protein/ml of resin. The desalted HIC-purified material is loaded onto a 9-cm (i.d.) Vantage A2 column (Millipore) containing 600 ml of Poros 50HS medium. The column is equilibrated with 25 mM sodium phosphate, pH 6.0.
2. The pooled peaks from the desalting column are loaded onto the cation-exchange column at a linear flow rate of 56 cm/h. The column is ished with 5 column volumes of the equilibration buffer. Protein is eluted with a 10-column volume linear gradient from 0 to 50% sodium phosphate, pH 6.0, and 0.5 M NaCl.
3. Three fractions at the start of the peak are collected separately, and the peak material above 0.25 AU is collected as a single fraction. Fractions also are collected for the eluate below 0.25 AU on the trailing edge of the peak. Fractions are analyzed by SDS-PAGE and only those containing a single band of rEGC SE SE are pooled.

### Column 4

1. The fourth chromatography step is a desalting of the Poros 50HS pool on Sephadex G25 fine medium. The Poros 50 pool is loaded onto a 20-cm (i.d.) column containing 12.8 L of media equilibrated in 25 mM sodium phosphate buffer, pH 6.0. A 20%-column volume load is applied at a linear flow rate of 30 cm/h.
2. One column volume of buffer is used to ish material after loading. All material with UV absorbency above baseline is collected into a precooled container.

### Column 5

1. The fifth chromatography step is a cation exchange on Poros 20HS medium (PerSeptive Bio-systems). The desalted rEGC SE pool is loaded onto a 9-cm (i.d.) Vantage A2 column containing 760 ml of Poros 20HS medium equilibrated in 25 mM sodium phosphate buffer, pH 6.0. The column is loaded at a linear flow rate of 56 cm/h.
2. Following the load, the column is ished with 5 column volumes of equilibration buffer (25 mM sodium phosphate buffer, pH 6.0). Proteins are eluted with a 10-column volume linear gradient from 0 to 50% 25 mM sodium phosphate, pH 6.0, and 0.5 M NaCl.
3. Three fractions are collected at the beginning of the peak elution (AU above 0.25) and continuing until the absorbance is below 0.25 AU. Fractions are analyzed by SDS-PAGE and only those fractions containing a single band of a rEGC SE are pooled.
4. The purified intermediate bulk product is sterile filtered using a 0.22 u Millipak 100-unit (Millipore), aliquoted into sterile Nalgene PETG bottles, and frozen at −70° C.

### Column 6

1. Purified intermediate bulk product underwent a final SEC step immediately prior to dilution and vialing. A 5% column volume load of purified rEGC SE is injected onto a 5-cm (i.d.) XK column (APB) packed with 1700 ml of Superdex 75 prep-grade resin (APB) using a superloop device. The separation is accomplished at a linear flow rate of 30 cm/h.
2. Peak fractions are collected, tested by HP-SEC, and pooled. Pooled fractions are then diluted in 50 mM glycine, pH 8.5, and 140 mM NaCl to a final target concentration of 80/ug/ml.
3. Diluted product is filtered through a 0.22-μm Millipore Millipak 20-unit filter and stored at 4° C. for less than 24 h prior to vialing.

### Fill and Finish

1. The 0.22-μ-filtered final drug product is filled into cleaned, sterile, depyrogenated 5-ml glass vials (West Company, Lionville, Pa.) with 13-mm butyl rubber stoppers (Wheaton, Millville, N.J.) and a flip-off, crimp-type seal (West Company). Vials are stored refrigerated at 2 to 8° C. for no longer than 48 h prior to controlled-rate freezing.
2. Final product storage is at or below −70° C.

### Analytical Testing Methods

Total protein analysis. Total protein concentrations of samples are determined using the BCA protein assay kit (Pierce Chemical Co., Rockford, Ill.) according to the manufacturer's instructions and using bovine serum albumin (Pierce) as the reference standard.
SDS-PAGE and Western blot analysis. Samples taken at various stages of purification are analyzed by SDS-PAGE using 4-20% gradient acrylamide gels (Novex, San Diego, Calif.) based on the method described by Laemmli. Samples are mixed with Tris-glycine SDS sample buffer containing β-mercaptoethanol as a reducing agent. Gels are stained with either Coomassie Brilliant Blue (Sigma Chemical Co.) or silver (APB). For Western blot analysis, proteins are transferred to a PVDF membrane (Novex) at 0.5 mA constant current in transfer buffer (25 mM Tris, 192 mM glycine, 20% methanol) for 1.5 h on ice. Blots are incubated in blocking buffer (PBS with 5% nonfat dry milk and 0.05% Tween 20) for 1 h at 4° C. and then treated with the primary antibody, a mouse monoclonal antibody to each rEGC SE, for an additional hour at 4° C. Blots are washed three times for 10 min in blocking buffer and then incubated for 1 h at 4° C. with a goat anti-mouse IgG conjugated to alkaline phosphatase (AP) (Kirkegaard & Perry Laboratories, Gaithersburg, Md.) diluted 1/5000 in blocking buffer. Immunoreactive proteins are visualized by incubation with NBT-BCIP (Life Technologies, Rockville, Md.) substrate for 5 min at ambient temperature.
Reversed-phase HPLC. Samples are analyzed by reversed-phase HPLC. Twenty-five-microgram samples are injected onto a 2.1×150-mm CIS column (Waters, Milford, Mass.). Protein is eluted from the C18 column with a gradient from 15% buffer A (0.1% TFA in ddH2O) to 65% buffer B (99.9% acetonitrile, 0.1% TFA). The separation is performed at ambient temperature, 1.0 ml/min, and is monitored at a wavelength of 210 nm. The separation is controlled and monitored with a Hewlett-Packard 1090 liquid chromatography workstation. Chromatograms are integrated, and the area under the rEGC SE peak is reported as a percentage of the total area detected.
SEC-HPLC. SEC is performed in phosphate-buffered saline, pH 7.4, with 0.5 M NaCl using a GS000SWx1. (7.8 mm×30 cm, 5-μm particle size) column (TosoHaas, Montgomeryville, Pa.). The flow rate for the separation is 0.5 ml/min for a 30-min run. Sample application is 25 μl at 1 mg/ml. The separation is run on a Waters HPLC controlled with Millenium software, with detection at a wavelength of 280 run. rEGC SE purity is calculated as the area under the rEGC SE peak as a percentage of the total peak area detected.
Capillary zonal electrophoresis and capillary isoelectric focusing. Each rEGC SE is analyzed by capillary zone electrophoresis (CZE) at 22° C. using a Beckman P/ACE 5510 instrument equipped with a 37-cm-long fused-silica capillary with a 50-μm i.d. The capillary is coated with 10% polyacrylamide for elimination of electroos-motic flow and adsorption of protein on the capillary wall. The running buffer is 100 mM acetic acid adjusted to pH 4.5 with TEA. The running voltage is 12 kV, and the current generated is 24 μA. The detection wavelength is 200 nm. Sample buffer is diluted to reduce the salt concentration. High salt concentration is shown to split the protein peak. Under these conditions, Each rEGC SE gives a single sharp peak at a migration time of about 10 min. Each rEGC SE is analyzed by capillary isoelectric focusing at 2° C. using a Beckman P/ACE 5510 instrument equipped with a 27-cm Beckman N-CHO capillary (50-μm i.d.). The anolyte is 90 mM phosphoric acid, and the catholyte is 40 mM NaOH. The ampholyte is a 50/50 mix of a Beckman ampholyte with a pH range of 3-10 and a pharmalyte with a pH range of 8-10.5. Focusing is conducted for 5 min at 10 kV followed by low-pressure mobilization under 10 kV. The protein and standards are detected at 280 nm. Two Bio-Rad (Hercules, Calif.) standards are used for pI calibration, one at pH 10.4 and the other at pH 8.4. The pI of each rEGC SE is determined by a two-point calibration curve using these two standards.

### Other Methods.

1. Gram-negative bacterial endotoxin levels are determined by the kinetic Limulus amebocyte lysate method (BioWhittaker, Walkersville, Md.). Endotoxin spike recovery and sample inhibition controls are also run.
2. Total DNA is determined with a DNA Threshold System (Molecular Devices, Sunnyvale, Calif.). Negative controls and DNA spike recovery samples are analyzed in parallel.
3. Host cell protein analysis is performed by ELISA using a commercially available kit (Cygnus Technologies, Plainville; MA). Residual host cell proteins are detected with a rabbit anti-E coli antiserum and a goat anti-rabbit IgG conjugated to alkaline phosphatase using a kit according to the manufacturer's instructions (Cygnus Technologies, Wrentham, Mass.). Negative and positive controls are included.
4. N-terminal sequencing is performed using an ABI 494 CLC protein sequencer equipped with an ABI 785A detector/ABI 1400 microgradient system/ABI 610 data analyzer. Mass spectrometry is performed by M-Scan (West Chester, Pa.). MALDI-TOF mass spectrometry is performed using a PerSeptive Biosystems Voyager Research Station coupled with a delayed extraction laser-desorption mass spectrometer.

Egc SEs are also produced biochemically. Most biochemical methods for purification of egc SEs utilize ion exchange materials such as CG-50, carboxymethyl-cellulose and the Sephadexes (gel filtration). Two examples will be given herein.

Staphylococcal aureus strain D8472E is inoculated into 500 ml of 3+1 in 2-liter flasks. The cultures are grown at 37° C. with aeration for 7 h, induced with 12 μg of CBAP/ml, and incubated an additional 11 h. Bacterial cells are removed by centrifugation, the culture supernatants are diluted 2.5-fold with distilled, deionized H₂O (ddH₂O), and the pH adjusted to 5.3 with HCl. CG-50 resin is prepared with the pH adjusted to pH 5.3. For a 1-liter volume of original culture, the swelled equivalent of 12.5 g of CG-50 is added to the diluted culture supernatants and stirred for 80 min at room temperature. After the resin settled, supernatants are removed, and a column (1.25-cm radius) is packed to a bed height of 20 cm (98-ml bed volume). The column is washed with 400 ml of ddH₂O at 3.5 ml/min. The column is eluted with 0.5 M sodium phosphate (pH 6.8)-0.2 M NaCl at 2 ml/min. All of the protein eluted in one peak. This bulk protein is dialyzed in 40 mM sodium phosphate, pH 5.4 (loading buffer), clarified, filter sterilized through a 0.45 μm-pore-size filter, and loaded onto an SP Sepharose column with dimensions of 16 mm by 20 cm (Pharmacia Biotech). The column was washed briefly with loading buffer and eluted at a rate of 0.25 ml/min with a pH gradient of pH 5.4 to 7.8 in 40 mM sodium phosphate. One-milliliter fractions were collected and assayed by sodium dodecyl sulfate-polyacrylamide gel electrophoresis (SDS-PAGE) and Western blot analysis for egc SEs. Egc SE-containing fractions are pooled, dialyzed in 20 mM sodium phosphate (pH 7)-150 mM NaCl, and applied to a Sephacryl S100 column (16 mm by 60 cm) equilibrated with the same buffer. Fractions collected demonstrating the broad peak are used for affinity chromatography to separate each egc SE.

Individual HiTrap columns (Amersham Pharmacia Biotech, Uppsala, Sweden) are prepared with immobilised rabbit anti-SEG, SEI, SEM, SEN, SEQ. PBS (8.1 mM Na₂HPO₄, 1.5 mM KH₂PO₄, 137 mM NaCl, 2.7 mM KCl, pH 7.3) is used as washing buffer and the antibody binding egc SEs are eluted using 0.1 M acetic acid (pH 3.0). The SEs are identified after purification using high performance liquid chromatography coupled to a mass spectrometer. The chromatography is carried out on a C₁₈ column (2 mm×250 mm) (VYDAC, Hesperia, Calif.) using a linear gradient from 10% to 60% acetonitrile in 0.1% trifluoroacetic acid over 30 minutes at 40° C. Mass determination is carried out using electrospray mass spectrometry (LCQ, Thermo Finnigan, San Jose, Calif.). Egc SEs found at the same retention time both before and after affinity purification are considered as egc SEs.

A second biochemical method for preparation of the egc SEs is as follows: Eighteen to 20 liters of culture supernatant fluid from Staphylococcal aureus strain D8472E is diluted with HP (1:5 to 1:10) and the pH adjusted to 5.6. G-S-50 resin (800 ml), preequilibrated to pH 5.6 in 0.03 M phosphate buffer (PB) is added and the mixture stirred for 1 hour. The resin is allowed to settle and the supernatant fluid decanted. The resin is placed in a column and the toxins eluted with 0.5 M PB, 0.5 M NaCl pH, 6.2. The concentrated, dialyzed toxins are placed in a column (5 cm χ 75 cm) of CM-Sepharose pretreated with 0.005 M PB. pH 5.6. The column is washed with the same buffer and the egc SEs eluted by treating the column stepwise with phosphate buffer 0.03 M, pH 6.0, 0.045 M, pH 6.25, 0.06 M, pH 6.5 and 0.12 M, pH 7.2. The fractions containing the egc SEs are combined, concentrated with polyethylene glycol (PEG) and dialyzed against 0.5 M NaCl, 0.05 M PB, pH 7.2. The concentrated egc SE solution (5 ml) is placed in a column of Sephacryl S-75 (pretreated with 0.5 M NaCl, 0.05 M PB, pH 7.2). The column is developed with the same buffer. The fractions containing the egc SEs are combined and dialyzed against 0.01 M PB, 0.15 M NaCl, pH 7.2 The egc SE concentration is about 1 mg/ml. The solution is then subjected to affinity chromatography to separate out each individual egc SE as given above in the first biochemical method

Samples of purified egc SEs made recombinantly or biochemically are further tested for purity biologic function, sterility and safety. Samples containing 5 and 10 mg/ml are tested in a double diffusion immunoprecipitation assay using known standards of egc SEs and mono specific antisera. A single precipitation line is noted which showed a line of identity with the known egc SE being assayed. Using a tritiated thymidine mitogenic assay with human peripheral blood mononuclear cells, each egc SE shows significant mitogenic activity comparable to that of the classic SEs. Each preparation is devoid of contaminating alpha hemolysin assessed in a rabbit erythrocyte hemolytic assay. The sterility of each preparation is demonstrated by negative cultures using (a) fluid thioglycollate medium and (b) soybean-casein digest A sample containing 1 mg/ml of SEB was tested for endotoxin contamination using Sigma E-toxate CAL assay. The final product is found to be free of endotoxin with a standard sensitivity of 0.1 ug endotoxin/mg egc SE. Safety testing in vivo is carried out in Hartley strain guinea pigs weighing less than 450 grams, and female C57 black mice, (Simonson Laboratories, Watsonville, Calif.), weighing less than 22 grams. Each animal is observed for 7 days with no significant change in condition or weight after intraperitoneal injection of 0.5 ml. of 50 ug/kg of each egc SE.

The amino acid sequences of SEG-SEU are shown below

| SEG (Baba,T. et al., Lancet 359, 1819-1827 (2002)) | |
|---|---|
| [SEQ ID NO: 9] | |
| 1 | MNKIFRVLTV SLFFFTFLIK NNLAYADVGV INLRNFYANY QPEKLQGVSS GNFSTSHQLE |
| 61 | YIDGKYTLYS QFHNEYEAKR LKDHKVDIFG ISYSGLCNTK YMYGGITLAN QNLDKPRNIP |
| 121 | INLWVNGKQN TISTDKVSTQ KKEVTAQEID IKLRKYLQNE YNIYGFNKTK KGQEYGYKSK |
| 181 | FNSGFNKGKI TFHLNNEPSF TYDLFYTGTG QAESFLKIYN DNKTIDAENF HLDVEISYEK |
| 241 | TE |
| SEG (Jarraud, S et al., J. Immunol. 166: 669-677 (2001)) | |
| (SEQ ID NO: 10) | |
| 1 | MKKLSTVIII LILEIVFHNM NYVNAQPDLK LDELNKVSDK NNKGTMGNVM NLYTSPPVEG |
| 61 | RGVINSRQFL SHDLIFPIEY KSYNEVKTEL ELENTELANN YKDKKVDIFG VPYFYTCIIP |
| 121 | KSEPDINQNF GGCCMYGGLT FNSSENERDK LIYVQVTIDN RQSLGFTITT NKNMVTIQEL |
| 181 | DYKARHWTKE KKLYEFDGSA FESGYIKFTE KNNTSFWFDL FPKKELVPFV PYKFLNIYGD |
| 241 | NKVVDSKSIK MEVFLNTH |
| SEH (Omoe, K. et al., J. Clin. Microbiol. 40: 857-862 (2002)) | |
| [SEQ ID NO: 11] | |
| 1 | EDLHDKSELT DLALANAYGQ YNHPFIKENI KSDEISGEKD LIFRNQGDSG NDLRVKFATA |
| 61 | DLAQKFKNKN VDIYGASFYY KCEKISENIS ECLYGGTTLN SEKLAQERVI GANVWVDGIQ |
| 121 | KETELIRTNK KNVTLQELDI KIRKILSDKY KIYYKDSEIS KGLIEFDMKT PRDYSFDIYD |
| 181 | LKGENDYEID KIYEDNKTLK SDDISHIDVN LYTKKKV |
| SEI (Kuroda, M. et al., Lancet 357 (9264), 1225-1240 (2001)) | |
| [SEQ ID NO: 12] | |
| 1 | MKKFKYSFIL VFILLFNIKD LTYAQGDIGV GNLRNFYTKH DYIDLKGVTD KNLPIANQLE |
| 61 | FSTGTNDLIS ESNNWDEISK FKGKKLDIFG IDYNGPCKSK YMYGGATLSG QYLNSARKIP |
| 121 | INLWVNGKHK TISTDKIATN KKLVTAQEID VKLRRYLQEE YNIYGHNNTG KGKEYGYKSK |
| 181 | FYSGFNNGKV LFHLNNEKSF SYDLFYTGDG LPVSFLKIYE DNKIIESEKF HLDVEISYVD |
| 241 | SN |
| SEJ (Zhang, S. et al., FEMS Microbiol. Lett. 168: 227-233 (1998)) | |
| [SEQ ID NO: 13] | |
| 1 | MKKTIFILIF SLTLTLLITP LVYSDSKNET IKEKNLHKKS ELSSITLNNL RHIYFFNEKG |
| 61 | ISEKIMTEDQ FLDYTLLFKS FFISHSQYND LLVQFDSKET VNKFKGKQVD LYGSYYGFQC |
| 121 | SGGKPNKTAC MYGGVTLHEN NQLYDTKKIP INLWIDSIRT VVPLDIVKTN KKKVTIQELD |
| 181 | LQARYYLHKQ YNLYNPSTFD GKIQKGLIVF HTSKEPLVSY DLFNVIGQYP DKLLKIYQDN |
| 241 | KIIESENMHI DIYLYTSLIV LISLPLVL |
| SEK (Baba, T., et aL, Lancet 359: 1819-1827 (2002)) | |
| [SEQ ID NO: 14] | |
| 1 | MKKLISILLI NIIILGVSNN ASAQGDIGID NLRNFYTKKD FINLKDVKDN DTPIANQLQF |
| 61 | SNESYDLISE SKDFNKFSNF KGKKLDVFGI SYNGQCNTKY IYGGITATNE YLDKPRNIPI |
| 121 | NIWINGNHKT ISTNKVSTNK KFVTAQEIDI KLRRYLQEEY NIYGHNGTKK GEEYGHKSKF |
| 181 | YSGFNIGKVT FHLNNNDTFS YDLFYTGDDG LPKSFLKIYE DNKTVESEKF HLDVDISYKE |
| 241 | TK |
| SEL (Kuroda ,M. et al., Lancet 357: 1225-1240 (2001)) | |
| [SEQ ID NO: 15] | |
| 1 | MKKRLLFVIV ITLFTFSSNH TVLSNGDVGP GNLRNFYTKY EYVNLKNVKD KNSPESHRLE |
| 61 | YSYKNDTLYA EFDNEYITSD LKGKNVDVFG ISYKYGSNSR TIYGGVTKAE NNKLDSPRII |
| 121 | PINLIINGKH QTVTTKSVST DKKNVTAQEI DVKLRKYLQD EFNIYGHNDT GKGKEYGTSS |
| 181 | KFYSGFDKGS VVFHMNDGSN FSYDLFYTGY GLPESFLKIY KDNKTVDSTQ FHLDVEISKR |
| SEM (Kuroda, M. et al., Lancet 357: 1225-1240 (2001)) | |
| [SEQ ID NO: 16] | |
| 1 | MKRILIIVVL LFCYSQNHIA TADVGVLNLR NYYGSYPIED HQSINPENNH LSHQLVFSMD |
| 61 | NSTVTAEFKN VDDVKKFKNH AVDVYGLSYS GYCLKNKYIY GGVTLAGDYL EKSRRIPINL |
| 121 | WVNGEHQTIS TDKVSTNKKL VTAQEIDTKL RRYLQEEYNI YGFNDTNKGR NYGNKSKFSS |
| 181 | GFNAGKILFH LNDGSSFSYD LFDTGTGQAE SFLKIYNDNK TVETEKFHLD VEISYKDES |
| SEN (Jarraud, S et al., J. Immunol. 166: 669-677 (2001)) | |
| (SEQ ID NO: 17) | |
| 1 | MKNSKVMLNV LLLILNLIAI CSVNNAYANE EDPKIESLCK KSSVGPIALH NINDDYINNR |
| 61 | RFTTVKSIVS TTEKFLDFDL LFKSINWLDG ISAEFKDLKE FSSSAISKEF LGKYVDIYGV |
| 121 | YYKAHCHGEH QVDTACTYGG VTPHENNKLS EPKNIGVAVY KDNVNVNVNT FIVTTDKKK |
| 181 | VYAQELDIKV RTKLNNAYKL YDRMTSDVQK GYIKFHSHSE HKESFYYDLF YIKGNLPDQY |
| 241 | LQIYNDNKTT IDSSDYHIDV YLFT |
| SEO (Jarraud, S et al., J. Immunol. 166: 669-677 (2001)) | |
| (SEQ ID NO: 18) | |
| 1 | MKNIKKLMRL FYIAAIIITL LCLINNNYVN AEVDKKDLKK KSDLDSSKLFN LTSYYTDITW |
| 61 | QLDESNKIST DQLNNYIILK NIDISVLKTS SLKVEFNSSD LANQFKGKNUD IYGLYFGNKC |
| 121 | VGLTEEKTSC LYGGVTIHDG NQLDEEKVIG VNGFKDGVQQ EGFVIKTKKAK VTVQELDTKV |
| 181 | RFKLENLYKI YNKDTGNIQK GCIFFHSHNH QDQSFYYDLY NVKGSVGAEFF QFYSDNRTVS |
| 241 | SSNYHIDVFL YKD |
| Ψent 1 (Jarraud, S et al., J. Immunol. 166: 669-677 (2001)) | |
| (SEQ ID NO: 19) | |
| 1 | MKLFAFIFIC VKSCSLLFML NGNPKPEQLN KASEFTGLMD NMRYLYDDKH VSETNIKSQE |
| 61 | KFLQHDLLFK INGSKILKTE FNNKSLSDKY KNKNVDLFGT NYYNQCYFSL DNMELNDGRL |
| 121 | IEKNVYVWRC GL |
| Ψent 2 (Jarraud, S et al., J. Immunol. 166: 669-677 (2001)) | |
| (SEQ ID NO: 20) | |
| 1 | MYGGVVYENE RNSLSFDIPT NKKNITAQEI DYKVRNYLLK HKNLYEFNSSP YETGYIKFIE |
| 61 | GSGHSFWYDL MPESGKKFYP TKYLLIYNDN KTVESKSINV EVHLTKK |
| SEP (Kuroda, M. et al., Lancet 357, 1225-1240 (2001)) | |
| [SEQ ID NO: 21] | |
| 1 | MSKMKKTAFT LLLFIALTLT TSPLVNGSEK SEEINEKDLR KKSELQGTAL GNLKQIYYN |
| 61 | EKAKTENKES HDQFLQHTIL FKGFFTDHSW YNDLTVDFDS KDIVDKYKGK KVDLYFAYYG |
| 121 | YQCAGGTPNK TACMYGGVTL HDNNRLTEEK KEPINLWLDG KQNTVPLETV KTNKKVTVQ |
| 131 | ELDLQARRYL QEKYNLYNSD VFDGKVQRGL IVFHTSTEPS VNYDLFGAQG QYSNTLLRIY |
| 241 | RDNKTINSEN MHIDIYLYTS |
| SEQ (Lindsay, J A et al., Mol. Microbiol. 29, 527-543 (1998)) | |
| [SEQ ID NO: 22] | |
| 1 | MPIWRCNIKK GAIKMNKIFR ILTVSLFFFT FLIKNNLAYA DVGVINLRNF YANYEPEKLQ |
| 61 | GVSSGNFSTS HQLEYIDGKY TLYSQFHNEY EAKRLKDHKV DIFGISYSGL CNTKYMGGI |
| 121 | TLANQNLDKP RNIPINLWVN GKQNTISTDK VSTQKKEVTA QEIDIKLRKY LQNEYNIYGF |
| 131 | NKTKKGGEYG YQSKFNSGFN KGKITFHLNN EPSFTYDLFY TGTGGAESFL KIYNDNKTID |
| 241 | AENFHLDVEI SYEKTE |
| SER Omoe, K et al., ACCESSION BAC97795 | |
| [SEQ ID NO: 23] | |
| 1 | MLNKILLLLF SVTFMLLFFS LHSVSAKPDP RPGELNRVSD YKKNKGTMGN VESLYKDKAV |
| 61 | IAENVKNTRQ FLGHDLIFPI PYSEYKEVKS EFINKKTADK FKDKRLDVFG IPYFYTCLVP |
| 121 | KNESREEFIF DGVCIYGGVT MHSTADSISK NIIVPVTVDN KQQFSFTIST NKKTVTVQEL |
| 181 | DYKVRNWLTN NKKLYEFDGS AYETGYIKFI EQNKDSFWYD LFPKKDLVPF IPYKFVNIYG |
| 241 | DNKTIDASSV KIEVHLTTM |
| SEU (Letertre, C et al J. Appl. Microbiol. 95, 38-43 (2003)) | |
| [SEQ ID NO: 24] | |
| 1 | MKLFAFIFIC VKSCSLLFML NGNPRPEQLN KASEFSGLMD NMRYLYDDKH VSETNIKAQE |
| 61 | KFLQHDLLFK INGSKIDGSK ILKTEFNNKS LSDKYKNKNV DLFGTNYYNQ CYFSADNMEL |
| 121 | NDGRLIEKTC MYGGVTEHDG NQIDKNNLTD NSHNILIKVY ENERNTLSFD ISTNMKNITA |
| 131 | QEIDYKVRNY LLKHKNLYEF NSSPYESGYI KFIEGNGHSF WYDMMPESGE KFYPTKYLLI |
| 241 | YNDNKTVESK SINVEVHLTK K |

### Streptococcal Pyrogenic Exotoxins (SpEs)

The SpE's SPEA, SPEB, SPEC, SPEG, SPEH, SME-Z, SME-Z2 and SSA are superantigens induce tumoricidal effects. SPEA, SPEB, SPEC have been known for some time and their structures and biological activities described in numerous publications.

SPEG, SPEH, and SPEJ genes were identified from the Streptococcus pyogenes M1 genomic database and described in detail in Proft, T et al., J. Exp. Med. 189: 89-101 (1999) which also describes SMEZ, SMEZ-2. This document also describes the cloning and expression of the genes encoding these proteins.

The smez-2 gene was isolated from the S. pyogenes strain 2035, based on sequence homology to the streptococcal mitogenic exotoxin z (smez) gene. SMEZ-2, SPE-G, and SPE-J are most closely related to SMEZ and SPEC, whereas SPEH is most similar to the SEs than to any other streptococcal toxin.

As described by Proft, T et al supra, rSMEZ, rSMEZ-2, rSPE-G, and rSPE-H were mitogenic for human peripheral blood T lymphocytes. SMEZ-2 appears to be the most potent SAg discovered thus far.

All these toxins, except rSPE-G, were active on murine T cells, but with reduced potency.

Binding to a human B-lymphoblastoid line was shown to be zinc dependent with high binding affinity of 15-65 nM. Analysis of competition for binding between toxins of this group revealed overlapping but discrete binding to subsets of class II molecules in the hierarchical order (SMEZ, SPE-C)>SMEZ-2>SPE-H>SPE-G. The most common targets for these SAgs were human Vβ2.1- and Vβ4-expressing T cells.

Streptococcus Pyrogenic Exotoxin A (SPEA)

SPEA can be purified from cultures of S. pyogenes as described by Kline et al., Infect. Immun. 64:861-869 (1996). Plasmids that include the spea1 gene which encode SPEA, and the expression and purification of recombinant SPEA (“rSPEA”) are described by Kline et al., supra. The native SPEA sequence is shown below:

| SPEA (Papageorgiou, A. C. et al. EMBO J. 18: 9-21 (1999)) | |
|---|---|
| [SEQ ID NO: 25] | |
| 1 | MENNKKVLKK MVFFVLVTFL GLTISQEVFA QQDPDPSQLH RSSLVKNLQN IYFLYEGDPV |
| 61 | THENVKSVDQ LLSHDLIYNV SGPNYDKLKT ELKNQEMATL FKDKNVDIYG VEYYHLCYLC |
| 121 | ENAERSACIY GGVTNHEGNH LEIPKKIVVK VSIDGIQSLS FDIETNKKMV TAQELDYKVR |
| 181 | KYLTDNKQLY TNGPSKYETG YIKFIPKNKE SFWFDFFPEP EFTQSKYLMI YKDNETLDSN |
| 241 | TSQIEVYLTT K |

Streptococcus Pyrogenic Exotoxin B (SPEB)

Purification of native SPEB is described by Gubba, S. et al., Infect. Immun. 66: 765-770 (1998). Expression and purification of recombinant SPEB are also described in this reference. The native SPEB sequence is shown below (Kapur, V. et al., Microb. Pathog. 15:327-346 (1993)): [SEQ ID NO:26]

| [SEQ ID NO: 17] | |
|---|---|
| 1 | MNKKKLGIRL LSLLALGGFV LANPVFADQN FARNEKEAKD SATTFIQKSA AIKAGARSAE |
| 61 | DIKLDKVNLG GELSGSNMYV YNISTGGFVI VSGDKRSPEI LGYSTSGSFD ANGKENIASF |
| 121 | MESYVEQIKE NKKLDTTYAG TAEIKQPVVK SLLDSKGIHY NQGNPYNLLT PVIEKVKPGE |
| 181 | QSFVGQHAAT GCVATATAQI MKYHNYPNKG LKDYTYTLSS NNPYFNHPKN LFAAISTRQY |
| 241 | NWNNILPTYS GRESNVQKMA ISELMADVGI SVDMDYGPSS GSAGSSRVQR ALKENFGYNQ |
| 301 | SVHQINRSDF SKQDWEAQID KELSQNQPVY YQGVGKVGGH AFVTDGADGR NFYHVNWGWG |
| 361 | GVSDGFFRLD ALNPSALGTG GGAGGFNGYQ SAVVGIKP |

Streptococcus Pyrogenic Exotoxin C (SPEC)

Methods of isolation and characterization of SPEC is carried out by the methods of Li, P L et al., J. Exp. Med. 186: 375-383 (1997). These references also describe T cell proliferation stimulated by this SAg and the analysis of its selectivity for TCR Vβ regions. The native sequence of SPEC (Kapur, V. et al., Infect. Immun. 60: 3513-3517 (1992)) is shown below: [SEQ ID NO:27]

| [SEQ ID NO: 18] | |
|---|---|
| 1 | MKKINIIKIV FIITVILIST ISPIIKSDSK KDISNVKSDL LYAYTITPYD YKDCRVNFST |
| 61 | THTLNIDTQK YRGKDYYISS EMSYEASQKF KRDDHVDVFG LFYILNSHTG EYIYGGITPA |
| 121 | QNNKVNHKLL GNLFISGESQ QNLNNKIILE KDIVTFQEID FKIRKYLMDN YKIYDATSPY |
| 181 | VSGRIEIGTK DGKHEQIDLF DSPNEGTRSD IFAKYKDNRI INMKNFSHFD IYLE |

Streptococcal Superantigen (SSA)

SSA is an ˜28-kDa superantigen protein isolated from culture supernatants as described by Mollick J et al., J. Clin. Invest. 92: 710-719 (1993) and Reda K et al., Infect. Immun. 62: 1867-1874 (1994). SSA stimulates proliferation of human T cells bearing Vβ1, Vβ3, Vβ5.2, and Vβ15 in an MHC class II-dependent manner. The first 24 amino acid residues of SSA are be 62.5% identical to SEB, SEC1, and SEC3. Purification and cloning of SSA is described in Reda K et al., Infect. Immun. 62: 1867-1874 (1994). The native sequence of SSA (Reda, K. B. et al., Infect. Immun. 64: 1161-1165 (1996)) is shown below: [SEQ ID NO:28]

| [SEQ ID NO: 19] | |
|---|---|
| 1 | MNKRIRILVV ACVVFCAQLL STSVFASSQP DPTPEQLNKS SQFTGVMGNL RCLYDNHFVE |
| 61 | GTNVRSTGQL LQHDLIFPIK DLKLKNYDSV KTEFNSKDLA AKYKNKDVDI FGSNYYYNCY |
| 121 | YSEGNSCKNA KKTCMYGGVT EHHRNQIEGK FPNITVKVYE DNENILSFDI TTNKKQVTVQ |
| 181 | ELDCKTRKIL VSRKNLYEFN NSPYETGYIK FIESSGDSFW YDMMPAPGAI FDQSKYLMLY |
| 241 | NDNKTVSSSA IAIEVHLTKK |

Streptococcal Pyrogenic Exotoxins G and H and SMEZ

The sequences of the more recently discovered Streptococcal exotoxin SAgs are provided below:

| SPEG (Fraser, J et al., Mol Med Today 6: 125-32 (2000)) | |
|---|---|
| [SEQ ID NO: 29] | |
| 1 | DENLKDLKRS LRFAYNITPC DYENVEIAFV TTNSIHINTK QKRSECILYV DSIVSLGITD |
| 61 | QFIKGDKVDV FGLPYNFSPP YVDNIYGGIV KHSNQGNKSL QFVGTLNQDG KETYLPSEVV |
| 121 | RIKKKQFTLQ EFDFKIRKFL MEKYNIYDSE SRYTSGSLFL ATKDSKHYEV DLFNKDDKLL |
| 181 | SRDSFFKRYK DNKIFNSEEI SHFDIYLKTY |
| SPEH (Proft, T. et al., J. Exp. Med. 189: 89-102 (1999)) | |
| [SEQ ID NO: 30] | |
| 1 | MRYNCRYSHI DKKIYSMIIC LSFLLYSNVV QANSYNTTNR HNLESLYKHD SNLIEADSIK |
| 61 | NSPDIVTSHM LKYSVKDKNL SVFFEKDWIS QEFKDKEVDI YALSAQEVCE CPGKRYEAFG |
| 121 | GITLTNSEKK EIKVPVNVWD KSKQQPPMFI TVNKPKVTAQ EVDIKVRKLL IKKYDIYNNR |
| 181 | EQKYSKGTVT LDLNSGKDIV FDLYYFGNGD FNSMLKIYSN NERIDSTQFH VDVSIS |
| SMEZ (Proft, T. et al., J. Exp. Med. 191: 1765-1776 (2000)) | |
| [SEQ ID NO: 31] | |
| 1 | LEVDNNSLLR NIYSTIVYEY SDTVIDFKTS HNLVTKKLDV RDARDFFINS EMDEYAANDF |
| 61 | KAGDKIAVFS VPFDWNYLSK GKVTAYTYGG ITPYQKTSIP KNTPVNLWIN RKQIPVPYNQ |
| 121 | ISTNKTTVTA QEIDLKVRKF LIAQHQLYSS GSSYKSGKLV FHTNDNSDKY SLDLFYTGYR |
| 181 | DKESIFKVYK DNKSFNIDKI GHLDIEIDS |
| SMEZ 2 (Arcus, V. L. et al., J. Mol. Biol. 299 (1), 157-168 (2000)) | |
| [SEQ ID NO: 32] | |
| 1 | GLEVDNNSLL RNIYSTIVYE YSDIVIDFKT SHNLVTKKLD VRDARDFFIN SEMDEYAAND |
| 61 | FKTGDKIAVF SVPFDWNYLS KGKVTAYTYG GITPYQKTSI PKNIPVNLWI NGKQISVPYN |
| 121 | ETSTNKTTVT AQEIDLKVRK FLIAQHQLS SGSSYKSGRL VFHTNDNSDK YSFDLFYVGY |
| 181 | RDKESIFKNY KDNKSFNIDK IGHLDIEIDS |

Yersinia pseudotuberculosis Mitogen (Superantigen) (YPM)

Cloning, expression and purification of YPM is described by Miyoshi-Akiyama, T. et al., J. Immunol. 154: 5228-5234 (1995).

The above reference described assays of YPM using lymphoid cells and murine L cells transfected with human HLA genes, including T cell proliferation and cytokine (IL2) secretion. The sequence of YPM is shown below

| (Carnoy, C. et al., J. Bacteriol. 184 (16), 4489-4499 (2002)) | |
|---|---|
| [SEQ ID NO: 33]: | |
| 1 | MKKKFLSLLT LTFFSGLALA ADYDNTLNSI PSLRIPNIET YTGTIQGKGE VCIRGNKEGK |
| 61 | SRGGELYAVL RSTNANADMT LILLCSIRDG WKEVKRSDID RPLRYEDYYT PGALSWIWEI |
| 121 | KNNSSEASDY SLSATVHDDK EDSDVLMKCP |

Staphylococcal Exotoxin Like Proteins (SET)

The identification characterization of the SETs (SET-1 and SET-2) and the cloning and purification of SET-1 is described in Williams, R. J. et al., Infect. Immun. 68: 4407-4414 (2000). This reference discloses the distribution of the set1 gene among Staphylococcal species and strains.

The set1 nucleotide sequences are deposited in the GenBank database under accession numbers AF094826 (set gene cluster fragment), AF188835 (NCTC 6571 set1 gene), AF188836 (FR1326 set1 gene), and AF188837 (NCTC 8325-4 set1 gene). Recombinant SET-1 protein stimulates production of the proinflammatory cytokines IL-1β, IL-6, and TNFα

| SET1 (Williams, R. J. et al., Infect. Immun. 68 (8), 4407-4415 | |
|---|---|
| (2000)) | |
| [SEQ ID NO: 34] | |
| 1 | MKLKTLAKAT LALSLLTTGV ITLESQAVKA AEKQERVQHL YDIKDLYRYY SAPSFEYSNI |
| 61 | SGKVENYNGS NVVRFNQKDQ NHQLFLLGKD KEQYKEGLQG KDVFVVQELT DPNGRLSTVG |
| 121 | GVTKKNNKTS ETKTHLLVNK VDGGNLDASI DSFLIQKEEI SLKELDFKIR QQLVEKYGLY |
| 181 | QGTSKYGKIT INLKDEKREV IDLSDKLEFE RMGDVLNSKD IKGISVTINQ I |
| SET2 Williams, R. J., et al., Infect. Immun. 68 (8), 4407-4415 | |
| (2000) | |
| [SEQ ID NO: 35] | |
| 1 | MKLKTLAKAT LALGLLTTGV ITSEGQAVQA AEKQERVQHL HDIRDLHRYY SSESFEYSNV |
| 61 | SGKVENYNGS NVVRFNPKDQ NHQLFLLGKD KEQYKEGLQG QNVFVVQELI DPNGRLSTVG |
| 121 | GVTKKNNKTS ETNTPLFVNK VNGEDLDASI DSFLIQKEEI SLKELDFKIR QQLVNNYGLY |
| 181 | KGTSKYGKII INLKDENKVE TDLGDKLQFE RMGDVLNSKD IRGISVTINQ I |
| SET3 (Williams, R. J. et al., Infect. Immun. 68 (8), 4407-4415 | |
| (2000)) | |
| [SEQ ID NO: 36] | |
| 1 | MKMTAIAKAS LALSILATGV ITSTAQTVNA SEHESKYENV TJDUFDKRDT YSPASKELKN |
| 61 | VTGYRSKGG KKHYLIFDKNR KFTRIQIFGK DIERIKKRKN PGLDIFVVKE AENRNGTVYS |
| 121 | YGGVTLLMQG AYYDYLSAPR FVIKKEVGAG VSVHVKRYYI YKEEISLKEL DFKLRQYLIQ |
| 181 | DFDLYKKFPK ASKIKVTMKD GGYYTFELNK KLQTNRMSDV IDGRNTEKIE ANIR |
| SET4 (Williams, R. J. et al., Infect. Immun. 68 (8), 4407-4415 | |
| (2000)) | |
| [SEQ ID NO: 37] | |
| 1 | MKLTALAKVT LALGILTTGT LTTEAHSGHA KQNQKSVNKH DKEALHRYYT GNFKEMKNIN |
| 61 | ALRHGKNNLR FKYRGMKTQV LLPBDEYRKY QQRRHTGLDV FFNQERRDKH DISYTVGGVT |
| 121 | KTNKTSGFVS TPRLNVTKEK GEDAFVKGYP YDIKKEEISL KELDFKLRKH LIEKYGLYKT |
| 181 | LSKDGRIKIS LKDGSFYNLD LRTKLKFKHM GEVIDSKQIK DIEVNLK |
| SET5 (Williams, R. J., et al., Infect. Immun. 68 (8), 4407-4415 | |
| (2000)) | |
| [SEQ ID NO: 38] | |
| 1 | MKLTAIAKAT LALGILTTGV MTAESQTVNA KVKLDETQRK YYTNMLKDYY SQESYESTNI |
| 61 | SVKSEDYYGS NVLNFNQRNK NFKVFLIGDD RNKYKELTHG RDVFAVPELI DTKGGIYSVG |
| 121 | GITKKNVRSV FGYVSHPGLQ VKKVDPKDGF SIKELFFIQK EEVSLKELDF KIRKMLVEKY |
| 181 | RLYKGASDKG RIVINMKDEK KHEIDLSEKL SFDRMFDVLD SKQIKNIEVN LN |

Preferred Plurality of Superantigens with Broad Vβ/α Profile for Therapeutic Use

A preferred construct for intravenous, intrathecal and intrapleural use comprises one or a plurality of different egc SEs in native form with a Vβ/Vα profile for the mixture exhibiting a minimum activation/recognition of 5 different Vβ/α-expressing T cell clones and a maximum of 24Vβ/α-expressing T cell populations after stimulation with individual SAgs. SEs of the egc complex are preferred which include native SEG, SEI, SEM, SEN, SEQ and SEs encoded by egc pseudogenes ENT1 and ENT2 including homologues, variants, fusion and chimeric proteins formed from members of the egc family. The latter SEs are administered by infusion, injection, instillation or implantation by any parenterally route including but not limited to intravenously, intrapleurally, intraperitoneally, intrapericardially, intrathecally, intravesicularly, subcutaneously, intradermally using doses of each superantigen in a range of 0.1 pg-1.5 ng for each treatment. While any mixture of one or a plurality of native egc superantigens or egc superantigen homologues or mixtures of egc native superantigens and egc superantigen homologues are useful provided they collectively activate/recognize a minimum of 5 different Vβ/α-expressing T cell clones or T cell populations after stimulation with individual SAgs. The native egc SEs or their homologues may also be combined with any native non-egc superantigen or homologue provided they collectively activate/recognize a minimum of 5 different Vβ/α-expressing T cell clones or T cell populations after stimulation with individual SAgs.

The preferred SEs are given preferably intravenously every 1-3 days for up to 30 days. For malignant pleural effusions, the preferred mixture is preferably administered intrapleurally every 3-7 days after removal of pleural fluid via thoracentesis until no there is no further fluid reaccumulation. Optionally, the same mixture is administered intravenously q1-3 days starting with the first intrapleural treatment and continuing until the effusion has failed to reaccumulate. In addition, patients with or without recurrence of pleural effusion may be treated with the same regimen at 3-6 month intervals. If the pleural space is inaccessible, the SAg mixture may be administered intravenously q1-3 days. The preferred SE composition can also be given intratumorally once weekly for 4-12 weeks and the cycle can be repeated every 2-6 months.

The preferred SEs or superantigens and/or homologues in the mixture are those to which humans do not make or make only marginal amounts neutralizing antibody. Egc SEs are known to have the property of rare association with the neutralizing antibodies in humans and other non-egc SAgs that share this property and would also be desirable therapeutic agents against cancer. If some superantigens contain epitopes which bind endogenous (to include preexisting) superantigen specific antibodies, these are deleted and/or substituted by alanine or amino acid epitopes to which the host does not have preexisting antibodies. For example, a dominant epitope on SEB recognized by anti-SEB antibodies is the sequence 225-234 (Nishi et al., J. Immunol. 158: 247-254 (1997) and an epitope on SEA recognized by anti-SEA antibodies is the sequence 121-149 (Hobieka et al., Biochem. Biophys. Res. Comm. 223: 565-571 (1996). Alternatively, SAgs such as Y. pseudotuberculosis or C. perfringens toxin A or to which humans do not have preexisting antibodies are used. Y. pseudotuberculosis SAg has, in addition, a natural RGD domain with useful tumor-localizing properties and this moiety will preferably be retained. In the absence of neutralizing antibodies against them, SAgs or SAg homologues may be fused recombinantly or biochemically to a tumor specific antibody, Fab or single chain Fv in order to improve their localization to tumor sites in vivo.

### Use of Cytokines to Prevent SAg-Driven Activation Induced T Cell Death (AICD) in Vivo IL-2 and IL-15

IL-2 and IL-15 have similar biological characteristics such as activation, proliferation, and cytokine release by various subsets of T, natural killer (NK), and B cells and share IL-2Rβ and -γ chains for signal transduction. Both cytokines bind to and signal through a common, intermediate affinity receptor complex composed of β (CD122) and γ chain receptor (CD132) sub-units. Each cytokine interacts with a unique, ligand-specific α chain receptor. Both IL-2 and IL-15 delivered with antigen significantly enhance B cell memory. IL-15 supports human B cell proliferation in combination with CD40L and regulates differentiation of B-1 cells into IgA-producing cells.

Despite similarities, it is now clear that IL-2 and IL-15 have very different origins, and at times oppositional function in T cell biology. IL-2 is produced by T cells, and IL-15 mRNA is expressed by a broad range of cell types including activated monocytes, dendritic cells, and fibroblasts but not T cells. IL-2 promotes T cell activation and proliferation, and signaling through the IL-2 receptor (IL-2R) complex whereas signaling through the IL-15R complex is necessary for the development of elements of the innate immune system. IL-2 inhibits memory CD8+ T cell proliferation and survival and plays a pivotal role in suppression and activation-induced cell death especially of activated lymphocytes. In contrast, IL-15 inhibits IL-2-mediated activation-induced cell death and contributes to the proliferation and maintenance of antigen-independent memory CD8+ T cell populations Indeed, the frequency of CD8+ T cells induced with IL-15, unlike the immunizations with IL-2 persists up to 14 months. Moreover, IL-2 inhibits trafficking of adoptively transferred T cells into intracranial or subcutaneous tumors. Finally, despite its short-term stimulatory effects, high-dose IL-2 can cause severe, dose-limiting toxicities in patients. Thus, IL-15, with its capacity to invoke sustainable cellular and humoral responses, is a superior cytokine adjuvant and can improve the in vivo antitumor activity of adoptively transferred CD8+ T cells.

### IL-7 and 23

IL-7 is produced by non-hematopoetic cells and IL-23, produced by APC. Myeloid cells, which are the natural source of IL-23, disappear rapidly in the in vitro cultures. Thus an exogenous source of these cytokines is required for in vitro culture activation. The combination of IL-2 and IL-7 provides rapid proliferation of CD8⁺ T cells and preserves their viability after completion of the initial mitogenic burst. This combination is effective because IL-7 receptor a chain is constitutively expressed on naïve and memory T cells but is downregulated on activated T cells. By contrast, the IL-2 receptor α chain is reciprocally expressed on activated cells in a transient manner. Thus, the combination of these two cytokines ensures continuous mitogenic signal transduction. IL-7 is crucial for development and homeostasis of T cells and is markedly increased following lymphodepletion. This provides a rationale for employing lymphodepletion as a strategy to augment adoptive immunotherapy. Indeed, depletion of CD8⁺ cells and use of cytokine combinations such as IL-7 and IL-23 favored the selective hyperexpansion of CD4⁺ cells that retained potent in vivo function.

IL-23 is a member of the IL-12 family of cytokines and contains the IL-12 p40 subunit that transduces signals through the shared IL-12β1 chain in addition to the unique p19 subunit. The IL-23 receptor is expressed on memory but not naive CD4⁺ cells, thus it is ideal for previously sensitized LN T cells.

CD4⁺ T cells have been investigated as a source of helper function for CD8⁺ cytolytic cells our previous experiments have clearly established their stand-alone potential against MHC class II negative tumors. In addition to their autonomous effector functions, CD4⁺ cells are required to generate a functional CD8⁺ memory response in vivo. In this regard, IL-23 stimulation of CD4⁺ cells is particularly useful because it induces IL-17, a proinflammatory cytokine. The latter promotes inflammation at sites of tumor killing and tumor antigen acquisition resulting in host sensitization and perpetuation of the anti-tumor response.

To prevent SAgs from producing activation-driven T cell death (ADTCD) in vivo after they are administered to the patient, cytokines, IL-7, IL-15 and IL-23 are delivered either before, simultaneously with or shortly after the SSAg. As noted previously, all SAgs may be used but the egc SAgs are used preferentially as one or a plurality. As noted in Example 8, these cytokines are used ex vivo to stimulate/prevent apoptosis in T cells or T cell subsets, NK cells or LAK cells. These cytokines are also administered before, simultaneously with and/or shortly after administration of adoptively transferred cells as given in Example 9. They are also administered before, during or after all of the egc SAg or derivatives, homologues and conjugates disclosed in this application. One, two or all three of these cytokines may be given in doses of 0.5 ng-200 ug/day. They may be given parenterally, by infusion or injection, intrathecally, intravenously, intratumorally, intramuscularly, intradermally, intrapleurally, intraperitoneally, intrapericardially, intravesicularly, intraarticularly and in soluble form or as locally sustained release formulations. The administration of the cytokine(s) may be repeated for 1-7 days after the initial administration of the SAg and in a similar fashion for each dose of SAg or SAg derivative, homologues or conjugates. These cytokines may also be used together with SAg fragments and fusion proteins as disclosed in this application. Likewise they are used together with immunocytes activated ex vivo by SAgs and they are administered to the patient simultaneously with the SAg-activated immunocytes.

### Superantigen Vaccines In Vivo

### Preventative or vs Therapeutic (Established Tumor)

SAg polypeptides of all types but preferably the egc SEs and egc derivatives, homologues, conjugates, fragments and fusion proteins as disclosed herein may also be used as a vaccine to immunize a host against a cancer previously present in a host or in a host who is susceptible to cancer, or in whom a cancer is likely to develop. For example, the egc SEs can be used alone for vaccination or they may be used in conjunction with an inactivated tumor cell or tumor cell lysate, tumor associated antigen or plurality of tumor associated antigens (collectively immunotherapeutic antigens). The cytokines IL-7, IL-15, IL-23 may also be administered before, during (including together with) or after each egc SE vaccination. The vaccine can be given parenterally, intravenously, intramuscularly or subcutaneously, intratumorally, intrathecally, intrapleurally, intraperitoneally, intravesicularly, intrapericardially, intralymphatically, intradermally by injection, infusion or implantation. The SAg vaccine may be given by implantation in a sustained release formulation as disclosed in the instant specification. The egc SAg preparations for vaccination may also be administered in nucleic acid form. Vehicles useful for SAgs and protocols for testing the egc SAg as vaccines are provided in Vaccine Protocols edited by Robinson et al., (1996); Gene Therapy Protocols edited by Robbins et al., Humana Press, Totowa, N.J. (1997)) which are incorporated in their entirety by reference. The vaccines are tested in animal tumor models as given in the section “Tumor Models and Procedures for Evaluating Anti-Tumor Effects Studies”

The egc SEs and immunotherapeutic antigens may be separate from each other or conjugated to each other as a biochemically prepared conjugate or fusion protein prepared recombinantly. They may administered in numerous different combinations, for example, as a conjugate/fusion protein or in the form of single solution containing both egc SE's and immunotherapeutic antigens or separate solutions containing unconjugated egc SAgs and immunotherapeutic antigens respectively or mixtures thereof. Egc superantigen doses range from 0.0001-1000 ng of each egc superantigen and immunotherapeutic antigens from 0.01 nanograms to 5 mg. In those embodiments of the subject methods in which the superantigens and immunotherapeutic antigens are administered separately, the immunotherapeutic antigen may be conjugated to a carrier. Vaccine treatment may be administered once weekly for 1-8 weeks with boosting at 3-6 month intervals. When the superantigens and immunotherapeutic antigens are administered separately, the time between administrations may be anywhere in the range of a few minutes to up to about one week, varying in accordance with the specific disease and the potency of the therapeutic agents employed. In a preferred embodiment, the egc SE's and immunotherapeutic antigens are administered in the form of a immunotherapeutic antigen-superantigen polymer.

The egc therapeutic antigen used with the egc SE's may also consist of a bacterial, parasitic or viral antigens, (preferably a library of antigens for each agent), associated with an infectious disease. The combination of these therapeutic antigens and egc superantigens either separately or as conjugates, fusion proteins and homologues are given on schedules and in doses similar to those used for egc SE's with immunotherapeutic tumor antigens given above. The egc SE's may also be administered alone for infectious diseases and tumors. The egc SE's have distinct and unique advantages over classic SE's as vaccines against cancer and infectious diseases as follows: 1. they are less toxic in humans, 2. stimulate a broader library of T cells clones, 3. only a small fraction of humans exhibit naturally occurring antibodies against them which neutralize their T cell activating properties.

The term “immunotherapeutic antigen” as used herein refers to a broad range of molecules that when used in conjunction with an egc SE in accordance with the methods and compositions of the invention, can produce a desirable therapeutic effect. A given molecule is said to be an “immunotherapeutic antigen” with respect to a specific disease or set of diseases. Immunotherapeutic antigens are usually proteins, but may belong to other classes of macromolecules, such as carbohydrates, nucleic acids, lipids, gangliosides, glycolipids, glycoproteins and the like. Immunotherapeutic antigens would produce a desirable therapeutic effect e.g., as in cancer treatment or infectious disease. Exemplary, but not exclusive of, categories of immunotherapeutic antigens are (1) tumor antigens, (2) pathogenic organism antigens. Immunotherapeutic antigens may be obtained from natural sources or from host cell genetically engineered to produce the immunotherapeutic antigen. Relatively small polypeptides may be fully or partially synthesized by man-made artificial or biochemical processes and techniques well known in the art,

One category of immunotherapeutic antigen is the tumor antigen. The term “tumor antigen” refers to tumor associated antigens (TAA) and other peptides that are capable of evoking an immune response in the host (Reisfeld et al., Adv. Immunol. 1991). These antigens also include mutated products of various oncogenes and P53 genes which are expressed in tumor cells. Common melanoma antigens recognized by T lymphocytes have been identified and may be used as immunotherapeutic antigens. Five genes coding for different melanoma antigens have been identified. For example, MAGE 1 and 3, expressed on melanoma and other tumor cells, are recognized by CTL in the context of HLA-A1 (Van der Bruggen, P., et al., Science, 254:1643, (1991), Gauler, B., et al., J. Exp. Med., 179:921, (1994)). They were identified by T cell clones from peripheral blood of patients who were immunized with mutagenized tumor. MART-1 (identical to Melan-A) (Kawakami, Y., et al., Proc. Nat'l. Acad. Sci., 91:3515, (1994), Coulie, P. G., et al., J. Exp. Med., 180:35, (1994)), gp100 (Kawakami, Y., et al., Proc. Nat'l. Acad. Sci., 91:6458, (1994)), and tyrosinase (Brichard, V., et al., J. Exp. Med., 178:48, (1993)) are melanocyte lineage-related antigens expressed on both melanoma and melanocytes. MART-1 and gp100 have been shown to be recognized by MHC-class I-restricted CTL in the context of HLA-A2, and tyrosinase in the context of HLA-A2 and HLA-A24 (Robbins, P. F., et al., Cancer Res., 54:3126, (1995).

Another category of immunotherapeutic antigens is the pathogenic organism antigen. Various peptides have also been found to be significant in stimulating a protective immune response in infectious diseases. Immunotherapeutic antigens useful for the treatment of infectious diseases may be obtained from pathogenic bacteria, viruses, parasites and eukaryotes. For example, hepatitis viral peptides, HIV envelope peptides, Plasmodium yoeli circumsporozoite peptide are capable of protecting the host against challenge with the infectious agent from which they are derived.

The immunotherapeutic antigens and egc SE's may be in the form of an antigen-superantigen polymer, as a noncovalent combination of monomers, or as a mixture of monomers and polymers. The active composition of the invention can vary depending on the type of antigen and superantigen employed, the relative amount of each component in the mixture or conjugate, and the mode of conjugation used in forming the antigen-superantigen polymers. In one embodiment, the immunotherapeutic antigen molecules are directly conjugated with superantigen molecules through the use of a cross-linking agent able to form covalent linkages between the antigen and superantigen. When homobifunctional cross-linkers are used to effect this type of linkage, large molecular weight complexes may be created due to polymerization. In such cases, the size of the antigen-superantigen polymer may be several hundred thousand daltons in size, and in some instances, they may be even millions of daltons in size. Conjugations done using the water-soluble 1-ethyl-3-(3-dimethylaminopropyl)carbodiimide (EDC), which facilitates amide bond formation between carboxylates and amines on proteins, can result in similar polymeric conjugates having molecular masses of many millions of daltons, often while maintaining complete solubility (particularly if the original monomers had good solubility properties). Large molecular weight polymers also may be formed if a carrier is used to attach immunotherapeutic antigens to superantigen molecules. If a protein is used as a carrier, the molecular size of such complexes will be greater than the combined mass of the antigen plus superantigen monomers by the amount and mass of carrier present. A polymeric carrier such as periodate-oxidized dextran, which has numerous aldehyde coupling sites on its polysaccharide chain, can be used to form very large complexes by coupling multiple antigen and superantigen molecules along its length.

In another aspect of this invention, the antigen-egc SE conjugate may be relatively small in molecular mass by controlled cross-linking using heterobifunctional reagents. For instance, when using a heterobifunctional cross-linker like SMCC., a superantigen may be modified with the NHS ester end of the reagent to form amide bond derivatives of the protein that terminate in maleimide groups. The number of maleimide groups incorporated into a superantigen may vary from about 1 per protein molecule to perhaps as many as 30-40 per protein molecule, depending on the molar ratio of SMCC-to-superantigen used in the initial modification reaction. Reacting a sulfhydryl-containing peptide antigen with such a maleimide-activated superantigen could yield conjugates containing anywhere from about 1 peptide molecule per superantigen up to 30-40 peptides per superantigen. In practice, the lower ratio would not be targeted because statistics would dictate a large percentage of superantigen with no peptides attached (reactions are never 100% efficient). Similarly, the upper end of this ratio would be avoided, as high levels of cross-linker modification may result in loss of superantigen activity or precipitation. Thus, the optimal ratio of peptide-to-superantigen for this conjugation scheme is somewhere between these extremes and highly dependent on the nature of solubility of the antigen and superantigen making up the conjugate.

Thus, another aspect of this invention is that polymer conjugates of antigen and superantigen can consist of widely different ratios of the two components depending on the mode of cross-linking employed. When using a carrier such as periodate-oxidized dextran to form the peptide-superantigen conjugate there is great facility to create low and high ratios of peptide-to-superantigen complexes. For instance, a peptide can be reacted with the activated dextran carrier with a superantigen added to the mixture at a very low molar ratio to form a conjugate with perhaps only one molecule of superantigen per 100 molecules of peptide. Even higher ratios of peptide-to-superantigen can be used to prepare such conjugates, thus allowing discrete adjustment of the enterotoxin component to avoid toxicity issues. Using a multivalent carrier such as dextran to form the final immunotherapeutic polymer therefore allows the better potential for optimization of the activity of the conjugate than direct linking of antigen and superantigen. Similar to this approach is conjugation through a liposome carrier, wherein the antigen and superantigen are coupled to phospholipid derivatives on the vesicle surface. The ratio of antigen-to-superantigen used during the coupling reaction dictates the relative activity of the conjugate in the intended application.

Therefore, the immunotherapeutic antigen-superantigen polymers described by this invention may have ratios of antigen-to-superantigen that vary from equivalence (1:1) to as high as 10.sup.6:1 or even 10.sup.8:1 or as low as 10.sup.−6:1 or even 10.sup.−8:1. Each antigen-superantigen polymer is optimized in the ratio of components as well as in the mode of conjugation to produce a immunotherapeutic agent of this invention having high activity in its intended application while maintaining low toxicity. Such optimization is critical due to the wide variety of antigens that can be employed—each antigen having its own unique physical properties and biological activities that must be considered when preparing the final immunotherapeutic agent.

Peptide-superantigen polymers may be formed using conventional crosslinking agents such as carbodiimides. Examples of carbodiimides are 1-cyclohexyl-3-(2-morpholinyl-(4-ethyl)carbodiimide (CMC), 1-ethyl-3-(3-dimethylaminopropyl)carbodiimide (EDC) and 1-ethyl-3-(4-azonia-4,4-dimethylpentyl)carbodiimide. Preferred crosslinking agents are selected from the group consisting of 1-cyclohexyl-3-(2-morpholinyl-(4-ethyl)carbodiimide, (1-ethyl-3-(3-dimethylaminopropyl carbodiimide (EDC) and 1-ethyl-3-(4-azonia-4,4-dimethylpentyl)carbodiimide.

Examples of other suitable crosslinking agents are cyanogen bromide, glutaraldehyde and succinic anhydride. In general any of a number of homobifunctional agents including a homobifunctional aldehyde, a homobifunctional epoxide, a homobifunctional imidoester, a homobifunctional N-hydroxysuccinimide ester, a homobifunctional maleimide, a homobifunctional alkyl halide, a homobifunctional pyridyl disulfide, a homobifunctional aryl halide, a homobifunctional hydrazide, a homobifunctional diazonium derivative and a homobifunctional photoreactive compound may be used. Also included are heterobifunctional compounds, for example, compounds having an amine-reactive and a sulfhydryl-reactive group, compounds with an amine-reactive and a photoreactive group and compounds with a carbonyl-reactive and a sulfhydryl-reactive group.

Specific examples of such homobifunctional crosslinking agents include the bifunctional N-hydroxysuccinimide esters dithiobis(succinimidylpropionate), disuccinimidyl suberate, and disuccinimidyl tartrate; the bifunctional imidoesters dimethyl adipimidate, dimethyl pimelimidate, and dimethyl suberimidate; the bifunctional sulfhydryl-reactive crosslink ers 1,4-di-[3′-(2′-pyridyldithio)propionamido]butane, bismaleimidohexane, and bis-N-maleimido-1,8-octane; the bifunctional aryl halides 1,5-difluoro-2,4-dinitrobenzene and 4.4′-difluoro-3,3′-dinitrophenylsulfone; bifunctional photoreactive agents such as bis-[b-(4-azidosalicylamido)ethyl]disulfide; the bifunctional aldehydes formaldehyde, malondialdehyde, succinaldehyde, glutaraldehyde, and adipaldehyde; a bifunctional epoxide such as 1,4-butaneodiol diglycidyl ether, the bifunctional hydrazides adipic acid dihydrazide, carbohydrazide, and succinic acid dihydrazide; the bifunctional diazoniums o-toluidine, diazotized and bis-diazotized benzidine; the bifunctional alkylhalides N,N′-ethylene-bis(iodoacetamide), N,N′-hexamethylene-bis(iodoacetamide), N,N′-undecamethylene-bis(iodoacetamide), as well as benzylhalides and halomustards, such as .alpha.,.alpha.′-diiodo-p-xylene sulfonic acid and tri(2-chloroethyl)amine, respectively.

Examples of other common heterobifunctional cross-linking agents that may be used to effect the conjugation of superantigen molecules to immunotherapeutic antigens that are peptides include, but are not limited to, SMCC succinimidyl-4-(N-maleimidomethyl)cyclohexane-1-carboxylate), MBAs (m-maleimidobenzoyl-N-hydroxysuccinimide ester), SLAB (N-succinimidyl(4-iodoacteyl)aminobenzoate), SMPB (succinimidyl-4-(p-maleimidophenyl)butyrate), GMBS(N-(.gamma.-maleimidobutyryloxy)succinimide ester), MPBH (4-(4-N-maleimidophenyl)butyric acid hydrazide), M2C2H (4-(N-maleimidomethyl)cyclohexane-1-carboxyl-hydrazide), SMPT (succin-imidyloxyarbonyl-.alpha.-methyl-.alpha.-(2-pyridyldithio)toluene), and SPDP (N-succinimidyl 3-(2-pyridyldithio)propionate). Crosslinking may be accomplished by coupling a carbonyl group to an amine group or to a hydrazide group by reductive amination.

Immunotherapeutic antigen-egc SE polymers also may be prepared by non-covalent attachment of monomers through ionic, adsorptive, or biospecific interactions. Complexes of peptide-superantigen with highly positively or negatively charged molecules may be done through salt bridge formation under low ionic strength environments, such as in deionized water. Large complexes can be created using charged polymers such as poly-(L-glutamic acid) or poly-(L-lysine) which contain numerous negative and positive charges, respectively. Adsorption of peptide-superantigen may be done to surfaces such as microparticle latex beads or to other hydrophobic polymers, forming non-covalently associated peptide-superantigen complexes effectively mimicking crosslinked or chemically polymerized protein. Finally, peptide-superantigen may be non-covalently linked through the use of biospecific interactions between other molecules. For instance, utilization of the strong affinity of biotin for proteins such as avidin or streptavidin or their derivatives could be used to form immunotherapeutic antigen-superantigen species. These biotin-binding proteins contain four binding sites that can interact with biotin in solution or be covalently attached to another molecule (Wilchek, M. et al., Anal Biochem. 171:1-32 (1988)). Superantigens or peptides can be modified to posses biotin groups using common biotinylation reagents such as the N-hydroxysuccinimidyl ester of D-biotin (NHS-biotin) which reacts with available amine groups on the protein. Biotinylated superantigens or peptides then can be incubated with avidin or streptavidin to create large complexes. The molecular mass of such polymers can be regulated through careful control of the molar ratio of biotinylated peptide to avidin or streptavidin. The incorporation of biotinylated superantigen molecules to this complex can be done as well.

The therapeutic egc SE-immunotherapeutic antigen composition can be prepared by crosslinking a mixture of peptide (or functional derivative) and a superantigen (or a functional derivative) with a carrier. The carrier preferably consists of a protein, a lipid or another polymer which can be covalently bonded to peptide (or derivative) and the egc SE. Preferred protein carriers include serum albumin, keyhole limpet hemocyanin, tetanus toxoid, ovalbumin, thyroglobulin, diphtheria toxoid, myoglobin, immunoglobulin and purified protein derivative of tuberculin. A non-protein polymer carrier may be a polysaccharide, a poly(amino acid), a poly(vinyl alcohol), a polyvinylpyrrolidone, a poly(acrylic acid), a polyurethane and a polyphosphazene. The immunotherapeutic antigen-superantigen polymer may be covalently bonded to a liposome.

In another embodiment of the invention, the egc SE's and immunotherapeutic antigens are administered ex vivo. Ex vivo therapy is particularly advantageous because it avoids many of the toxic effects that may be associated with certain superantigens and immunotherapeutic antigens. In ex vivo therapy, lymphocytes are removed from a patient (or compatible donor) and exposed to a superantigen and an immunotherapeutic antigen. The superantigens and immunotherapeutic antigens may be administered to the lymphocytes in numerous different combinations. The superantigens and immunotherapeutic antigens may be administered together in the form of a solution containing both the superantigens and immunotherapeutic antigens. Alternatively, the superantigens and immunotherapeutic antigens may be administered separately. In those embodiments of the subject methods in which the superantigens and immunotherapeutic antigens are administered separately, the immunotherapeutic antigen may be conjugated to a carrier. In a preferred embodiment of the invention, the superantigens and immunotherapeutic antigens are administered in the form of a immunotherapeutic antigen-superantigen polymer of the invention. After exposure to the immunotherapeutic antigen-superantigen polymers or other superantigen containing compositions of the invention, the exposed lymphocytes may introduced into the patient. Lymphocytes for use in ex vivo therapy may be either purified or not. The use of filtration on suitable absorbing columns or fluorescence activated cell sorting may be used to obtain blood cells enriched for particular lymphocyte populations having markers of interest.

Examples of viral antigens that may be used as immunotherapeutic antigens are as follows:

| | |
|---|---|
| PEPTIDE | |
| Influenza A | |
| virus | |
| HIV 1 | SEQUENCE |

| | | | |
|---|---|---|---|
| Gag | 51-65 | LETSEGCRQILGQLQ | [SEQ ID NO: 45] |
| | 205-219 | ETINEEAAEWDRVHP | [SEQ ID NO: 46] |
| | 219-233 | HAGPIAPGQMREPRG | [SEQ ID NO: 47] |
| | 265-279 | KRWIILGLNKIVRMY | [SEQ ID NO: 48] |
| | 378-391 | MQRGNFRNQRKIVK | [SEQ ID NO: 49] |
| | 418-433 | KEGHQMKDCTERQANF | [SEQ ID NO: 50] |
| Env | 105-117 | HEDIISLWDQSLK | [SEQ ID NO: 51] |
| | 312-327 | IRIQRGPGRAFVTIGK | [SEQ ID NO: 52] |
| | 428-445 | FINMWQEVGKAMYAPPIS | [SEQ ID NO: 53] |
| | 474-489 | RPGGGDMRDNWRSELY | [SEQ ID NO: 54] |
| | 510-521 | VVQREKRAVGIG | [SEQ ID NO: 55] |
| | 584-604 | RILAVERYLKDQQLLGIWGCS | [SEQ ID NO: 56] |
| | 827-843 | YVAEGTDRVIEVVQGACR | [SEQ ID NO: 57] |
| | 846-860 | RHIPRRIRQGLERIL | [SEQ ID NO: 58] |
| Nef | 66-80 | VGFPVTPQVPLRPMT | [SEQ ID NO: 59] |
| | 79-94 | MTYKAAVDLSHFLKEK | [SEQ ID NO: 60] |
| | 113-128 | WIYHTQGYFPDWQNYT | [SEQ ID NO: 61] |
| | 132-147 | GVRYPLTFGWCYKLVP | [SEQ ID NO: 62] |
| | 137-145 | LTFGWCYKL | [SEQ ID NO: 63] |
| | 160-174 | ENTSLLHPVSLHGMD | [SEQ ID NO: 64] |
| Vif. | 1-15 | MENRWQVMIVWWVDR | [SEQ ID NO: 65] |
| | 25-40 | VKHHMYVSGKARGWFY | [SEQ ID NO: 66] |
| | 46-60 | SPHPRISSEVHIPLG | [SEQ ID NO: 67] |
| | 60-72 | GDARLVITTYWGL | [SEQ ID NO: 68] |
| | 71-85 | GLHTGERDWHLGQGV | [SEQ ID NO: 69] |
| Rev | 1-16 | MAGRSGDSDEDLLKAV | [SEQ ID NO: 70] |
| | 18-30 | LIKFLYQSNPPPN | [SEQ ID NO: 71] |
| | 37-50 | ARRNRRRRWRERQR | [SEQ ID NO: 72] |
| Vpra | 1-14 | MEQAPEDQGRQREP | [SEQ ID NO: 73] |
| | 55-68 | AGVEAIIRILQQLL | [SEQ ID NO: 74] |
| | 68-80 | LFIHFRIGCRHSR | [SEQ ID NO: 75] |

Examples of Hepatitis virus peptides are as follows:

HBV-Derived Ideal HLAA2.1-Binding Motifs

| | |
|---|---|
| PEPTIDE | SEQUENCE |

| | | |
|---|---|---|
| 1. HbcAg18-27 | FLPSDFFPSV | [SEQ ID NO: 76] |
| 2. HbsAg201-210 | SLNFLGGTTV | [SEQ ID NO: 77] |
| 3. HbsAg251-259 | LLCLIFLLV | [SEQ ID NO: 78] |
| 4. HBsAg260-269 | LLDYQGMLPV | [SEQ ID NO: 79] |
| 5. HBsAg335-343 | WLSLLVPFV | [SEQ ID NO: 80] |
| 6. HBsAg338-347 | LLVPFVQWFV | [SEQ ID NO: 81] |
| 7. HBsAg348-357 | GLSPTVWLSV | [SEQ ID NO: 82] |
| 8. HBsAg378-387 | LLPIFFCLWV | [SEQ ID NO: 83] |

Any tumor associated antigen or infectious immunogen capable of eliciting a protective immune response may be used as an immunogen. The immunogen and egc SE may be administered separately or as a conjugate or fusion proteins comprising both immunogen and superantigen. Examples would be the egc SE's with melanoma antigens MAGE 1, tyrosinase and other MART-1 peptides, the SW 205 antigen in colon carcinoma, MUC1 in breast and lung tumor associated antigens. Likewise viral peptides may be utilized in hepatitis or HIV infection. Indeed, any peptide, glyolipid, glycoprotein, ganglioside, ceramide or variations thereof considered capable of inducing protective immunity against cancer or a causative organism in an infectious disease is useful in this invention. Antigenic material can be derived from or modeled after antigenic moieties of the causative agents of any infectious disease including but not limited to malaria, leishmaniasis, tuberculosis, streptococcal and staphylococcal-induced diseases and sepsis.

Egc SE-immunotherapeutic antigens may be prepared recombinantly as fusion proteins by methods well established in the art. Fusion proteins may consist of egc SE's fused to polypeptides or peptides such as tumor antigen (MART-1 for example), hepatitis or HIV peptides, heat shock proteins, infectious organism peptides. The genes for these therapeutic peptides agents are known and fusion proteins with egc SE's could be produced in transformed bacteria, or yeast using methodology well established in the art. Considerations applicable to the chemical conjugates would also be relevant to fusion proteins, namely avoidance of steric hindrance of the antigenic and superantigenic binding sites.

### Functional Homologues and Derivatives of Superantigen Proteins or Peptides

The present invention contemplates, in addition to native egc SAgs, the use of egc homologues of native SAgs that have the requisite biological activity to be useful in accordance with the invention.

Thus, in addition to native egc SAg protein and nucleic acid compositions described herein, the present invention encompasses functional derivatives, among which homologues are preferred. Homologues of the egc SEs are preferred. However, biologically active homologues of other staphylococcal enterotoxins, streptococcal exotoxins. Y. pseudotuberculosis superantigen YPM, C. perfringens toxin A, M. arthritides superantigens are included if humans do not have preexistent neutralizing antibodies against them. By “functional derivative” is meant a “fragment,” “variant,” “mutant,” “homologue,” “analogue,” or “chemical derivative. Homologues include fusion proteins, chimeric proteins and conjugates that include a SAg portion fused to or conjugated to a fusion partner polypeptide or peptide. A functional derivative retains at least a portion of the biological activity of the native protein which permits its utility in accordance with the present invention. Such biological activity includes stimulation of T cell proliferation and/or cytokine secretion, stimulation of T cell-mediated cytotoxic activity, as a result of interactions of the SAg composition with T cells preferably via the TCR Vβ or Vα region.

A “fragment” refers to any shorter peptide. A “variant” refers to a molecule substantially similar to either the entire protein or a peptide fragment thereof. Variant peptides may be conveniently prepared by direct chemical synthesis of the variant peptide, using methods well-known in the art.

A homologue refers to a natural protein, encoded by a DNA molecule from the same or a different species. Homologues, as used herein, typically share at least about 50% sequence similarity at the DNA level or at least about 18% sequence similarity at the amino acid level, with a native protein.

An “analogue” refers to a non-natural molecule substantially similar to either the entire molecule or a fragment thereof

A “chemical derivative” contains additional chemical moieties not normally a part of the peptide. Covalent modifications of the peptide are included within the scope of this invention. Such modifications may be introduced into the molecule by reacting targeted amino acid residues of the peptide with an organic derivatizing agent that is capable of reacting with selected side chains or terminal residues.

A fusion protein comprises a native SAg, a fragment or a homologue fused by recombinant means to another polypeptide fusion partner, optionally including a spacer between the two sequences. Preferred fusion partners are antibodies, Fab fragments, single chain Fv fragments. Other fusion partners are any peptidic receptor ligand, cytokine, extracellular domain (“ECD”) of a costimulatory molecule and the like.

The recognition that the biologically active regions of the SEs, for example, are substantially homologous, i.e., that the sequences are substantially similar, enables prediction of the sequences of synthetic peptides which will exhibit similar biological effects in accordance with this invention (Johnson, L. P. et al., Mol. Gen. Genet. 203:354-356 (1986).

The following terms are used in the disclosure of sequences and sequence relationships between two or more nucleic acids or polypeptides: (a) “reference sequence”, (b) “comparison window”, (c) “sequence identity”, (d) “percentage of sequence identity”, and (e) “substantial identity”

As used herein, “reference sequence” is a defined sequence used as a basis for sequence comparison. A reference sequence may be a subset or the entirety of a specified sequence; for example, as a segment of a full-length cDNA or other polynucleotide sequence, or the complete cDNA or polynucleotide sequence. The same is the case for polypeptides and their amino acid sequences.

As used herein, “comparison window” includes reference to a contiguous and specified segment of a polynucleotide or amino acid sequence, wherein the sequence may be compared to a reference sequence and wherein the portion of the sequence in the comparison window may comprise additions or deletions (i.e., gaps) compared to the reference sequence (which does not comprise additions or deletions) for optimal alignment of the two sequences. Generally, the comparison window is at least 20 contiguous nucleotides or amino acids in length, and optionally can be 30, 40, 50, 100, or longer. Those of skill in the art understand that to avoid a high similarity to a reference sequence due to inclusion of gaps in the sequence a gap penalty is typically introduced and is subtracted from the number of matches.

Methods of alignment of nucleotide and amino acid sequences for comparison are well-known in the art. For comparison, optimal alignment of sequences may be done using any suitable algorithm, of which the following are examples:
(a) the local homology algorithm (“Best Fit”) of Smith and Waterman, Adv. Appl. Math. 2: 482 (1981);
(b) the homology alignment algorithm (GAP) of Needleman and Wunsch, J. Mol. Biol. 48: 443 (1970); or
(c) a search for similarity method (FASTA and TFASTA) of Pearson and Lipman, Proc. Natl. Acad. Sci. 85 2444 (1988);

In a preferred method of alignment, Cys residues are aligned. Computerized implementations of these algorithms, include, but are not limited to: CLUSTAL in the PC/Gene program by Intelligenetics, Mountain View, Calif., GAP, BESTFIT, BLAST, FASTA, and TFASTA in the Wisconsin Genetics Software Package, Genetics Computer Group (GCG) (Madison, Wis.). The CLUSTAL program is described by Higgins et al., Gene 73:237-244 (1988); Higgins et al., CABIOS 5:151-153 (1989); Corpet et al., Nuc Acids Res 16:881-90 (1988); Huang et al., CABIOS 8:155-65 (1992), and Pearson et al., Methods in Molecular Biology 24:307-331 (1994).

A preferred program for optimal global alignment of multiple sequences is PileUp (Feng and Doolittle, J Mol Evol 25:351-360 (1987) which is similar to the method described by Higgins et al. 1989, supra).

The BLAST family of programs which can be used for database similarity searches includes: NBLAST for nucleotide query sequences against database nucleotide sequences; XBLAST for nucleotide query sequences against database protein sequences; BLASTP for protein query sequences against database protein sequences; TBLASTN for protein query sequences against database nucleotide sequences; and TBLASTX for nucleotide query sequences against database nucleotide sequences. See, for example, Ausubel et al., eds., Current Protocols in Molecular Biology, Chapter 19, Greene Publishing and Wiley-Interscience, New York (1995) or most recent edition. Unless otherwise stated, stated sequence identity/similarity values provided herein, typically in percentages, are derived using the BLAST 2.0 suite of programs (or updates thereof) using default parameters. Altschul et al., Nuc Acids Res. 25:3389-3402 (1997).

As is known in the art, BLAST searches assume that proteins can be modeled as random sequences. However, many real proteins comprise regions of nonrandom sequence which may include homopolymeric tracts, short-period repeats, or regions rich in particular amino acids. Alignment of such regions of “low-complexity” regions between unrelated proteins may be performed even though other regions are entirely dissimilar. A number of low-complexity filter programs are known that reduce such low-complexity alignments. For example, the SEG (Wooten et al., Comput. Chem. 17:149-163 (1993)) and XNU (Claverie et al., Comput. Chem, 17:191-201 (1993)) low-complexity filters can be employed alone or in combination.

As used herein, “sequence identity” or “identity” in the context of two nucleic acid or amino acid sequences refers to the residues in the two sequences which are the same when aligned for maximum correspondence over a specified comparison window. It is recognized that when using percentages of sequence identity for proteins, a residue position which is not identical often differs by a conservative amino acid substitution, where a substituting residue has similar chemical properties (e.g., charge, hydrophobicity, etc.) and therefore does not change the functional properties of the polypeptide. Where sequences differ in conservative substitutions, the % sequence identity may be adjusted upwards to correct for the conservative nature of the substitution, and be expressed as “sequence similarity” or “similarity” (combination of identity and differences that are conservative substitutions). Means for making this adjustment are well-known in the art. Typically this involves scoring a conservative substitution as a partial rather than as a full mismatch, thereby increasing the percentage sequence identity. Thus, for example, where an identical amino acid is given a score of “1” and a non-conservative substitution is given a score of “0” zero, a conservative substitution is given a score between 0 and 1. The scoring of conservative substitutions is calculated, e.g., according to the algorithm of Meyers et al., CABIOS 4:11-17 (1988) as implemented in the program PC/GENE (Intelligenetics, Mountain View, Calif., USA).

As used herein, “percentage of sequence identity” refers to a value determined by comparing two optimally aligned sequences over a comparison window, wherein the portion of the nucleotide or amino acid sequence in the comparison window may comprise additions or deletions (i.e., gaps) as compared to the reference sequence (which lacks such additions or deletions) for optimal alignment, such as by the GAP algorithm (supra). The percentage is calculated by determining the number of positions at which the identical nucleotide or amino acid residue occurs in both sequences to yield the number of matched positions, dividing that number by the total number of positions in the window of comparison and multiplying the result by 100, thereby calculating the percentage of sequence identity.

The term “substantial identity” of two sequences means that a polynucleotide or polypeptide comprises a sequence that has at least 60%, preferably at least 70%, more preferably at least 80%, even more preferably at least 90%, and most preferably at least 95% sequence identity to a reference sequence using one of the alignment programs described herein using standard parameters. Values can be appropriately adjusted to determine corresponding identity of the proteins encoded by two nucleotide sequences by taking into account codon degeneracy, amino acid similarity, reading frame positioning, etc.

One indication that two nucleotide sequences are substantially identical is if they hybridize to one other under stringent conditions. Because of the degeneracy of the genetic code, a number of different nucleotide codons may encode the same amino acid. Hence, two given DNA sequences could encode the same polypeptide but not hybridize under stringent conditions. Another indication that two nucleic acid sequences are substantially identical is that the polypeptide encoded by the first nucleic acid is immunologically cross reactive with the polypeptide encoded by the second nucleic acid. Clearly, then, two peptide or polypeptide sequences are substantially identical if one is immunologically reactive with antibodies raised against the other. A first peptide is substantially identical to a second peptide, if they differ only by a conservative substitution. Peptides which are “substantially similar” share sequences as noted above except that nonidentical residue positions may differ by conservative substitutions.

Thus, in one embodiment of the present invention, the Lipman-Pearson FASTA or FASTP program packages (Pearson, W. R. et. al., 1988, supra; Lipman, D. J. et al, Science 227:1435-1441 (1985)) in any of its older or newer iterations may be used to determine sequence identity or homology of a given protein, preferably using the BLOSUM 50 or PAM 250 scoring matrix, gap penalties of −12 and −2 and the PIR or SwissPROT databases for comparison and analysis purposes. The results are expressed as z values or E ( ) values. To achieve a more “updated” z value cutoff for statistical significance, preferably corresponding to a z value >10 based on the increase in database size over that of 1988, in a FASTA analysis using the equivalent 2001 database, a significant z value would exceed 13.

A more widely used and preferred methodology determines the percent identity of two amino acid sequences or of two nucleic acid sequences after optimal alignment as discussed above, e.g., using BLAST. In a preferred embodiment of this approach, a polypeptide being analyzed for its homology with native SAg is at least 20%, preferably at least 40%, more preferably at least 50%, even more preferably at least 60%, and even more preferably at least 70%, 80%, or 90% as long as the reference sequence. The amino acid residues (or nucleotides) at corresponding positions are then compared. Amino acid or nucleic acid “identity” is equivalent to amino acid or nucleic acid “homology”.

In a preferred comparison of a putative SAg homologue polypeptide and a native SAg protein, the percent identity between two amino acid sequences is determined using the Needleman and Wunsch alignment algorithm (incorporated into the GAP program in the GCG software package (available at the URL www.gcg.com), using either a Blossom 62 matrix or a PAM250 matrix, and a gap weight of 16, 14, 12, 10, 8, 6, or 4 and a length weight of 1, 2, 3, 4, 5, or 6. In yet another embodiment, the percent identity between the encoding nucleotide sequences is determined using the GAP program in the GCG software package (also available at above URL), using a NWSgapdna.CMP matrix and a gap weight of 40, 50, 60, 70, or 80 and a length weight of 1, 2, 3, 4, 5, or 6. In another embodiment, the algorithm of Meyers et al., supra (incorporated into the ALIGN program, version 2.0), is implemented using a PAM120 weight residue table, a gap length penalty of 12 and a gap penalty of 4.

The wild-type (or native) SAg-encoding nucleic acid sequence or the SAg protein sequence can further be used as a “query sequence” to search against a public database, for example, to identify other family members or related sequences. Such searches can be performed using the NBLAST and XBLAST programs, supra (see Altschul et al. (1990) J. Mol. Biol. 215:403-10). BLAST nucleotide searches can be performed with the NBLAST program, score=100, wordlength=12 to identify nucleotide sequences homologous to native SAgs. BLAST protein searches can be performed with the XBLAST program, score=50, wordlength=3 to identify amino acid sequences homologous to identify polypeptide molecules homologous to a native SAg. To obtain gapped alignments for comparison purposes, Gapped BLAST can be utilized as described in Altschul et al. (1997, supra). Default parameters of XBLAST and NBLAST can be found at the NCBI website (www.ncbi.nlm.nih.gov)

Using the FASTA programs and method of Pearson and Lipman, a preferred SAg homologue is one that has a z value >10. Expressed in terms of sequence identity or similarity, a preferred SAg homologue for use according the present invention has at least about 20% identity or 25% similarity to a native SAg. Preferred identity or similarity is higher. More preferably, the amino acid sequence of a homologue is substantially identical or substantially similar to a native SAg sequence as those terms are defined above.

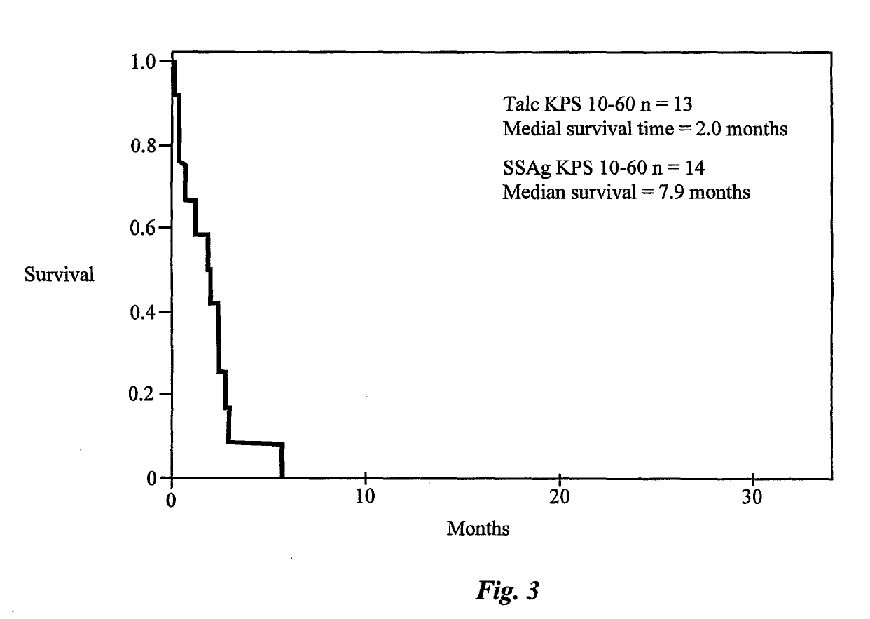

One group of substitution variants (also homologues) are those in which at least one amino acid residue in the peptide molecule, and preferably, only one, has been removed and a different residue inserted in its place. For a detailed description of protein chemistry and structure, see Schulz, G. E. Principles of Protein Structure Springer-Verlag, New York, 1978, and Creighton, T. E., Proteins: Structure and Molecular Properties, W.H. Freeman & Co., San Francisco, 1983, which are hereby incorporated by reference. The types of substitutions which may be made in the protein or peptide molecule of the present invention may be based on analysis of the frequencies of amino acid changes between a homologous protein of different species, such as those presented in Table 1-2 of Schulz et al. (supra) and FIG. 3-9 of Creighton (supra). Based on such an analysis, conservative substitutions are defined herein as exchanges within one of the following five groups:

1. Small aliphatic, nonpolar or slightly polar residues: Ala, Ser, Thr (Pro, Gly);
2. Polar, negatively charged residues and their amides: Asp, Asn, Glu, Gln;
3. Polar, positively charged residues: His, kg, Lys;
4. Large aliphatic, nonpolar residues: Met, Leu, Ile, Val (Cys); and
5. Large aromatic residues: Phe, Tyr, Trp.

The three amino acid residues in parentheses above have special roles in protein architecture. Gly is the only residue lacking any side chain and thus imparts flexibility to the chain. Pro, because of its unusual geometry, tightly constrains the chain. Cys can participate in disulfide bond formation which is important in protein folding. Tyr, because of its hydrogen bonding potential, has some kinship with Ser, Thr, etc.

More substantial changes in functional or immunological properties are made by selecting substitutions that are less conservative, such as between, rather than within, the above five groups, which will differ more significantly in their effect on maintaining (a) the structure of the peptide backbone in the area of the substitution, for example, as a sheet or helical conformation, (b) the charge or hydrophobicity of the molecule at the target site, or (c) the bulk of the side chain. Examples of such substitutions are (a) substitution of gly and/or pro by another amino acid or deletion or insertion of Gly or Pro; (b) substitution of a hydrophilic residue, e.g., Ser or Thr, for (or by) a hydrophobic residue, e.g., Leu, Ile, Phe, Val or Ala; (c) substitution of a Cys residue for (or by) any other residue; (d) substitution of a residue having an electropositive side chain, e.g., Lys, Arg or His, for (or by) a residue having an electronegative charge, e.g., Glu or Asp; or (e) substitution of a residue having a: bulky side chain, e.g., Phe, for (or by) a residue not having such a side chain, e.g., Gly.

The deletions and insertions, and substitutions according to the present invention are those which do not produce radical changes in the characteristics of the protein or peptide molecule. However, when it is difficult to predict the exact effect of the substitution, deletion, or insertion in advance of doing so, one skilled in the art will appreciate that the effect will be evaluated by routine screening assays, for example direct or competitive immunoassay or biological assay of T cell function as described herein. Modifications of such proteins or peptide properties as redox or thermal stability, hydrophobicity, susceptibility to proteolytic degradation or the tendency to aggregate with carriers or into multimers are assessed by methods well known to the ordinarily skilled artisan.

### Chemical Derivatives

Covalent modifications of the SAg proteins or peptide fragments thereof, preferably of SEs or peptide fragments thereof, are included herein. Such modifications may be introduced into the molecule by reacting targeted amino acid residues of the protein or peptide with an organic derivatizing agent that is capable of reacting with selected side chains or terminal residues. This may be accomplished before or after polymerization.

Cysteinyl residues most commonly are reacted with a-haloacetates (and corresponding amines), such as 2-chloroacetic acid or chloroacetamide, to give carboxymethyl or carboxyamidomethyl derivatives. Cysteinyl residues also are derivatized by reaction with bromotrifluoroacetone, α-bromo-(5-imidozoyl)propionic acid, chloroacetyl phosphate, N-alkylmaleimides, 3-nitro-2-pyridyldisulfide, methyl 2-pyridyl disulfide, p-chloromercuribenzoate, 2-chloromercuri-4-nitrophenol, or chloro-7-nitrobenzo-2-oxa-1,3-diazole.

Histidyl residues are derivatized by reaction with diethylprocarbonate at pH 5.5-7.0 because this agent is relatively specific for the histidyl side chain. Para-bromophenacyl bromide also is useful; the reaction is preferably performed in 0.1 M sodium cacodylate at pH 6.0.

Lysinyl and amino terminal residues are reacted with succinic or other carboxylic acid anhydrides. Derivatization with these agents has the effect of reversing the charge of the lysinyl residues. Other suitable reagents for derivatizing α-amino-containing residues include imidoesters such as methyl picolinimidate; pyridoxal phosphate; pyridoxal; chloroborohydride; triniobenzenesulfonic acid; 0-methylisourea; 2,4 pentanedione; and transaminase-catalyzed reaction with glyoxylate.

Arginyl residues are modified by reaction with one or several conventional reagents, among them phenylglyoxal, 2,3-butanedione, 1,2-cyclohexanedione, and ninhydrin. Derivatization of arginine residues requires that the reaction be performed in alkaline conditions because of the high pK of the guanidine functional group. Furthermore, these reagents may react with the groups of lysine as well as the arginine epsilon-amino group.

The specific modification of tyrosyl residues per se has been studied extensively, with particular interest in introducing spectral labels into tyrosyl residues by reaction with aromatic diazonium compounds or tetranitromethane. Most commonly, N-acetylimidizol and tetranitromethane are used to form O-acetyl tyrosyl species and 3-nitro derivatives, respectively.

Carboxyl side groups (aspartyl or glutamyl) are selectively modified by reaction with carbodiimides as noted above. Aspartyl and glutamyl residues are converted to asparaginyl and glutaminyl residues by reaction with ammonium ions.

Glutaminyl and asparaginyl residues may be deamidated to the corresponding glutamyl and aspartyl residues. Alternatively, these residues are deamidated under mildly acidic conditions. Either form of these residues falls within the scope of this invention.

Other modifications include hydroxylation of proline and lysine, phosphorylation of hydroxyl groups of seryl or threonyl residues, methylation of the a-amino groups of lysine, arginine, and histidine side chains (T. E. Creighton, Proteins: Structure and Molecule Properties, W.H. Freeman & Co., San Francisco, pp. 79-86 (1983)), acetylation of the N-terminal amine, and, in some instances, amidation of the C-terminal carboxyl groups.

Such derivatized moieties may improve the solubility, absorption, biological half life, and the like. The moieties may alternatively eliminate or attenuate any undesirable side effect of the protein and the like. Moieties capable of mediating such effects are disclosed, for example, in Remington's Pharmaceutical Sciences, 16th ed., Mack Publishing Co., Easton, Pa. (1980).

### Superantigen Homologues

The variants or homologues of native SAg proteins or peptides including mutants (substitution, deletion and addition types), fusion proteins (or conjugates) with other polypeptides, are characterized by substantial sequence homology to
(a) the long-known SE's—SEA, SEB, SEC1-3, SED, SEE and TSST-1;
(b) long-known SpE's;
(c) more recently discovered SE's (SEG, SEH, SEI, SEJ, SEK, SEL, SEM, SEN, SEQ, SEP, SER, SEU, SETs 1-5); or
(d) non-enterotoxin superantigens (YPM, M. arthritides superantigen).
   Preferred homologues were disclosed above.

Table 1 below lists a number of native SEs and exemplary homologues (amino acid substitution, deletion and addition variants (mutants) and fragments) with z values >10 (range: z=16 to z=136) using the Lipman-Pearson algorithm and FASTA. These homologues also induce significant T lymphocyte mitogenic responses that are generally comparable to native SE's.

In addition, as shown in Table 2, several of these homologues also promote antigen-nonspecific T lymphocyte killing in vitro by a mechanism termed “superantigen-dependent cellular cytotoxicity” (SDCC) or, in the case of SAg-mAb fusion proteins, “superantigen/antibody dependent cellular cytotoxicity (SADCC).

According to the present invention, other SE homologues (e.g., z>10 or, in another embodiment, having at least about 20% sequence identity or at least about 25% sequence similarity when compared to native SEs), exhibiting T lymphocyte mitogenicity, SDCC or SADCC, are useful anti-tumor agents when administered to a tumor bearing host via any intrathecal route.

### Tumors in Sheaths Encasing Organs

The appearance of tumors in sheaths (“theca”) encasing an organ often results in production and accumulation of large volumes of fluid in the organ's sheath. Examples include (1) pleural effusion due to fluid in the pleural sheath surrounding the lung, (2) ascites originating from fluid accumulating in the peritoneal membrane and (3) cerebral edema due to metastatic carcinomatosis of the meninges. Such effusions and fluid accumulations generally develop at an advanced stage of the disease.

### Intrathecal Superantigens for Treatment of Malignant Ascites and Malignant Pleural Effusions

The present invention contemplates the use of one or a plurality of SAgs or SETs in any form. This includes but is not limited to staphylococcal enterotoxins A, B, C, D, E, F, G, H, I, J, K, L, M, N, O, P, Q, R, U, SpE's, YPM, M. arthritides SAg, C. perfringens exotoxin for direct administration into cavities or spaces, e.g., peritoneum, thecal space, pericardial and pleural space containing tumor. Preferably, the host does not have preexistent neutralizing antibodies against them. The mixture of native egc staphylococcal enterotoxins G, I, M, N and O or egc superantigen homologues or mixtures of native egc superantigens and egc homologues to which humans rarely make neutralizing antibodies is preferred. The plurality of SAgs should activate/recognize TCR at least 5 different Vβ/Vα expressing T cells in human T cell populations after stimulation with individual SAgs using in vitro assays measuring T cell RNA/DNA Vβ/Vα expression or T cell surface expression of Vβ/α. Examples of these assays are well established in the art (Monday et. al., J. Immunol. 1624550-4550 (1999); Choi et al., Proc. Natl. Acad. Sci. 86: 8941-8945 (1989; Orwin et al., Infect. Immun. 69: 360-366 (2001); Choi et al., Proc Natl Acad Sci 88:8357-61 (1991)); Kotb et al., Immunomethods 2: 33-39 (1993); Deringer et al., Mol Microbiol. 22: 523-534 (1996); Mehrotra et al., J. Clin. Microbiol. 38: 1032-1035 (2000); Deringer et al., Infect. Immun. 65: 4048-4054 (1997); Monday et al., J. Clin. Microbiol. 37: 3411-3414 (1999); Becker et al., J. Clin. Microbiol. 41: 1434-1439 (2003)) which are herein incorporated by reference in their entirety.

TABLE 1
SE-Homologues Induce T Lymphocyte Mitogenesis

| | T Lymphocyte | |
|---|---|---|
| | Mitogenic Response b | Reference |
| SE Homologue a | (ED50) c | (SPECIES) |
| SEA (native) | 1 | Abrahmsen et al., EMBO J. 14: |
| SEA D227A | 1057 | 2978-2986 (1995); |
| SEA F47A | 52 | HUMAN |
| SEA H225A | 1272 | |
| SEA K123A/D132G | 2 | |
| SEA N128A | 2 | |
| SEA K55A | 1 | |
| SEA H50A | 4 | |
| SEA D45A | 1 | |
| SEA H187A | 11 | |
| SEA E191A/N195A | 1 | |
| SEA C96S | 12 | Grossman et al., J. Immunol. |
| SEA C106Q | 13 | 147: 3274-3281 (1991) |
| SEA C96, 106G | 10 | MOUSE |
| SEA K14E | 1 | Bavari et al., J. Infect. Dis. 174: |
| SEA Y64A | 100 | 338-345 (1996) |
| SEA Y92A | 100 | HUMAN |
| SEB (native) | 1 | Briggs et al., Immunol. 90: 169- |
| SEB H166A/V169E | 5 | 175 (1997) |
| SEB H166A | 1.3 | MOUSE |
| SEB V169A | 10 | |
| SEB V169E | 5 | |
| SEB V169K | 10 | |
| SEB (native) | 1 | Alakhov et al., Eur. J. Biochem. |
| SEB (1-13, 2-13) | 7.6 | 209: 823-828 (1992) |
| | | HUMAN |
| SEB (native) | 1 | Leder et al., J. Exp. Med. 187: |
| SEB L20T | 1.2 | 823-833 (1998) |
| SEB V26Y | 1 | MOUSE |
| SEB Y91B | 1.8 | |
| SEC3 (native) | 1 | |
| SEC3 Y26A | 7 | |
| SEC3 N60A | 6 | |
| SEC3 Y90A | 8 | |
| SEC3 G106A | 6 | |
| SEC1 (native) | 1 | Hoffman et al., Infect. Immun. 62: |
| SEC 1818 (delete 7-9) | 1 | 3396-3407 (1994) |
| SEC 1819 (delete 6-10) | 1 | HUMAN |
| SEC 1820 (delete 9-13) | 1 | |
| SEC 1821 (delete 9-18) | 53 | |
| SEC Mr (20-80) | 4.3 | Spero et al., J. Biol. Chem. 24: |
| | | 8787-8791 (1978) |
| | | MOUSE |
| SED (native) | 1 | Sundstrom et al., EMBO J. |
| SED F42A | ~100 | 15: 6832-6840 (1996) |
| SED D182A | ~5000 | HUMAN |
| SED 218A | ~1 | |
| SED D222A | ~100,000 | |
| SEE (native) | 1 | Lamphear et al., J. Immunol. |
| SEE-Ala (20-24) | 1 | 156: 2178-2185 (1996) |
| SEE-Ala (200-207 | 1 | HUMAN |
| SEE-Ala (20-24/200-207) | 1.7 | |
| | | Mollick et al., J. Exp. Med. 283- |
| SEA (native) | 1 | 293 (1993) |
| SEA-SEE (200-207) | 1 | HUMAN |
| SEE-SEA (70-71) | 1 | |
| SEA-SEE (200-207) | | |
| TSST-1 (native) | 1 | Kum et al., J. Infect. Dis. 174: |
| G31R | 800 | 1261-1270 (1996) HUMAN |
| SEA-C215 mAb Fab | 1 | Antonnson et al., J. Immunol. |
| Fusion Protein | | 158: 4245-4251 (1997) |
| SEE-C215 mAb Fab | 10 | HUMAN |
| Fusion protein | | |
| SEE/AA-C215 mAb Fab | 1 | |
| Fusion protein | | |
| SEE/A-C-C215 mAb Fab | 10 | |
| Fusion protein | | |
| SEE/A-F-C215 mAb Fab | 10 | |
| Fusion protein | | |
| SEE/A-H-C215 mAb Fab | 10 | |
| Fusion protein | | |
| SEA/E-BDEG-mAb Fab | 2 | |
| Fusion protein | | |
| SEE/A-AH-215 mAb Fab | 2 | |
| Fusion protein | | |

LEGEND FOR TABLE 1
a z values for homologues range from 16-136.
b Summary of Methods in all the above studies: human peripheral blood mononuclear cells (PBMC) or mouse spleen or lymph node lymphocytes were incubated with native SE or homologue (mutant) in complete medium supplemented with fetal calf serum (5 or 10% v/v) and antibiotics in wells of 96-well microplates in 200 μl volumes. In some cases, enriched or purified T lymphocytes from these populations were tested. Between 0.2 × 10⁵ and 8 × 10⁵ cells/well were used. Incubation was at 37° C. in humidified air/95% CO₂ for periods of between 66 hours and 84 hours (depending on whether unfractionated or purified T lymphocytes were being used). T lymphocyte mitogenic responses was routinely measured as radiolabeled [3H]-thymidine (“TdR”) incorporation during the final 4-24 hrs of incubation. Cells were always harvested from the microplates onto glass fiber filters which were dried and placed in a liquid scintillation counter for evaluation of incorporated radiolabel.
c Each SE or homologue was tested over a range of concentrations and the results were plotted as counts/min (cpm) of [3H]TdR taken up (after subtraction of background cpm of cells incubated in medium alone, which rarely exceeded several hundred cpm) on the ordinate vs. log concentration of the SE or homologue on the abscissa. For each agent tested, the concentration at which [3H]TdR incorporation was 50% of maximum (the ED50), which falls in the linear part of the sigmoid dose-response curve, has been provided in the publication or interpolated visually and approximated (value preceded by “~” symbol) from the published graphs. The ED50 of the native SE was arbitrarily set to 1, so an ED50 of 10 for a homologue indicates that the homologue causes half-maximal mitogenic responsiveness at a 10-fold higher concentration.

TABLE 2
SE Homologues Induce T Lymphocyte
Mitogenesis and Anti-Tumor Effects In Vitro

| | | | SADCC³ |
|---|---|---|---|
| | T Lymphocyte | | (% of native SE) |
| | Mitogenic Response¹ | SDCC² | Abrahmsen et al., |
| SE Homologue | (ED50) | (ED50) | WO96/01650 |
| Data from: Abrahmsen et al., | | | |
| EMBO J. 14:2978-2986 (1995) | | | |
| SEA (native) | 1 | 1 | 100 |
| SEA D227A | 1057 | 132 | 100 |
| SEA F47A | 52 | 4 | 100 |
| SEA H225A | 1272 | 130 | nd |
| SEA K123A/D132G | 2 | 2 | 100 |
| SEA N128A | 2 | 3 | 100 |
| SEA K55A | 1 | 1 | nd |
| SEA H50A | 4 | 2 | 100 |
| SEA D45A | 1 | 1 | nd |
| SEA H187A | 11 | 9 | 100 |
| SEA E191A/N195A | 1 | 1 | nd |
| Data from Sundstrom et al., | | | |
| EMBO J. 15:6832-6840 (1996) | | | |
| SED (native) | 1 | 1 | |
| SED F42A | ~100 | ~5 | |
| SED D182A | ~5000 | ~50 | |
| SED H218A | ~1 | ~1 | |
| SED D222A | ~50,000 | ~50 | |
| Data from Nilsson et al., | | | |
| J. Immunol. 163:6686-6693 (1999) | | | |
| SEH (native) | 1 | 1 | |
| SEH D167 | 10 | 5 | |
| SEH D203A | 7 | 5 | |
| SHE D208A | 300 | 10 | |

Legend for Table 2:
¹Lymphocyte proliferation assays:
(a) Abrahmsen et al., 1995: Peripheral blood mononuclear cells (PBMC)
from heparinized blood of normal donors were isolated by density
centrifugation over Ficoll-Hypaque. Following this, 2 × 10⁵ PBMC/
0.2 ml complete medium were incubated in microplates with varying
amounts of SEA or SEA mutants for 72 h and tested for mitogenic
responses (proliferation) by incorporation of [³H]-thymidine during
the last 4 h of culture. The SEA mutant concentration resulting in
half-maximum proliferation (ED50) was related to the ED50 of the native
SE, arbitrarily set to 1 (see column 2). Thus, the SEA homologue
concentration to induce half maximal response was related to the same
values induced by native SEA.
(b) Sundstrom et al, 1996: 10⁵ human PBMC prepared as above were
incubated at 37° C. in 0.2 ml complete medium in U-shaped microplate
wells with varying amounts of native SED or SED mutants for 96 hrs.
Proliferation was estimated by incorporation of [³H] thymidine
added during the final 24 hrs. ED50 values were estimated by interpolating
the curves in this publication.
(c) Nilsson et al., 1999: 2 × 10⁵ human PBMC were prepared as
above incubated in flat bottom microwells in 0.2 ml volumes at 37° C.
for 72 h with varying amounts of native SEH and variants. Each well was
pulsed with 0.5 μCi [³H] thymidine for 4 h. Cells were harvested and
proliferation measured as incorporation of [³H] thymidine. The ED50
values of the SEH variants were related to the ED50 of native SEH
which was 0.2 pM.
²SDCC = Superantigen dependent mediated cellular cytotoxicity.
This assay measures the ability of an SE (whether native or mutant)
to target cytotoxic T lymphocytes onto MHC class II+ target cells
resulting in their lysis. The same conditions were used in the above
publications. The cytotoxicity of SE (wt) and homologues against MHC
class II+ Raji cells was analyzed in a standard 4 or 6 hour ⁵¹Cr_release
assay, using SE-specific T cell lines that had been stimulated in vitro
(with the wild-type SE) as effector cells. Briefly, 2.5 × 10³ ⁵¹Cr-labeled
Raji cells were incubated in 0.2 ml medium (RPMI, 10% FCS) in
microwells in the presence effector cells at an effector:target cell ratio
of 30 and in the presence (or absence for negative controls) of the SE's or
homologues. After incubation, 0.1 ml of medium was withdrawn and
counted in a gamma counter to determine isotope release. % specific
cytotoxicity was calculated as
$100 \times [ \frac{( c . p . m . experimental release - c . p . m . background release )}{( c . p . m . total release - c . p . m . background release )} ] .$
The SE homologue concentration resulting in half-maximum cytotoxicity
(ED50) was related to the ED50 of the native SE, arbitrarily set to 1.
Thus, the SE homologue concentration needed to promote half maximal
cytotoxicity was related to the same values induced by the native or
wild SE. ED50 values were provided by the authors, or, in the case
of the Lundstrom reference, they were estimated by interpolating the
curves in this publication (shown as approximate using the ~ symbol.
³SADCC = Superantigen-tumor specific antibody mediated cellular
cytotoxicity. This is similar to SDCC but involves an antibody component
in the form of a fusion protein that directs the specificity of the targeting.
Here, this assay measure the ability of a fusion protein comprising
an SE (native or mutant) fused to an antibody Fab fragment to target
activated cytotoxic T lymphocytes onto tumor cells expressing the tumor
antigen (colon cancer antigen) against which the antibody (C215) is
specific. This targeting leads to tumor cell lysis, as above. The
cytotoxicity of C215Fab-SEA(wt), C215Fab-SEA(m), SEA(wt) and SEA
mutants against C215+ MHC class II(neg colon carcinoma cells
SW 620 was analyzed in a standard 4 hour ⁵¹Cr3+−release assay,
using in vitro stimulated SEA specific T cell lines as effector cells.
Briefly, ⁵¹Cr3+-labeled SW 620 cells were incubated at 2.5 × 10³
cells per 0.2 ml medium {RPMI, 10% FCS) in microtiter wells at effector
to target cell ratio 30:1 in the presence or absence (control) of the
additives. Percent specific cytotoxicity was calculated as for SDCC assays.

The SAg preparation is administered into a fluid space containing tumor cells or adjacent to membranes such as pleural, peritoneal, pericardial and thecal spaces containing tumor. These sites display malignant ascites, pleural and pericardial effusions or meningeal carcinomatosis. The SAg composition is preferably administered after partial or complete drainage of the fluid (e.g., ascites, pleural or pericardial effusion) but it may also be administered directly into the undrained space containing the effusion, ascites and/or carcinomatosus. In general, doses for each SAg in the SAg composition may vary from 0.1 picograms to 1.5 nanograms and are given every 3 to 10 days. SAg may be administered intrathecally every 3-10 days until there is no reaccumulation of the ascites or effusion. Therapeutic responses are considered to be no further accumulation of four weeks after the last intrapleural administration. See Example 1 for further description of treatment.

Superantigens with Staphylococcal Leukocidins

### Background

Staphylococcus aureus produces numerous virulence factors, including the bicomponent toxins, Panton-Valentine leucocidin (PVL), leukocidins and gamma hemolysin. PVL is of particular importance because of its high cytolytic specificity against human polymorphonuclear cells and macrophages. As early as 1894, a leukotoxic activity produced by S. aureus was observed by Van der Velde. Subsequently, Panton and Valentine differentiated the PVL from hemolysins in a S. aureus strain obtained from a case of human furunculosis. Gladstone et al., noted a selective leukotoxic effect of PVL against human and rabbit leukocytes but not against leukocytes obtained from mouse, sheep and guinea pig. In rabbits, PVL injected intradermally induced a potent dermonecrotic effect with edema and erythema; when given systemically, it produced granulocytopenia followed by a marked granulocytosis. In humans, PVL-producing strains are associated with furuncles, abscesses as well as pyodermic infections with dermonecrosis and a severe and highly lethal necrotizing pneumonia.

Approximately 99% of S. aureus strains, produce γ-hemolysin while 2% of these strains co-produce PVL. In addition to PVL and γ-hemolysin, other staphylococcal leukocidins such as LukE-LukD belong to the bi-component leukocidin family. With the availability of the primary sequence and cloning of all genes encoding these bi-component toxins, the number of proteins belonging to this family has grown to at least 11. The group as a whole is referred to as “synergohymenotropic toxins.” Two genetic loci for bifunctional toxins have been identified. The first locus, encoding PVL, consists of two cotranscribed open reading frames, LukS-PV and LukF-PV. The second, encoding γ-hemolysin consists of two transcription units, an HlgA-like protein (a class S component) and two cotranscribed open reading frames, HlgC and HlgB, (class F components).

PVL and γ-hemolysin are composed of five separate and complete proteins termed “S” and “F” based on their elution by chromatography, F (fast eluted, 32 kDa) and S (slow eluted, 38 kDa). Class S and class F proteins act synergistically on the target cell membrane to form membrane pores. All are secreted separately as lytically inactive components. Class S components consist of LukS-PV, HlgA (32 kDa), HlgC (32 kDa) with 63 to 75% identity, and class F components include LukF-PV, HlgB (34 kDa) with 70% identity. The PVL class F component (LukF-PV) may be shared in common with γ-hemolysin. The target cell specificities of both bi-component toxins are mainly determined by the class S (Hlg2 for γ-hemolysin and LukS-PV for PVL) proteins.

There are seven possible functional combinations of S and F components. All seven are leukocytolytic, however, the couples HlgC/LukF-PV and LukS-PV+HlgB show only leukotoxic properties. Two, LukS-PV/LukF-PV and HlgA-LukF-PV, also display dermonecrotic activity on rabbit skin. The two γ-hemolysin combinations, HlgA/HlgC and HlgA/HlgB, and the hybrid couple, HlgA+LukF-PV, induce both leukocytolysis and hemolysis.

Several phages carry PVL genes in PVL-positive strains of S. aureus. Vijver et al. found lysogenic conversion in S. aureus by a group A phage leading to leukocidin production. Subsequently, a temperate phage, φPVL, (41,401 bp with 3′-staggered cohesive ends of nine bases) carrying the PVL genes from a lysate of mitomycin C-treated S. aureus was identified. PVL-like genes in S. aureus strain P83 are carried by a prophage designated φPY83-pro (45,636 bp and a core sequence of 10 base pairs). Various temperate phages harboring PVL genes such as φSLT (42,942 bp with 29-bp attachment core sequences with 62 open reading frames), are also capable of converting non-PVL secreting strains of S. aureus to PVL secreting strains and support the widely held notion of horizontal transmission of PVL genes by temperate phages.

The key event leading to pore formation of the staphylococcal leukocidal toxins is the assembly of a heptameric β barrel structure from the monomer pairs of S and F proteins, (e.g., LukS-PV/LukF-PV, HlgA/HlgB, HlgC/HlgB) which are secreted as water-soluble molecules rich in β-sheet structure. Leukocidin attack on the cellular membrane begins with the recognition of a specific receptor on the target leukocyte by one of the soluble class S molecules (e.g., LukS). Polymorphonuclear leucocytes and monocytes are capable of binding tens of thousands of LukS molecules specifically with high affinity. Binding of S components to cell membranes is requisite before binding of F components can take place. The binding of S and F monomers to each other on the target cell membrane leads to the production of the a key circular heptameric structure. In the pre-pore state, the N terminus of the heptamer is folded against the core to shelter the small hydrophobic surface and the pre-stem domain is folded into three short antiparallel β-strands with the hydrophobic residues positioned against the protein core. However, after an unknown trigger, these two regions of the heptamer undergo concomitant conformational changes and reassemble as a β-barrel which is the active pore-forming configuration of the toxin. Hydrophilic and hydrophobic residues form the β-strands of the new stem domain generating a hydrophobic exterior, in contact with lipids, and a hydrophilic interior, the water-filled channel.

The permeability of the pore depends on the concentration of divalent cations in the extracellular medium. With concentrations of calcium lower than 1 mM, the lesions induced are big enough to allow the leakage of intracellular components which eventually causes cell death by osmotic shock. In the presence of concentrations of calcium higher than 1 mM, the lesions induced by PVL are initially ion-sized pores, permeable to different divalent cations. Incorporation of the second F component with formation of the β-barrel molecular complex perpendicular to the plane of the membrane creates aspecific pores which allow an influx of ethidium.

Calcium influx induced by the bifunctional toxins leads to up-regulation of CD1 1b/CD18 glycoprotein in human polymorphonuclear leukocytes (PMNs). Intracellular events such as degranulation, secretion, activation of phospholipase A2, release oxygen metabolites, IL-18 and leukotriene B4 from human neutrophilic granulocytes producing DNA fragmentation and chemotaxis of neutrophils and eosinophils follows rapidly. Purified PVL also induces a pronounced release of histamine and the enzymes β-glucuronidase and lysozyme from human basophilic granulocytes. When PVL is injected intradermally in rabbits, a severe inflammatory lesion is produced with histopathologic evidence of capillary dilation, polymorphonuclear infiltration and karyorrhexis leading to skin necrosis.

The present invention envisons the use of PVL nucleic acids in plasmid form administered intratumorally into viable human tumors. It is envisioned that following expression of these genes in the tumor sites that the tumor will undergo a necrotizing tumoricidal response.

The genes for both the S and F components are situated in frame preferably with one or a plurality of egc SEs more preferably SEG and/or SEI genes. These genes are cloned into the same vector (preferably the pH β Apr1-neo) which contains the beta actin promoter or another promoter functionally capable of activating bacterial genes in eukaryotic cells. This is described in U.S. patent application Ser. No. 09/870,759 which is incorporated in entirety by reference. In the rabbit VX 2 carcinoma model, VX2 fragments are implanted in the lateral thigh female rabbits 3-4 kg in body weight are used and as described in U.S. Pat. No. 6,340,461 which is herein incorporated in entirely by reference. The plasmid DNA is injected intratumorally every week for 12 weeks at multiple sites throughout the tumor via a 25 gauge needle in doses employed are 10-60 ug of plasmid DNA for tumors <8 cm³ and 30-100 ug plasmid DNA for tumors >8 cm³. The treatment is started when the tumors have grown to at least 6-8 cm³ Control animals are implanted with tumor and undergo treatment with empty vector alone. Spontaneous canine mammary carcinoma, melanoma and osteosarcoma are treated and followed in substantially in the same fashion except that for tumors <18 cm³ intratumoral treatment is administered at multiple sites in doses 400 ug plasmid DNA and for tumors >18 cm³ intratumoral treatment is given in doses of 800 ug plasmid DNA. Treated and control tumors measurements are compared and evaluated statistically by methods well established in the art. Median survival of treated and control groups is also determined at an arbitrary time points such as 30, 60, 90 and 120 days after starting treatment and the groups compared statistically by established methodology as described in U.S. Pat. No. 6,340,461 incorporated in entirety by reference.

The PVL proteins in doses of 0.1-20 ug are also administered intratumorally or intravenously to induce a tumoricidal effect. The S and F components in doses of 0.1-20 ug respectively are introduced simultaneously or sequentially every 1-3 days for 3-8 weeks. If delivered sequentially, the S component is guided to its target by conjugation to a tumor targeting device such as a tumor specific antibody or egf receptor ligand preferably with higher affinity for the tumor than the S or F component has for the PVL receptor. To promote tumor localization, the PVL receptor on PMNs may be temporarily blocked by preadministration of ganglioside GM1. The secondary F component is then delivered which targets the S component localized on the surface of the tumor cells. The animal models and controls above are employed for these studies as well as the followup and statistical assessment of the tumor measurements and survival.

The problem of preexisting antibodies to PVL components is overcome by using the nucleic acid form of the PVL and structurally modifying the S and F proteins to eliminate the dominant epitopes in these molecules. The S and F proteins from a homologous species such as humans may be used in rabbits. The human PVL may retain the receptor binding properties of the rabbit PVL components but is not recognized or recognized with weak affinity by the rabbit anti-PVL antibodies. Moreover, some antibodies specific for S or F components are known to actually promote the PVL effect rather than neutralizing it. These will be identified in each subject to determine whether they are functionally neutralizing or proinflammatory with respect to PVL.

### Fusion Partners for Native SEs or SE Homologues

Antibodies

Fusion protein partners for the egc SAg or egc homologues include tumor specific antibodies, preferably F(ab′)2, Fv or Fd fragments thereof, that are specific for antigens expressed on the tumor. In another embodiment, a fusion partner is a polypeptide ligand for a receptor expressed on tumor cells. These antibodies, fragments or receptor ligands may be in the form of synthetic polypeptides. The nucleic acid form of the antibody is envisioned which is useful as a fusion construct with the SAg DNA.

One advantage of certain antibody constructs of the present fusion polypeptides is prolonged half-life and enhanced tissue penetration. Intact antibodies in which the Fc fragment of the Ig chain is present will exhibit slower blood clearance than their Fab′ fragment counterparts, but a fragment-based fusion polypeptide will generally exhibit better tissue penetrating capability.

Preferentially, the tumor targeting structure in the superantigen conjugate (e.g., tumor specific antibody, Fab or single chain Fv fragments or tumor receptor ligand) has a greater affinity for the tumor than the SAg in the conjugate has for the class II molecule thus preventing the SAg from binding all MHC class II receptors and favoring binding of the conjugate to the tumor. In the case of SEB, the dominant epitope for neutralizing antibodies 225-234 is recombinantly or biochemically bound to the tumor targeting molecule e.g., tumor specific antibodies, Fas or Fv fragments. In so doing, it sterically interferes with the recognition of the dominant epitope by preexisting antibodies.

To further enhance the affinity of the tumor specific antibody in the conjugate for tumor cells in vivo, tumor specific antibodies are used which are specific for more than one antigenic structures on the tumor, tumor stroma or tumor vasculature or any combination thereof. The tumor specific antibody or F(ab′)₂, Fab or single chain Fv fragments are mono or divalent like IgG, polyvalent for maximal affinity like IgM or chimeric with multiple tumor (tumor stroma or tumor vasculature) specificities. Thus, when the SAg-MoAb conjugate is administered in vivo, it will preferentially bind to tumor cells rather than to endogenous SE antibodies or MHC class II receptors.

To reduce affinity of the SAg-mAb conjugate for endogenous MHC class II binding sites, the high affinity Zn++ dependent MHC class II binding sites in SEA, SEC2, SEC3, SED, SPEA, SPEC, SPEG, SPEH, SMEZ, SMEZ2, M. arthritides are deleted or replaced by inert sequence(s) or amino acid(s). These structural alterations in SE or SPEA reduce the affinity for MHC class II receptors from a Kd of 10⁻⁷ or 10⁻⁸ to 10⁻⁵. SEB, SEC and SSA and other SEs or SPEs do not have a high affinity Zn++dependent MHC class II binding site but have multiple low affinity MHC class II binding sites (Kd 10⁻⁵-10⁻⁷). In these cases, alteration of the MHC class II binding sites is not always necessary to further reduce affinity for MHC class II receptors; at the very least mutation of one or two of the low affinity MHC class II binding sites will suffice in most instances.

Most importantly, tumor specific antibodies, Fab, F(ab′)₂ or single chain Fab or Fv fragments in the SAg-mAb conjugate have a higher affinity for tumor antigens (Kd 10⁻¹¹-10⁻¹⁴ or lower) than for the superantigen has for MHC class II binding sites (Kd 10⁻⁵ to 10⁻⁷) and its dominant epitope has for superantigen specific antibodies (Kd 10⁻⁷ to 10⁻¹¹). In this way, the conjugate will bind preferentially to the tumor target in vivo rather than preexisting antibodies or MHC class II receptors.

Antibody fragments are obtained using conventional proteolytic methods. Thus, a preferred procedure for preparation of F(ab′)₂ fragments from IgG of rabbit and human origin is limited proteolysis by the enzyme pepsin. Rates of digestion of an IgG molecule may vary according to isotype; conditions are chosen to avoid significant amounts of completely degraded IgG as is known in the art.

Fab fragments include the constant domain of the light chain (CL) and the first constant domain (CH1) of the heavy chain. Fab′ fragments differ from Fab fragments by the addition of a few residues at the C-terminus of CH1 domain including one or more cysteine(s) from the antibody hinge region. F(ab′)₂ fragments were originally produced as pairs of Fab′ fragments that have hinge cysteines between them. Other chemical couplings of antibody fragments are also known.

An “Fv” fragment is the minimum antibody fragment that contains a complete antigen-recognition and binding site. This region consists of a dimer of one heavy chain and one light chain variable domain in tight, con-covalent association. It is in this configuration that the three hypervariable regions of each variable domain interact to define an antigen-binding site on the surface of the VH-VL dimer. Collectively, the six hypervariable regions confer antigen-binding specificity to the antibody. However, even a single variable domain (or half of an Fv comprising only three hypervariable regions specific for an antigen) has the ability to recognize and bind antigen, although at a lower affinity than the entire binding site.

“Single-chain Fv” or “scFv” antibody fragments comprise the VH and VL domains of antibody, wherein these domains are present in a single polypeptide chain. Generally, the Fv polypeptide further comprises a polypeptide linker between the VH and VL domains that enables the scFv to form the desired structure for antigen binding.

The following documents, incorporated by reference, describe the preparation and use of functional, antigen-binding regions of antibodies: U.S. Pat. Nos. 5,855,866; 5,965,132; 6,051,230; 6,004,555; and 5,877,289.

“Diabodies” are small antibody fragments with two antigen-binding sites, which fragments comprise a heavy chain variable domain (VH) connected to a light chain variable domain (VL) in the same polypeptide chain (VH and VL). By using a linker that is too short to allow pairing between the two domains on the same chain, the domains are forced to pair with the complementary domains of another chain and create two antigen-binding sites. Diabodies are described in EP 404,097 and WO 93/11161, incorporated herein by reference. “Linear antibodies”, which can be bispecific or monospecific, comprise a pair of tandem Fd segments (VH-CH1-VH-CH1) that form a pair of antigen binding regions.

An antibody fragment may be further modified to increase its half-life by any of a number of known techniques. Conjugation to non-protein polymers, such PEG and the like, is also contemplated

The antibody fusion partner for use in the present invention may be specific for tumor cells, tumor stroma or tumor vasculature. Antigens expressed on tumor cells that are suitable targets for mAb-SAg fusion protein therapy include erb/neu, MUC1, 5T4 and many others. Antibodies specific for tumor vasculature bind to a molecule expressed or localized or accessible at the cell surface of blood vessels, preferably the intratumoral blood vessels, of a vascularized tumor. Such molecules include endoglin (TEC-4 and TEC-11 antibodies), a TGFβ. receptor, E-selectin, P-selectin, VCAM-1, ICAM-1, PSMA, a VEGF/VPF receptor, an FGF receptor, a TIE, an αvβ3 integrin, pleiotropin, endosialin and MHC class II proteins. Such antibodies may also bind to cytokine-inducible or coagulant-inducible products of intratumoral blood vessels. Certain preferred agents will bind to aminophospholipids, such as phosphatidylserine or phosphatidylethanolamine.

A tumor cell-targeting antibody, or an antigen-binding fragment thereof, may bind to an intracellular component that is released from a necrotic or dying tumor cell. Preferably such antibodies are mAbs or fragments thereof that bind to insoluble intracellular antigen(s) present in cells that may be induced to be permeable, or in cell ghosts of substantially all neoplastic and normal cells, but are not present or accessible on the exterior of normal living cells of a mammal.

Anti-tumor stroma antibodies bind to a connective tissue component, a basement membrane component or an activated platelet component; as exemplified by binding to fibrin, RIBS (receptor-induced binding site) or LIBS (ligand-induced binding site).

Fusion protein optionally include linkers or spacers. Numerous types of disulfide-bond containing linkers are known that can be successfully employed to fuse the SAg to an antibody or fragment, certain linkers are preferred based on differing pharmacological characteristics and capabilities. For example, linkers that contain a disulfide bond that is sterically “hindered” are preferred, due to their greater stability in vivo, thus preventing release of the SAg moiety prior to binding at the site of action.

Preferably one or a plurality of fusion proteins is administered each with a different SAg or SAg homologue with a broad and minimally overlapping Vβ/α profile. Preferably the total Vβ/Vα profile of the fusion proteins exhibits recognition of at least 5 different Vβ/α-expressing T cell clones and a maximum of 24Vβ/α-expressing T cell clones or induce Vβ/Vα expression in human T cells (using well described in vitro RNA/DNA or T cell surface expression assays) after stimulation with individual SAgs. The preferred mixture of fusion proteins comprises native superantigens or more preferably any one or plurality of native egc superantigens or their functional superantigen homologues or mixtures of native egc superantigens and their superantigen homologues as the SAg component of the fusion protein(s).

Coaguligand

Superantigens may be conjugated to, or operatively associated with, polypeptides that are capable of directly or indirectly stimulating coagulation, thus forming a “coaguligand” (Barinaga M et al., Science 275:482-4 (1997); Huang X et al., Science 275:547-50 (1997); Ran S et al., Cancer Res 1998 Oct. 15; 58(20):4646-53; Gottstein C et al., Biotechniques 30:190-4 (2001)).

In coaguligands, the antibody may be directly linked to a direct or indirect coagulation factor, or may be linked to a second binding region that binds and then releases a direct or indirect coagulation factor. The “second binding region” approach generally uses a coagulant-binding antibody as a second binding region, thus resulting in a bispecific antibody construct. The preparation and use of bispecific antibodies in general is well known in the art, and is further disclosed herein.

Coaguligands are prepared by recombinant expression. The nucleic acid sequences encoding the SAg are linked, in-frame, to nucleic acid sequences encoding the chosen coagulant, to create an expression unit or vector. Recombinant expression results in translation of the new nucleic acid, to yield the desired protein product.

Where coagulation factors are used in connection with the present invention, any covalent linkage to the SAg should be made at a site distinct from the functional coagulating site. The compositions are thus “linked” in any operative manner that allows each region to perform its intended function without significant impairment. Thus, the SAg binds to and stimulates T cells, and the coagulation factor promotes blood clotting.

Preferred coagulation factors are Tissue Factor (“TF”) compositions, such as truncated TF (“tTF”), dimeric, multimeric and mutant TF molecules. tTF is a truncated TF that is deficient in membrane binding due to removal of sufficient amino acids to result in this loss. “Sufficient” in this context refers to a number of transmembrane amino acids originally sufficient to insert the TF molecule into a cell membrane, or otherwise mediate functional membrane binding of the TF protein. The removal of a “sufficient amount of transmembrane spanning sequence” therefore creates a tTF protein or polypeptide deficient in phospholipid membrane binding capacity, such that the protein is substantially soluble and does not significantly bind to phospholipid membranes. tTF thus substantially fails to convert Factor VII to Factor VIIa in a standard TF assay yet retains so-called catalytic activity including the ability to activate Factor X in the presence of Factor VIIa.

U.S. Pat. No. 5,504,067, specifically incorporated herein by reference, describes tTF proteins. Preferably, the TFs for use herein will generally lack the transmembrane and cytosolic regions (amino acids 220-263) of the protein. However, the tTF molecules are not limited to those having exactly 219 amino acids.

Any of the truncated, mutated or other TF constructs may be prepared in dimeric form employing the standard techniques of molecular biology and recombinant expression, in which two coding regions are arranged in-frame and are expressed from an expression vector. Various chemical conjugation technologies may be employed to prepare TF dimers. Individual TF monomers may be derivatized prior to conjugation.

The tTF constructs may be multimeric or polymeric, which means that they include 3 or more TF monomeric units. A “multimeric or polymeric TF construct” is a construct that comprises a first monomeric TF molecule (or derivative) linked to at least a second and a third monomeric TF molecule (or derivative). The multimers preferably comprise between about 3 and about 20 such monomer units. The constructs may be readily made using either recombinant techniques or conventional synthetic chemistry.

TF mutants deficient in the ability to activate Factor VII are also useful. Such “Factor VII activation mutants” are generally defined herein as TF mutants that bind functional Factor VII/VIIa, proteolytically activate Factor X, but substantially lack the ability to proteolytically activate Factor VII.

The ability of such Factor VII activation mutants to function in promoting tumor-specific coagulation is requires their delivery to the tumor vasculature and the presence of Factor VIIa at low levels in plasma. Upon administration of a conjugate of a Factor VII activation mutant, the mutant will be localize within the vasculature of a vascularized tumor. Prior to localization, the TF mutant would be generally unable to promote coagulation in any other body sites, on the basis of its inability to convert Factor VII to Factor VIIa. However, upon localization and accumulation within the tumor region, the mutant will then encounter sufficient Factor VIIa from the plasma in order to initiate the extrinsic coagulation pathway, leading to tumor-specific thrombosis. Exogenous Factor VIIa could also be administered to the patient to interact with the TF mutant and tumor vasculature.

Any one or more of a variety of Factor VII activation mutants may be prepared and used in connection with the present invention. The Factor VII activation region generally lies between about amino acid 157 and about amino acid 167 of the TF molecule. Residues outside this region may also prove to be relevant to the Factor VII activating activity. Mutations are inserted into any one or more of the residues generally located between about amino acid 106 and about amino acid 209 of the TF sequence (WO 94/07515; WO 94/28017; each incorporated herein by reference).

A variety of other coagulation factors may be used in connection with the present invention, as exemplified by: the agents set forth below. Thrombin, Factor V/Vα and derivatives, Factor VIII/VIIIa and derivatives, Factor IX/IXa and derivatives, Factor X/Xa and derivatives, Factor XI/XIa and derivatives, Factor XII/XIIa and derivatives, Factor XIII/XIIIa and derivatives, Factor X activator and Factor V activator may be used in the present invention.

The preferred coaguligand is fused in frame with nucleic acids encoding a SAg of any type or in combination, although one or a plurality of native SAgs in the enterotoxin gene cluster (egc) SEG, SEI, SEM, SEN, SEQ or one or more of a native egc superantigen or egc superantigen homologue or a mixture of native egc superantigens and egc superantigen homologues is/are preferred. Other native SAg or SAg homologues such as SEA, SEB, SEC, SED, SEE, SEQ, SER, SEU, TSST-1 and Y. pseudotuberculosis used alone or in combinations among themselves or with egc superantigens are also useful. The collective Vβ/Vα profile of the final preparation of single or multiple SAg-coaguligands exhibiting a minimum activation/recognition of 5 different Vβ/α-expressing T cell clones or induction of Vβ/Vα expression in human T cells (using well described in vitro RNA/DNA or T cell surface expression assays) after stimulation with individual SAgs.

The coaguligand conjugates described above are implanted or administered by injection, infusion or instillation, via any parenteral route to include intratumorally, intrathecally (e.g., intraperitoneally, intrapleurally, intravesicularly, intrapericardially) by infusion or injection in conventional or sustained release vehicles using standard protocols or those exemplified herein. Frequency of administration may be every 3-7 days. The SAgs in the conjugates are administered in doses of each superantigen in a of range of 0.1 pg-1.5 ng for each treatment. Cytokines from a group consisting of IL-15 (0.15-8 mg/kg), IL-7 (0.5 ug/day), IL-23 (0.1-200 ug/day) are given 1 to 7 days weekly for 1-4 weeks after each dose of egc SAg-coaguligand with or without high-dose IL-2 therapy consisting of 720,000 units per kg bolus i.v. infusion every 8 hours to tolerance after each dose of coaguligand.

### Cytokines as Fusion Partners

Cytokines are an effective partner for SAgs. Various cytokines, such as IL-2, IL-3, IL-7, IL-12, and IL-18, may be used.

A preferred fusion polypeptide comprises a SAg fused to anti-apoptotic cytokines. SAg stimulation of T cells can result in activation-driven cell death. Several cytokines and bacterial lipopolysaccharide (LPS) are known to interfere with this process (Vella et al., Proc. Natl. Acad. Sci. 95: 3810-3815 (1998)). IL-3, IL-7, IL-15, IL-17, IL-23, IL-27 prevent SAg-stimulated T cells from undergoing apoptosis in vivo and in vitro and promote T cell development and proliferation. In addition, because of their ability to promote selective proliferation by Th1 T cells, IL-12 and IL-18 are desirable. IL-18 is preferred for intratumoral injection because it induces tumor suppressive cytokines IFNγ and TNFα and IL-1β, and rescues cytotoxic T cells from apoptosis.

Accordingly, SAg-mAb conjugate as described above is fused recombinantly to the extracellular domains of one or more cytokines from a group consisting of IL-2. IL-7 or IL-3 or IL-12 or IL-15 or IL-17, IL-18, IL-23, IL-27. Other anti-T cell apoptosis agents such as LPS preparations of low virulence or a lipid A component (modified to induce less toxicity) are also effective antiapoptotic agents when conjugated biochemically to the superantigen-MoAb (or F(ab′)2, Fab, Fd or single chain Fv fragments) conjugate or if administered concomitantly with the SAg. Nucleic acids encoding the cytokine of choice is fused in frame with nucleic acids encoding the SAg. These conjugates are administered parenterally, intrathecally and/or intratumorally, intrapericardially, intravesicularly, intrapleurally, intralymphatically by infusion, instillation or injection in dosages of 0.01 pg to 0.1 ug for each SAg conjugate used in the fusion protein. The quantity of each cytokine in the egc SAg-cytokine fusion protein (consisting preferably of one or more of IL-7, IL-15, IL-23) ranges from 0.5 ng-200 ug.

Preferably one or a plurality of cytokine-containing fusion proteins is administered each with a different SAg with a broad and minimally overlapping Vβ/α profile. Preferably the total Vβ/Vα profile of the mixture of fusion protein exhibits activation/recognition of at least 3-5 different Vβ/α-expressing T cell clones or induce T cell Vβ/Vα expression (in well described in vitro RNA/DNA or T cell surface expression assays as given above) after stimulation with individual SAgs. The preferred SAgs in the fusion proteins are native egc SEs or egc homologues or combinations of native egc SEs and egc homologues.

### Costimulatory Molecules as Fusion Partners

Superantigens Conjugated to OX40L or 4-1BBL

A preferred fusion polypeptide comprises a SAg fused recombinantly to a potent costimulatory molecule, preferably the ECD of a transmembrane costimulatory protein. Examples of such costimulatory molecules are the OX-40 ligand (Godfrey et al., J. Exp. Med. 180: 757-762 (1994); Gramaglia I et al., J. Immunol. 161: 6510-6517 (1998); Maxwell J R et al., J. Immunol. 164: 107-112 (2000) or 4-1BB ligand (Kown B S et al., Proc. Natl. Acad. Sci. USA 86:1963-67 (1989); Shuford W W et al., J. Exp. Med. 186: 47-55 (1997) and CD-38 (Jackson D G et al., J. Immunol. 144: 2811-2817 (1990); Zilber et al., Proc. Natl. Acad. Sci. USA 97: 2840-2845 (2000). The preparation of such fusion proteins is achieved by recombinant methods in which nucleic acids encoding SAgs are fused in frame to nucleic acids encoding the ECD of the costimulatory molecule such as OX-40L (Godfrey et al., J. Exp. Med 180:757-762 (1994)) or 4-1BBL (Goodwin et al. Eur. J. Immunol. 23: 2631-2641 (1993); Melero I. et al., Eur. J. Immunol. 28: 1116-1121 (1998)).

| OX40 Ligand (Hikami, K., et al., Genes Immun. 1 (8), 521-522 (2000)) | |
|---|---|
| [SEQ ID NO: 84] | |
| 1 | MERVQPLEEN VGNAARPRFE RNKLLLVASV IQGLGLLLCF TYICLHFSAL QVSHRYPRIQ |
| 61 | SIKVQFT |
| 4-1BB Ligand (Alderson, M. R. et al., Eur. J. Immunol. 24 (9), | |
| 2219-2227 (1994)) | |
| [SEQ ID NO: 85] | |
| 1 | MEYASDASLD PEAPWPPAPR ARACRVLPWA LVAGLLLLLL LAAACAVFLA CPWAVSGARA |
| 61 | SPGSAASPRL REGPELSPDD PAGLLDLRQG MFAQLVAQNV LLIDGPLSWY SDPGLAGVSL |
| 121 | TGGLSYKEDT KELVVAKAGV YYVFFQLELR RVVAGEGSGS VSLALHLQPL RSAAGAAALA |
| 181 | LTVDLPPASS EARNSAFGFQ GRLLHLSAGQ RLGVHLHTEA RARHAWQLTQ GATVLGLFRV |
| 241 | TPEIPAGLPS PRSE |

It is preferred to delete from the conjugates or fusion polypeptides of the present invention any SAg epitope that binds to SAg-specific antibodies, including preexisting or natural antibodies). Such epitopes are deleted or substituted by Ala or by amino acid sequences not recognized by preexisting host antibodies. For example, a dominant epitope of SEB that is recognized by anti-SEB antibodies is the sequence at residues 225-234 (Nishi et al., J. Immunol. 158: 247-254 (1997). An epitope of SEA that is recognized by anti-SEA antibodies is the sequence at residues 121-149 (Hobieka et al., Biochem. Biophys. Res. Comm. 223: 565-571 (1996). Alternatively, to avoid issues with such preexisting immunity, SAgs such as YPM or C. perfringens toxin A to which humans do not have preexisting antibodies are selected. YPM, in addition, a natural RGD domain which gives it tumor localizing properties. The SE may be modified to reduce toxicity by altering its MHC class II binding affinity (e.g., SEA D227A-high affinity Zn++ dependent binding site).

Preferably, the tumor targeting structure in SAg conjugate (e.g., tumor specific antibody or fragment, or a tumor receptor ligand) has greater affinity for the tumor than the affinity of the SAg in the conjugate for the MHC class II molecule thus preventing the SAg from binding “promiscuously” to all MHC class II molecules receptors and favoring binding to the tumor. In the case of SEB, the dominant epitope for neutralizing antibodies, residues 225-234, is recombinantly or biochemically conjugated to the tumor targeting molecule (e.g., tumor specific antibody, etc.) so that it can sterically interfere with the recognition of the dominant epitope by preexisting antibodies in the host.

To further enhance the affinity of the tumor specific antibody in the fusion polypeptide for tumor cells in vivo, one preferably selects a tumor specific antibody that is specific for more than one antigenic structures of the tumor, the tumor stroma or the tumor vasculature (or any combination). The tumor specific antibody or antigen-binding fragment thereof can be made mono or divalent (like IgG), polyvalent like IgM to increase avidity or chimeric with multiple tumor specificities as described above. Thus, when the SAg-mAb conjugate is administered in vivo, it will preferentially bind to tumor cells rather than to endogenous anti-SAg antibodies or MHC class II receptors.

To reduce affinity of the SAg-mAb conjugate for endogenous MHC class II binding sites, the high affinity Zn++ dependent MHC class II binding site present in a number of SAgs (SEA, SEC2, SEC3, SED, SPEA, SPEC, SPEG, SPEH, SMEZ, SMEZ2, M. arthritides SAg) is deleted or replaced by an “inert” sequence(s) or amino acid. Such structural alterations in SE or SPEA are known to reduce the affinity for MHC class II from a Kd of 10⁻⁷ or 10⁻⁸ to a Kd of 10-5. SEB, SEC and SSA and other SAgs do not have such a high affinity Zn++-dependent MHC class II binding site but have multiple low affinity MHC class II binding sites (Kd of 10⁻⁵-10⁻⁷). In these cases, alteration of the MHC class II binding sites is not always necessary to further reduce affinity for MHC class II; mutation of one or two of the low affinity MHC class II binding sites will suffice in most instances.

Most importantly, tumor specific antibodies or their fragments in a SAg-mAb conjugate have higher affinities for tumor antigens (Kd of 10⁻¹¹-10⁻¹⁴ or lower) than (a) the affinity of the SAg for MHC class II binding sites (Kd 10⁻⁵ to 10⁻⁷) or (b) the affinity a dominant SAg epitope for a SAg-specific antibody (Kd 10⁻⁷ to 10⁻¹¹). Because of this, the conjugate will bind preferentially to the tumor target in vivo SAg-OX-40 ligand (OX-40L) or 4-1BB ligand (4-1BBL) are fused to a tumor specific targeting structure using recombinant SAgs. A most preferred construct combines the ECD of OX-40L or 4-1BBL with a high affinity tumor specific Fv antibody fragments. The nucleic acids encoding the ECD of OX-40L (Godfrey et al., supra or 4-1BBL (Goodwin et al., Eur. J. Immunol. 23: 2631-2641 (1993); Melero I. et al., Eur. J. Immunol. 28: 1116-1121 (1998)) are fused in frame with nucleic acids encoding a SAg of any type or in combination although one or a plurality native egc SAgs, SEG, SEI, SE, SEM, SEN, SEQ or functional superantigen homologues thereof or mixtures of native egc superantigens and egc homologues are preferred. Other native SAg or SAg homologues such as SEA, SEB, SEC, SED, SEE, SEQ, SER, SEU, TSST-1 and Y. pseudotuberculosis used alone or in combinations among themselves or with native egc SAgs or their SAg homologues are also useful. One or a plurality of fusion proteins is administered each consisting of a different SAg with the total Vβ/Vα profile of the final preparation exhibiting a minimum activation/recognition of 3-5 different Vβ/α-expressing T cell clones or induction of Vβ/Vα expression in human T cells (in well described in vitro RNA/DNA or T cell surface expression assays) after stimulation with individual SAgs. The SAgs in the conjugates are administered in doses of each superantigen in a range of 0.1 pg-1.5 ng for each treatment.

The SAg may be structurally modified to reduce antigenicity by deleting a dominant epitope and to reduce toxicity by altering its MHC class II binding affinity as described above. The tumor targeting structure may include but is not limited to a tumor receptor ligand or tumor-specific antibody or a fragment thereof. Preferably, the affinity of the tumor targeting structure is of higher affinity than is the affinity of the modified SAg for MHC class II. High affinity scFv constructs specific for the OX-40 receptor and 4-1BB receptor may be used in place of the OX40L and 4-1BBL in the SAg-tumor targeting construct.

The SE-OX-40L (or 4-1BB) conjugates described above are implanted or administered parenterally, intratumorally, intrathecally (e.g., intraperitoneally, intrapleurally, intrapericardially, intravesicularly) by infusion, instillation or injection in conventional or sustained release vehicles using standard protocols or those exemplified herein. Frequency of administration may be every 3-7 days for 1-6 weeks per cycle which may be repeated every 2-6 months. These conjugates and fusion proteins are administered with one or more cytokines from a group consisting of IL-7, IL-15, IL-23, IL-27 in doses given in the previous section.

### Biochemical Cross-Linkers

In the above fusion polypeptides or conjugates, the SAgs may be linked directly to a fusion partner or fused/conjugated via certain preferred biochemical linker or spacer groups. For chemical conjugates, cross-linking reagents are preferred and are used to form molecular bridges that bond together functional groups of two different molecules. Heterobifunctional crosslinkers can be used to link two different proteins in a step-wise manner while preventing unwanted homopolymer formation. Such cross-linkers are listed in Table 3, below.

Hetero-bifunctional cross-linkers contain two reactive groups one (e.g., N-hydroxy succinimide) generally reacting with primary amine group and the other (e.g., pyridyl disulfide, maleimides, halogens, etc.) reacting with a thiol group. Compositions to be crosslinked therefore generally have, or are derivatized to have, a functional group available. This requirement is not considered to be limiting in that a wide variety of groups can be used in this manner. For example, primary or secondary amine groups, hydrazide or hydrazine groups, carboxyl, hydroxyl, phosphate, or alkylating groups may be used for binding or cross-linking.

The spacer arm between the two reactive groups of a cross-linker may be of various length and chemical composition. A longer, aliphatic spacer arm allows a more flexible linkage while certain chemical groups (e.g., benzene group) lend extra stability or rigidity to the reactive groups or increased resistance of the chemical link to the action of various agents (e.g., disulfide bond resistant to reducing agents). Peptide spacers, such as Leu-Ala-Leu-Ala, are also contemplated.

It is preferred that a cross-linker have reasonable stability in blood. Numerous known disulfide bond-containing linkers can be used to conjugate two polypeptides. Linkers that contain a disulfide bond that is sterically hindered may give greater stability in vivo, preventing release of the agent prior to binding at the desired site of action.

A most preferred cross-linking reagents for use in with antibody chains is SMPT, a bifunctional cross-linker containing a disulfide bond that is “sterically hindered” by an adjacent benzene ring and methyl groups. Such steric hindrance of the disulfide bond may protect the bond from attack by thiolate anions (e.g., glutathione) which can be present in tissues and blood, and thereby help in preventing decoupling of the conjugate prior to the delivery to the target, preferably tumor, site. SMPT cross-links functional groups such as —SH or primary amines (e.g., the ε-amino group of Lys).

TABLE 3
Hetero-Bifunctional Cross-linkers

| | | Spacer arm length | |
|---|---|---|---|
| Linker | Advantages and Applications | after cross linking | |
| Succinimidyloxycarbonyl-α-(2- | Greater stability | 11.2 | A |
| pyridyldithio)toluene (SMPT) 1 | | | |
| N-succinimidyl 3-(2- | Thiolation | 6.8 | A |
| pyridyldithio)propionate (SPDP) 2 | | | |
| Sulfosuccinimidyl-6-[α-methyl-α-(2- | Extended spacer arm; Water-soluble | 15.6 | A |
| pyridyldithio)toluamido]hexanoate | | | |
| (Sulfo-LC-SPDP)1 | | | |
| Succinimidyl-4-(N- | Stable maleimide reactive group; | 11.6 | A |
| maleimidomethyl)cyclohexane-1- | conjugation of enzyme or other | | |
| carboxylate (SMCC) 1 | polypeptide to antibody | | |
| Succimimidyl-4-(N- | Stable maleimide reactive group; water- | 11.6 | A |
| maleimidomethyl)cyclohexane- | soluble | | |
| carboxylate (Sulfo-SMCC) 1 | | | |
| m-Maleimidobenzoyl-N- | Enzyme-antibody conjugation; hapten- | 9.9 | A |
| hydroxysuccinimide (MBS) 1 | carrier protein conjugation | | |
| m-Maleidmidobenzoyl-N- | Water-soluble | 9.9 | A |
| hydroxysulfosuccinimide (Sulfo- | | | |
| MBS) 1 | | | |
| N-Succinimidyl(4- | Enzyme-antibody conjugation | 10.6 | A |
| iodacetyl)aminobenzoate (SIAB) 1 | | | |
| Sulfosuccinimidyl(4- | Water-soluble | 10.6 | A |
| iodoacetyl)aminobenzoate (Sulfo- | | | |
| SIAB) 1 | | | |
| Succinimidyl-4-(p- | Enzyme-antibody conjugation; extended | 14.5 | A |
| maleimidophenyl)butyrate (SMPB) 1 | spacer arm | | |
| Sulfosuccinimidyl-4-(p- | Extended spacer arm | 14.5 | A |
| maleimidophenyl)butyate (Sulfo- | Water-soluble | | |
| SMPB) 1 | | | |
| 1-ethyl-3-(3- | Hapten-Carrier conjugation | 0 | |
| dimethylaminopropyl)carbodiimide | | | |
| hydrochloride N- | | | |
| Hydroxysulfosuccinimide | | | |
| (EDC/Sulfo-NHS) 3 | | | |
| p-Azidobenzoyl hydrazide (ABH) 4 | Reacts with sugar groups | 11.9 | A |

1 Reactive toward primary amines, sulfhydryls
2 Reactive toward primary amines
3 Reactive toward primary amines, carboxyl groups
4 Reactive toward carbohydrates, nonselective

Hetero-bifunctional photoreactive phenylazides containing a cleavable disulfide bond, for example, sulfosuccinimidyl-2-(p-azido salicylamido)-ethyl-1,3′-dithiopropionate. The N-hydroxy-succinimidyl group reacts with primary amino groups and the phenylazide (upon photolysis) reacts non-selectively with any amino acid residue.

Other useful cross-linkers, not considered to contain or generate a protected disulfide, include SATA, SPDP and 2-iminothiolane. The use of such cross-linkers is well known in the art.

Once conjugated, the conjugate is separated from unconjugated SAg and fusion partner polypeptides and from other contaminants. A large a number of purification techniques are available for use in providing conjugates of a sufficient degree of purity to render them clinically useful. Purification methods based upon size separation, such as gel filtration, gel permeation or high performance liquid chromatography, will generally be of most use. Other chromatographic techniques, such as Blue-Sepharose separation, may also be used.

### Increased Vulnerability of Superantigen-Exposed Tumor Cells to Chemotherapy

After exposure of carcinoma cells to SEs (or PBMCs activated by SAgs) in vivo and in vitro histologic changes appear to include osmotic swelling of the cytoplasm, cytoplasmic vacuoles, nuclear fragmentation, loss of intercellular boundaries and membrane disruption. Functionally, the tumor cells show permeability dysfunction reflected in a decrease in transepithelial resistance as well as a bidirectional increase in ion, small molecule and water transport. This effect endures for 4-48 hours after SE exposure and is enhanced by T cell cytokines induced by SAgs such as TNFα and IFNγ.

Conserved SAg fragments comprising amino acid sequences 147-163 of SEA-SEE previously described are useful in promoting the tumor killing effects of chemotherapy on tumor cells. These conserved sequence comprises an epitope on SEB which binds to tumor cell transcytosis receptor/transporter and induces a breakdown in barrier permeability resulting in osmotic swelling of tumor cells and transcytosis of SEs across the tumor cells. Functionally, the cells are freely permeable to carbachol and dextran and are unable to secrete chloride ions. Histologically, the carcinoma cells are swollen and hypertrophied and display cytoplasmic vacuoles and nuclear fragmentation.

One of the inventors has observed that tumor cells exposed to SAgs are also freely permeable to chemotherapeutic agents. With a dysfunctional permeability barrier, chemotherapeutic agents readily diffuse into the tumor cells leading rapidly to drug-induced apoptosis. This effect is not seen is cells which are not treated with SEs. Predictably, under these conditions, chemotherapy used, intrathecally, intrapleurally, intraperitoneally, intravesicularly intrapericardially or intratumorally induces killing of SAg-exposed tumor cells in doses that are well below the therapeutic range.

Structurally, amino acid sequence 147-163 of egc SEs show significant homology to the SE epitope on SEB which is known to interact with the transcytosis receptor/transporter on carcinoma cells. The homology of the egc SEs and other SEs with the 147-163 amino acid sequence of SEB is shown below.

Classical SEs: Sequences 147-163

| | | |
|---|---|---|
| SEA | KKNVTVQELDLQARRYL | (SEQ ID NO: 86) |
| SEB | KKKVTAQELDYLTRHYL | (SEQ ID NO: 87) |
| SEC1 | KKSVTAQELDIKARNFSL | (SEQ ID NO: 88) |
| SEC2 | KKSVTAQELDIKARNF | (SEQ ID NO: 89) |
| SEC3 | KKSVTAQELDIKA | (SEQ ID NO: 90) |
| SED | KKNVTVQELDAQARRYL | (SEQ ID NO: 91) |
| SEE | KKEVTVQELDLQARHYL | (SEQ ID NO: 92) |
| TSST-1 | KKQLAISTLDFEIRHQL | (SEQ ID NO: 93) |

Egc SEs: Sequences 147-163

| | | |
|---|---|---|
| SEG | KNMVTIQELDYKARHW | (SEQ ID NO: 94) |
| SEG | KKEVTAQEIDIKLRKY | (SEQ ID NO: 95) |
| SEI | KKLVTAQEIDVKLRRYL | (SEQ ID NO: 96) |
| SEM | KKLVTAQEIDTKLRRYL | (SEQ ID NO: 97) |
| SEN | KKKVYAQELDIKVRTK | (SEQ ID NO: 98) |
| SEO | KAKVTVQELDTKVRFKL | (SEQ ID NO: 99) |

Any SE which exhibits up to 40% homology with the conserved sequence of SEB is useful to induce chemotherapeutic sensitivity in epithelial tumor cells by activating the transcytosis receptor. Demonstration the SE-induced barrier permeability dysfunction in tumor cells and their susceptibility to chemotherapy is given in Example 7.

### Chemotherapeutic and Other Agents

Chemotherapeutic agents can be used before, together with or after intrathecal, intratumoral or parenterally/systemically-administered SAg to enhance the tumor-killing effect. Chemotherapy may also be administered before, after or together with one or a plurality of native SAgs or SAg fragments, homologues or fusion proteins. The SAgs is delivered by injection, instillation or infusion by any route including intravenously, intramuscularly, intradermally, intravesicularly, intrathecally, intrapleurally, intrapericardially, subcutaneously, intraperitoneally, and any other parenteral route. The egc SAgs are preferred SAg(s) for use in native form, and/or as homologues, fragments and fusion proteins. Chemotherapy is administered by infusion, instillation or injection by any parenteral route such as intrathecally, intratumorally, intravenously, intratumorally intramuscularly, intradermally, intravesicularly, intrathecally, intrapleurally, intrapericardially, subcutaneously, intraperitoneally concomitantly with SAg. Preferably chemotherapy is given together with SAg after 2-7 weeks of treatment with the SAgs or its homologues, fragments, fusion proteins or mixtures thereof alone (See Examples 3, 4, 6, 7). Anti-cancer chemotherapeutic drugs useful in this invention include but are not limited to antimetabolites, anthracycline, vinca alkaloid, anti-tubulin drugs, antibiotics and alkylating agents. Representative specific drugs that can be used alone or in combination include cisplatinum (CDDP), adriamycin, dactinomycin, mitomycin, caminomycin, daunomycin, doxorubicin, tamoxifen, taxol, taxotere, vincristine, vinblastine, vinorelbine, etoposide (VP-16), 5-fluorouracil (5FU), cytosine arabinoside, cyclophosphamide, thiotepa, methotrexate, camptothecin, actinomycin-D, mitomycin C, aminopterin, combretastatin(s) and derivatives and prodrugs thereof.

A variety of chemotherapeutic and pharmacological agents may be given separately or conjugated to a therapeutic protein of the invention. Exemplary antineoplastic agents that have been conjugated to proteins include doxorubicin, daunomycin, methotrexate and vinblastine. Moreover, the attachment of other agents such as neocarzinostatin, macromycin, trenimon and α-amanitin has been described. See U.S. Pat. Nos. 5,660,827; 5,855,866; and 5,965,132; each incorporated by reference herein. Those of ordinary skill in the art will know how to select appropriate agents and doses, although, as disclosed, the doses of chemotherapeutic drugs are preferably reduced when used in combination with SAgs according to the present invention.

Another newer class of drugs that are also termed “chemotherapeutic agents” comprises agents that induce apoptosis. Any one or more of such drugs, including genes, vectors, antisense constructs, siRNA constructs, and ribozymes, as appropriate, may be used in conjunction with SAgs.

Other agents useful herein are anti-angiogenic agents, such as Avastin, angiostatin, endostatin, vasculostatin, canstatin and maspin. Avastin or Bevacizumab is a recombinant humanized monoclonal antibody directed against vascular endothelial growth factor (VEGF). Human VEGF mediates neo-angiogenesis in normal and malignant vasculature. It is overexpressed in most malignancies, and high levels have correlated with a greater risk of metastasis. Avastin or bevacizumab binds VEGF and prevents its interaction with receptors (Flt-1 and KDR) on the surface of endothelial cells. Avastin 5 mg/kg intravenously is given every 14 days until disease progression is detected. The initial dose of Avastin is delivered over 90 minutes as an IV infusion. SAgs, preferably egc SEs, are administered before, during or after avastin and usually given once or twice weekly for up to 10 weeks.

Chemotherapeutic agents are administered as single agents or multidrug combinations, in full or reduced dosage per treatment cycle. They can be administered before, during or after intrathecal or intratumoral, intravesicular and parenteral SAg composition. In a preferred schedule, the chemotherapeutic agent is administered within 36 hours of the last of two to four treatments of SAg compositions administered intrathecally (intrapleurally) or intratumorally or intravenously.

The combined use of the preferred SAg compositions with low dose, single agent chemotherapeutic drugs is particularly preferred although this will work with all other SAgs as well. Indeed, this synergy of SAgs with chemotherapy allows the use of the more toxic superantigens in lower and subtoxic doses as a means of priming a tumor for killing by chemotherapy. The choice of chemotherapeutic drug in such combinations is determined by the nature of the underlying malignancy. For lung tumors, cisplatinum is preferred. For breast cancer, a microtubule inhibitor such as taxotere is the preferred. For malignant ascites due to gastrointestinal tumors, 5-FU is preferred. “Low dose” as used with a chemotherapeutic drug refers to the dose of single agents that is 10-95% below that of the approved dosage for that agent (by the U.S. Food and Drug Administration, FDA). If the regimen consists of combination chemotherapy, then each drug dose is reduced by the same percentage. A reduction of >50% of the FDA approved dosage is preferred although therapeutic effects are seen with dosages above or below this level, with minimal side effects.

Tumors to treat with SAgs (±chemotherapeutics) using intratumoral injection are preferably at least 6 cm³ and visible by x-ray, CT, ultrasound, bronchoscopy, laparoscopy, culdoscopy. Intratumoral localization of the agent being delivered is facilitated with fluoroscopic, CT or ultrasound guidance. Representative tumors that are treatable with this approach include but are not limited to hepatocellular carcinoma, lung tumors, brain tumors, head and neck tumors and unresectable breast tumors. Multiple tumors at different sites may be treated by intrathecal or intratumoral SAg.

The chemotherapeutic agent(s) selected for therapy of a particular tumor preferably is one with the highest response rates against that type of tumor. For example, for non-small cell lung cancer (NSCLC), cisplatinum-based drugs have been proven effective. Cisplatinum may be given parenterally or intratumorally. When given intratumorally, cisplatinum is preferentially in small volume around 1-4 ml although larger volumes can also work. The smaller volume is designed to increase the viscosity of the cisplatinum containing solution in order to minimize or delay the clearance of the drug from the tumor site. Other agents useful in NSCLC include the taxanes (paclitaxel and docetaxel), vinca alkaloids (vinorelbine), antimetabolites (gemcitabine), and camptothecin (irinotecan) both as single agents and in combination with a platinum agent.

The optimal chemotherapeutic agents and combined regimens for all the major human tumors are set forth in Bethesda Handbook of Clinical Oncology, Abraham J et al., Lippincott William & Wilkins, Philadelphia, Pa. (2001); Manual of Clinical Oncology, Fourth Edition, Casciato, D A et al., Lippincott William & Wilkins, Philadelphia, Pa. (2000) both of which are herein incorporated in entirety by reference.

In one embodiment, these recommended chemotherapeutic agents are used alone or combined with other chemotherapeutics in subtherapeutic or full doses. Alternatively, they may be administered parenterally by infusion, instillation or injection in doses 10-95% below the FDA recommended therapeutic dose. For intratumoral administration, the dose of a chemotherapeutic drug or biologic agent is preferably reduced 10- to 50-fold below the FDA-recommended dose for parenteral administration. Chemotherapy in full or reduced dose can be administered parenterally by injection, instillation or infusion parenterally by any route such as intrathecally, intratumorally intravenously, intramuscularly, intradermally, intravesicularly, intrathecally, intrapleurally, intrapericardially, subcutaneously, intraperitoneally concomitant with, before or after the SAg.

Cisplatinum has been widely used to treat cancer, with effective parenteral doses of 20 mg/m² for 5 days every three weeks for a total of three courses. Preferred dose per treatment for cisplatinum given intratumorally is 5-10 mg whereas for intrathecal use 20-80 mg may be administered. Intratumoral cisplatinum may be given every 7-14 days for 10-20 treatments whereas intrathecal cisplatinum may be given every 2-6 weeks for 10-20 treatments. Cisplatinum delivered in small volumes, e.g., 5-10 mg/1-3 ml saline is extremely viscous and may be retained in the tumor for a sustained period acting much like a controlled release drug from an inert surface. This is indeed one preferred mode of administration of cisplatinum when administered intratumorally with or without the superantigen.

When used before, together with or after egc SE administration, doses of chemotherapy may be 10-95% below the FDA recommended therapeutic dose. For intratumoral administration, the dose of a chemotherapeutic drug or biologic agent is preferably reduced 10- to 50-fold below the FDA-recommended dose for parenteral administration. Cisplatinum has been widely used to treat cancer, with effective doses of 20 mg/m² for 5 days every three weeks for a total of three courses. Preferred dose per treatment for intratumoral use of cisplatinum is 5-10 mg whereas for intrathecal use 20-80 mg may be administered. Intratumoral cisplatinum may be given every 7-14 days for 10-20 treatments whereas intrathecal cisplatinum may be given every 2-6 weeks for 10-20 treatments. Cisplatinum delivered in small volumes, e.g., 5-10 mg/1-3 ml saline is extremely viscous and may be retained in the tumor for a sustained period acting much like a controlled release drug from an inert surface. This is indeed the preferred mode of administration of cisplatinum when administered intratumorally with or without the superantigen. However the chemotherapy is also effective when given in non-viscous form either before, together with or after egc SAg therapy. Indeed, we have administered cisplatinum in non-viscous form intratumorally together with SAg which has induced a complete remission of a large (22 cm²) lung mass. This result was surprising since animal models showed that intratumoral injection of non-viscous cisplatin induced no significant anti-tumor effects (Smith et al., Anticancer Drugs 6: 717-726 (1995). Preferably, cisplatinum is administered together with the superantigen in the same syringe.

Other agents and therapies that are operable together with or after parenteral (e.g., intratumoral, intrapleural, intraperitoneal, intravesicular, intravenous) SAg include, radiotherapeutic agents, antitumor antibodies with attached anti-tumor drugs such as plant-, fungus-, or bacteria-derived toxin or coagulant, ricin A chain, deglycosylated ricin A chain, ribosome inactivating proteins, sarcins, gelonin, aspergillin, restricticin, a ribonuclease, a epipodophyllotoxin, diphtheria toxin, or Pseudomonas exotoxin. Additional cytotoxic, cytostatic or anti-cellular agents capable of killing or suppressing the growth or division of tumor cells include anti-angiogenic agents, apoptosis-inducing agents, coagulants, prodrugs or tumor targeted forms, tyrosine kinase inhibitors (Siemeister et al., 1998), antisense strategies, RNA aptamers, siRNA and ribozymes against VEGF or VEGF receptors (Saleh et al., 1996; Cheng et al., 1996; Ke et al., 1998; Parry et al., 1999; each incorporated herein by reference).

Any of a number of tyrosine kinase inhibitors are useful when administered before, together with, or after, intratumoral SAg. These include, for example, the 4-aminopyrrolo[2,3-d]pyrimidines (U.S. Pat. No. 5,639,757). Further examples of small organic molecules capable of modulating tyrosine kinase signal transduction via the VEGF-R2 receptor are the quinazoline compounds and compositions (U.S. Pat. No. 5,792,771). Tarceva or Erlotinib attaches to EGF receptors and thereby blocks the of EGF-mediated activation of tyrosine kinase. Tarceva 150 mg daily is administered before during or after parenteral (intrathecal, intrapleural and/or intravenous) SAg treatment (See Examples 1-7) and continued until disease progression or unacceptable toxicity occurs.

Other agents which may be employed in combination with SAgs are steroids such as the angiostatic 4,9(11)-steroids and C21-oxygenated steroids (U.S. Pat. No. 5,972,922). Thalidomide and related compounds, precursors, analogs, metabolites and hydrolysis products (U.S. Pat. Nos. 5,712,291 and 5,593,990) may also be used in combination with SAgs and other chemotherapeutic drugs agents to inhibit angiogenesis. These thalidomide and related compounds can be administered orally.

Certain anti-angiogenic agents that cause tumor regression may be administered before, together with, or after, intrathecal, intrapleural, intratumoral, intravenous or parenteral SAg. These include the bacterial polysaccharide CM101 (currently in clinical trials as an anti-cancer drug) and the antibody LM609. CM101 has been well characterized for its ability to induce neovascular inflammation in tumors. CM101 binds to and cross-links receptors expressed on dedifferentiated endothelium that stimulate the activation of the complement system. It also initiates a cytokine-driven inflammatory response that selectively targets the tumor. CM101 is a uniquely antiangiogenic agent that downregulates the expression VEGF and its receptors. Thrombospondin (TSP-1) and platelet factor 4 (PF4) may also be used together with or after intratumoral SAg. These are both angiogenesis inhibitors that associate with heparin and are found in platelet α granules.

Interferons and metalloproteinase inhibitors are two other classes of naturally occurring angiogenic inhibitors that can be used before, together with or after intratumoral SAg. Vascular tumors in particular are sensitive to interferon; for example, proliferating hemangiomas are successfully treated with IFNα. Tissue inhibitors of metalloproteinases (TIMPs), a family of naturally occurring inhibitors of matrix metalloproteases (MMPs), can also inhibit angiogenesis and can be used in combination (before, during or after) the SAgs.

Adoptive Immunotherapy: Use of Egc SE's Alone or with Immunocyte Survival-Promoting Agents to Prevent AICD of egc SE-Induced Effector T Cells.

The egc SAgs are used to stimulate T cells ex vivo for adoptive transfer into tumor bearing hosts. The egc SAgs are used to stimulate a broad spectrum of Vβ clones in order to achieve a maximal tumoricidal effect. Thus the egc SAgs are used as a plurality in order to activate at least 5 and up to 23 T cell Vβ clones. The Vβ profiles of all SAgs are shown in Table 16. The egc SAgs are preferred because collectively they stimulate 12 Vβ clones. They may supplemented with one or more of SEA, SEB, SEC1-3, SED, SEE, TSST-1 SEH, SEJ, SEK, SEP, SEQ, SEU in order to obtain the broadest array of Vβ stimulation. In addition, the T cells stimulated by SAg ex vivo are coincubated with cytokines IL-7, IL15, IL-23 in order to sustain longevity and function of SAg-induced effector CD8+ cells and CD4+ T cells. The very same cytokines are also be administered to the patients for 1-10 days after each infusion of SAg-activated T cells. A number of cell types can be used as the source of T cells. When cells from lymph nodes are used, all types of lymph nodes are contemplated (inguinal, mesenteric, superficial distal auxiliary, etc.). For ex vivo stimulation, they are removed aseptically and single cell suspensions are prepared by teasing under sterile conditions. Cell preparations then may be filtered (e.g., through a layer of nylon mesh), centrifuged and subjected to a gentle lysing procedure, if necessary.

Tumor-draining lymph node cells may be stimulated in vitro using a number of protocols. For example, a sufficiently large number of lymph node cells (i.e., a number adequate to show a tumoricidal reaction upon reinfusion) are exposed to superantigens (e.g., SEA, SEB, etc.) and diluted in synthetic culture media (e.g., RPMI 1640 with typical supplements) for the appropriate period of time (e.g., two days). Any number of standard culture techniques can be employed (e.g., 24-well plates in an incubator at 37° C. in a 5% CO₂ atmosphere). Cytokines IL-7, IL-15 and IL-23 are coincubated with the SAgs and T cells in order to preserve cellular function and prevent activation induced T cell death.

Following the incubation, the stimulated cells are harvested and washed with synthetic media containing no superantigens. At this point, the cells may be cultured further with other agents if desired (e.g., optionally with IL-2 to further expand their numbers). In any event, the cells are counted to determine the degree of proliferation and resuspended in appropriate media for therapy.

The stimulated cells are reintroduced to the host by a number of approaches. Preferably, they are injected intravenously. Optionally, the host is treated with one or more agents to promote the in vivo function and survival of the stimulated T cells (e.g., IL-2, IL-15, IL-7, IL-23). IL-15 is preferred.

Of course, the stimulated cells may be reintroduced in a variety of pharmaceutical formulations. These may contain such normally employed additives as binders, fillers, carriers, preservatives, stabilizing agents, emulsifiers, and buffers. Suitable diluents and excipients are, for example, water, saline, and dextrose. Methods of isolation, purification and stimulation of various cell types including lymph nodes, spleen and tumor infiltrating lymphocytes from both mice and humans are given in Examples 8.

### Pharmaceutical Compositions and Administration

One or a plurality of any SAg, SAg homologues, fragments, mutants, fusion proteins and conjugates (SAg agents) or mixtures thereof are administered by injection, infusion, instillation or implantation. A mixture of native egc SEs or any one or a plurality of native egc or functional egc homologues or mixtures of native egc superantigens and egc homologues are preferred. Any mixture of SAgs or SAg homologues would suffice provided they activate/recognize or induce T cell expression of at least 3-5 different TCR Vβ/Vαs in well described in vitro RNA/DNA or T cell surface expression assays in human T cell populations after stimulation/incubation with individual SAgs. Preferably, neutralizing antibodies against the selected SAg to be used are not present in the sera of patients.

The SAgs may be administered parenterally preferably intravenously by infusion, instillation or injection but also may be implanted or injected intratumorally, intrapleurally, intrathecally, intrapericardially, intravesicularly, subcutaneously, intralymphatically, intraarticularly, intradermally, intracranially, intraarticularly or intramuscularly. They may be administered in a controlled release formulation. SAg agents may be administered intrathecally in patients with malignant intrathecal fluid accumulation due to primary or metastatic tumors, e.g., malignant pleural effusions in patients with lung cancer or metastatic breast, gastric or ovarian cancer. SAg agents may also be administered intrathecally to patients with intrathecal and parenchymal tumor (e.g., involvement of pleura and lung parenchyma) but little or no fluid accumulation in the cavitary space. SAg agents may also be administered intrathecally to patients without malignant involvement or fluid accumulation in the cavitary space or its membranes but with primary or metastatic tumor of the organ (e.g., lung, stomach) and/or lymph nodes. For example, SAg agents may be administered intrapleurally to patients with primary lung cancer or lung metastases from other primary tumors (e.g., breast, ovary, gastric) without malignant involvement of the pleura or pleural space. In each of the above examples, intrathecal administration of the SAg agents may be administered simultaneously or sequentially with one or a plurality of the SAgs administered intratumorally, intralymphatically or intravenously.

SAg agents are administered every 3-10 days for up to three months. Dosages of individual SAg agents used for intrathecal, intratumoral, intralymphatic and intravenous administration range from 0.1 pg-1.5 ng.

SAg agents are also administered intratumorally to stimulate a T cell-based inflammatory response, including release of tumoricidal cytokines and induction of cytotoxic T cells. The amount of SAg agents administered to a single tumor site ranges from about 0.05-1 ng/kg body weight. The intratumoral dose of a cytotoxic drug administered to the tumor site will generally range from about 0.1 to 500, more usually about 0.5 to 300 mg/kg body weight, depending upon the nature of the drug, size of tumor, and other considerations.

When used to boost the titer of SAg specific antibodies, SAg agents may be incorporated in an adjuvant vehicle such as alum or Freund's incomplete adjuvant. These compositions are administered prior to, during or after intrathecal and/or intratumoral administration of the SAg agents.

They are administered subcutaneously, intramuscularly and intradermally by injection or infusion in doses ranging from 0.1 pg/kg to 1 ng/kg. To induce a maximum immune response, boosters with the SAg agents and vehicle at 1-6 month intervals are given.

The pharmaceutical compositions of the present invention will generally comprise an effective amount of at least a SAg composition dissolved or dispersed in a pharmaceutically acceptable carrier or aqueous medium. Combined therapeutics are also contemplated, and the same type of underlying pharmaceutical compositions may be employed for both single and combined medicaments. The intratumoral composition can be administered to the tumor by needle or catheter via percutaneous entry or via endoscopy, bronchoscopy, culdoscopy or other modes of direct vision including directly at the time of surgery. The composition can be localized into the tumor with CT and/or ultrasound guidance.

With each drug in each tumor, experience will provide an optimum level. One or more administrations may be employed, depending upon the lifetime of the drug at the tumor site and the response of the tumor to the drug. Administration may be by syringe, catheter or other convenient means allowing for introduction of a flowable composition into the tumor. Administration may be every three days, weekly, or less frequent, such as biweekly or at monthly intervals.

The phrases “pharmaceutically or pharmacologically acceptable” refer to molecular entities and compositions that do not produce an adverse, allergic or other untoward reaction when administered to an animal, or a human, as appropriate. Veterinary uses are equally included within the invention and “pharmaceutically acceptable” formulations include formulations for both clinical and/or veterinary use.

As used herein, “pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. For human administration, preparations should meet sterility, pyrogenicity, general safety and purity standards as required by U.S. Food and Drug Administration. Supplementary active ingredients can also be incorporated into the compositions.

“Unit dosage” formulations are those containing a dose or sub-dose of the administered ingredient adapted for a particular timed delivery. For example, exemplary “unit dosage” formulations are those containing a daily dose or unit or daily sub-dose or a weekly dose or unit or weekly sub-dose and the like.

### Injectable Formulations

The SAg composition of the present invention are preferably formulated for parenteral administration, e.g., introduction by injection, infusion or instillation directly into an affected organ cavity (intrathecal, intrapleural, intrapericardial or intravesicular administration) or tumor (intratumorally). They may also be administered intravenously, intramuscularly, intradermally, intraperitoneally, intrapleurally, intraarticularly. Means for preparing aqueous compositions that contain the SAg compositions are known to those of skill in the art in light of the present disclosure. Typically, such compositions can be prepared as injectables, either as liquid solutions or suspensions; solid forms suitable for using to prepare solutions or suspensions upon the addition of a liquid prior to injection can also be prepared; and the preparations can also be emulsified.

The pharmaceutical forms suitable for injectable use include sterile aqueous solutions or dispersions and sterile powders for the extemporaneous preparation of sterile injectable solutions or dispersions. In all cases, the form should be sterile and fluid to the extent that syringability exists. It should be stable under the conditions of manufacture and storage and should be preserved against the contaminating action of microorganisms, such as bacteria and fungi.

The SAg compositions can be formulated into a sterile aqueous composition in a neutral or salt form. Solutions as free base or pharmacologically acceptable salts can be prepared in water. Pharmaceutically acceptable salts, include the acid addition salts (formed with the free amino groups of the protein), and those that are formed with inorganic acids such as, for example, hydrochloric or phosphoric acids, or such organic acids as acetic, trifluoroacetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

Suitable carriers include solvents and dispersion media containing, for example, water. In many cases, it will be preferable to include isotonic agents, for example, sugars or sodium chloride.

Sterile injectable solutions are prepared by incorporating the active agents in the required amount in the appropriate solvent with various of the other ingredients enumerated above, as desired, followed by filtered sterilization. Generally, dispersions are prepared by incorporating the various sterilized active ingredients into a sterile vehicle that contains the basic dispersion medium and the required other ingredients from those enumerated above.

In the case of sterile powders for the preparation of sterile injectable solutions, the preferred methods of preparation are vacuum-drying and freeze-drying techniques that yield a powder of the active ingredient, plus any additional desired ingredient from a previously sterile-filtered solution thereof.

Suitable pharmaceutical compositions in accordance with the invention will generally include an amount of the SAg composition admixed with an acceptable pharmaceutical diluent or excipient, such as a sterile aqueous solution, to give a range of final concentrations, depending on the intended use. The techniques of preparation are generally well known in the art as exemplified by Remington's Pharmaceutical Sciences, 16th Ed. Mack Publishing Company, 1980, or most recent edition, incorporated herein by reference. Endotoxin contamination should be kept minimally at a safe level, for example, less that 0.5 ng/mg protein. Moreover, for human administration, preparations should meet sterility, pyrogenicity, general safety and purity standards as required by the U.S. Food and Drug Administration. Upon formulation, the therapeutic compositions are administered in a manner compatible with the dosage formulation and in such amount as is therapeutically effective.

Once in an acceptable pharmaceutical form, SAg are administered intrathecally including but not limited to intrapleurally, intraperitoneally, intrapericardially, intravesicularly and/or intratumorally and optionally intra-lymph node and/or parenterally (e.g., intravenously, intramuscularly, subcutaneously) by injection, instillation or infusion. SAg are also delivered simultaneously or sequentially via one or more routes, e.g., parenterally, intrapleurally, intraperitoneally, intrapericardially, intraarticularly, intratumorally and/or intravenously. SAg are also administered simultaneously or sequentially in the same or different vehicles, adjuvants and sustained release formulations.

### Sustained Release Formulations

SAg formulations are easily administered in a variety of dosage forms, including “slow release” capsules or “sustained release” preparations or devices. Slow release formulations, generally designed to result in a constant drug level over an extended period, are used to deliver a SAg composition as described herein. Such slow release formulations are implanted intrathecally or intratumorally. Controlled release formulations are prepared using polymers to complex or absorb the therapeutic compositions—SAgs, SAg homologues, chemotherapeutic agents or combined formulations of a SAg/homologue and a chemotherapeutic agent(s). The rate of release is regulated by (1) selection of appropriate macromolecules, for example polyesters, polyamino acids, polyvinyl, pyrrolidone, ethylenevinylacetate, methylcellulose, carboxymethylcellulose, and protamine sulfate, (2) the concentration of the macromolecules and (3) the method of incorporation of the active agents into the formulation.

Another method to control the duration of action of the present controlled release preparations is to incorporate the SAgs, SAg homologues and/or chemotherapeutic drugs into particles of a polymeric material such as polyesters, polyamino acids, hydrogels, for example, poly(2-hydroxyethyl-methacrylate) or poly(vinylalcohol); polylactides (e.g., U.S. Pat. No. 3,773,919); copolymers of L-glutamic acid and γ-ethyl-L-glutamate; non-degradable ethylene-vinyl acetate; degradable lactic acid-glycolic acid copolymers, such as the Lupron Depot™ (injectable microspheres of lactic acid-glycolic acid copolymer and leuprolide acetate); and poly-D-(−)-3-hydroxybutyric acid.

Alternatively, instead of incorporating the bioactive/pharmaceutically active agents into polymeric particles, the active agents may rather be entrapped in microcapsules prepared by interfacial polymerization. Examples include hydroxymethylcellulose or gelatin-microcapsules and poly(methylmethacrylate)-microcapsules, respectively, or in colloidal drug delivery systems, for example, liposomes, albumin microspheres, microemulsions, nanoparticles, and nanocapsules or in macroemulsions. Such techniques are disclosed in Remington's Pharmaceutical Sciences (1980 or most recent edition). Nanoparticles consisting of SAg, SAg homologue and/or chemotherapeutic agents are delivered intrathecally or intratumorally via insufflation using a gas or air propellant.

While polymers such as ethylene-vinyl acetate and lactic acid-glycolic acid enable release of molecules for over 100 days, certain hydrogels release proteins for shorter time periods. For example, it is known that when encapsulated antibodies remain in the body for a prolonged period, they may denature or aggregate as a result of exposure to moisture at 37° C., thus reducing biological activity. Rational strategies are available for stabilization, and they depend on the mechanism involved. For example, if the aggregation mechanism involves intermolecular S—S bond formation through thio-disulfide interchange, stabilization is achieved by modifying sulfhydryl residues, lyophilizing from acidic solutions, controlling moisture content, using appropriate additives, developing specific polymer matrix compositions, and the like.

A particularly attractive sustained release preparation for use herein comprises collagen and an effective amount of SAg (or homologue) and a cytotoxic drug, as described by Luck et al., RE35,748 and Roskos et al., U.S. Pat. No. 6,077,545. More detail on preparation is given in Example 2.

The collagen composition can be used in the treatment of a wide variety of tumors including carcinomas, sarcomas and melanomas. Specific types of tumors include such basal cell carcinoma, squamous cell carcinoma, melanoma, soft tissue sarcoma, solar keratoses, Kaposi's sarcoma, cutaneous malignant lymphoma, Bowen's disease, Wilm's tumor, hepatomas, colorectal cancer, brain tumors; mycosis fungoides, Hodgkin's lymphoma, polycythemia vera, chronic granulocytic leukemia, lymphomas, oat cell sarcoma, etc. The collagen and other composition will be administered to a tumor to provide a cytotoxic amount of drug at the tumor site. The amount of cytotoxic drug administered to the tumor site will generally range from about 0.1 to 500 mg/kg body weight, more usually about 0.5 to 300 mg/kg, depending upon the nature of the drug, size of tumor, and other considerations. Vasoconstrictive agents will generally be present in from 1 to 50% (w/w) of the therapeutic agent. In view of the wide diversity of tumors, nature of tumors, effective concentrations of drug, relative mobility and the like, a definitive range cannot be specified. With each drug in each tumor, experience will provide an optimum level. One or more rounds of administration may be employed, depending upon the lifetime of the drug at the tumor site and the response of the tumor to the drug. Administration may be by syringe, catheter or other convenient means allowing for introduction of a flowable composition into the tumor. Administration may be every three days, weekly, or less frequent, such as biweekly or at monthly intervals.

Illustrative of the manner of sustained administration would be administration of cis-diaminodichloroplatinum (CDDP). Drug concentrations in the sustained release preparation may vary from 0.01 to 50 mg/ml. Injection may be at one or more sites depending on the size of the lesion. Needles of about 1-2 mm diameter are convenient. For multiple injection, templates with predrilled holes may be employed. The drug dose will normally be less than 100 mg/m² body surface area.

The present invention is particularly advantageous against those tumors or lesions that are clinically relevant because of high frequency. The compositions provide therapeutic gain with tumors greater than 100 mm³, more particularly, greater than 150 mm³, in volume.

Administration by controlled release of SAg and/or a chemotherapeutic drug may be used advantageously in conjunction with other forms of therapy. The tumors or lesions may be irradiated prior and/or subsequent to SAg administration by controlled release. Dose rates may vary from about 20 to 250 rad/min, usually 50 to 150 rad/min, depending on the lesion, period of exposure, and the like. Hyperthermia (heat) may be used as an adjunctive treatment. Treatment will usually involve heating the tumor and its surrounding tissue to a temperature of about 43° for between about 5 and 100 min.

### Intratumoral Administration

SAg or plurality of different SAgs and SET or a plurality of SETs or one or more of biologically active homologues, variants, fragments of SAg or fusion polypeptides or conjugates comprising SAgs as described herein is/are used for direct intratumoral treatment of a tumor mass. SAgs include Staphylococcal enterotoxins A, B, C1, C2, C3. D, E, F, G, H, I, J, K, L, M, N, O, P, Q, R, U, SpE's, YPM, M. arthritides, C. perfringens exotoxin for direct intratumoral treatment of tumor masses. A mixture of native egc superantigens or egc homologues or a mixture of native egc superantigens and their egc homologues is preferred although any one or a mixture of SAgs or SAg homologues would suffice provided the preparation activates/recognizes or induces expression of at least 5 different T cell Vβ/Vαs in previously described in vitro RNA/DNA or T cell surface expression assays after stimulation/incubation with individual SAgs. Preferably the SAgs used do not have neutralizing antibodies against them in patient sera.

Tumor masses in any organ or site are treated; the mass is palpated and/or visualized on x-ray, CT scan, MRI or ultrasound. Intratumoral administration is performed, where possible, with fluoroscopic, CT or ultrasound guidance.

For intratumoral administration, the dose of a chemotherapeutic drug or biologic agent is preferably reduced 10- to 50-fold below the FDA-recommended dose for parenteral administration. Preferred dose per treatment for intratumoral cisplatinum is 5-10 mg. every 7-14 days for 10-40 treatments. Cisplatinum delivered in small volumes, e.g., 5-10 mg/1-5 ml saline is extremely viscous and is retained in the tumor for a sustained period acting much like a controlled release drug released from an inert surface. This is indeed the preferred mode of administration of cisplatinum when administered intratumorally with or without the superantigen. Preferably cisplatinum is administered together with the superantigen in the same syringe.

The SAgs is dissolved in a conventional vehicle such as saline or it is incorporated into a controlled release formulation (mixture or suspension) preferably biodegradable. All of the biocompatible and biodegradable and controlled release formulations described herein are useful. These formulations also include but are not limited to, ethylene-vinyl acetate (EVAc: Elvax 40W, Dupont), bioerodible polyanhydrides, polyimino carbonate, sodium alginate microspheres and hydrogels. Dosages of each SAg in the mixture range from 0.1 pg-1.5 ng. The poly-(D-, L- or DL-lactic acid/polyglycolide) copolymers are preferred.

For intratumoral administration, the SAg composition is preferably administered once weekly, and this schedule is continued until the tumor has shrunk significantly. Generally 3-10 treatments are sufficient. In some cases, the tumor expands in size during such intratumoral SAg therapy. This is a result of SAg-stimulated accumulation of inflammatory cells and edema. Despite this enlargement, histological examination of such tumors during this phase shows evident tumoricidal effects with inflammatory cell infiltrates.

In the case of an enlarging tumor or a slowly regressing tumor when SAg therapy is given alone, conventional chemotherapy is administered to promote tumor killing. A chemotherapeutic agent is preferably administered intratumorally alone or together with SAg. Importantly, the chemotherapeutic agent should be given in doses well below those prescribed for systemic use of the same agent. Preferably, intratumoral chemotherapy will comprise use of a selected single agent which is known in the art to be effective against a particular tumor. Moreover, intratumoral combination chemotherapy wherein each agent is given in a reduced dose can also be used. Full-dose or reduced-dose systemic chemotherapy can also be used together with, or shortly after, intratumoral SAg therapy. As with intrathecal administration described herein, intratumoral delivery may be carried out in an outpatient setting as it requires no hospitalization.

The intratumoral therapy with a SAg and or a SAg homologue is used to treat a wide variety of neoplastic lesions. Indeed, an improvement in 5-year survival from 16% to 26% of small cell lung cancer was produced by increase in local control accomplished by altering the fractionation of radiation therapy (Turisi et al., N. Eng. J. Med. 340: 265-270 (1999)). Illustrative tumors amenable to intratumoral therapy with SAgs include carcinomas, sarcomas and melanomas, including such as basal cell carcinoma, squamous cell carcinoma, soft tissue sarcoma, solar keratosis, Kaposi's sarcoma, cutaneous malignant lymphoma, Bowen's disease, Wilm's tumor, neuroblastoma, gliomas astrocytomas, hepatoma, colorectal cancer, brain tumors, mycosis fungoides, Hodgkin's lymphoma, polycythemia vera, chronic granulocytic leukemia, lymphomas, oat cell sarcoma, breast carcinoma etc. The intratumoral SAg is of particular advantage for tumors or lesions which are among the most important clinically because of their frequency. The compositions and methods disclosed herein provide therapeutic gain with tumors exceeding 100 mm³ in volume, even tumors exceeding 150 mm³.
Superantigens with Radiation Therapy

Local radiation to any tumor sites or the mediastinum using the traditional standard dose of 60-65 gy is given concomitant with parenteral (e.g., intrathecal, intravenous, intravesicular, intrapleural intralymphatic or intratumoral) SAg. The radiotherapy is also be given before, during or after the SAg therapy but in either case there is a hiatus of no more than 30 days between the start of SAg therapy and the start or conclusion of radiotherapy. The median survival of patients given this type of radiotherapy alone is 5% at one year whereas the combined modality improves the median survival to more than two years.

In general, local radiation therapy alone has minimal efficacy in contributing to long-term disease control in advanced carcinomas. While radiation is an effective palliative measure to relieve symptoms, only a very small minority of patients achieve long-term survival when treated with radiation alone. However, radiation synergizes with SAg therapy in shrinking tumors and prolonging survival. Radiation is given to bulky or symptomatic lung lesions before, during or after SAg therapy. Preferably it is started 1-2 weeks before SAg treatment and continued simultaneously with SAg for 1-4 weeks until the full courses of SAg and radiation are completed. It may also be started after SAg treatment preferably within 24 hours of the last SAg treatment. Radiation may also be given to a malignant lesion or a tumous body cavity before, together with or after the site has been injected with SAg intratumoraly or intrathecally and/or systemic/parenteral chemotherapy. It may also be administered to a malignant lesion or site not injected specifically with SAg. In this case the SAg may be given systemically or intrathecally but not directly to the radiated tumor mass or site. As an example of the synergy between SAg therapy and radiation therapy, a 82 year old man with a non-small cell lung cancer and no prior treatment, presented with a 1200 cc left pleural effusion pleural fluid cytotolgy positive for adenocarcinoma) and a left mediastinal mass. He was given a 6 week course of fractionated radiation (total dose: 60 gy) to the mediastinal mass for submassive hemoptysis. Two weeks after starting radiation, egc SE (250-500 pg) was given intrapleurally every week for 6 weeks and intravenously every day for 4 weeks. Both the pleural effusion and mediastinal mass remitted completely. The malignant pleural effusion recurred 6 months after completing treatment and was retreated with egc SE (100 pg) intravenously every day for 30 days. His effusion remitted within one month and he remained in complete remission from his cancer for 30 months thereafter. Regimens for the use of intratumoral SAg and intratumoral and/or systemic use of chemotherapy are described in previous sections on chemotherapy and in Examples 1-7. Radiation may also be used with chemotherapy in these settings together with systemic and/or intratumoral SAg and intratumoral or systemic chemotherapy.

Radiation techniques are preferably continuous rather than split. Hyperfractionated radiation, employing multiple daily fractions of radiation are preferred to conventionally fractionated radiation. Radiation doses varies from 40-70 gy although a dose between 60 and 70 gy dose is preferred. It is contemplated that radiation doses considered to be subtherapeutic and up to 70% below the conventional doses are also useful when used before, during or after a course of SAg therapy.

### Tumor Models and Procedures for Evaluating Anti-Tumor Effects Studies

The various SAg compositions described herein are tested for therapeutic efficacy in several well established rodent models which are considered to be highly representative of a broad spectrum of human tumors. These approaches are described in detail in Geran, R. I. et al., “Protocols for Screening Chemical Agents and Natural Products Against Animal Tumors and Other Biological Systems (Third Edition)”, Canc. Chemother. Reports, Pt 3, 3:1-112, which is hereby incorporated by reference in its entirety.

### A. Calculation of Mean Survival Time (MST)

MST (days) is calculated according to the formula:

$$\frac{S + AS ( A \text{-} 1 ) - ( B + 1 ) NT}{S ( A \text{-} 1 ) - NT}$$

Day: Day on which deaths are no longer considered due to drug toxicity. For example, with treatment starting on Day 1 for survival systems (such as L1210, P388, B16, 3LL, and W256): Day A=Day 6; Day B=Day beyond which control group survivors are considered “no-takes.”
S: If there are “no-takes” in the treated group, S is the sum from Day A through Day B. If there are no “no-takes” in the treated group, S is the sum of daily survivors from Day A onward.
S(A-1): Number of survivors at the end of Day (A-1).
Example: for 3LE21, S(A-1)=number of survivors on Day 5.
NT: Number of “no-takes” according to the criteria given in Protocols 7.300 and 11.103.

### B. T/C Computed for All Treated Groups

$$T \text{/} C = \frac{M S T of treated group}{M S T of control group} \times 100$$

Treated group animals surviving beyond Day Bare eliminated from calculations (as follows):

| No. of survivors in treated | Percent of “no-takes” | |
|---|---|---|
| group beyond Day B | in control group | Conclusion |
| 1 | Any percent | “no-take” |
| 2 | <10 | drug inhibition |
| | ³10 | “no-takes” |
| ³3 | <15 | drug inhibitions |
| | ³15 | “no-takes” |

Positive control compounds are not considered to have “no-takes” regardless of the number of “no-takes” in the control group. Thus, all survivors on Day B are used in the calculation of T/C for the positive control. Surviving animals are evaluated and recorded on the day of evaluation as “cures” or “no-takes.”

Calculation of Median Survival Time (MedST)

MedST is the median day of death for a test or control group. If deaths are arranged in chronological order of occurrence (assigning to survivors, on the final day of observation, a “day of death” equal to that day), the median day of death is a day selected so that one half of the animals died earlier and the other half died later or survived. If the total number of animals is odd, the median day of death is the day that the middle animal in the chronological arrangement died. If the total number of animals is even, the median is the arithmetical mean of the two middle values. Median survival time is computed on the basis of the entire population and there are no deletion of early deaths or survivors, with the following exception:

C. Computation of MedST from Survivors

If the total number of animals including survivors (N) is even, the MedST (days) (X+Y)/2, where X is the earlier day when the number of survivors is N/2, and Y is the earliest day when the number of survivors (N/2)−1. If N is odd, the MedST (days) is X.

D. Computation of MedST from Mortality Distribution

If the total number of animals including survivors (N) is even, the MedST (days) (X+Y)/2, where X is the earliest day when the cumulative number of deaths is N/2, and Y is the earliest day when the cumulative number of deaths is (N/2)+1. If N is odd, the MedST (days) is X. “Cures” and “no-takes” in systems evaluated by MedST are based upon the day of evaluation. On the day of evaluation any survivor not considered a “no-take” is recorded as a “cure.” Survivors on day of evaluation are recorded as “cures” or “no-takes,” but not eliminated from the calculation.

E. Calculation of Approximate Tumor Weight from Measurement of Tumor Diameters with Vernier Calipers

The use of diameter measurements (with Vernier calipers) for estimating treatment effectiveness on local tumor size permits retention of the animals for lifespan observations. When the tumor is implanted sc, tumor weight is estimated from tumor diameter measurements as follows. The resultant local tumor is considered a prolate ellipsoid with one long axis and two short axes. The two short axes are assumed to be equal. The longest diameter (length) and the shortest diameter (width) are measured with Vernier calipers. Assuming specific gravity is approximately 1.0, and Pi is about 3, the mass (in mg) is calculated by multiplying the length of the tumor by the width squared and dividing the product by two. Thus,

$$Tumor weight ( mg ) = \frac{length ( mm ) \times ( width [ mm ] ) 2}{2} or \frac{L \times ( W ) 2}{2}$$

The reporting of tumor weights calculated in this way is acceptable inasmuch as the assumptions result in as much accuracy as the experimental method warrants.

### F. Calculation of Tumor Diameters

The effects of a drug on the local tumor diameter may be reported directly as tumor diameters without conversion to tumor weight. To assess tumor inhibition by comparing the tumor diameters of treated animals with the tumor diameters of control animals, the three diameters of a tumor are averaged (the long axis and the two short axes). A tumor diameter T/C of 75% or less indicates activity and a T/C of 75% is approximately equivalent to a tumor weight T/C of 42%.

G. Calculation of Mean Tumor Weight from Individual Excised Tumors

The mean tumor weight is defined as the sum of the weights of individual excised tumors divided by the number of tumors. This calculation is modified according to the rules listed below regarding “no-takes.” Small tumors weighing 39 mg or less in control mice or 99 mg or less in control rats, are regarded as “no-takes” and eliminated from the computations. In treated groups, such tumors are defined as “no-takes” or as true drug inhibitions according to the following rules:

| Percent of | Percent | |
|---|---|---|
| small tumors | of “no-takes” | |
| in treated group | in control group | Action |
| ≦17 | Any percent | no-take; not used in calculations |
| 18-39 | <10 | drug inhibition; use in calculations |
| | ≧10 | no-takes; not used in calculations |
| ≧40 | <15 | drug inhibition; use in calculations |
| | ≧15 | Code all nontoxic tests “33” |

Positive control compounds are not considered to have “no-takes” regardless of the number of “no-takes” in the control group. Thus, the tumor weights of all surviving animals are used in the calculation of T/C for the positive control (T/C defined above) SDs of the mean control tumor weight are computed the factors in a table designed to estimate SD using the estimating factor for SD given the range (difference between highest and lowest observation). Biometrik Tables for Statisticians (Pearson E S, and Hartley H G, eds.) Cambridge Press, vol. 1, table 22, p. 165.

### II. Specific Tumor Models

### A. Lymphoid Leukemia L1210

Summary: Ascitic fluid from donor mouse is transferred into recipient BDF1 or CDF1 mice. Treatment begins 24 hours after implant. Results are expressed as a percentage of control survival time. Under normal conditions, the inoculum site for primary screening is i.p., the composition being tested is administered i.p., and the parameter is mean survival time. Origin of tumor line: induced in 1948 in spleen and lymph nodes of mice by painting skin with MCA. J Natl Cancer Inst. 13:1328, 1953.

| | |
|---|---|
| Animals | One sex used for all test |
| | and control animals in one experiment. |
| Tumor Transfer | Inject ip, 0.1 ml of diluted ascitic fluid containing |
| | 10⁵ cells |
| Propagation | DBA/2 mice (or BDF1 or CDF1 for one generation). |
| Time of Transfer | Day 6 or 7 |
| Testing | BDF1 (C57BL/6 × DBA/2) or CDF1 (BALB/c × |
| | DBA/2) |
| Time of Transfer | Day 6 or 7 |
| Weight | Within a 3-g range, minimum weight of 18 g for males |
| | and 17 g for females. |
| Exp Size (n) | 6/group; No. of control groups varies according to |
| | number of test groups. |

### Testing Schedule

| DAY | PROCEDURE |
|---|---|
| 0 | Implant tumor. Prepare materials. Run positive control in every |
| | odd-numbered experiment. Record survivors daily. |
| 1 | Weigh and randomize animals. Begin treatment with |
| | therapeutic composition. Typically, mice receive 1 μg of the test |
| | composition in 0.5 ml saline. Controls receive saline alone. |
| | Treatment is one dose/week. Any surviving mice are sacrificed |
| | after 4 wks of therapy. |
| 5 | Weigh animals and record. |
| 20 | If there are no survivors except those treated with positive control |
| | compound, evaluate |
| 30 | Kill all survivors and evaluate experiment. |

Quality Control Acceptable control survival time is 8-10 days. Positive control compound is 5-fluorouracil; single dose is 200 mg/kg/injection, intermittent dose is 60 mg/kg/injection, and chronic dose is 20 mg/kg/injection. Ratio of tumor to control (T/C) lower limit for positive control compound is 135%.
Evaluation: Compute mean animal weight on Days 1 and 5, and at the completion of testing compute T/C for all test groups with >65% survivors on Day 5. A T/C value 85% indicates a toxic test. An initial T/C 125% is considered necessary to demonstrate activity. A reproduced T/C 125% is considered worthy of further study. For confirmed activity a composition should have two multi-dose assays that produce a T/C 125%.

### B. Lymphocytic Leukemia P388

Summary: Ascitic fluid from donor mouse is implanted in recipient BDF1 or CDF1 mice. Treatment begins 24 hours after implant. Results are expressed as a percentage of control survival time. Under normal conditions, the inoculum site for primary screening is ip, the composition being tested is administered ip daily for 9 days, and the parameter is MedST. Origin of tumor line: induced in 1955 in a DBA/2 mouse by painting with MCA. Scientific Proceedings, Pathologists and Bacteriologists 33:603, 1957.

| | |
|---|---|
| Animals | One sex used for all test |
| | and control animals in one experiment. |
| Tumor Transfer | Inject ip, 0.1 ml of diluted ascitic fluid containing |
| | 10⁶ cells |
| Propagation | DBA/2 mice (or BDF1 or CDF1 for one generation). |
| Time of Transfer | Day 7 |
| Testing | BDF1 (C57BL/6 × DBA/2) or CDF1 (BALB/c × |
| | DBA/2) |
| Time of Transfer | Day 6 or 7 |
| Weight | Within a 3-g range, minimum weight of 18 g for males |
| | and 17 g for females. |
| Exp Size (n) | 6/group; No. of control groups varies according to |
| | number of test groups. |

### Testing Schedule

| DAY | PROCEDURE |
|---|---|
| 0 | Implant tumor. Prepare materials. Run positive control in every |
| | odd-numbered experiment. Record survivors daily. |
| 1 | Weigh and randomize animals. Begin treatment with therapeutic |
| | composition. Typically, mice receive 1 μg of the test |
| | composition in 0.5 ml saline. Controls receive saline alone. |
| | Treatment is one dose/week. Any surviving mice are sacrificed |
| | after 4 wks of therapy. |
| 5 | Weigh animals and record. |
| 20 | If there are no survivors except those treated with positive control |
| | compound, evaluate |
| 30 | Kill all survivors and evaluate experiment. |

Acceptable MedST is 9-14 days. Positive control compound is 5-fluorouracil: single dose is 200 mg/kg/injection, intermittent dose is 60 mg/kg/injection, and chronic dose is 20 mg/kg/injection. T/C lower limit for positive control compound is 135% Check control deaths, no takes, etc.
Quality Control Acceptable MedST is 9-14 days. Positive control compound is 5-fluorouracil: single dose is 200 mg/kg/injection, intermittent dose is 60 mg/kg/injection, and chronic dose is 20 mg/kg/injection. T/C lower limit for positive control compound is 135%. Check control deaths, no takes, etc.
Evaluation: Compute mean animal weight on Days 1 and 5, and at the completion of testing compute T/C for all test groups with >65% survivors on Day 5. A T/C value of 85% indicates a toxic test. An initial T/C of 125% is considered necessary to demonstrate activity. A reproduced T/C 125% is considered worthy of further study. For confirmed activity a composition should have two multi-dose assays that produce a T/C 125%.

### C. Melanotic Melanoma B16

Summary: Tumor homogenate is implanted ip or sc in BDF1 mice. Treatment begins 24 hours after either ip or sc implant or is delayed until an sc tumor of specified size (usually approximately 400 mg) can be palpated. Results expressed as a percentage of control survival time. The composition being tested is administered ip, and the parameter is mean survival time. Origin of tumor line: arose spontaneously in 1954 on the skin at the base of the ear in a C57BL/6 mouse. Handbook on Genetically Standardized Jax Mice. Jackson Memorial Laboratory, Bar Harbor, Me., 1962. See also Ann NY Acad Sci 100, Parts 1 and 2, 1963.

| | |
|---|---|
| Animals | One sex used for all test |
| | and control animals in one experiment. |
| Propagation Strain | C57BL/6 mice |
| Tumor Transfer | Implant fragment sc by trochar or 12-g needle or |
| | tumor homogenate* every 10-14 days into axillary |
| | region with puncture in inguinal region. |
| Testing Strain | BDF1 (C57BL/6 × DBA/2) |
| Time of Transfer | Excise sc tumor on Day 10-14 from donor mice |
| | and implant as above |
| Weight | Within a 3-g range, minimum weight of 18 g for |
| | males and 17 g for females. |
| Exp Size (n) | 10/group; No. of control groups varies according to |
| | number of test groups. |

*Tumor homogenate: Mix 1 g or tumor with 10 ml of cold balanced salt solution, homogenize, and implant 0.5 ml of tumor homogenate ip or sc. Fragment: A 25-mg fragment may be implanted sc.

### Testing Schedule

| DAY | PROCEDURE |
|---|---|
| 0 | Implant tumor. Prepare materials. Run positive control in every |
| | odd-numbered experiment. Record survivors daily. |
| 1 | Weigh and randomize animals. Begin treatment with |
| | therapeutic composition. Typically, mice receive 1 μg of the |
| | test composition in 0.5 ml saline. Controls receive saline alone. |
| | Treatment is one dose/week. Any surviving mice are |
| | sacrificed after 8 wks of therapy. |
| 5 | Weigh animals and record. |
| 60 | Kill all survivors and evaluate experiment. |

Quality Control Acceptable control survival time is 14-22 days. Positive control compound is 5-fluorouracil: single dose is 200 mg/kg/injection, intermittent dose is 60 mg/kg/injection, and chronic dose is 20 mg/kg/injection. T/C lower limit for positive control compound is 135% Check control deaths, no takes, etc.
Evaluation: Compute mean animal weight on Days 1 and 5, and at the completion of testing compute T/C for all test groups with >65% survivors on Day 5. A T/C value of 85% indicates a toxic test. An initial T/C of 125% is considered necessary to demonstrate activity. A reproduced T/C 125% is considered worthy of further study. For confirmed activity a composition should have two multi-dose assays that produce a T/C 125%.
Metastasis after IV Injection of Tumor Cells

10⁵ B16 melanoma cells in 0.3 ml saline are injected intravenously in C57BL/6 mice. The mice are treated intravenously with 1 g of the composition being tested in 0.5 ml saline. Controls receive saline alone. The treatment is given as one dose per week. Mice sacrificed after 4 weeks of therapy, the lungs are removed and metastases are enumerated.

### C. 3LL Lewis Lung Carcinoma

Summary: Tumor may be implanted sc as a 2-4 mm fragment, or im as a 2×10⁶-cell inoculum. Treatment begins 24 hours after implant or is delayed until a tumor of specified size (usually approximately 400 mg) can be palpated. The composition being tested is administered ip daily for 11 days and the results are expressed as a percentage of the control. Origin of tumor line: arose spontaneously in 1951 as carcinoma of the lung in a C57BL/6 mouse. Cancer Res 15:39, 1955. See, also Malave, I. et al., J. Nat'l. Canc. Inst. 62:83-88 (1979).

| | |
|---|---|
| Animals | One sex used for all test |
| | and control animals in one experiment. |
| Propagation Strain | C57BL/6 mice |
| Tumor Transfer | Inject cells im in hind leg or implant fragment sc in |
| | axillary region with puncture in inguinal region. |
| | Transfer on day 12-14 |
| Testing Strain | BDF1 (C57BL/6 × DBA/2) or C3H mice |
| Time of Transfer | Same as above |
| Weight | Within a 3-g range, minimum weight of 18 g for |
| | males and 17 g for females. |
| Exp Size (n) | 6/group for sc implant, or 10/group for im implant.; |
| | No. of control groups varies according to number |
| | of test groups. |

### Testing Schedule

| DAY | PROCEDURE |
|---|---|
| 0 | Implant tumor. Prepare materials. Run positive control in |
| | every odd-numbered experiment. Record survivors daily. |
| 1 | Weigh and randomize animals. Begin treatment with |
| | therapeutic composition. Typically, mice receive 1 μg of the |
| | test composition in 0.5 ml saline. Controls receive saline |
| | alone. Treatment is one dose/week. Any surviving mice are |
| | sacrificed after 4 wks of therapy. |
| 5 | Weigh animals and record. |
| Final day | Kill all survivors and evaluate experiment. |

Quality Control: Acceptable im tumor weight on Day 12 is 500-2500 mg. Acceptable im tumor MedST is 18-28 days. Positive control compound is cyclophosphamide: 20 mg/kg/injection, qd, Days 1-11. Check control deaths, no takes, etc.
Evaluation: Compute mean animal weight when appropriate, and at the completion of testing compute T/C for all test groups. When the parameter is tumor weight, a reproducible T/C of 42% is considered necessary to demonstrate activity. When the parameter is survival time, a reproducible T/C of 125% is considered necessary to demonstrate activity. For confirmed activity a composition must have two multi-dose assays

### D. 3LL Lewis Lung Carcinoma Metastasis Model

This model has been utilized by a number of investigators. See, for example, Gorelik, E. et al., J. Nat'l. Canc. Inst. 65:1257-1264 (1980); Gorelik, E. et al., Rec. Results Canc. Res. 75:20-28 (1980); Isakov, N. et al., Invasion Metas. 2:12-32 (1982) Talmadge J. E. et al., J. Nat'l. Canc. Inst. 69:975-980 (1982); Hilgard, P. et al., Br. J. Cancer 35:78-86 (1977)).

Mice: male C57BL/6 mice, 2-3 months old. Tumor: The 3LL Lewis Lung Carcinoma was maintained by sc transfers in C57BL/6 mice. Following sc, im or intra-footpad transplantation, this tumor produces metastases, preferentially in the lungs. Single-cell suspensions are prepared from solid tumors by treating minced tumor tissue with a solution of 0.3% trypsin. Cells are washed 3 times with PBS (pH 7.4) and suspended in PBS. Viability of the 3LL cells prepared in this way is generally about 95-99% (by trypan blue dye exclusion). Viable tumor cells (3×10⁴-5×10⁶) suspended in 0.05 ml PBS are injected into the right hind foot pads of C57BL/6 mice. The day of tumor appearance and the diameters of established tumors are measured by caliper every two days. Typically, mice receive 1 μg of the composition being tested in 0.5 ml saline. Controls receive saline alone. The treatment is given as one or two doses per week.

In experiments involving tumor excision, mice with tumors 8-10 mm in diameter are divided into two groups. In one group, legs with tumors are amputated after ligation above the knee joints. Mice in the second group are left intact as nonamputated tumor-bearing controls. Amputation of a tumor-free leg in a tumor-bearing mouse has no known effect on subsequent metastasis, ruling out possible effects of anesthesia, stress or surgery. Surgery is performed under Nembutal anesthesia (60 mg veterinary Nembutal per kg body weight).

### Determination of Metastasis Spread and Growth

Mice are killed 10-14 days after amputation. Lungs are removed and weighed. Lungs are fixed in Bouin's solution and the number of visible metastases is recorded. The diameters of the metastases are also measured using a binocular stereoscope equipped with a micrometer-containing ocular under 8× magnification. On the basis of the recorded diameters, it is possible to calculate the volume of each metastasis. To determine the total volume of metastases per lung, the mean number of visible metastases is multiplied by the mean volume of metastases. To further determine metastatic growth, it is possible to measure incorporation of ^{125I}dUrd into lung cells (Thakur, M. L. et al., J. Lab. Clin. Med. 89:217-228 (1977). Ten days following tumor amputation, 25 mg of ¹²⁵IdUrd is inoculated into the peritoneums of tumor-bearing (and, if used, tumor-resected mice. After 30 min, mice are given 1 mCi of ¹²⁵IdUrd. One day later, lungs and spleens are removed and weighed, and a degree of ¹²⁵IdUrd incorporation is measured using a gamma counter.

Statistics: Values representing the incidence of metastases and their growth in the lungs of tumor-bearing mice are not normally distributed. Therefore, non-parametric statistics such as the Mann-Whitney U-Test may be used for analysis.

Study of this model by Gorelik et al. (1980, supra) showed that the size of the tumor cell inoculum determined the extent of metastatic growth. The rate of metastasis in the lungs of operated mice was different from primary tumor-bearing mice. Thus in the lungs of mice in which the primary tumor had been induced by inoculation of large doses of 3LL cells (1-5×10⁶) followed by surgical removal, the number of metastases was lower than that in nonoperated tumor-bearing mice, though the volume of metastases was higher than in the nonoperated controls. Using ¹²⁵IdUrd incorporation as a measure of lung metastasis, no significant differences were found between the lungs of tumor-excised mice and tumor-bearing mice originally inoculated with 10⁶ 3LL cells. Amputation of tumors produced following inoculation of 10⁵ tumor cells dramatically accelerated metastatic growth. These results were in accord with the survival of mice after excision of local tumors. The phenomenon of acceleration of metastatic growth following excision of local tumors had been observed by other investigators. The growth rate and incidence of pulmonary metastasis were highest in mice inoculated with the lowest doses (3×10⁴-10⁵ of tumor cells) and characterized also by the longest latency periods before local tumor appearance. Immunosuppression accelerated metastatic growth, though nonimmunologic mechanisms participate in the control exerted by the local tumor on lung metastasis development. These observations have implications for the prognosis of patients who undergo cancer surgery.

### E. Walker Carcinosarcoma 256

Summary: Tumor may be implanted sc in the axillary region as a 2-6 mm fragment, im in the thigh as a 0.2-ml inoculum of tumor homogenate containing 10⁶ viable cells, or ip as a 0.1-ml suspension containing 10⁶ viable cells. Treatment of the composition being tested is usually ip. Origin of tumor line: arose spontaneously in 1928 in the region of the mammary gland of a pregnant albino rat. J Natl Cancer Inst 13:1356, 1953.

| | |
|---|---|
| Animals | One sex used for all test |
| | and control animals in one experiment. |
| Propagation Strain | Random-bred albino Sprague-Dawley rats |
| Tumor Transfer | S.C. fragment implant is by trochar or 12-g needle |
| | into axillary region with puncture in inguinal area. |
| | I.m. implant is with 0.2 ml of tumor homogenate |
| | (containing cells) into the thigh. I.p. implant |
| | is with 0.1 ml 10⁶ viable suspension (containing |
| | 10⁶ viable cells) Day 7 for im or ip implant; Days |
| | 11-13 for sc implant |
| Testing Strain | Fischer 344 rats or random-bred albino rats |
| Time of Transfer | Same as above |
| Weight | 50-70 g (maximum of 10-g weight range within |
| | each experiment) |
| Exp Size (n) | 6/roup; No. of control groups varies according to |
| | number of test groups. |

| Test | Prepare drug | Administer | Weigh animals | Evaluate on |
|---|---|---|---|---|
| system | on day: | drug on days: | on days | days |
| 5WA16 | 2 | 3-6 | 3 and 7 | 7 |
| 5WA12 | 0 | 1-5 | 1 and 5 | 10-14 |
| 5WA31 | 0 | 1-9 | 1 and 5 | 30 |

In addition the following general schedule is followed

| DAY | PROCEDURE |
|---|---|
| 0 | Implant tumor. Prepare materials. Run positive control in every |
| | odd-numbered experiment. Record survivors daily. |
| 1 | Weigh and randomize animals. Begin treatment with |
| | therapeutic composition. Typically, mice receive 1 μg of the test |
| | composition in 0.5 ml saline. Controls receive saline alone. |
| | Treatment is one dose/week. Any surviving mice are |
| | sacrificed after 4 wks of therapy. |
| Final | Kill all survivors and evaluate experiment. |
| day | |

Quality Control Acceptable i.m. tumor weight or survival time for the above three test systems are: 5WA16: 3-12 g.; 5WA12: 3-12 g.; 5WA31 or 5WA21: 5-9 days.
Evaluation: Compute mean animal weight when appropriate, and at the completion of testing compute T/C for all test groups. When the parameter is tumor weight, a reproducible T/C 42% is considered necessary to demonstrate activity. When the parameter is survival time, a reproducible T/C 125% is considered necessary to demonstrate activity. For confirmed activity

### F. A20 Lymphoma

10⁶ murine A20 lymphoma cells in 0.3 ml saline are injected subcutaneously in Balb/c mice. The mice are treated intravenously with 1 g of the composition being tested in 0.5 ml saline. Controls receive saline alone. The treatment is given as one dose per week. Tumor growth is monitored daily by physical measurement of tumor size and calculation of total tumor volume. After 4 weeks of therapy the mice are sacrificed.

Having now generally described the invention, the same will be more readily understood through reference to the following examples which are provided by way of illustration, and are not intended to be limiting of the present invention, unless specified.

### Example 1

### Detection and Identification of egc Staphylococcal Enterotoxins

### Lymphocyte Proliferation Assay

Several Samples of the agent B36873 were assayed for their ability to induce proliferation of human T lymphocytes by standard 4-day mitogenicity assay (Poindexter N J, Schlievert P M et. al., J. Infect. Dis. 151:65-72 (1985). Human peripheral blood mononuclear cells (PMBCs) were isolated from heparinized blood of healthy human donors by fractionated by centrifugation through a Ficoll-Hypaque™ PLUS gradient (Amersham Biosciences, Uppsalla Sweden). Lymphocytes were washed and suspended to a concentration of 1.0×10⁶ cells/ml in RPMI 1640 medium (Gibco/Invitrogen Corporation, Grand Island, N.Y.) containing 2% fetal bovine serum, 2 mM glutamine, 200 U sodium penicillin G per ml, and 200 μg of streptomycin sulfate per ml. Suspended cells were distributed into 96-well plates, 200 μl per well, then sample, 50 μl per well, was added. Plates were incubated at 37° C., 6% CO₂, for 72 hours before 1 μCi of [3H]-thymidine (Moravek, Brea, Calif.) was added to each well. After 18 to 24 hours, cells were harvested with a Packard BioScience Filtermate Harvester (Meriden, Conn.). Radiolabeled DNA was measured using a Packard BioScience TopCount NXT Scintillation and Luminescence Counter.

### Recombinant Production and Purification of egc (Enterotoxin Gene Complex) Staphylococcal Enterotoxins Used for Flow Cytometry Analysis of T-Cell Vβ Repertoire

The methods used for production and purification of the egc toxins is that described by Jarraud et al., J. Immunol. 166:669-77 (2001) and herein on pages 19-22. Briefly, each toxin was expressed in E. coli using the pMALc2 expression vector (New England Biolab) or pET43 vector (Novagen) following the manufacturer's instruction, resulting in recombinant toxins. Those egc SE's prepared the pMALc2 vector had a maltose binding domain on the C-terminus end of each superantigen. This addition did not interfere with the superantigenic activity of each toxin. The detection of superantigenic activity was done by T cell proliferation assay using PBL from healthy donors. Flow cytometry was performed after incubating the PBL cells with each of the egc toxins. The Vβ profile was determined by flow cytometry using commercially available monoclonal antibody (Beckman/Coulter/Immunotech, Marseille, France) tagged to either phycoerytrin (PE), fluorescein isothiocyanate (FITC), or to PE and FITC, which allowed to explore a large Vβ panel. Analysis was performed by four-color flow cytometry (FACSCan; Becton-Dickinson).

### Assessment of Superantigenicity of Agent B36873 by Vβ Profile

Two methods were used to assay for the ability of agent B36873 to stimulate T cells via the Vβ region.

### Flow Cytometric Analysis of T-Cell Vβ Repertoire

Human peripheral blood mononuclear cells (Hu-PBMCs) obtained from two healthy donors were isolated from heparinized venous blood by density gradient sedimentation over Ficoll (PANCOLL, PAN™ Biotech GmbH, Aidenbach, Germany). Cells were washed three times in Hanks Balanced Salt Solution (HBSS, Sigma-Aldritch) and resuspended in RPMI 1640 (Gibco, Invitrogen Corporation) supplemented with 10% heat-inactivated Fetal Bovine Serum (FBS, Gibco, Invitrogen Corporation), 20 mM HEPES buffer, 2 mM L-Glutamine (Sigma-Aldritch), 100 μg/ml of streptomycin, 100 μg/ml of penicillin (Gibco-Invitrogen Corporation). Cells (at 2-5 10⁶ cells/ml) were cultured for three days in the presence of staphylococcal enterotoxins SEA, SEB, SEC, SED, SEE, TSST-1, SEG, SEI, SEM, SEN, SEQ and samples of agent B36873 (0.1-250 ng/ml) in 24-well plates (Falcon®Becton-Dickinson, USA). After three days, cells were washed resuspended in cells culture medium (RPMI 1640 supplemented) complemented with 20 U/ml of Hu-IL2 (Eurobio, France) for three days, then washed and allowed by 3 days in fresh cells culture medium complemented with 50 U/ml of Hu-IL2. Finally, cells were washed and resuspended in fresh cells culture medium complemented with 100 U/ml of Hu-IL2. For flow cytometry studies, Hu-PBMCs were collected before marking and the Vβ profile was performed by using the IOTest® Beta Mark (PN IM3497 Immunotech, Marseille, France). This kit comprised height vials marked A to H each containing three Vβ families tagged to either phycoerytrin (PE), fluorescein isothiocyanate (FITC), or to PE and FITC and permitted to explore a large the Vβ panel (1; 2; 3; 4; 5.1; 5.2; 5.3; 7.1; 7.2; 8; 9; 11; 12; 13.1; 13.2; 13.6; 14; 16; 17; 18; 20; 21.3; 22; 23). Each vial was diluted (1/10) in staining solution filtered in 0.22 μm filter (PBS, 5% Bovine Serum Albumin (BSA), 0.5 M EDTA, 0.02% sodium azide, 10% human normal serum, anti-CD3 conjugated with cyanin-5-phycoerytrin (C5P)). Cells were separated in 8 tubes, centrifuged and medium was eliminated by double aspiration. Then 10 μl of each vial dilution was added to each tube and incubated for 20 minutes at 4° C. in the dark. After the incubation period, cells were washed by PBS and resuspended in staining solution. Analysis was performed by four-color flow cytometry (FACSCan; Becton Dickinson). The manufacturer's parameters description for FACS adjustments were followed. The multiparameter data files were analyzed with the Cellquest program (Becton Dickinson). Negative (cells culture medium) and positive (Phaseolus vulgaris agglutinin; PHA; 10 μg/ml) controls were used to verify the specificity of this method.

### RT-PCR Analysis of T-Cell Vβ Repertoire

PBMCs were isolated from venous blood of healthy donors. Heparin treated (14 U/ml blood) blood were fractionated by gradient centrifugation over Ficoll-Paque Plus (Amersham Pharmacia Biotech, AB) to isolate PBMCs according to procedures described previously. The PBMCs were washed and resuspended in RPMI 1640 supplemented with 2% FBS, 100 U penicillin G, and 100 μg/ml streptomycin (complete RPMI medium), and incubated overnight at 37° C. and 5% CO₂ in plastic petri dishes. Non-adherent lymphocyte-enriched PBMCs were collected, washed, and resuspended at a final concentration of 2.5×10⁶ cells/ml. To determine whether expansion of specific huVβ-bearing T cells occurred following stimulation by purified staphylococcal enterotoxins (e.g., SEA, SEB, SEC1 SEE, TSST-1, SEG and SEI), each toxin (final concentration of 1 μg/ml) was added to a 3 ml aliquot of the enriched lymphocyte cell suspension. Likewise samples of agent B36873 in 50 μl volumes were tested. Cell cultures were incubated for 4 days. Control cultures, stimulated by adding a soluble murine mAb specific for the human CD3 molecules (33 ng/ml final concentration; Sigma, St. Louis, Mo., C-7048) were used to quantify basal levels of huVβ expansion.

Total RNA was extracted from approximately 5×10⁶ cells/ml SAgs stimulated PBMCs using Trizol reagent (Life Technologies, Gaithersburg, Md.). Two μg of total RNA were used to generate first-strand cDNA using Superscript II reverse transcriptase (Life Technologies, Gaithersburg, Md.) and oligo dT primers (Life Technologies, Gaithersburg, Md.). The reverse transcription was performed in 20 μl reaction followed by manufacturer's direction.

### 1.3 RT-PCR Quantification

Primers for amplification were designed by Primer Express version 2.0 (PE Applied Biosystems), based on published sequence for huVβ and glyceraldehydes-3-phosphate dehydrogenase (G3PDH), and are listed below. The reaction was performed in a final volume of 25 μl of SYBR Green I dye master mix (PE Applied Biosystems, Foster City, Calif.), 2 pmoles of forward and reverse primers, and 5 μl of 10 times diluted cDNA.

RT-PCR was performed using ABI Prism 7500 (PE Applied Biosystems, Foster City, Calif.). Thermocycling conditions consisted of an initial denaturation at 50° C. for 10 min and 95° C. for 10 min, followed by 40 cycles at 95° C. for 15 s, 60° C. for 1 min. The fluorescent data were acquired during each extension phase. After 40 cycles a melting curve was generated by slowly heating the reaction at 0.1° C./s from 60° C. to 95° C., while the fluorescence was measured continuously.

External cDNA standards for G3PDH were constructed by TA cloning of PCR fragments into pCR4-TOPO vector (Life Technologies, Gaithersburg, Md.) according to manufacturer's recommendation. PCR fragment was purified by gel electrophoresis followed by excision of the band of the correct molecular weight. The sequence identities of cloned fragments were verified by DNA sequencing on an ABI Prism 3100 Genetic Analyzer (PE Applied Biosystems). The concentration of cloned plasmid was determined by OD260 and the number of copies/ml of plasmid was calculated as following formula:

$$Copies \text{/} ml = \frac{6.023 \times {10}^{23} \times C \times {OD}_{260}}{MW t}$$

Where C=5×10⁻⁵ g/ml for DNA and MWt=molecular weight of plasmid including PCR product (base pairs×6.58×10² g).
Standard curves were generated from 10 fold serial diluted external cDNA standard ranged from 10⁸ copies/μL to 10² copies/μL.

After completion of the RT-PCR amplification, data was analyzed with the Sequence Detector Systems version 1.2.2 (PE Applied Biosystems). To synchronize each experiment, the baseline was set automatically by software. The increase in intensity of fluorescence of the reporter dye (ΔRn) was plotted against the cycle number. The threshold cycle (C_{T}) was calculated by the sequence detection software as the cycle number at which the ΔRn crossed the baseline. Quantification of the sample was calculated from C_{T} by interpolation from standard curve which plot a linear regression line by plotting the logarithm of template concentration against the corresponding C_{T}. The quality of the standard curve was determined by the slope and correlation coefficient (R²).

Calculations to determine the extent of huVβ expansion were done as described by Deringer et al Infect. Immun. 10, 4048-4054 (1997) with slight modification. Briefly, the values for each specific huVβ product were normalized by G3PDH value. The normalized huVβ values were used to determine the percentage of each of 23 different amplified huVβs in stimulated cultures. Results were expressed as an expansion index value that is defined as the ratio of the percentage of each huVβ in a SAgs stimulated culture compared with the percentage of the same huVβ in an identical culture stimulated with anti-CD3 (basal levels of huVβ).

TABLE 1

Primers used for Vβ profile

| | | |
|---|---|---|
| Name | Forward primer (‘5 to 3′) | Reverse primer (‘5 to 3′) |

| | | |
|---|---|---|
| 33PDH | TGG ACC ACC AAC TGC TTA GC | GGC ATG GAC TGT GGT CAT GAG |
| | (SEQ ID NO: 100) | (SEQ ID NO: 101) |
| Vβ 1 | GAA GCA GGC CCA GTG GAT | CGC TGT CCA GTT GCT GTG AT |
| | (SEQ ID NO: 102) | (SEQ ID NO: 103) |
| Vβ 2 | GAG TCT CAT GCT GAT GGC AAC T | TCT CGA CGC CTT GCT CGT AT |
| | (SEQ ID NO: 104) | (SEQ ID NO: 105) |
| Vβ 3 | TCC TCT GTC GTG TGG CCT TT | TCT CGA GCT CTG GGT TAC TTT CA |
| | (SEQ ID NO: 106) | (SEQ ID NO: 107) |
| Vβ 4 | GGC TCT GAG GCC ACA TAT GAG | TTA GGT TTG GGC GGC TGA T |
| | (SEQ ID NO: 108) | (SEQ ID NO: 109) |
| Vβ 5 | GCT CCA GGC TGC TCT GTT G | TTT GAG TGA CTC CAG CCT TTA CTG |
| | (SEQ ID NO: 110) | (SEQ ID NO: 111) |
| Vβ 6 | GGC AGG GCC CAG AGT TTC | GGG CAG CCC TGA GTC ATC T |
| | (SEQ ID NO: 112) | (SEQ ID NO: 113) |
| Vβ 7 | AAAAG TGT GCC AAG TCG CTT CTC | TGC AGG GCG TGT AGG TGA A |
| | (SEQ ID NO: 114) | (SEQ ID NO: 115) |
| Vβ 8 | TGC CCG AGG ATC GAT TCT C | TCT GAG GGC TGG ATC TTC AGA |
| | (SEQ ID NO: 116) | (SEQ ID NO: 117) |
| Vβ 9 | TGC CCG AGG ATC GAT TCT C | TCT GAG GGC TGG ATC TTC AGA |
| | (SEQ ID NO: 118) | (SEQ ID NO: 119) |
| Vβ 11 | CAT CTA CCA GAC CCC AAG ATA CCT | ATG GCC CAT GGT TTG AGA AC |
| | (SEQ ID NO: 120) | (SEQ ID NO: 121) |
| Vβ 12 | GTT CTT CTA TGT GGC CCT TTG TCT | TCT TGG GCT CTG GGT GAT TC |
| | (SEQ ID NO: 122) | (SEQ ID NO: 123) |
| Vβ 13.1 | TGG TGC TGG TAT CAC TGA CCA A | GGA AAT CCT CTG TGG TTG ATC TG |
| | (SEQ ID NO: 124) | (SEQ ID NO: 125) |
| Vβ 13.2 | TGT GGGG CAG GTC CAG TGA | TGT CTT CAG GAC CCG GAA TT |
| | (SEQ ID NO: 126) | (SEQ ID NO: 127) |
| Vβ 14 | GCT CCT TGG CTA TGT GGT CC | TTG GGT TCT GGG TCA CTT GG |
| | (SEQ ID NO: 128) | (SEQ ID NO: 129) |
| Vβ 15 | TGT TAC CCA GAC CCC AAG GA | TGA CCC TTA GTC TGA GAA CAT TCC A |
| | (SEQ ID NO: 130) | (SEQ ID NO: 131) |
| Vβ 16 | CGG TAT GCC CAA CAA TCG AT | CAG GCT GCA CCT TCA GAG TAG A |
| | (SEQ ID NO: 132) | (SEQ ID NO: 133) |
| Vβ 17 | CAA CCA GGT GCT CTG CTG TGT | GAC TGA GTG ATT CCA CCA TCC A |
| | (SEQ ID NO: 134) | (SEQ ID NO: 135) |
| Vβ 18 | GGA ATG CCA AAG GAA CGA TTT | TGC TGG ATC CTC AGG ATG CT |
| | (SEQ ID NO: 136) | (SEQ ID NO: 137) |
| Vβ 20 | AGG TGC CCC AGA ATC TCT CA | GGA GCT TCT TAG AAC TCA GGA TGA A |
| | (SEQ ID NO: 138) | (SEQ ID NO: 139) |
| Vβ 21 | GCT GTG GCT TTT TGG TGT GA | CAG GAT CTG CCG GTA CCA GTA |
| | (SEQ ID NO: 140) | (SEQ ID NO: 141) |
| Vβ 22 | TGA AAG CAG GAC TCA CAG AAC CT | TCA CTT CCT GTC CCA TCT GTG T |
| | (SEQ ID NO: 142) | (SEQ ID NO: 143) |
| Vβ 23 | TTC AGT GGC TGC TGG AGT CA | CAG AGT GGC TGT TTC CCT CTT T |
| | (SEQ ID NO: 144) | (SEQ ID NO: 145) |
| Vβ 24 | ACC CCT GAT AAC TTC CAA TCC A | CCT GGT GAG CGG ATG TCA A |
| | (SEQ ID NO: 146) | (SEQ ID NO: 147) |

### Gene Content of S. Aureus Strain D8237E

Organisms from S. Aureus strain D8472E were harvested after 24 hours of growth. One batch was treated with 50% ethanol and a second was untreated.

Using polymerase chain reaction (PCR), the presence of 22 specific staphylococcal virulence genes (including 16 superantigenic toxins, 3 hemolysins, and 3 leukocidins), as described previously (Jarraud et al., (2001) supra; Jarraud et al., (2002)) was determined. Amplification of gyrA was used as a quality control of each DNA extract and the absence of PCR inhibitors. S. aureus strains Fri 913 (sea, see, sec, tst, lukE lukD, sek, sel, sep, and hlg), Fri 1151m (sed, sej, lukE lukD, hlgv, and hlb), ATCC 14458 (CCM5757) (seb, lukE lukD, sek, and hlgv), NCTC 7428 (sec, tst, lukM, seg, sei, sem, sen, seo, lukE lukD, hlgv, and hlb), A92 0211 (seg, sei, sem, sen, seo, eta, etb, lukE lukD, and hlgv), RN6390 (lukE lukD, hlgv, hlb, and agr1), RN6607 (sed, seg, sei, sem, sen, seo, lukE lukD, hlg, and agr2), RN8465 (seg, sei, sem, sen, seo, tst, hlg, and agr3), RN4850 (seg, sei, sem, sen, seo, eta, etb, lukE lukD, hlgv, and agr4), RN 6911 (lukE lukD, hlgv, hlb, agr null), E-1 (seg, sei, sem, sen, seo, lukE lukD, eta, hlgv, edinB and C), ATCC 49775 (seg, sei, sem, sen, seo, lukS lukF, and hlg), and ATCC 51811 (FRI 569) (seh, lukE lukD, hlb, and hlgv) were used as positive controls for PCR. PCR products were separated by electrophoresis in 1% agarose gels.

In a second method, multiplex PCR was used for DNA analysis of S. Aureus strain D8472E. DNA was extracted by standard methods using phenol, chloroform and ethanol precipitation. Multiplex PCR was used to detect the presence of staphylococcal enterotoxin genes, (Monday S R, Bohach G A J. Clin. Microbiol. 37:3411-3414. (1999) which is incorporated in entirety herein by reference. Primers sets used to detect genes encoding seg, sei, sek, sel, sem, sen, seo, and seq were provided by Davida S. Smyth, Dublin Ireland. The following primers were used. (SEQ ID NOS: 148-167)

| | | GenBank | | |
|---|---|---|---|---|
| Gene | Primer sequence (5′ and 3′)^{a} | accession no.^{b} | Location^{c} | Size^{d} |
| sea | GCA GGG AAC AGC TTT AGG C | M18970 | 126-144 | 520 |
| | GTT CTG TAG AAG TAT GAA ACA CG | | 646-624 | |
| seb-sec | ATG TAA TTT TGA TAT TCG CAG TG | M11118 (seb) | 28-48 | 643 |
| | TGC AGG CAT CAT ATC ATA CCA | | 690-670 | |
| sec | CTT GTA TGT ATG GAG GAA TAA CAA | X05815 | 407-430 | 283 |
| | TGC AGG CAT CAT ATC ATA CCA | | 690-670 | |
| sed | GTG GTG AAA TAG ATA GGA CTG C | M28521 | 368-389 | 384 |
| | ATA TGA AGG TGC TCT GTG G | | 752-734 | |
| see | TAC CAA TTA ACT TGT GGA TAG AC | M21319 | 446-468 | 170 |
| | CTC TTT GCA CCT TAC CGC | | 616-599 | |
| seg | CGT CTC CAC CTG TTG AAG G | AF064773 | 317-335 | 327 |
| | CCA AGT GAT TGT CTA TTG TCG | | 644-624 | |
| seh | CAA CTG CTG ATT TAG CTC AG | U11702 | 245-264 | 360 |
| | GTC GAA TGA GTA ATC TCT AGG | | 603-583 | |
| sei | CAA CTC GAA TTT TCA ACA GGT AC | AF064774 | 325-347 | 465 |
| | CAG GCA GTC CAT CTC CTG | | 790-773 | |
| sej | CAT CAG AAC TGT TGT TCC GCT AG | AF053140 | 471-493 | 142 |
| | CTG AAT TTT ACC ATC AAA GGT AC | | 612-590 | |
| 1sl | GCT TGC GAC AAC TGC TAC AG | J02615 | 48-67 | 559 |
| | TGG ATC CGT CAT TCA TTG TTA A | | 606-587 | |
| 16S rRNA | GTA GGT GGC AAG CGT TAT CC | X68417 | 545-564 | 228 |
| | CGC ACA TCA GC GTC AG | | 773-758 | |

### Results

### T Cell Proliferation of Agent B36873

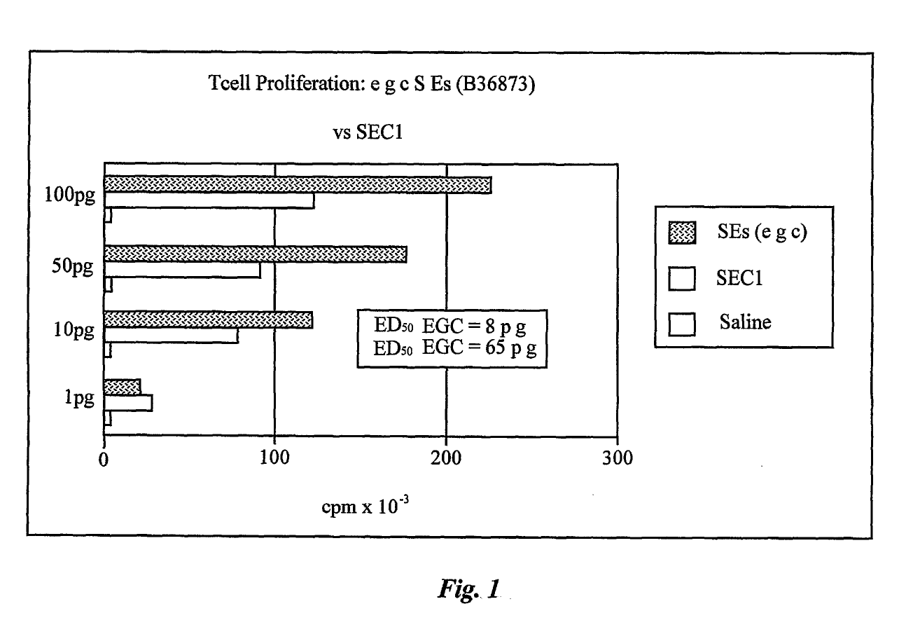

A dose of 1-100 picrograms of the agent B36873 induced strong mitogenicity in human peripheral blood mononuclear cells in vitro exceeding that of native staphylococcal enterotoxin C (FIG. 1).

### Vβ Profile of Agent B36873

Using the flow cytometry method, samples 1 and 2 of B36873 stimulated Vβ3, 5, 7, 21 and with the RT-PCR method, the samples of B36873 activated Vβ 1, 3, 5, 6, 7, 21, 23. The Vβ profiles for the individual egc SEs are given in Table 4. Vβ clones activated by the B36873 samples that match the complete or partial Vβ repertoire of the recombinant egc SEs are given in red (Table 4).

In a more detailed analysis using Immunoscope/flow cytometry, B36873 activated Vβ 1, 3, 5.1, 5.2, 5.3, 7.1, 9, 21.3, 23 which matched fully or in part, the Vβ profiles for all five of the recombinant egc SEs prepared with two different vectors in two different strains of E. Coli. The Vβ profile of agent 36873 matched each of the 5 recombinant egc SEs as shown in red (Table 5).

TABLE 4

| | Egc Genes & TCR Vβ Profiles | | | | |
|---|---|---|---|---|---|
| Test Article | SEG | SEI | SEO | SEM | SEN |
| S. Aureus (Strain D8472E) | Egc Genes | | | | |
| (Unrtreated/ | POSITIVE/POSITIVE* | | | | |
| Ethanol-Treated) | | | | | |
| Egc Toxins: Recombinant | Tcell Vβ Profiles | | | | |
| (Plasmid pMal-c2/E. Coli) | | | | | |
| Flow Cytometry/ | 12, 13.6, | 1, 5, 6, 23 | 5, 7 | 6, 21 | 9 |
| Immunoscope | 14 | | | | |
| RT-PCR | 3, 12, | 1, 5, 6, 23 | 5 | 6, 21 | |
| | 3.2, 14 | | | | |
| Egc Agent B36873 | | 1, 5, 6, 23 | 5, 7 | 6, 21 | |
| B36873-2 (RT-PCR) | | | | | |
| B36873-2 | 3 | 5, 23 | 5, 7 | 21 | |
| (Flow Cytometry) | | | | | |
| B36873-1 | 3 | 5, 23 | 5, 7 | 21 | |
| (Flow Cytometry) | | | | | |

*NO Genes for classical SEs (SEA, SEB, SEC1, SEC2, SEC3, SED, SEE, TSST1, SEH, SEK, SEJ, SEK, SEL) were identified.

TABLE 5
Vβ Profiles of Recombinant egc SE's vs
B36873 (Immunoscopy/Cytometry)

| | | | | | |
|---|---|---|---|---|---|
| Test Article | SEG | SEI | SEO | SEM | SEN |

Agent B36873

| | | | | | |
|---|---|---|---|---|---|
| # 1 | 3, 5.1, 5.3 | 5.1, 5.2, 5.3 | 5.1, 7.1 | 5.1, 5.3, 23 | 5.1, 5.3 |
| # 2 | 5.1, 5.3 | 5.1, 5.2, 5.3 | 5.1, 7.1, 21.3 | 5.1, 7.1, 5.3, 21.3 | 5.1, 5.3, 9, 21.3 |
| # 3 | 5.3 | 5.2, 5.3 | 5.1, 7.1, 21.3, 23 | 5.1, 7.1, 21.3, 23 | 5.3, 21.3 |

Recombinant Egc Toxin
Bacterial strain/Plasmid

| | | | | | |
|---|---|---|---|---|---|
| E. Coli BL21DE3 | 3, 5.1, 5.3, | 5.1, 5.2, 5.3, | 5.1, 7.1, 21.3, 23.3 | 5.1, 5.3, 7.1, 21.3, | 5.1, 5.3, 9, 21.3 |
| (plasmid: pET43.1) | 12, 13.1, | 1, 14, 23.3 | | 23.3, 1, 3, 8, 14 | |
| AND | 13.2, 14 | | | | |
| E. Coli TG1 | | | | | |
| (plasmid: pMAL-c2) | | | | | |

### Analysis of S. Aureus Strain D8472E Superantigen Genes

DNA extracted from S. Aureus strain D D8472E, analyzed by two methods, expressed staphylococcal enterotoxin genes seg, sei, sem, sen, seo of the enterotoxin gene cluster (egc). No other enterotoxin genes were present (Tables 4 & 6). Genes for all members of the egc SEs were found in strain D8472E with the notable absence of genes for any other staphylococcal enterotoxin, and the Vβ profile of agent B36873 derived from parent strain D8472E exhibited matches with the complete or partial Vβ repertoire of all 5 members of the egc family namely SEG, SEI, SEM, SEQ (Tables 4 & 5).

These finding strongly point to the presence of only egc SEs in agent B36873 and the absence of any other SEs.

TABLE 6
SE Genes in Parent Strain of
Drug B32563

| | |
|---|---|
| | STRAIN D274E: |
| | ENTEROTOXIN |
| SE TYPE | GENES DETECTED |

EGC SE'S

| | |
|---|---|
| G | POSITIVE |
| I | POSITIVE |
| M | POSITIVE |
| O | POSITIVE |
| N | POSITIVE |

CLASSIC SE'S

| | |
|---|---|
| A | NEGATIVE |
| B | NEGATIVE |
| C | NEGATIVE |
| D | NEGATIVE |
| E | NEGATIVE |
| TST | NEGATIVE |
| J | NEGATIVE |
| K | NEGATIVE |
| L | NEGATIVE |

### Example 2

### Intrathecal (Intrapleural) Injection of egc SAgs in Patients with Malignant Pleural Effusions

### SAg-Treated Patients

From February 1999 to October 2002, 14 consecutive and unselected patients with NSCLC and MPE were treated with egc SAg. Patients were required to have non-small cell lung cancer (NSCLC) with, at least, 350 cc of pleural fluid. Systemic chemotherapy and all other biological-response modifying agents with antitumor activities were discontinued, at least one month prior to initiating treatment. Radiotherapy was allowed provided it was not focused on the site of the pleural effusion. Pleural effusions were confirmed by chest radiograph, chest CT and ultrasonography. The diagnosis of MPE was established by positive pleural fluid cytology in all patients. Karnofsky performance scores (KPS) before and after treatment were recorded for all cases. Irrespective of KPS, all patients satisfying the above criteria were eligible for this study.

Before each course of treatment, patients received a complete physical examination, CBC, serum chemistry, liver function tests, urine analysis, ECG, and pulmonary function tests. Each patient had chest radiograph and sonography before starting treatment to document the presence of pleural fluid. Samples of blood and pleural fluid were obtained by venipuncture and thoracentesis, respectively, before and six hours after selected procedures. Chest radiographs and sonographics were monitored for each patient before and monthly for the first 3 months after treatment and then bimonthly until completion of the study. Computerized tomographic studies of the lung were done before treatment on patients 3 and 6.

### Treatment

Once a MPE was documented, thoracentesis was performed after sonographic localization. With the patient in the sitting position, the site was localized by sonography. An 18-gauge needle was introduced into the pleural space and fluid was withdrawn through a three-way stopcock. In general. approximately 50-75% of the total effusion was removed. Immediately thereafter, SAg 100-400 pg in 10-20 cc of normal saline was delivered into the pleural cavity over one minute through the same needle used for thoracentesis. Thoracentesis and SAg administration were repeated every 3 to 7 days until there was minimal or no reaccumulation of pleural fluid after 10 days. A total of 52 intrapleural SAg treatments was administered. The mean number of intrapleural treatments required before there was minimal or no fluid reaccumulation was 3.71±1.3 (SD). Along with intrapleural SAg, 6 patients also received intravenous SAg daily for 30, 21, 21, 14, 6 and 3 days, respectively, commencing at the time of the first intrapleural administration of SAg. Table 6 shows the schedules, dosages and routes of administration of SAg treatment in each of the 14 patients.

TABLE 7
SAg Treatment in Patients with Malignant
Pleural Effusions from NSCLC

| 14. | 47/M | AdenoCa | SAg 250 pg IP q3-4 days × 3 |
|---|---|---|---|
| Age/Sex | | Tumor Histology | SAg Regimen |
| 1. | 82/M | AdenoCA. | Initial Rx: SAg 250 pg IP q1wk × 3 wks |
| | | | SAg 500 pg IP q1wk × 3 wks |
| | | | SAg 100 pg IV qd × 30 days |
| | | | Repeat Rx at 6 mos: SAg 100 pg IV qd × |
| | | | 30 days |
| 2. | 67/M | AdenoCA | SAg 250 pg IP q1 wk × 4 wks |
| | | | SAg 50 pg IV qd × 21 days |
| 3. | 66/M | SqCCA | SAg 250 pg IP q1 wk × 4 wks |
| 4. | 61/M | AdenoCA | SAg 250 pg IP q1 wk × 4 wks |
| | | | SAg 500 pg IV qd × 14 days |
| 5. | 47/M | AdenoCA | SAg 200 pg IP q I wk × 3 |
| 6. | 73/M | SqCCA | SAg 250 pg IP q 3-4 days × 5 |
| 7. | 68/M | AdenoCA | SAg 250 pg IP q 3-4 days × 4 |
| 8. | 69/M | AdenoCA | SAg 250 pg IP q 1 wk × 3 |
| 9. | 56/M | AdenoCA | SAg 250 pg IP Q 3-4 days × 5 |
| | | | SAg 100 pg IV qd × 21 days |
| 10. | 65/M | SqCCA | SAg 250 pg IP q 3-4 days × 3 |
| | | | SAg 100 pg IV qd × 14 days |
| 11. | 79/M | AdenoCA | SAg 100 pg IP q 1 wk × 2 |
| 12. | 71/M | SqCCA | SAg 250 pg IP q 1 wk × 5 |
| 13. | 46/M | AdenoCA | SAg 250 pg IP × 1 |
| | | | SAg 100 pg IV qd × 3 days |

Patients were monitored for adverse effects in hospital for 24 hours after treatment and then followed at 3-6 day intervals for recurrence of pleural fluid by physical examination, ultrasound and chest radiograph.

### Evaluation of Response

Pleural effusions were assessed on serial chest radiographs. For pleural effusions, complete response (CR) was defined as the absence of any reaccumulation of pleural fluid confirmed by chest radiograph and sonography at 30 days. A partial response (PR) was defined as reaccumulation of pleural fluid that did not induce symptoms or require repeat thoracentesis at 30 days. Short-term responses were recorded at 30 days and long-term responses at 90 days after the completion of egc SAg treatment. Failure was defined as reaccumulation of fluid that caused dyspnea and required an additional thoracentesis. Lung tumors present on chest radiographs were measured and graded according to the WHO guidelines. In addition, the patients were monitored continuously for adverse effects, which were graded according to the National Cancer Institute Common Toxicity Criteria. Karnofsky performance status (KPS) was recorded for all patients before and 30 days after the completion of egc SAg treatment without prior knowledge of the pretreatment KPS scores of the talc-treated group.

Patients Treated with Talc Poudrage

Between 1993 and Jun. 1, 1998, 18 consecutive, unselected patients with symptomatic MPE from NSCLC (stage IIIb) referred to the Interventional Pulmonary Service of the University of California at San Diego Medical Center underwent pleurodesis by thoracoscopic insufflation of sterile, asbestos-free U.S. Pharmacopaeia-approved talc powder. The diagnosis was established by positive pleural fluid cytology on thoracentesis or evidence of NSCLC on pleural biopsy prior to referral. Followup to the date of death was obtained on 17 patients and survival duration was measured from the date of first treatment with talc poudrage via thoracotomy to the date of death. KPS scores were recorded before treatment without knowledge of the pre-treatment KPS scores of the SAg-treated group. A study of this patient population was reported previously (Burrows C M, Mathews W C, Colt H G. Chest. 2000 117: 73-78) and extended herein as a basis of comparison with the SAg-treated patients.

### Hematologic Studies

Peripheral blood and pleural fluid were sampled before and 6 to 24 hours after SAg treatment in 10 patients for determination of total and differential cell counts.

### Statistical Evaluation

The programs in the S-plus statistical software package, professional edition 6 for windows (Insightful Corporation, Seattle, Wash.) were used for data analysis. Patient survival duration was measured from the first day of SAg and talc poudrage treatment. Kaplan-Meir survival curves were derived with the survival analysis program developed at the Mayo Clinic and incorporated in the S-plus package. Estimates of survival probabilities and median survival time were obtained from the SAg-treated and talc-treated groups. Confidence intervals were based on the log-hazard scale. A log rank test was performed comparing the survival duration of the egc SAg treatment group with the talc-treated group. The Kaplan-Meir analysis was used to compute the median time to progression of measurable tumors and pleural effusions. A nonparametric method, the Wilcoxon rank sum statistic, was used to compare Karnofsky scores between two groups. The Cox proportional hazards model was used to evaluate whether the initial volume of pleural effusion was related to survival in both egc SAg and talc poudrage-treated groups. Peripheral blood and pleural fluid determinations of total and differential cell counts before and after egc SAg treatment were pooled from 10 patients and analyzed using at test.

### Results

### Patient Characteristics

Fourteen unselected, consecutive patients with MPE from NSCLC were treated with egc SAg, All were males with a median age of 67.5 years (range, 46-82). Eight had COPD or coronary artery disease. Of the NSCLC, 10 were adenocarcinomas and 4 were squamous cell carcinomas. Six of 14 patients received prior radiation or chemotherapy. In the remaining 8 patients, MPE was the first sign of their malignancy. In 11 patients, the MPE was associated with a radiographically-evident tumor mass. Serum chemistries and liver function tests were normal in all patients before treatment. At intitial presentation, all patients were dyspneic and six had submassive hemoptysis. Pleural effusions were left-sided in 11 patients and were associated with ascites and pericardial effusion in two and one patient, respectively. The median initial volume of the pleural effusions was 600 cc (range 350-1100 cc) and the median pretreatment Karnofsky performance status (KPS) was 40.0 (range 30-60) (Table 8).

In general, the egc SAg and talc poudrage-treated groups had similar demographics and clinical characteristics, tumor histology and staging by TMN subset (Table 8). A similar percentage in each group presented de novo without prior chemotherapy or radiation (p=1.5) and the median pretreatment KPS scores in both groups were not statistically different (p=0.74). In addition to their pleural effusion, 12 of 14 patients in the egc SAg-treated group and 17 of 18 patients in the talc-treated group had radiographically detectable lung tumors or tumor related lesions. Although the talc-treated group had a larger median initial pleural effusion volume than the SAg-treated group (p=0.001), all patients in both groups had symptomatic pleural effusions (Table 8).

### Toxicity

Adverse effects associated with egc SAg treatment are shown in Table 9. In general, egc SAg was well tolerated. The most common adverse event was fever ranging from 37.4°-39.8° C. (grade 2) that was unrelated to egc SAg dosage. Peak fever was 38° C. in 5 patients and 39.8° C. in 6 patients and lasted for 24-36 hours. In 2 patients, fever persisted for more than 36 hours and was relieved by indomethacin suppository. Minimal ipsilateral chest pain occurred in 3 patients and abated spontaneously. There was no evidence of respiratory distress, congestive heart failure or significant changes in hepatic or renal function during or after treatment. No stage 3 or 4 toxicity was observed in any case.

### Responses of the MPE and Tumor to SAg

Responses of MPE and tumors to egc SAg treatment are provided in Table 10 With respect to MPE, 11 patients had a CR and 3 had a PR. Twelve of 14 patients did not have recurrent effusion for more than 90 days after their last egc SAg treatment with a median time to recurrence of 5 months (3-23). Pleural fluid samples obtained 6 to 24 hours after SAg treatment from two patients demonstrated tumor cell degeneration that was not evident in pretreatment samples. One month after egc SAg treatment, the median pre-treatment KPS of 40 (range, 10-60) improved to a median KPS of 70 (40-90) (p=0.005) in association with resolution of the effusions. Patients 1 and 3 were retreated with SAg for recurrence of their MPE. In patient 1, a recurrent left pleural effusion six months after his first SAg treatment was retreated with intrapleural egc SAg every 3-4 days for 4 doses after which the effusion resolved and has not returned. Patient 1 has been disease-free for 27 months after starting his second course of treatment and is alive and 36 months from the first SAg treatment (Table 10). Patient 3 had a recurrent pleural and pericardial effusion 15 months after his first treatment and was retreated twice with intrapleural and intrapericardial egc SAg. However, the patient refused additional treatment and hence, the effect of this limited retreatment could not be evaluated. Patient 4 had a recurrent pleural effusion 4 months after starting egc SAg and was not retreated. Recurrent effusion was noted in patients 2 and 6 at the time of death, 11 and 8 months, respectively, after starting egc SAg treatment. Notably, patient 11 (pretreatment KPS 10) with a persistent hydropneumothorax who failed intrapleural chemotherapy two months earlier and had an indwelling chest tube in place draining >600 cc/24 h. Following the second intrapleural egc SAg injection, air leakage and catheter drainage ceased and his chest tube was successfully removed.

Pretreatment tumor masses were measurable in 12 patients at the start of egc SAg treatment. One patient showed a CR lasting 27 months after his last SAg treatment and 11 patients exhibited a median time to progression of their tumor mass of 4 months (2.5-14) (Table 10). In general, progression of tumor mass (median 4 months) was noted before recurrence of the pleural effusion (median 5 months).

### Survival

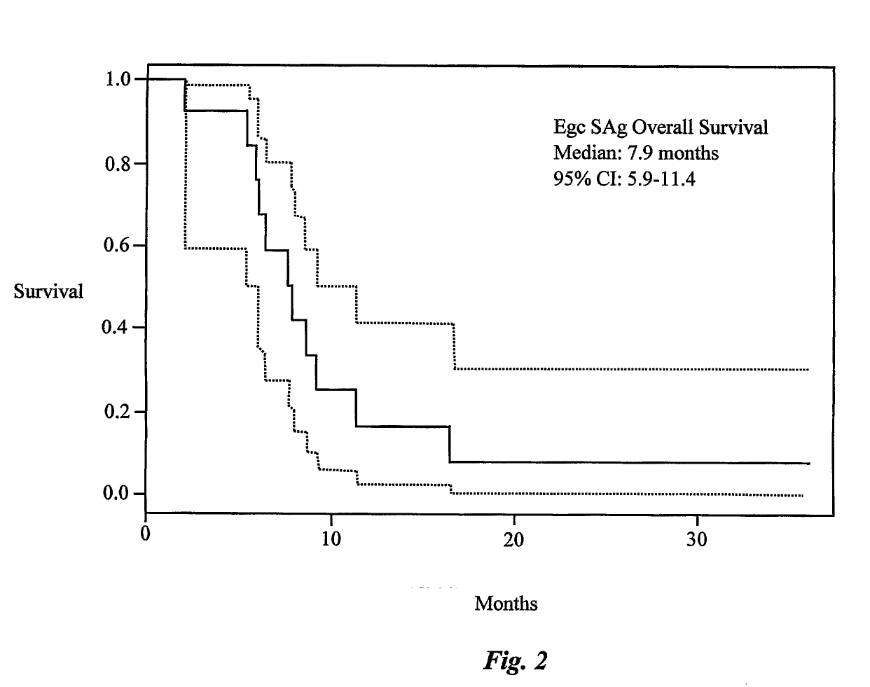

The median survival for all 14 patients in the egc SAg-treated group was 7.9 months (range 2-32 month) (95% CI, 5.4-11.4 months) compared to the median survival of 2.5 (range 0.1-57 months) (95% CI, 1-3.1 months) for 18 unselected, consecutive patients with MPE from NSCLC treated with talc poudrage (p=0.044) (FIG. 2). Thirteen of 18 patients from the latter group with a pretreatment KPS range of 10-60 (median 30) and distribution comparable to the 14 patients in the egc SAg-treated group (KPS range 10-60, median 40) (p=0.5), had a median survival of 2.0 months, (95% CI 0.4-2.9), that was significantly different from 7.9 months for the SAg-treated group (p=0.0023) (FIG. 3). Patients in the egc SAg-treated group survived on the order of 3-4 times longer than those treated with talc poudrage (FIGS. 1 & 2). Twelve of 14 patients in the egc SSAg-treated group survived more than 4 months, 9 more than 6 months, 4 more than 9 months and one patient is still alive, 36 months after starting therapy. In contrast, only 1 of 13 patients in the talc-treated group survived longer than 4 months and none survived more than 6 months (FIG. 3). Twelve-month survival for the egc SSAg-treated group was 14% versus 0% for the talc poudrage-treated group (FIG. 2). Survival in both egc SAg and talc-treated groups could not be predicted from pretreatment pleural fluid volume (p=0.26 and p=1 respectively).

### Route of Administration of SAg

Eight patients received intrapleural (IP) egc SAg only and 6 patients received IP egc SAg together with daily intravenous (IV) SAg (Table 1). Despite receiving significantly more egc SAg, the group receiving IP and IV therapy showed no significant difference in survival compared to the group receiving only IP treatment (p=0.3) (Tables 4 and 8).

### Hematologic Changes in the Blood and Pleural Fluid

Peripheral white blood cell and neutrophil counts increased significantly 6 to 24 hours after treatments in all patients (both p<0.05) (Table 9). Total nucleated cells, neutrophil and lymphocyte counts in pleural effusions increased significantly 6 to 24 hours after treatment (all p<0.05). While lymphocytes did not change significantly in peripheral blood following egc SAg treatment, there was a significant increase in lymphocyte count in the pleural fluid after egc SAg treatment.

TABLE 10
Characteristics of Patients Prior to Treatment with
SSAg and Talc Poudrage

| | Egc-SAg | Talc | |
|---|---|---|---|
| Characteristic | n = 14 | n = 18* | Significance |
| Primary Lung Cancer Histology | | | |
| Adenocarcinoma | 10 | 17 | ns |
| Squamous cell carcinoma | 4 | 1 | |
| Stage by TMN Subset (%) | | | |
| T4 N0-3: IIIb | 100 | 100 | ns |
| Median age and range (yr) | 67.5 (47-82) | 68.5 (51-80) | ns |
| No Prior Chemotherapy/Radiation (%) | 57 | 56 | ns |
| COPD/ASHD (%) | 57 | 44 | ns |
| Pre-treatment KPS (%) | | | All ns |
| 70-90 | 0 | 28 | |
| 10-60 | 100 | 72 | |
| Median KPS | 40 | 50 | |
| Median KPS (10-60) | 40 | 30 | |
| Symptomatic Effusion (%) | 100 | 100 | ns |
| Median Initial Pleural Fluid | 600 | 1600 | p = 0.001 |
| Volume Removed (cc) | (350-1400)** | (750-4500) | |
| Chest Radiographic Findings: # of lesions (%) | | | All ns |
| Parenchymal/Hilar Tumor | 11 (79) | 11 (61) | |
| Bronchial Obstruction/Atelectasis | 4 (29) | 9 (43) | |
| Nodular/Interstitial Infiltrates | 1 (7) | 2 (10) | |

*All 18 patients were evaluable for histology, tumor stage, age, pre-treatment KPS, symptomatic effusions. Seventeen of 18 patients were evaluable for prior chemotherapy/radiation, ASHD or COPD, pleural effusion volume and chest radiographic lesions.
**The pleural volumes removed and quantitated during the first thoracentesis or thoracoscopy in the SSAg and talc-treated groups respectively. The volume removed in the SSAg-treated group represented 50-75% to the total effusion volume estimated by post-thoracentesis ultrasound and chest radiographs.
ns = not significant

TABLE 9
TOXICITY of egc SAg TREATMENT

| | Severity of Adverse Event¹ | | |
|---|---|---|---|
| Adverse | Grade (%) | | |
| Event | 0 | 1 | 2 |
| Fever | 8 (57) | 5 (36) | 6 (43) |
| Chills | 10 (71) | 2 (14) | None |
| Pain | 11 (79) | 3 (21) | None |
| Dyspnea | 14 (100) | None | None |
| Leukopenia | 14 (100) | None | None |

¹There was no grade 3 or 4 toxicity

TABLE 10
Results of egc-SAg Treatment

| | Pleural Effusion: | Lung Tumor: | Survival |
|---|---|---|---|
| | Response & | Response & | Duration |
| Patient # | Duration | Duration | (months) |
| 1 | CR: 6 months | CR: 29 months. | 36 |
| | (Initial treatment) | | |
| | CR: 27 months | | |
| | (Recurrent pleural | | |
| | effusion retreated | | |
| | at 6 months, see Table 1) | | |
| 2 | CR: 11 months. | NC: 9 months, | 11.4 |
| 3 | CR: 15 months. | NC: 14 months | 16.6 |
| 4 | CR: 7 months. | NC: 4 months | 8.6 |
| 5 | CR: 5 months. | NC: 3 months | 5.9 |
| 6 | CR: 8 months. | NC: 6 months | 9.2 |
| 7 | CR: 4 months | NC: 2.5 months | 5.3 |
| 8 | CR: 4 months. | NC: 5 months | 7.9 |
| 9 | CR: 7 months | NC: 4 months | 7.7 |
| 10 | CR: 5 months | NC: 3 months | 5.4 |
| 11 | PR: 5 months | No hilar or | 6.7 |
| | | parenchymal | |
| | | tumor | |
| 12 | PR: I month | NC: 1 month | 2 |
| 13 | PR: 3 months. | No lung hilar or | LTF* |
| | | parenchymal | |
| | | tumor | |
| 14 | CR: 5 months. | NC: 2 months | LTF |
| | CR: 11; PR: 3 | CR: 1; NC: 11 | Median: 7.9 |
| | Median Time to | Median Time | mos (2-36) |
| | Progression: | to Progression: | |
| | 5.0 mos (3-23) | 4.0 mos (2.5-14) | |

*LTF = Lost to followup;
CR = Complete Response;
PR = Partial Response;
NC = No change

TABLE 11
Peripheral Blood Leukocyte Counts and Pleural Fluid
Nucleated Cell Counts in Patients Treated with egc-SAg

| | | |
|---|---|---|
| WBC/μl | Neutrophils/μl | Lymphocytes/μl |

Peripheral Blood (n = 10)

| | | | |
|---|---|---|---|
| Pre-treatment | 5,200 ± 0.398 | 3,285 ± 2.50 | 1,850 ± 0.144 |
| Post- | 8,533 ± 1.534* | 6,455 ± 1.535* | 1,916 ± 0.587 |
| treatment^{‡} | | | |

Pleural Fluid (n = 10)

| | | | |
|---|---|---|---|
| Pre-treatment | 761 ± 0.150 | 553 ± 0.150 | 201 ± 0.134 |
| Post- | 1,178 ± 0.381* | 661 ± 0.185* | 541 ± 0.167* |
| treatment^{‡} | | | |

*significant at p < 0.05
^{‡}Peripheral blood and pleural effusion cell counts 6-24 hour after initial treatment.

### Discussion

We found that intrapleural administration of egc SAgs to 14 unselected, consecutive patients with MPE from NSCLC resulted in resolution of the MPE within 1 month after treatment with persistence for more than 90 days in 12 of 14 patients. In several cases, resolution lasted for as long as 6, 8, 12 and 15 months with a median time to recurrence of 5 months. The response rate (100%) for resolution of the MPE exceeded that for talc, bleomycin, doxycycline and indwelling catheter drainage that are commonly used for local palliation of MPE from NSCLC. None of the latter treatments has been shown to improve survival.

While MPEs resolved with egc SAgs, it appeared that a substantial number of the patients also survived longer than would be expected than if the egc SAgs only induced palliation. The median survival of 7.9 months in the NSCLC patients with MPE included 3 patients who survived more than 350 days. At the time of this report, one patient is still alive 36 months after starting treatment.

The median survival of 7.9 months for all 14 consecutive, unselected egc SAgs-treated patients stood in comparison to a median survival of 2.5 months for all 18 consecutive, unselected patients with MPE from NSCLC treated with talc poudrage at UCSD from 1993-1998 (Burrows et al., supra). Thirteen of these 18 patients with a pretreatment KPS range of 10-60, median 30 and distribution statistically similar to that of the entire SAgs-treated group had a median survival of 2.0 months. Additional groups of 61 and 35 patients with MPE from NSCLC treated with indwelling catheter drainage and doxycycline pleurodesis in a multicenter trial led by investigators from MD Anderson Cancer Center from 1994-1996 had median survivals of 2.0 months (Putnam et al supra 1999) and 3.0 months (Putnam et al., supra 2000), respectively. A meta-analysis of 156 patients with MPE from lung cancer showed a median survival of 3.0 months (Heffner J E Chest 117: 79-86 (2000)). Compared to current historical controls treated with the best available palliative measures (talc, doxycycline and indwelling catheter drainage), SAgs-treated patients had a 2.4 to 4 fold greater survival.

The median survival of 7.9 months for the 14 egc SAgs-treated patients was surprising in view of the low median pretreatment KPS score of 40 or ECOG 3 (disabled, bedridden >50% waking hours) for this group. Nine patients with KPS of 40 and below had a median survival of 8.6 months. Platinum-based chemotherapy is generally not recommended for patients with KPS ≦70 (ECOG ≧2) since it induces a greater level of toxicity compared to those with KPS ≧70 (ECOG 0-1). Recent chemotherapeutic regimens used in stage IIIb patients (with pleural effusion) selected for ECOG 0-1, KPS ≧70 have shown an improved median survival of approximately 8 months comparable to the survival reported herein for SAgs-treated patients with a median KPS of 40 (ECOG 3). As a single agent egc SAg appears to be capable of inducing an MPE response rate exceeding talc with less morbidity and a survival duration in a group with poor performance status (KPS 40) comparable to cisplatinum-based chemotherapy in patients with better performance (KPS ≧70). Thus, egc SSAg treatment may be useful in stage IIIb patients with MPE, KPS 40 or ECOG 3 who are ineligible for chemotherapy. Notably, pretreatment KPS scores <70 (range 30-60) in 8 patients improved to a KPS status of ≧70 (ECOG 2) after a single course of egc SAg treatment suggesting that patients considered ineligible for chemotherapy might become eligible after SSAg therapy

Notwithstanding the limitations of comparing populations in different countries with different medical systems, the egc SAgs and talc poudrage-treated groups had certain similarities. Both groups comprised unselected and consecutive patients with MPE from NSCLC. Median age, TMN subset grouping and pretreatment KPS scores were not statistically different. In both groups, the vast majority had stage IIIb lung cancer due to adenocarcinoma, symptomatic pleural effusions were present at the time of first treatment and there was a comparable degree of parenchymal tumor or tumor related lung lesions. Fifty seven percent of egc SAgs- and 56% of talc-treated patients had not received prior cancer chemotherapy and/or radiation treatment consistent with the experience of Schrump and coworkers (Schrump D S, Nguyen D M. Malignant pleural and pericardial effusions. Cancer, Principles and Practice of Oncology, DeVita, V, Hellman, S, Rosenberg, S A, eds. Lippincott Williams & Wilkins, Philadelphia, Pa. 2001, pp. 2729-2744) and Maghfoor and colleagues (Maghfoor I, Doll D C, Yarbro J W. Effusions in Clinical Oncology. Clinical Oncology, 2nd Edition Abeloff, M D, Armitage J O, Lichter M, Niederhuber J E. Eds. Churchill Livingstone, New York, N.Y., 2000, pp. 922-949) who noted that 46-64% of NSCLC patients present with MPE as the first sign of malignancy. The median survival for MPE from NSCLC is compatible with findings in previous reports of similar patients in China showing no better survival rates than in western populations. Given the similarity of the two populations, a comparison of survival rates was considered to be reasonable.

The only toxicity of egc SAgs treatment was fever which never exceeded grade 2. Fever was easily managed with conventional antipyretics. There was no grade 3 or 4 toxicity and all patients were discharged from the hospital within 24 hours after the procedure. Notable was the absence acute respiratory distress syndrome as has been observed following talc insufflation or instillation and hypotension and non-cardiogenic pulmonary edema that has been reported after treatment of cancer patients with preparations containing staphylococcal superantigens, enterotoxins A and B. In addition to the lack of significant toxicity, egc SAgs may offer potential advantages over approved palliative agents used for MPE in requiring minimal hospitalization while also avoiding thoracotomy, chest tube insertion and prolonged in-hospital chest tube drainage.

Following intrapleural egc SAgs, the pleural fluid showed significant accumulation of lymphocytes in addition to neutrophils. In contrast, the response to acute pleural injury caused by infection or induced by inflammatory or sclerosing agents in rabbits is manifest by a neutrophil influx which persists in the pleural fluid as long as the injury is maintained. If the effusion persists, lymphocytes predominate, however, if the injury ceases, blood monocytes transiently become more prevalent. The acute neutrophilia and lymphocytosis noted in the SAgs-treated patients may be ascribed, in part, to superantigen induction of T-cell lymphotactin and IL-8, which are chemotactic for lymphocytes and neutrophils, respectively, at the site of superantigen administration.

Superantigens derive their name from the shared property of activating a high proportion of T cells via binding to the T cell receptor Vβ region. Each superantigen activates a unique cluster of Vβs on the T cell receptors of CD4+ and CD8+ T-cells. The preparation used in these studies was free of toxicity noted with the use of other preparations containing staphylococcal enterotoxins A and B.

The resolution of pleural effusions and prolonged survival of NSCLC patients with egc SAg therapy may be ascribed, in part, to a SAg-induced tumoricidal reaction in the pleura and pleural space. This is supported by tumoricidal effects noted in pleural fluid cytology samples obtained from several patients 6-24 hours after treatment with SAgs. Superantigens are known to induce a population of CD4+ and CD8+ effector T cells expressing CD44 and CD62_{low}, capable of trafficking to tumor sites and killing tumor cells directly or via release of tumoricidal cytokines and chemokines. Intrapleurally administered SAgs may traffic primarily to regional lymphatic lacunae via stoma and foramina in the macula cribiformis and ultimately drain into the parasternal, costal, bronchial and mediastinal lymph nodes (Takashi M et al., J Thorac Cardiovasc Surg 120: 437-447 (2000)) where they activate effector and migratory T-cells expressing CD44 and CD62low T cells (DeGrendele H C et al., Science. 278: 672-674 (1997); DeGrendele H C et al., J Immunol. 159:2549-53 (1997); Siegleman M H et al., J Clin Invest. 105: 683-690 (2000); Miethke T et al., J Immunol. 151:6777-82 (1993); Kagamu H et al., J Immunol. 160:3444-52 (1998); Von Andrian U H et al., New Eng J Med. 343: 1020-1033 (2000)). These same effector T-cells translocate into the pleural space where their cytotoxic effect is exerted on carcinoma cells with or without surface-bound superantigen. Likewise, tumoricidal effector cells generated in the mediastinal lymphatics may limit the growth of parenchymal or hilar tumor to account for the stability of lung tumor masses in the SAgs-treated cases. SAg-specific antibodies present naturally in the blood of most humans and considered to be an impediment to superantigen-induced tumor killing when administered intravenously, may actually contribute to killing of tumor cells displaying surface bound SAg by complement-mediated tumor lysis and/or antibody-dependent cellular cytotoxicity.

As a safe outpatient procedure, egc SAg therapy appears to offer considerable cost reduction compared to the presently available agents which while also providing symptomatic relief and a significant survival benefit. Table 12 shows the comparative cost effectiveness with palliative treatments.

TABLE 12

| | AGENT COST OF | TOTAL | |
|---|---|---|---|
| TREATMENT | TREATMENT | COST | COST DRIVERS |
| Talc insufflation | $0.15-0.50 | $30,996 | OR Facilities, Thoracic Surgeon, |
| | (2.5-10 g) | | Respiratory Therapy, Hospitalization, |
| | | | Indwelling Chest Tube, Complications |
| | | | (ARDS) |
| Talc slurry | $0.15-0.50 | $25,000 | Hospital days, Respiratory Therapy, |
| | (2.5-10 g) | | Indwelling Chest Tube, Complications |
| Bleomycin | $1104 | $20,000 | High Agent cost, Hospitalization, |
| | | | Indwelling Chest Tube, Toxicity Potential |
| | | | with Chemotherapy Low Response Rate, |
| | | | High Recurrence Rate |
| Egc | $300 | $2000-$10,000 | NONE of the following: OR facility |
| Superantigens | | | Thoracic Surgeon, Hospitalization, |
| | | | Respiratory Therapy, Indwelling Chest |
| | | | Tube & Drainage, |

Patients with MPEs from small cell carcinoma of the lung, uterine sarcoma and melanoma were treated with intrapleural egc SAg and showed resolution of their MPEs for 1.5-4 months. Thus, it would be expected that egc SAg therapy is applicable to a substantial number of malignant pleural effusions from tumors other than NSCLC including breast carcinoma, stomach, esophageal and colon carcinoma, ovarian and uterine tumors, melanomas, mesotheliomas, liver tumors, lymphomas and metastatic tumors to the lung of any kind with or without associated pleural effusion.

Furthermore, intrapleural egc SAg maybe effective against parenchymal lung tumors of any kind with or without pleural effusions. The evidence for this is the regression of parenchymal lung tumor noted in one case and stabilization of measurable lung masses for up to 4 month after intrapleural SAg treatment.

In addition, these finding suggest that there the intrapleural route may be effective in treating asymptomatic MPEs from NSCLC which may be present in up to 50% or MPEs from NSCLC. In lung cancer in particular, the presence of a malignant pleural effusion from NSCLC portends a prognosis of two months survival (irrespective of initial effusion volume). Egc SAg is also applicable to patients with small asymptomatic malignant pleural effusions irrespective of origin or initial pleural fluid volume. Thus, small symptomatic or asymptomatic MPEs originating from lung, breast, stomach, esophagus, colon, kidneys, ovary, uterus (or any other origin) as well as melanoma, lymphomas and mesotheliomas would be expected to benefit from this treatment which will prolong survival in these groups.

### Example 3

### Treatment of Lung Adenocarcinoma by Intratumoral Injection of SAgs Followed by Intratumoral Chemotherapy

### Patient and Treatment Plan

The patient is a 75 year old man with a large adenocarcinoma in the left midlung field. He received intratumoral administration of egc SAgs (0.1 pg-1.5 ng) containing once weekly for 7 weeks.

During weeks 8-11, the patient received weekly intratumoral injections of egc SAgs together with cisplatinum (10 mg) intravenously. Chest x-rays were done before treatment and 1 week after the conclusion of the last dose of intratumoral SAg/Cisplatinum.

Criteria for response are as set forth by the International Union Against Cancer and are given in more detail below. Briefly, a complete response is defined as no measurable disease. A partial response is as a 50% reduction of the bidirectional diameter of measurable tumor.

Results: One week after concluding the course of intratumoral egc SEs followed by intratumoral egc SEs+cisplatinum, the patient's chest x-ray and CT scan showed complete disappearance of the pulmonary nodule which measured 20 cm³ before commencing treatment. The lesion showed progressive reduction in size on ultrasound during the SAgs treatment phase. Morbidity consisted of a low grade temperature for 3-4 weeks after commencing SAgs therapy, fatigue and anorexia not requiring treatment. These symptoms abated with continued treatment. CBC, renal and liver functions tests did not change significantly after treatments.
Discussion: The egc SEs administered alone intratumorally for 7 weeks followed by a 3 week course of a combination of the egc SE and low dose cisplatinum, given intratumorally, induced complete remission. The dose of cisplatinum used is more than 10-fold lower than the mean recommended dose administered systemically per cycle. Side effects of the egc SE treatment were minimal, and cisplatinum caused no toxicity. This patient subsequently received two cycles of systemic cisplatinum and mitomycin C and remained in complete remission 7 months later.

### Example 4

### Clinical Trial of Intratumoral or Systemic egc SAgs, Immunocyte Survival-Promoting Cytokines and Low Dose Chemotherapy in Humans Patients

All patients treated have histologically confirmed malignant masses confirmed by biopsy or cytology are entered. Malignant diseases including carcinomas, sarcomas, melanomas, gliomas, neuroblastomas, lymphomas and leukemia. The malignant disease has failed to respond or is advancing despite conventional therapy. Patients in all stages of malignant disease involving any organ system are included. Staging describes both tumor and host, including organ of origin of the tumor, histologic type, histologic grade, extent of tumor size, site of metastases and functional status of the patient. For a general classification includes the known ranges of Stage 1 (localized disease) to Stage 4 (widespread metastases), see Abraham J et al., Bethesda Handbook of Clinical Oncology, Lippincott, Williams & Wilkins, Philadelphia, Pa., 2001. Patient history is obtained and physical examination performed along with conventional tests of cardiovascular and pulmonary function and appropriate radiologic procedures. The malignant masses are visible on x-ray or CT scan and are measurable with calipers. They have not been undergoing any other anticancer treatment for at least one month and have a clinical KPS of at least 50.

Egc SEs are used as the prototypical SAgs (but other SAgs, conjugates, derivatives fusion proteins and homologues as described herein are used in other patients in comparable doses, yielding similar results). SAgs are administered intratumorally or parenterally (intravenously, intramuscularly, intrathecally, intrapleurally, intrapericardially, subcutaneously, intravesicularly, intralymphatically, intraperitoneally, subcutaneously, intraarticularly) or orally in doses of 0.01 pg-1.5 ng for each egc SAg in the preparation every 2-7 days for up to 10 doses. The final SAgs preparation preferably contain one or a mixture of different egc SEs with a Vβ/Vα profile for the final preparation exhibiting a minimum activation of 5 different Vβ/α-expressing T cell clones and a maximum of 24 Vβ/α-expressing T cell clones. The preferred egc SAg mixture comprises native egc SEs or egc SE homologues or more preferably, SEG, SEI, SEM, SEQ, SEN or SE homologues or a mixture of native egc SEs and egc SE homologues which are administered by infusion, injection, instillation or implantation. One or a plurality of native egc SEs or egc SE homologues may be mixed with one or more native non-egc SAgs or non-SAg homologues or mixtures of native non-egc superantigens and their non-egc superantigen homologues are useful provided they activate/recognize a minimum of 5 different Vβ/α-expressing T cell clones or T cell populations after stimulation with individual SAgs.

For intratumoral administration, the tumors are injected under direct vision at surgery, bronchoscopy, endoscopy, peritoneoscopy, culdocopy. Tumors are accessible to percutaneous injection with CT, ultrasound or stereotaxis used to localize and guide the injected composition into the tumor.

Intratumoral or systemic (parenteral) chemotherapy preferably comprises the use of a selected single agent which is known in the art to be effective against a particular tumor. Intratumoral or systemic combination chemotherapy wherein each agent is given in a full or reduced dose can also be used. Preferably, total intratumoral or systemic dose of a chemotherapeutic agent per cycle is 3-7 fold below that of the mean recommended dose of a systemic chemotherapeutic agent per cycle. Recommended mean dosages for single and individual chemotherapeutic agents for human tumors are well known in the art and given in Abraham et al., supra. The intratumoral or systemic dose of a cytotoxic drug administered to the tumor site generally ranges from about 0.1 to 500, more usually about 0.5 to 300 mg/kg body weight, depending upon the nature of the drug, size of tumor, and other considerations. The intratumoral or systemic chemotherapy is given after at least 1-7 weekly of intratumoral SAgs injections and within 36 hours after the previous SAgs treatment. The egc SEs and chemotherapy are given at the same time and continued every 7 days for at least 3 treatments and up to 6 weekly treatments if the tumor is shrinking and there is no dose limiting toxicity. The treatment is continued until there is evidence of tumor progression or complete remission.

Systemic single or combination chemotherapy is also used in full doses or in doses 10-95% below the mean recommended therapeutic dose for a single agent alone or for each chemotherapeutic agent in a mixture of chemotherapeutic agents. While a range of 10-95% reduction is useful, chemotherapeutic doses 50% below the recommended mean dose per cycle are used most often. Systemic or intratumoral chemotherapy is given together at the same time or preferably started within 36 hours after second to the seventh weekly intratumoral treatment with the egc SE composition alone. The egc SE/chemotherapy combination is preferably continued together for at least 3 weekly injections (range: 1-8 weeks) or longer if the tumor is shrinking and there is no dose limiting toxicity.

Immediately after each egc SAg administration systemically, parenterally or intratumorally, patients receive one or more of cytokines selected from a group consisting of one or more of IL-15 (0.15-8 mg/kg), IL-7 (0.5 ug/day), IL-23 (0.1-200 ug/day), with or without high-dose IL-2 therapy consisting of 720,000 units per kg bolus i.v. infusion every 8 hours to tolerance. The cytokines are given by injection, infusion or instillation via any parenteral route (including the same site as the egc SEs) including but not limited to intrathecally, intrapleurally, intrapericardially, intraperitoneally, intravenously, intramuscularly, intratumorally, intracranially, intraarticularly, intralymphatically, intradermally The cytokines are given twice daily for 3-7 days after each egc SAg injection. They are also effective when given before (preferably 1 hour) and at the same time (including together with) as each egc SAg administration.

In the case of a lung tumor, a typical treatment consists of percutaneous or transbronchial injection of a lung tumor nodule intratumorally with egc SEs 0.1 pg-1.0 ng of each egc SAg in the egc SE composition every 7 days for 7 weeks followed by egc SEs 0.1 pg-10.5 ng of each egc SE in the egc SE composition with cisplatinum 10 mg intratumorally or systemically every 7 days for three weeks. The chemotherapy is used alone (i.e. without the egc SAg composition or together with egc SAg for the last three treatments). For large tumors exceeding 40 cm² (two dimensions), injections are given at more than one site in the tumor mass using doses that cumulatively do not exceed that of a single dose per cycle. Likewise, additional malignant nodules or masses are treated in the same fashion as large single nodules. Alternatively, additional nodules are treated sequentially following the completion of one cycle in a single mass.

Representative doses of single agent chemotherapeutic agents used in an average sized adult for intratumoral injection against the more common tumors are, (1) Breast carcinomas: Doxorubicin (14-30 mg/treatment×3), Taxol (30 mg/treatment×3); (2) Colo-rectal cancer: 5-Fluorouricil (180 200 mg/treatment×3); Lung cancer: Cisplatinum (4-10 mg/treatment×3). The drugs are administered intratumorally in 1 ml normal saline over a 1 minute period or systemically over the FDA recommended time period. The same chemotherapeutic agents are administered systemically in full FDA-recommended doses or preferably in dose 10-50% below that of the full recommended systemic dose per treatment.

Patient Evaluation: Assessment of response of the tumor to the therapy is made once per week during therapy and 30 days thereafter using CT or x-ray visualization. Depending on the response to treatment, side effects, and the health status of the patient, treatment is terminated or prolonged from the standard protocol given above. Tumor response criteria are those established by the WHO and RECIST (Response Evaluation Criteria in Solid Tumors) summarized below (also Abraham et al., supra)

| RESPONSE | DEFINITION |
|---|---|
| Complete | Disappearance of all evidence of disease |
| remission (CR) | |
| Partial | □50% decrease in the product of the two |
| remission (PR) | greatest perpendicular tumor diameters; no new lesions |
| Less than partial | 25%-50% decrease in tumor size, stable for at least |
| remission (<PR) | 1 month |
| Stable disease | <25% reduction in tumor size; no progression or |
| | new lesions |
| Progression | ≧25% increase in size of any one measured |
| | lesion or appearance of new lesions despite |
| | stabilization or remission of disease in other |
| | measured sites |

The efficacy of the therapy in a patient population is evaluated using conventional statistical methods, including, for example, the Chi Square test or Fisher's exact test. Long-term changes in and short term changes in measurements are evaluated separately.

### Results

A total of 810 patients are patients treated. The number of patients for each tumor type and the results of treatment are summarized in Table 11. Positive tumor responses are observed in 80-90%% of the patients with breast, gastrointestinal, lung, prostate, renal and bladder tumors as well as melanoma and neuroblastoma as follows:

Six hundred and sixty five patients with all tumors exhibit objective clinical responses for an overall response rate of 82%. Tumors generally start to diminish and objective remissions are evident after four weeks of combined SAgs and chemotherapy. Responses endure for an average of 24 months.

Toxicity consists of mild short-lived fever, fatigue and anorexia not requiring treatment. The incidence of side effects (as % of total treatments) are as follows: chills—10; fever—10; pain—5; nausea—5; respiratory—3; headache—3; tachycardia—2; vomiting—2; hypertension—2; hypotension—2; joint pain—2; rash—2; flushing—1; diarrhea—1; itching/hives—1; bloody nose—1; dizziness—<1; cramps—<1; fatigue—<1; feeling faint—<1; twitching —<1; blurred vision—<1; gastritis<1; redness on hand—<1. Fever and chills are the most common side effects observed. Side effects are somewhat less frequent in patients treated with intratumoral SAgs plus low dose single agent chemotherapy compared with SAgs and full dose systemic chemotherapy. Side effects are less prevalent with the intratumoral SAgs-chemotherapy regimen compared with SAgs and full dose systemic chemotherapy regimen but this is not statistically different. CBC, renal and liver functions tests do not change significantly after treatments.

TABLE 11

| | | | % of Patients |
|---|---|---|---|
| | No. | Response | Responding |
| All Patients | 567 | CR | 70 |
| | 70 | PR | 8.6 |
| | 28 | <PR | 3.4 |
| By Tumor Type: | | | |
| Breast adenocarcinoma | 100 | CR + PR + <PR | 80% |
| Gastrointestinal carcinoma | 100 | CR + PR + <PR | 85% |
| Lung Carcinoma | 150 | CR + PR + <PR | 90% |
| Brain glioma/astrocytoma | 50 | CR + PR + <PR | 80% |
| Prostate Carcinoma | 100 | CR + PR + <PR | 80% |
| Lymphoma/Leukemia | 80 | CR + PR + <PR | 75% |
| Head and Neck Cancer | 80 | CR + PR + <PR | 75% |
| Renal and Bladder Cancer | 50 | CR + PR + <PR | 90% |
| Melanoma | 50 | CR + PR + <PR | 80% |
| Neuroblastoma | 50 | CR + PR + <PR | 80% |

### Example 5

### Treatment Plan and Outcome Prediction Using Intrapleural Superantigens

Patients have with malignant pleural effusions confirmed by biopsy or pleural fluid cytology and have not been undergoing any other anticancer treatment for at least one month and have a clinical Karnofsky status of at least 60-70%. The final egc SE preparation should preferably contain one or a mixture of different egc SEs with a Vβ/Vα profile for the final preparation exhibiting a minimum activation of 5 different Vβ/α-expressing T cell clones and a maximum of 24 Vβ/α-expressing T cell clones. The preferred egc SAg mixture comprises native egc SEs or egc SE homologues or more preferably, SEG, SEI, SEM, SEQ, SEN or SE homologues or a mixture of native egc SEs and egc SE homologues which are administered by infusion, injection, instillation or implantation using doses of each egc SE in a of range of 0.1 pg-1.5 ng for each treatment. One or a plurality of native egc SEs or egc SE homologues is/are mixed with one or more native non-egc SAgs or non-SAg homologues or mixtures of native non-egc superantigens and their non-egc superantigen homologues are useful provided they activate/recognize a minimum of 5 different Vβ/α-expressing T cell clones or T cell populations after stimulation with individual SAgs.

Egc SEs are administered intrapleurally once or twice weekly immediately after drainage of the effusion via conventional thoracentesis. This procedure is performed once or twice weekly in an outpatient or office setting. Treatment is continued once weekly until effusion does not recur. An objective response is recognized as no clinically significant reaccumulation of pleural fluid 30 days after treatment (DeCamp M M et al., Chest 112: 291S-295S (1997); Fenton K N et al., Am J. Surg. 170: 69-74 (1995)). The egc SEs may also be given parenterally (intravenously, intramuscularly, intradermally, subcutaneously intratumorally, intrapleurally, intrathecally, intrapericardially, intravesicularly, subcutaneously, intralymphatically, intraarticularly, intradermally or intramuscularly) by infusion, instillation, injection, or implantation simultaneously or sequentially with intrapleural therapy. For parenteral therapy, the egc SEs are given daily or every 2-7 days for 30 days (each cycle is repeated at 2-8 month intervals) in doses of each egc SE in a of range of 0.1 pg-1.5 ng for each treatment.

Immediately after each egc SAg administration systemically, parenterally (e.g., intrapleurally), patients receive cytokines selected from a group consisting of one or more of IL-15 (0.15-8 mg/kg), IL-7 (0.5 ug/day), IL-23 (0.1-200 ug/day), with or without high-dose IL-2 therapy consisting of 720,000 units per kg bolus i.v. infusion every 8 hours to tolerance. The cytokines are given twice daily for 3-7 days after each egc SAg injection. The cytokines are administered individually (e.g., IL-15 alone) or as a plurality by injection, infusion or instillation intrathecally (intrapleurally, intravesicularly, intrapericardially, intraperitoneally), intralymphatically, intravenously, intramuscularly, intradermally, intraarticularly together with before or shortly after (e.g., minutes to 48 hours) the intrathecal administration of SE or egc SEs.

There are 90 evaluable patients with malignant pleural effusions treated with intrapleural egc SAgs. All patients have stage IIIb or stage 1V lung cancer. There are 50 evaluable patients with malignant ascites. Eighty five patients with pleural effusions exhibit objective clinical responses for a response rate of 94.5%. Effusion reaccumulation (at weekly intervals) progressively diminished after each egc SE treatment. Patients required an average of three treatments before there is no significant reaccumulation. However, several patients require only one treatment to eliminate fluid reaccumulation. Forty five patients with malignant ascites show objective responses for a response rate of 90%.

Toxicity in both malignant pleural effusion and ascites consists of mild short-lived fever, fatigue and anorexia not requiring treatment. CBC, renal and liver functions tests did not change significantly after treatments.

The egc SEs have better therapeutic efficacy for malignant pleural effusions and ascites than existing agents (talc, bleomycin, doxycycline) without the discomfort and complications associated with an indwelling draining chest tube. In the case of pleural effusion, It is also 90% more cost-effective compared to existing therapy since it is carried out in an outpatient facility and does not involve the major costs associated with hospitalization, i.e., chest tube insertion, operating and recovery room, indwelling chest tube drainage and respiratory therapy.

### Example 6

### Anti-Tumor Effects of Intratumoral SAgs and Chemotherapeutic Agents Administered in Viscous Form of Controlled Release Formulation

The egc SAgs and chemotherapeutic agents are prepared in controlled release formulations as follows.

Preparation of Controlled Release Formulation.

The preparation of the preferred biodegradable controlled release formulation for intratumoral administration of egc SEs and cisplatinum as a preferred single agent for use in patients with NSCLC is described. Cisplatinum is a representative chemotherapeutic agent; other chemotherapeutic agents preferred for a given type of tumor be prepared an used similarly with slight variations that are within the skill of the art.

Cisplatinum for Injection, USP (Platinol®, 10 mg vial) manufactured by Bristol Laboratories or lyophilized CDDP manufactured by Faulding (David Bull Laboratories, Australia) is used. Aqueous collagen gel, 6.5% is obtained from Collagen Corporation (Palo Alto, Calif.), 0.3 ml nominal fill in 1 ml plastic syringes. The gel comprises a highly purified, telopeptide-free bovine Type I collagen, 6.5% (w/w); sodium phosphates, 0.1 M; sodium chloride, 0.045 M; and has a nominal pH of 7.2. Optionally epinephrine is used as a solution (1 mg/ml). Polysorbate 80 is obtained from PPG Industries. Carboxymethylcellulose sodium (NaCMC) is obtained from Aqualon. 0.9% Sodium Chloride for injection, USP (10 ml vial) (“saline”) and sterile water for injection (‘WFI’), USP (10 ml vial) may be obtained from common sources (e.g., Abbott Laboratories).

Diluent contain polysorbate 80, 1.0 mg; edetate disodium dihydrate, 0.1 mg; USP carboxymethylcellulose 0.5 mg; sodium metabisulfite, 0.2 mg; glacial acetic acid USP, 0.49 mg; sodium acetate, anhydrous, 0.15 mg; WFI up to 1 ml. HCl and/or NaOH may be added if necessary, to adjust pH to 4.0. USP epinephrine, 0.160 mg is also optional. A second diluent contains all of the above ingredients except for carboxymethylcellulose.

The viscous form of cisplatinum is prepared by diluting 5-10 mg in 1-4 cc of diluent. The resulting solution is very viscous and can serve as a controlled release formulation upon injection into tumor. This is the preferred method of administration of this drug. Egc SAgs is present in the same solution as cisplatinum as a viscous mixture. In this way both cisplatinum and egc SAgs are injected into the tumor at the same time.

The gel is prepared by combining the various components in a sterile environment. Upon admixture of the bovine collagen matrix and other agents, a uniform dispersion is obtained. For collagen and collagen derivatives, the material is provided as a uniform dispersion of collagen fibrils in an aqueous medium, where the collagenous material ranges in concentration from about 5 mg/ml to not more than about 100 mg/ml. The drug and or egc SEs may then be added to the collagenous dispersion using agitation to ensure uniform dispersion of the active agents. Other materials, as appropriate, may be added concomitantly or sequentially. After ensuring the uniform dispersion of the various components in the mixture, the mixture is sterilized and sealed in appropriate containers.

Vials containing either 10 mg or 25 mg of lyophilized CDDP are reconstituted by adding either 1.6 ml or 4.0 ml of diluent, respectively, to yield a suspension of CDDP. SAgs are similarly reconstituted in sterile saline and added in desired concentration to the CDDP solution. Gels containing CDDP/SAgs are prepared in final volumes of 2.0 ml or 5.0 ml. Final gels contained 4.0 mg/ml CDDP with egc SEs consisting of 0.1 pg-1.5 ng of each egc SE in the mixture are prepared. The final SAgs preparation preferably contains one or a mixture of different egc SEs with a Vβ/Vα profile for the final preparation exhibiting a minimum activation of 5 different Vβ/α-expressing T cell clones and a maximum of 24 Vβ/α-expressing T cell clones. The preferred SAg mixture comprises native egc SEs or egc SE homologues or more preferably, SEG, SEI, SEM, SEQ, SEN or SE homologues or a mixture of native egc SEs and egc SE homologues which are administered by infusion, injection, instillation or implantation. One or a plurality of native egc SEs or egc SE homologues are mixed with one or more native non-egc SAgs or non-SAg homologues or mixtures of native non-egc superantigens and their non-egc superantigen homologues are useful provided they activate/recognize a minimum of 5 different Vβ/α-expressing T cell clones or T cell populations after stimulation with individual egc SAgs or non-egc SAgs and their homologue. Optionally, 0.1 mg/ml of epinephrine, with or without a 2% collagen matrix is administered intratumorally with the cisplatinum/SAg preparation.

Intratumoral Therapy: Therapy preferably comprises the use of a selected single agent ((chemo- or biotherapeutic) which is known in the art to be effective against a particular tumor, e.g., cisplatinum/carboplatin for NSCLC, doxorubicin/taxotere for breast carcinoma, 5-Fluoruricil for colorectal carcinoma, etc. Intratumoral combination chemotherapy wherein each agent is given in a reduced dose are also used. The intratumoral injection of egc SEs, 0.1 pg-1.5 ng, is given once weekly for 2-7 weeks. The chemotherapy is started within 36 hours of the last dose of egc SEs and then every 7 days for 3 treatments. egc SEs can also be given together with the chemotherapy or beginning with the first injection or second injection of chemotherapy.

The dose of a chemotherapeutic drug or biologic agent used for intratumoral administration, is reduced 10- to 50-fold below the mean FDA-recommended dose for parenteral administration in a single cycle. Chemotherapeutic concentrations in the sustained release preparation range from 0.01 to 50 mg/ml. Chemotherapy is given within 36 hours after the 7th intratumoral egc SE injection and continued once weekly for at least three weeks. It is extended to six or more weeks if the tumor is diminishing in size and there is no dose limiting toxicity. Injection of the dose is given at more than one site in tumors exceeding 40 cm2. In this case the dose is divided into two or more portions with the cumulative dose per treatment not to exceed that for a single site full dose.

Illustrative of the manner of sustained administration is intratumoral administration of cis-diaminodichloroplatinum (CDDP) in controlled release formulation for which the recommended intratumoral dose per weekly injection is 0.05-0.1 mg/kg with a total dose range dose of 12-30 mg per cycle. The final SAgs preparation preferably contains one or a mixture of different egc SEs with a Vβ/Vα profile for the final preparation exhibiting a minimum activation of 5 different Vβ/α-expressing T cell clones and a maximum of 24 Vβ/α-expressing T cell clones. The preferred SAg mixture comprises native egc SEs or egc SE homologues or more preferably, SEG, SEI, SEM, SEQ, SEN or SE homologues or a mixture of native egc SEs and egc SE homologues which are administered by infusion, injection, instillation or implantation in doses of each superantigen in a of range of 0.1 pg-1.5 ng for each treatment. One or a plurality of native egc SEs or egc SE homologues may be mixed with one or more native non-egc SAgs or non-SAg homologues or mixtures of native non-egc superantigens and their non-egc superantigen homologues are useful provided they activate/recognize a minimum of 5 different Vβ/α-expressing T cell clones or T cell populations after stimulation with individual SAgs.

Egc SEs are administered intratumorally once weekly for 2-7 weeks followed by CDDP (4-10 mg) weekly for 3 weeks. The tumors are accessed via percutaneous injection using CT, ultrasound or stereotaxis to localize and guide the injected composition into the tumor. In certain instances, the tumors are injected under direct vision at surgery, or via bronchoscopy, thoracoscopy, endoscopy, peritoneoscopy, cystoscopy, arthroscopy or culdocopy. Immediately after each egc SAg administration systemically, parenterally or intratumorally, patients receive one or more of cytokines selected from a group consisting of one or more of IL-15 (0.15-8 mg/kg), IL-7 (0.5 ug/day), IL-23 (0.1-200 ug/day), with or without high-dose IL-2 therapy consisting of 720,000 units per kg bolus i.v. infusion every 8 hours to tolerance. The cytokines are given twice daily for 3-7 days after each egc SAg injection. The cytokines can be delivered individually (e.g., IL-15) or as a plurality before, at the same time or after the SAg administration. IL-15 is preferred. They can be given together with the SAg into the same or different site, e.g., intrathecally, intratumorally, intrapleurally, intrapericardially, intravesicularly, subcutaneously, intralymphatically, intraarticularly, intradermally, intravenously or intramuscularly or by any other parenteral route by infusion, injection, instillation or implantation.

TABLE 13

| | | | % of Patients |
|---|---|---|---|
| | No. | Response | Responding |
| All Patients | 657 | CR | 80 |
| | 23 | PR | 3 |
| | 8 | <PR | 1 |
| By Tumor Type: | | | |
| Breast adenocarcinoma | 150 | CR + PR + <PR | 90% |
| Gastrointestinal carcinoma | 150 | CR + PR + <PR | 90% |
| Lung Carcinoma | 150 | CR + PR + <PR | 95% |
| Brain glioma/astrocytoma | 50 | CR + PR + <PR | 85% |
| Prostate Carcinoma | 100 | CR + PR + <PR | 85% |
| Lymphoma/Leukemia | 80 | CR + PR + <PR | 80% |
| Head and Neck Cancer | 80 | CR + PR + <PR | 80% |
| Renal and Bladder Cancer | 50 | CR + PR + <PR | 95% |
| Melanoma | 50 | CR + PR + <PR | 85% |
| Neuroblastoma | 50 | CR + PR + <PR | 85% |

Results: A total of 910 patients are patients treated. The number of patients for each tumor type and the results of treatment are summarized in Table 13. Positive tumor responses are observed in as high as 85-95% of the patients with breast, gastrointestinal, lung, prostate, renal and bladder tumors as well as melanoma and neuroblastoma as follows.

Seven hundred and seventy three patients of 910 entered with all tumors exhibit objective clinical responses for an overall response rate of 84%. Tumors generally start to diminish and objective remissions are evident after four weeks of combined egc SE-chemotherapy. Responses endure for an mean of 36 months.

Toxicity consists of mild short-lived fever, fatigue and anorexia not requiring treatment. The incidence of side effects (as % of total treatments) are as follows: chills—12; fever—15; pain—6; nausea—3; respiratory—2; headache—2; tachycardia—4; vomiting—4; hypertension—1; hypotension—2; joint pain—3; rash—1; flushing—4; diarrhea—2; itching/hives—1; bloody nose—1; dizziness—<1; cramps—<1; fatigue—<1; feeling faint—<1; twitching—<1; blurred vision—<1; gastritis<1; redness on hand—<1. Fever and chills are the most common side effects observed. Toxic effects usually associated with systemically administered chemotherapeutic agents are not observed. For example, neurotoxicity, hematologic toxicity, and ototoxicity associated with systemically administered cisplatinum are not observed. The bone marrow depression commonly observed with parenterally administered chemotherapy such as antimetabolites, e.g., 5-fluorouricil, methotrexate; alkylating agents, e.g., cyclophosphamide, ifosamide; tumor antimicrobials, e.g., doxorubicin, mitomycin C; plant alkaloids, e.g., taxol, taxotere; other agents, e.g., cisplatinum, carboplatin, irinotecan 5-fluorouricil, taxol, taxotere is not seen with intratumoral administration of the these agents in controlled release formulation. Unique toxicities of single agents such as cardiomyopathy with the anthracycline antibiotics doxorubicin, daunorubicin, hemorrhagic cystitis with cyclophosphamide and ifosamide, neurotoxicity with 5-fluorouricil, neuropathy and arrythmias with taxol, severe diarrhea with Irinotecan, interstitial pneumonia and hemolytic-uremic syndrome with mitomycin C are not observed when administered intratumorally as controlled release formulations. Of note, with all intratumoral chemotherapy in this form, there is minimal hematologic toxicity of the intratumorally administered chemotherapeutic agents and no significant renal and liver toxicity.

### Example 7

### Enhanced Chemotherapy-Induced Tumor Killing by Exposure of Tumor to SAgs

Tumor cells, exposed directly to SAg in vitro or media from PMBCs activated by SAg (SEP), exhibit a physiologic vulnerability to the tumoricidal effects chemotherapy. In a model system below, histopathologic osmotic swelling of tumor cells is noted after exposure to SEs or SEP mirrors the functional disruption of bidirectional barrier resistance as well as small ion, molecule and water flux across the tumor cell membrane leading to diffusion of chemotherapy into the cell and consequent tumor cell apoptosis.

### Solutions

1. T84 medium: 1:1 (v/v) Dulbeco's modified Eagle's medium (DMEM)/Ham's F12 medium supplemented with 2% (v/v) NaHCO₃, 200 mM L-glutamine, 2% (v/v) penicillin-streptomycin, 1.5% (w/v) HEPES, 10% (v/v) fetal calf serum (PCS) as culture medium. Keep sterile and store at 4° C., viable ˜3 mo.
2. 10× Hank's balanced salt solution (HBSS): 80.0 g NaCl, 4.0 g KCl, 0.9 g Na₂HPO₄-7H₂O, 0.6 g KH₂PO₄, 3.5 g NaHCO₃, 1.4 g CaCl₂, 1.0 g MgCl₂-6H₂0, 1.0 g MgS0₄-7H₂0, 10.0 g D-glucose. Add dH₂O to 1 L, autoclave (121° C., 15 min) or filter to sterilize. Store at 4° C. All buffers and media are available from Sigma (St. Louis, Mo.) or Gibco-BRL/Life Technologies Inc. (Rockville, Md.).

### Cell Types

1. Epithelial cells: Human (T84, HT-29, CaCo-2, SW460) and rodent (MODE K, IEC-6, IEC-18, KATO III) immortalized, transformed, or tumor cell lines are commercially available (American type culture collection [ATCC]; Manassas, Va.),

### Cell Culture

1. Cell-culture medium: DMEM, minimal essential medium (MEM), Ham's F12; supplements: fetal calf serum (PCS), antibiotics (penicillin-streptomycin), sodium bicarbonate, sodium pyruvate, HEPES, L-glutamine, 0.25% Trypsin-EDTA.

### Apparatus

1. Cell-culture apparatus: desktop centrifuge (swing bucket, accepts 15- and 50-mL tubes), laminar flow hood with aspirator, heated CO₂ incubator, heated water bath, standard or inverted microscope.
2. Voltmeter with chopstick electrodes (Millicell-ERS, Millipore, Bedford, Mass.) to monitor transepithelial resistance (TER).
3. Ussing chambers (World Precision Instruments (WPI), Sarasota, Fla.) and Voltage Clamp (DVC-100; WPI), including tubing and agar bridges, matched pre-amplifiers and calomel electrodes, heating pump, aeration regulator, chart recorder, or computerized acquisition system.

### Cell Culture

T84 human colon-carcinoma cells are grown in 75 cm² tissue-culture flasks under standard growth conditions (37° C., 5% CO₂) using T84 media replaced twice weekly.
1. Cells are passaged at confluence (usually 7-10 d after seeding), returning approx 1.5×10⁷ cells to a new flask.
2. Cells are counted using a standard haemocytometer slide and microscope.
10⁶ cells/filter are typically seeded onto transwell filter supports, using a filter size of 1 cm² (12-well plate) in 1 mL media, with 1.5-2.0 mL media added to the basal chamber. While being cultured on transwells, media is changed the day after seeding and every 24-48 h thereafter. By this method, T84 cells generally take 5-10 d to set up a maximally tight electrical resistance (1000-3000 Ω/cm2).

### Immune Cell Isolation

Peripheral blood mononuclear cells (PBMC) and lamina propria (LPMC) cells for activation by SAg. PBMC are isolated from donor blood using a Ficoll-Paque density gradient.
1. 10 mL of venus blood is collected in heparinized tubes.
2. The following steps must be conducted under sterile conditions.
3. Transfer blood to a 50-mL plastic tube containing 10 mL pre-warmed (37° C.) sterile PBS.
4. An underlay is prepared using a Pasteur pipet to slowly deliver 10 mL Ficoll solution to the bottom of the tube.
5. Being careful not to disturb the layering, tubes are centrifuged for 40 min at 300 g (brakes off). Red blood cells and other plasma constituents pellet to the bottom, while the heavier PBMC form a yellowish-coloured layer (‘buffy coat’) at the interface of the Ficoll-PBS/plasma gradient.
6. Remove PBMC by careful pipetting (use a small pipet, e.g., 5 mL), and transfer to a 15-mL tube.
7. Add warm PBS at a ratio of 1:4 (v/v), and centrifuge at 250 g for 10 min (brakes on).
8. Wash pelleted cells by re-suspending in warm PBS and repeat. Repeated washes increase cell purity but decrease yield.
9. Resuspend PBMC in culture medium (T84 media for co-culture experiments), count and adjust to the desired cell density (10⁶ cells/mL).

It is possible to investigate how the epithelium itself responds to culture with SEB or SAg-activated immune cells or CM by treatment with an inhibitory agent (e.g., steroids, or inhibitors of specific intracellular signaling molecules). The agent is added directly to the transwell, usually basally. Transepithelial resistance is monitored throughout the SEB or SAg incubation period (see below: assessment of barrier function). At the end of the SEB or SAg incubation period, the filter-grown epithelium is used to assess the impact of SEB or SAg or various cytokines or media from PBMCs that are incubated with SEs on ion transport. Additionally, epithelia is processed for examination by electron microscopy, immunocytochemistry/immunocytoflourescence, Western blot, and any other standard cellular, molecular, or enzymatic assays.

### Histologic and Immunohistologic Tests

The filter grown epithelium is transferred to cytolyte which results in rapid fixation of the cells. Cytolyte fixed specimens are used to prepare “Thinprep” cytology slides for morphologic study and the remaining fixed material is used to prepare cell-blocks. The cell block sections for histology are used for morphologic analysis and sections are used for ancillary studies, such as immunostaining (to assess biologic/physiologic responses, expression of apoptosis markers and indicators of proliferation).

### Assessment of Epithelial Ion Transport

Assessing Epithelial Ion Transport with Ussing Chambers

Vectorial ion transport and banner function is assayed using the Ussing chamber. The epithelial monolayer (on filter supports) is mounted between joining halves of the leucite chamber, both serosally and mucosally bathed by identical pre-oxygenated physiological buffers that nullify any hydrostatic or chemi-osmotic gradients. Kreb's buffer supplemented with 10 mM glucose is used. Each chamber half has two ports for agar bridges, which are connected via a reservoir of saturated KCl to either calomel reference electrodes or silver/silver chloride electrodes for measuring potential difference and injecting current, respectively. The pair of bridges placed closest to the epithelium monitor the spontaneous potential difference generated by the cells, while the bridges more distant from the epithelium are used for the injection of current. The chambers are oxygenated by a gas-lift and maintained at 37° C. by a heated water circulatory system. In the voltage-clamp setting, the potential difference (PD) across the epithelium is maintained at 0 volts, and the current that must be injected to maintain the 0 voltage is the short-circuit current, or Isc (in μA/cm2). Current is injected in response to active ion-transport events, and thus, the Isc is reflective of the net charge movement across the monolayer. Electrolyte transport creates the driving force for directed water movement, which, in the intestine, can result in a diarrheal response or constipation. The Isc indicates net charge movement but does not reveal the identity of the charge carrying ion.

In addition to continuous monitoring of baseline (or tonic) Isc, stimulated Isc responses are assessed by recording the peak change in Isc, or area under the curve, in response to pro-secretory (e.g., forskolin [Fsk], cholera toxin) or pro-absorptive (e.g., neuropeptide Y [NPY]) agents added directly into the appropriate side of the Ussing chamber. The Isc responsiveness is presented as μA/cm². Because of variability between cell passages, the data is normalized to time-matched naive epithelial monolayer responses and presented as percent of control events.

### Assessing Epithelial Ion Transport Using Voltmeter and Chopstick Electrodes

In the absence of the Ussing chamber-voltage clamp apparatus, a calculated Isc is obtained using a voltmeter and chopstick electrodes and taking readings directly from the transwell plate.

1. The electrodes are equilibrated by selecting voltage on the voltmeter and placing the electrodes in the buffer of choice for 1 h.
2. Prepare a 12-well plate as follows: the top row wells contain Kreb's+glucose, the middle row Kreb's buffer+0.01 M Fsk, and the bottom row Kreb's+0.1 M carbachol (CCh) (Fsk and CCh allow assessment of Cl˜ secretion in response to cAMP and Ca2+ mobilization, respectively. One 12-well plate so prepared is used for 4 epithelial monolayers.
3. Place the 12-well plate on a heating pad and allow to warm.
4. Aspirate apical and basal media from the transwells to be tested (maximum 4 at a time) and add Kreb's buffer to the apical compartment.
5. To equilibrate the monolayer, transwells are transferred to the top row of the prepared 12-well plate and voltage and resistance values are recorded at time 0 and 5 min later. The voltmeter should be set to read resistance while electrodes are being transferred between wells (or at anytime when they are not in buffer).
6. Take readings again after 10 min, the voltage reading should be steady before continuing with the experiment.
7. Transfer all transwells to the middle row containing the Fsk and record voltage and resistance at 5 min intervals until a peak response is determined.
8. Move only 2 wells at a time into the CCh solution and record voltage and resistance every 30 s for 4 min until the peak response is surpassed.
9. Repeat for the remaining 2 epithelial monolayers. To calculate Isc apply Ohm's Law: Voltage (V)=Current (I)×Resistance (R)

### Assessing Barrier Properties

### Transepithelial Electrical Resistance (TER)

TER indicates the passive flux of ions across the preparation and is generally considered a reflection of the leakiness of the tight junctions. Monolayers are mounted in the Ussing chamber and the voltage clamp is set in the bipolar mode, which allows the voltage to be jumped from 0 volts to, for example, 1 milli-volt at pre-set intervals. The change in Isc that occurs in response to 1 mV change in potential difference allows for the calculation of resistance via the Ohmic relationship. This procedure is referred to as the differential pulse technique and is the most sensitive measure of TER.

Alternatively, the Millipore voltmeter and chopstick electrodes are also an effective means to monitor TER. This procedure, although slightly less sensitive than the Ussing chamber has the advantages of: first, TER is monitored daily under sterile conditions in order to define when epithelial preparations are suitable for experimentation, e.g., TER >800 Ω/cm2; and second, paired analyses is performed since TER is monitored before and after co-culture with SAg-activated immune cells or exposure to CM.

### Small-Molecule Flux

In addition to TER, epithelial barrier function is assessed by monitoring the flux of “marker” molecules that are radiolabeled (e.g., ⁵¹Cr-EDTA), fluorescently labeled (e.g., dextrans; Molecular Probes, Eugene, Oreg.) or have an associated assayable enzymatic activity (e.g., horseradish peroxidase). Flux experiments are performed when the epithelium is mounted in the Ussing chamber or in transwell plates. The study of barrier function is accomplished using ⁵¹Cr-EDTA, 3H-mannitol or ³H-inulin—all small molecules that primarily cross the epithelium via the paracellular pathway. The probe molecule is added to either the basolateral (serosal) or apical (lumenal) compartment (now the “hot” side) and any potential osmotic effects countered by adding an equal volume (at the same concentration) of the nonlabeled probe to the “cold” side.

1. Epithelial monolayers are mounted in the Ussing chambers and allowed to establish a stable Isc and TER (−10-15 min).
2. The probe (e.g., 6.5 μCi ³H-mannitol) is added to the luminal buffer and equilibrates for 30 min.
3. Samples (500μι) are taken from the cold side at 20- or 30-min intervals (and replaced with the appropriate cold buffer) over a 90-min period.
4. Samples (50 μL) are taken from the hot side at the beginning and end of the experiment to calculate probe-specific activity.
5. Radioactivity is determined by counting in a γ- or scintillation counter and the flux presented as: 1) counts (or degradations) per minute (cpm); 2) percent cpm crossing the monolayer compared to initial cpm on the hot side; 3) flux rates calculated in amount.h.cm² by standard formulae; or 4) in the case of ionic fluxes, converted to μEquivalents.h.cm².
Clearly, increased transepithelial flux of the probe indicates increased epithelial permeability

### Results

### Histologic Studies

Histologic studies of epithelium and tumor cells incubated with and without SEB or other SAgs for 24 hours show definitive differences. In contrast to the untreated cell, the SEB-treated cells show cytoplasmic and nuclear swelling, granulation of cytoplasm and nuclear granules. The cell membranes are not noticeably disrupted. Histologic examination of the SEB or SAg-treated cells further incubated for 8 hours with cisplatinum show extensive apoptotic changes and nuclear degeneration that are not observed in the cells treated with cisplatinum alone.

Barrier Properties Transepithelial Resistance The change in transepithelial resistance (TER) in T84 monolayers incubated with SEB, cisplatinum and SEB/cisplatinum is shown in the Table 134 below:

TABLE 14
Treatment of T84 Monolayers with SEB and Cisplatinum

| T84 Treatment | TER (Ω/cm²⁾ |
|---|---|
| Untreated | 1100 |
| SEB 1 ug/ml 24 h | 1000 |
| SEB 10 ug/ml 24 h | 1000 |
| Cisplatin 0.1 ug/ml 12 h | 800 |
| Cisplatin 1.0 ug/ml 12 h | 750 |
| SEB-Cisplatin 24 h/12 h | 100 (range: 83-118) |

### Barrier Properties Transepithelial Flux

The change in transepithelial flux of the inert marker ⁵¹Cr-EDTA across T84 monolayers that are incubated with SEB, cisplatinum and SEB/cisplatinum is shown in the Table 15 below.

TABLE 15
Treatment of T84 Monolayers with SEB and Cisplatinum

| | ⁵¹Cr-EDTA flux |
|---|---|
| T84 Treatment | (nml · h · cm²) |
| Untreated | 2.0 |
| SEB 1 ug/ml 24 h | 2.7 |
| SEB 10 ug/ml 24 h | 2.8 |
| Cisplatin 0.1 ug/ml 12 h | 2.8 |
| Cisplatin 1.0 ug/ml 12 h | 2.8 |
| SEB-Cisplatin 24 h/12 h | 7.0 (range: 5.8-9) |

### Barrier Properties: Epithelial Ion Transport

The change in short circuit current evoked by carbachol and forskolin across T84 monolayers that are incubated with SEB, cisplatinum and SEB/cisplatinum (Table 16).

TABLE 16
Treatment of T84 Monolayers with SEB and Cisplatinum

| | Δ Isc to | Δ Isc to |
|---|---|---|
| T84 Treatment | CCh (μA/cm2) | Fsk (% of control) |
| Untreated | 25 | 93 |
| SEB 1 ug/ml 24 h | 23 | 95 |
| SEB 10 ug/ml 24 h | 23 | 95 |
| Cisplatin 0.1 ug/ml 12 h | 20 | 90 |
| Cisplatin 1.0 ug/ml 12 h | 20 | 90 |
| SEB-Cisplatin 24 h/12 h | 5 (range: 1-10) | 15 (range: 9-21) |

Clinically, one or preferably a plurality of SAgs and/or egc SAg's (0.0001 ng to 1.5 ug) are given parenterally, intrapleurally, intratumorally, intrathecally, intravescicularly, intradermally, subcutaneously, intravenously by injection or infusion or implantation before or after chemotherapy. In an example involving a patient with lung cancer, chemotherapy is started on the first day of the first week with cisplatinum and taxotere. On the first day of the second week and third weeks, egc SEs are administered parenterally, intrathecally or intrapleurally. On the first day of the forth week, the identical three week chemotherapy-immunotherapy program is repeated. The three week cycles are repeated for a total of 3-6 cycles as given in Table 17.

Alternatively, the egc SAgs is administered before chemotherapy. In one example, the SAg is administered on day 1 and the chemotherapy is administered preferably up to 48 hours later. The egc SAgs is also administered every 2-7 days for 2-10 weeks after which chemotherapy is administered. Preferably the chemotherapy is given within 48 hours after the last egc SAg treatment.

TABLE 17

| | Egc SAg/ | Taxane/ |
|---|---|---|
| | Chemo | Platinum |
| Treatment Regimen | (n = 150) | n = 96 |
| Paclitaxel 175/m²: Cisplatin 75 mg/m² | 6 | 4 |
| Paclitaxel 175/m²: Carboplatin AUC of 6 | 35 | 52 |
| Docetaxel 75 mg/m²: Cisplatin 75 mg/m² | 15 | 17 |
| Docetaxel 75 mg/m²: Carboplatin AUC of 6 | 44 | 26 |
| Average Number of Cycles (Min. Max) | | |
| Mean # cycles (Min. Max) | 3.4 (1, 8) | 3.2 (1, 6) |
| Taxane/Platinum Dose Reduction | | |
| % of patient requiring dose reduction | 25% | 23% |
| SAg Administration Dosing | | |
| SAg doses (Min. Max) | 6.4 (1, 16) | |
| % of SAg patients requiring dose reduction | 10% | |

In a clinical trial, 246 patients with stage 1V non small cell lung cancer were randomized to receive either the egc SAg/taxane-cisplatinum regimen or taxane-cisplatinum. Results at 1 year showed a progression-free survival of 49% in the egc SAg/taxane-cisplatinum group versus 21% in the taxane-cisplatinum group.

### Example 8

### Adoptive Immunotherapy with SAg & Cytokines

### Isolation of Host Cells: Lymph Nodes

As noted previously, the invention involves, in one embodiment, a method wherein host cells are removed and stimulated outside the body, i.e., ex vivo, with stimulating antigens. These cells are isolated from a variety of sources. In this example, they are obtained from the lymph nodes.

Inguinal, mesenteric, or superficial distal axillary lymph nodes are removed aseptically. Single cell suspensions are prepared by teasing (e.g., with 20-gauge needles) followed by pressing mechanically with the blunt end of a 10-ml plastic syringe plunger in buffer under sterile conditions. The cell preparations are filtered through a layer of No. 100 nylon mesh (Nytex; TETKO Inc., Elmsford, N.Y.), centrifuged and washed. Red cells, if evident, are lysed by treatment with ammonium chloride-potassium lysing buffer (8.29 g NH₄Cl, 1.0 g KHCO₃, and 0.0372 g EDTA/liter, pH 7.4). The cells are washed twice with buffer and resuspended for stimulation.

### Isolation Of Host Cells: Spleen Cells

In this example, the host cells are obtained from the human spleen. Either a left subcostal incision or midline incision is used for resection. The spleen is mobilized initially by dividing the ligamentous attachments, which are usually avascular. The short gastric vessels then are doubly ligated and transected. This permits ultimate dissection of the splenic hilus with individual ligation and division of the splenic artery and vein.

The sequence of technical maneuvers necessary to remove the spleen varies somewhat, depending on the surgeon's election to approach the splenic hilum either anteriorly or posteriorly. The anterior approach is somewhat slower.

### Anterior Method.

On entering the abdomen, the stomach should be thoroughly emptied by suction through a nasogastric tube already in place, if this maneuver has not been accomplished preoperatively. An opening is made in the gastrosplenic omentum in an avascular area, and by retracting the stomach upward and anteriorly through this opening the upper part of the pancreas is visualized. The tortuous splenic artery is seen along its upper margin; it is, at the option of the surgeon, ligated.

The next step in the procedure is division of the lower two-thirds of the gastrosplenic omentum. This is accomplished by dividing the vascular omentum between clamps and ligating the cut ends subsequently. The gastrosplenic omentum is frequently infiltrated with a considerable amount of adipose tissue and tends to slip away from clamps, especially if traction is applied to the instruments. The upper portion of this omentum also contains the vasa brevia and large venous tributaries joining the left gastroepiploic vein. To avoid hemorrhage from these sources, suture ligation rather than simple ligatures is utilized in this area. Access to the upper portion of the gastrosplenic omentum is difficult with the spleen in situ, and for this reason it is best divided with the later stage after mobilization of the splenic hilum.

Following division of the splenic vasculature, the splenorenal, the splenocolic, and the splenophrenic ligaments are divided. All except the last mentioned are generally avascular and pose no particular technical problems in division. The remnants of the splenophrenic ligament left behind may have to be underrun with running chromic catgut suture for hemostasis. The spleen is displaced from the abdomen and delivered through the incision. The only remaining attachments still in place is the upper third of the gastrosplenic ligament which is now carefully divided between ligatures, completing the splenectomy procedure.

### Posterior Method.

The posterior approach of removing the spleen is much more expeditious than the anterior approach, but blood loss is usually more substantial than in the anterior approach. After entering the abdomen the surgeon makes an incision in the avascular splenorenal ligament and then inserts three fingers behind the hilum of the spleen which is easily mobilized by blind dissection. Hemorrhage from the splenic hilum during this process is avoided by placing the incision on the splenorenal ligament closer to the kidney and away from the spleen. By rapidly dividing the splenophrenic and the splenocolic ligaments, the spleen is delivered through the incision. Any hemorrhage from the splenic hilum or from the ruptured spleen itself is very easily controlled at this point by manual compression of the splenic hilum or placement of a noncrushing clamp, taking care not to injure the tail of the pancreas. The gastrosplenic ligament and the presplenic fold when present is now divided and suture ligated in a deliberate manner.

### Spleen Cell Suspensions.

Spleen cells are mechanically dissociated by using the blunt end of a 10-ml plastic syringe in buffer. The cell suspension is passed through a single layer of 100-gauge nylon mesh (Nitex; Lawshe Industrial Co., Bethesda, Md.) and centrifuged, and the RBC are lysed by resuspension of the cell pellet in ammonium chloride/potassium lysing buffer, (8.29 g of NH₄Cl, 1.0 g KHCO₃ and 0.0372 g of EDTA/L pH 7.4; Media Production Section, National Institutes of Health, Bethesda, Md.). The cells are again filtered through nylon mesh, washed two times, and resuspended in culture medium.

### Isolation of Host Cells: Infiltrating Cells

In this example, the host cells are obtained from tumor infiltrating lymphocytes. Lymphocytes infiltrating tumors are obtained using standard techniques. Solid tumors (freshly resected or cryopreserved) are dispersed into single cell suspensions by overnight enzymatic digestion >e.g., stirring overnight at room temperature in RPMI 1640 medium containing 0.01% hyaluronidase type V, 0.002% DNAse type I, 0.1% collagenase type IV (Sigman, St. Louis), and antibiotics. Tumor suspensions are then passed over Ficoll-Hypaque gradients (Lymphocyte Separation Medium, Organon Teknika Corp., Durham, N.C.). The gradient interfaces contain viable tumor cells and mononuclear cells are washed, adjusted to a total cell concentration of 2.5 to 5×10⁵ cells/ml and cultured in complete medium. Complete medium comprises RPMI 1640 with 10% heat-inactivated type-compatible human serum, penicillin 50 IU/ml and streptomycin 50 ug/ml (Biofluids, Rockville, Md.), gentarnicin 50 ug/ml (GIBCO Laboratories, Chagrin Falls, Ohio), amphotericin 250 ng/ml (Fungizone, Squibb, Flow Laboratories, McLean, Va.), HEPES buffer 10 mM (Biofluids), and L-glutamine 2 mM (MA Bioproducts, Walkersville, Md.). Conditioned medium from 3- to 4-day autologous or allogeneic lymphokine-activated killer (LAK) cell cultures is added at a final concentration of 20% (v/v). Recombinant IL-2 (kindly supplied by the Chiron Corporation, Emeryville, Calif.) is added at a final concentration of 1000 ug/ml.

Cultures are maintained at 37° C. in a 5% CO₂-humidified atmosphere. A variety of tissue culture vessels are employed, including 24-well plates (Costar, Cambridge, Mass.). 175 cm.sup.2 flasks (Falcon; Becton Dickinson, Oxnard, Calif.), 850 cm² roller bottles (Corning Glass Works, Corning, N.Y.), and 750 cm² gas-permeable culture bags (Fenwal Laboratories, Division of Travenol Laboratories, Deerfield, Ill.). Cultures are fed weekly by harvesting, pelletting and resuspending cells at 2.5.times.10⁶ cells/ml in fresh medium. Over an initial period (e.g., 2 to 3 weeks) of culture, the lymphocytes selectively proliferate, while the remaining tumor cells will typically disappear completely.

To make LAK cell cultures, peripheral blood lymphocytes (PBL) are obtained from patients or normal donors. After passage over Ficoll-Hypaque gradients, cells are cultured at a concentration of 1.times. 10⁶/ml in RPMI 1640 medium with 2% human serum, antibiotics, glutamine, and HEPES buffer. Recombinant IL-2 is added at 1000 microunits/ml. Cultures are maintained for 3 to 7 days in a humidified 5% CO₂ atmosphere at 37° C.

### Ex Vivo Stimulation

This example describes an approach to stimulate host cells in vitro with SAgs for reinfusion. Tumor-draining lymph node (LN) cells are stimulated in vitro in a procedure with an optional second step. Note that T cells, NKT cells, LAK cells may be used for stimulation and are obtained from many host sources including but not limited to tumor infiltrating lymphocytes, peripheral blood, lymph nodes, spleen, and cultured and/or separated T cell lines and clones with naïve, memory, activated and/or effector or cytotoxic phenotypes. The T cells are previously immunized against tumor antigens and/or transfected with tumor specific TCRs and incubated with one or more cytokines such as IL-15 (10 ng/ml), IL-23 (2 ng/ml), IL-7 (0.1-10 ng/ml) or combinations thereof. For mice, the following is a representative example of in vitro stimulation methodology.

Step One. For stimulation, 4×10⁶ lymph node cells, in 2 ml of culture medium containing egc SAgs, with or without IL-2 (4 U/ml), but with one or more cytokines IL-15 (10 ng/ml), IL-7 (0.1-10 ng/ml), IL-23 (2 ng/ml) and/or IL-7 (0.1-10 ng/ml) are incubated in a well of 24-well plates at 37° C. in a 5% CO₂ atmosphere for 2 days. The culture media comprises RPMI 1640 medium supplemented with 10% heat inactivated fetal calf serum, 0.1 mM nonessential amino acids, 1.0 uM sodium pyruvate, 2 mM freshly prepared L-glutamine, 100 ug/ml streptomycin, 100 U/ml penicillin, 50 ug/ml gentamicin, 0.5 ug/ml fungizone (all from GIBCO, Grand Island, N.Y.) and 5.times. 10⁻⁵ M 2-ME (Sigma). The cells are harvested and washed.
Step Two. The initially stimulated cells are further cultured at 3×10⁵/well in 2 ml of culture media with or without human recombinant IL-2, 10 ng/ml (rhIL-2; Chiron) but with 10 ng/ml rhIL-15 (PeproTech, Rocky Hill, N.J.), and optionally IL-7 (0.1-10 ng/ml), and/or IL-23 (2 ng/ml). After 3 days incubation, the cells are collected, washed, counted to determine the degree of proliferation, and resuspended in media suitable for intravenous (i.v.) administration (e.g., physiological buffered saline solutions). The cell preparations are infused with IL-15.

### Preparation and Culture Activation of TDLN CD62L^{low} Cells

The TDLN cells are incubated with 100 μl anti-CD62L microbeads per 10⁸ cells and applied to MACS columns (Miltenyi Biotech, Auburn Calif.) and the flow through fraction is collected. For CD4⁺ hyperexpansion, the CD62L^{low} subset is depleted of CD8⁺ cells by MACS on day 0 and day 36 of culture activation. CD62L^{low} cells, containing approximately 50% TCR⁺ and 50% B220⁺ subsets, are suspended in complete medium (CM) and incubated for 2 days at 4×10⁶ per well with egc SAgs in 24 well culture plates together with one or more cytokines IL-7, IL-15, IL-23. Activated cells are washed, counted, and suspended at 0.5×10⁵/ml in CM with or without IL-2 (4 U/ml) (Chiron Corp. Emeryville, Calif.), but with one or more cytokines including IL-15 (10 ng/ml), and optionally rmIL-7 (10 ng/ml) and/or rhIL-23 (2 ng/ml) (each from R&D Systems, Minneapolis, Minn.) and then diluted to 10⁵/ml on day 5 of activation. On days 9 and 15, the cell concentration is adjusted to 2×10⁵/ml. For experiments with two cycles of SAg stimulation, T cells are incubated with egc SAgs for 14 hrs on day 15 plus one or more of cytokines IL-7, IL-15, IL-23 and used for adoptive therapy on day 23. For long-term expansion, cultures are maintained for 23 days after the initial egc SAg stimulation in CM with or without IL-2 (4 U/ml), but with one or more of IL-7 (10 ng/ml), IL-23 (2 ng/ml) and/or IL-7 (0.1-10 ng/ml) then are likewise stimulated with egc SAg and the above cytokines for 14 hrs on day 23 and every 7 days thereafter.

### Human TIL Activated In Vitro

For humans TIL a representative protocol for in vitro stimulation is as follows: Starting with an average of 3.4×10⁷ TILs preselected for high activity and diversity of antigen recognition, cultures are expanded during the 14-day rapid expansion protocol to an average 4.1×10¹⁰ cells on the day of infusion, which represents an average 1,320-fold expansion for each culture (range 181- to 2,623-fold), which corresponded to 7-12 cell doublings in the 14 days. Because TIL populations are grown from tumors and not cloned it is often possible to administer the cells after just a single expansion with egc SAgs with or without IL-2 but with one or more cytokines IL-15 (10 ng/ml), IL-23 (2 ng/ml) and/or IL-7 (0.1-10 ng/ml).

T cells identified from TIL with high activity, diversity of tumor antigen recognition and a specific Vβ phenotype are stimulated by SAg(s) specific for that Vβ phenotype in order to obtain massive and selective expansion of a tumor specific T cell clone.

Initiation and Expansion of TIL Microcultures from Tumor Fragments

Each tumor specimen is dissected free of surrounding normal tissue and necrotic areas. Small chunks of tumor (usually 8-16) measuring about 1 to 2 mm in each dimension are cut with a scalpel from different areas around the tumor specimen. A single tumor fragment is placed in each well of a 24-well tissue culture plate with 2 mL of complete medium (CM) plus egc SAgs and one or more of cytokines IL-15 (10 ng/ml), IL-23 (2 ng/ml) and/or IL-7 (0.1-10 ng/ml). CM consists of RPMI 1640, 25 mmol/L HEPES pH 7.2, 100 U/mL penicillin, 100 μg/mL streptomycin, 2 mmol/1-glutamine, and 5.5×10⁻⁵ mol/L β-mercaptoethanol, supplemented with 10% human serum. The plates are placed in a humidified 37° C. incubator with 5% CO₂ and cultured until lymphocyte growth is evident. Each fragment is inspected about every other day using a low-power inverted microscope to monitor the extrusion and proliferation of lymphocytes. Whether or not lymphocyte growth is visible, half of the medium is replaced in all wells no later than 1 week after culture initiation. Typically, about 1 to 2 weeks after culture initiation, a dense lymphocytic carpet covers a portion of the plate surrounding each fragment. When any well becomes almost confluent, the contents are mixed vigorously, split into two daughter wells and filled to 2 mL per well with CM plus 6000 IU/mL IL-2. Subsequently, the cultures are split to maintain a cell density of 0.8-1.6×10⁶ cells/mL, or half of the media is replaced at least twice weekly. Each initial well is considered to be an independent TIL culture and maintained separately from the others.

TIL Cultures Derived from Single-Cell Digests

For the generation of bulk TIL, cultures by enzymatic digestion of tumor explants, each solid tumor specimen is dissected free of surrounding normal tissue and necrotic areas. The tumor is sliced with a scalpel into small pieces (approximately 2 mm on each side). The tumor fragments are immersed in a mixture of collagenase, hyaluronidase, and DNAse in serum-free RPMI 1640, and incubated overnight with gentle agitation. The single-cell slurry is passed through sterile wire mesh to remove undigested tissue chunks. The digested single-cell suspensions are washed twice in HBSS, viable cells are purified on a single step Ficoll gradient, and cells are resuspended for plating. Multiple wells of a 24-well plate are seeded with 1×10⁶ viable cells in 2 mL CM with one or more cytokines such as IL-15 (10 ng/ml), IL-23 (2 ng/ml) and/or IL-7 (0.1-10 ng/ml) with or without 6000 IU/mL IL-2. The plates are placed in a humidified 37° C. incubator with 5% CO₂. Whether or not lymphocyte growth is visible, half of the medium is replaced in all wells no later than 1 week after culture initiation. When any well become nearly confluent, the contents are mixed vigorously, split into two daughter wells, and filled to 2 mL per well with CM plus 6000 IU/mL IL-2. Subsequently, half the media is replaced at least twice weekly, or the cultures are split to maintain a cell density of 0.8 to 1.6×10⁶ cells/mL. Some of the TIL from digests are derived from multiple original wells that are regularly mixed and eventually pooled for assessment of activity. Other TIL from digests are derived from individual wells of a 24-well plate. For these cultures, all progeny cells from any individual well are treated as an independent TIL culture and were maintained separately from the descendants of any other original well. In this way, multiple cultures are obtained from the same initial single-cell suspension.

### TIL Cultures and Tumor Cell Lines Derived by Physical Disaggregation of Tumor

### Samples

Some TIL are derived by a method of physical disaggregation of melanoma fragments using a device called a Medimachine (Becton Dickenson) with 50 μm medicon chambers, which are mini sterile and disposable homogenizers. Fragments of tumor about 2 mm per side are prepared by dissection of biopsy specimens free from normal and necrotic tissue. Several fragments at a time are physically disaggregated by a 30-second Medimachine treatment, which disaggregate the tumor chunks using mechanical shear provided by a rotating disk that forces the tumor chunks across a small grater inside the medicon. The resulting slurry of single cells and small cell aggregates is washed once, and resuspended in CM. The cell suspension is layered onto a two-step gradient with a lower step of 100% Ficoll, and a middle step of 75% Ficoll and 25% CM. After centrifugation at 2000 rpm (about 1100 g) for 20 minutes, the interfaces are collected. The lower interface containing the lymphocyte-enriched fraction is processed separately from the upper interface containing the melanoma-enriched cells. Each fraction is washed twice. The lower, TIL-enriched fraction is plated in 24-well plates, and individual TIL cultures are generated exactly as for the single-cell suspensions derived by enzymatic degradation. The upper, tumor-cell-enriched fraction is plated at approximately 2×10⁵ cells/mL in RPMI-based media containing 10% defined fetal calf serum (Hyclone, Logan, Utah) without IL-2. The Medimachine method is highly efficient at generating tumor cell lines, and about 50% of tumors processed by this approach are successfully converted to long-term cell lines.

### Rapid Expansion Protocol and Preparation of Cells for Infusion

Active TIL cultures were expanded to treatment levels using a rapid expansion protocol (REP) as previously described. Briefly, the REP used egc SAgs with or without IL-2 in the presence of one or more survival-promoting cytokines IL-15 (10 ng/ml), IL-23 (2 ng/ml) and/or IL-7 (0.1-10 ng/ml) with or without IL-2, irradiated, allogeneic feeder cells at a 200:1 ratio of feeder cells to responding TIL cells. PBMC feeder cells obtained from normal volunteers by apheresis were thawed, washed, resuspended in CM, and irradiated (50 Gy). PBMC (2×10⁸), egc SAgs (0.05-10 pg of each) in CM (75 mL), AIM V media (GIBCO/BRL, 75 mL), and TIL effector cells (1×10⁶) and one or more cytokines IL-15 (10 ng/ml), IL-23 (2 ng/ml) and/or IL-7 (0.1-10 ng/ml) are combined, mixed, and aliquoted to a 175 cm² tissue culture flask. Flasks are incubated upright at 37° C. in 5% CO₂. On day 2 one or more cytokines IL-15 (10 ng/ml), IL-23 (2 ng/ml) and/or IL-7 (0.1-10 ng/ml) is/are added with or without 6000 IU/mL of IL-2. If the SAgs and or egc SAgs induce sufficient proliferation by themselves, the IL-2 may be omitted but one or more of the T cell survival-promoting cytokines are retained. On day 5, 120 mL of culture supernatant is removed by aspiration (cells are retained on the bottom of the flask) and media is replaced with a 1:1 mixture of CM/AIM V containing IL-15 (10 ng/ml), IL-23 (2 ng/ml) and/or IL-7 (0.1-10 ng/ml) with or without 6000 IU/mL IL-2. On day 6 and every day thereafter, cell concentration is determined and cells are split into additional flasks or transferred to Baxter 3-L culture bags with additional medium containing IL-15 (10 ng/ml), IL-23 (2 ng/ml) and/or IL-7 (0.1-10 ng/ml) with or without 6000 CU/mL IL-2 as needed to maintain cell densities around 1×10⁶ cells/mL. About 14 days after initiation of the REP, cells are harvested from culture bags and prepared for patient treatment. Harvesting is accomplished using a Baxter/Fenwal continuous centrifuge cell harvester system. The cells are then washed in 0.9% sodium chloride and resuspended in 45 to 150 mL of 0.9% sodium chloride with 2.5% human albumin. Samples were removed from the infusion product for QC testing, aliquots were cryopreserved for future experimental analysis, and the remaining cells are infused into the patient by intravenous administration over 30 minutes.

TIL cultures that exhibited specific tumor cell recognition are expanded for treatment by using one or two cycles of a rapid expansion protocol with irradiated allogeneic feeder cells, SAgs or SAg homologues specific for the Vβ phenotype of the expanded clone or clones, and one or more cytokines consisting of IL-15 (10 ng/ml), IL-23 (2 ng/ml) and/or IL-7 (0.1-10 ng/ml) with or without/6,000 units per ml of IL-2. Vβ phenotypes of the SAgs is shown in the Table 18.

Native SAgs and SAg mutants with more constricted Vβ phenotypes than native SEs are useful to stimulate small groupings of tumor recognizing T cells expressing the appropriate Vβ profile. For example, SEA activates human T cell Vβ clones 1, 5, 6, 7, 18 while SEA substitution mutants SEA-D227A and SEA-F47A activate a narrower group of Vβ clones 5, 6, 7, 18 and 6, 7 respectively. Native and mutant SAgs may be combined ex vivo to activate the tumor specific Vβ clones identified after TIL isolation and/or ex vivo stimulation of T cells with a library of isolated tumor specific antigens, tumor lysates or inactivated whole tumor cells. This selective Vβ expansion with SE's may be carried out in the presence of cytokines IL-15 (preferably) and optionally IL-7 and/or IL-23 ex vivo. To prevent AICD in vivo, one or a plurality of the same cytokines (IL-15 preferably) may be used as described above before during or after adoptive transfer (infusions) of ex vivo activated T cells.

TABLE 18

| SAg | Human TCR VB Specificity |
|---|---|
| SEA | 1.1, 5.3, 6.3, 6.4, 6.9, |
| | 7.3, 7.4, 9.1, 23.1 |
| SEB | 1.1, 3.2, 6.4, 15.1 |
| SEC1 | 3.2, 6.4, 6.9, 12, 15.1 |
| SEC2 | 12. 13, 14, 15, 17, 20 |
| SEC3 | 5.1, 12 |
| SED | 1.1, 5.3, 6.9, 7.4, 8.1, |
| | 12.1 |
| SEE | 5.1, 6.3, 6.4, 6.9, 8.1 |
| SEG | 3, 12, 13.2, 13.6, 14 |
| SEH | |
| SEI | 1.1, 5.1, 5.3, 6, 23 |
| SEJ | |
| SEK | 5.1, 5.2, 6.7 |
| SEL | |
| SEM | 6, 21 |
| SEN | 9 |
| SEO | 5, 7 |
| SEO | |
| SEP | |
| SEQ | 2.1, 5.1, 21.3 |
| TSST | 2.1 |
| SPEA | 2.1, 12.2, 14.1, 15.1 |
| SPEC | 2.1, 3.2, 12.5, 15.1 |
| SPEG | 2.1, 4.1, 6.9, 9.1, 12.3 |
| SPEH | 2.1, 7.3, 9.1, 23.1 |
| SPEI | 6.9, 9.1, 18.1, 22 |
| SPEJ | 2.1 |
| SPEL/K | 1.1, 5.1, 23.1 |
| SPEM | 1.1, 5.1, 23.1 |
| SSA | 1.1, 3 15 |
| SMEZ1 | 2.1, 4.1, 7.3, 8.1 |
| SMEZ2 | 4.1, 8.1 |
| YPMA | 3, 9, 13.1, 13.2 |
| YPMB | 3.9 13.1, 13.2 |
| MAM | 6, 8 |

### Cytokine Release Assays

TIL activity and specificity are determined by analysis of cytokine secretion. TIL and control T-cell lines are washed twice prior to coculture assay to remove IL-2. TIL cells (1×10⁵) are plated per well of a 96-well flat-bottom plate with 1×10⁵ stimulator cells. TIL cultures are generally stimulated with at least two HLA-A2⁻ melanoma cell lines (888-mel and 938mel) or cell lines from other tumors including lung, colon, breast, stomach, ovarian carcinomas and at least two HLA-A2⁺ tumor cell lines (526-mel and 624mel). When available, TIL are also stimulated with an autologous tumor cell line or a thawed aliquot of cryopreserved single-cell tumor digests (fresh tumor). For some TIL, the TAP-deficient T2 cell line is pulsed with melanoma antigen peptides including MART-1:27-35 (referred to as MART) or gp100:209-217 (referred to as g209). After overnight coculture, supernatants are harvested and IFN-γ secretion is quantified by ELISA (Pierce/Endogen, Woburn, Mass.). All cytokine release assays are routinely controlled with the JB2F4 T-cell clone specific for the MART-1:27-35 antigen and the CK3H6 T cell clone specific for the gp100:209-217 antigen.

### TCR CDR3 Size Pattern Analysis

The T-cell receptor (TCR) complementarity determining region (CDR)₃ of TIL and PBMC are investigated using the Immunoscope approach. Briefly, total RNA is extracted from pretreatment PBMC or TIL using RNAeasy columns (Qiagen, Valencia, Calif.), and reverse transcribed into cDNA using oligo-dT primers and AMV (Roche, Mannheim, Germany). cDNA is amplified using BV and BC specific primers, and the unlabeled PCR product is copied in 5-cycle run-off reactions using a nested fluorescent BC primer. Aliquots are analyzed on an Applied Biosystems 377 DNA sequencer and size patterns obtained with the aid of the Immunoscope software.

### FACS Analysis

T cells are washed and resuspended at 1×10⁷ cells/mL in FACS buffer consisting of PBS+5% fetal calf serum. Staining with anti-CD8 antibody and a panel of TCR Vβ specific antibodies (Beckman/Coulter/Immunotech) or HLA-A2/MART-1:26-55(27L) iTAG te-tramer complexes (Beckman/Coulter/Immunomics) was carried out according to the manufacturer's recommendations. Cells are washed twice in FACS buffer and analyzed using a FACSCaliber (BD Biosciences) with live/dead cell gating based on propidium iodide exclusion. FACS results are analyzed with Cellquest software (Becton Dickenson, San Jose, Calif.).

### Adoptive Immunotherapy Protocol in Mice & Humans & Outcomes

As noted previously, the present invention involves stimulating cells ex vivo with SAgs, allowing them to differentiate into tumor specific immune effector cells. The cells are then reintroduced into the same host to mediate anticancer therapeutic effects.

In this example, 8 to 12 week old female C57BL/6J (B6) mice (Jackson Laboratory, Bar Harbor, Me.) are injected i.v. with approximately 3×10⁵ MCA 205 or 207 tumor cells (i.e., methylcholanthrene-induced tumors of B6 origin provided by Dr. James Yang, Surgery Branch, National Cancer Institute, Bethesda, Md.) suspended in 1 ml of media to initiate pulmonary metastases. Subcutaneous tumors are established by inoculation of 1.5×10⁶ cells. Intracranial tumors are established by transcranial inoculation of 10⁵ tumor cells at a depth of 4 mm. Mice bearing 3-day s.c. or i.c. tumors or 10-day pulmonary metastases are treated with 5 Gy nonmyeloablative total body irradiation (TBI) delivered from a ¹³⁷Cs irradiator prior to intravenous transfer of the T cells whereas mice with 3-day pulmonary tumors are not irradiated. The antitumor efficacy of SAg-stimulated cells is assessed by reinfusion. The intravenous transfer of the ex vivo SAg-stimulated T cells is accompanied by parenteral (e.g., ip, iv, subcutaneous, intradermal, intratumoral, intrathecal, intravesicular, intrapleural, intralymphatic) injections of one or more cytokines such as IL-2 (15,000 U IL-2 in 0.5 ml buffered saline twice daily for 4 consecutive days), IL-7, IL-15 or IL-23 (12-200 ug twice daily for 6 doses) to promote the in vivo function and survival of the stimulated cells. IL-15 is preferred. For pulmonary tumors, mice are euthanized on day 20 post inoculation, the lungs are insufflated with India ink and the number of surface tumor nodules is enumerated using a dissecting microscope. Subcutaneous tumors are measured in two perpendicular dimensions three times per week and mice with progressive tumors are euthanized when the product of dimensions exceeded 200 mm². Mice bearing intracranial tumors are monitored daily for survival or are euthanized when neurologic symptoms such as decreased grooming and decreased spontaneous movement are apparent.

For human therapy each patient is treated with myeloablative chemotherapy starting 7 days before cell administration, consisting of 2 days of cyclophosphamide at 60 mg/kg of body weight, followed by 5 days of fludarabine at 25 mg/m². On the day after the final dose of fludarabine, when circulating lymphocyte and neutrophil counts decline to <20/mm³, each patient receives an i.v. infusion of autologous lymphocytes, 10⁻⁹-10⁻¹¹, over 30-60 min. After cell infusion, patients receive one or more cytokines selected from a group consisting of IL-15 (0.15-8 mg/kg), IL-7 (0.5 ug/day), IL-23 (0.1-200 ug/day), with or without high-dose IL-2 therapy consisting of 720,000 units per kg bolus i.v. infusion every 8 hours to tolerance. The cytokines are given twice daily for 3-7 days after each egc SAg injection. The cytokines are also administered before, at the same time or after cell administration intrathecally, intrapleurally, intrapericardially, intravesicularly, intramuscularly, intralymphatically, intraarticularly, intratumorally, subcutaneously, intradermally or by any other parenteral route by infusion, injection, instillation or implantation. Some patients with mixed or responding lesions will receive an additional course of cell transfer therapy.

TABLE 19

| | T cell | | | |
|---|---|---|---|---|
| | infused × | | | % of Patients |
| | 10⁻¹⁰ | No. of Patients | Response | Responding |
| All Patients | | 216 | CR | 80 |
| | | 8 | PR | 3 |
| | | 3 | <PR | 1 |
| By Tumor Type: | | | | |
| Breast adenocarcinoma | 2.5 | 30 | CR + PR + <PR | 90% |
| Gastrointestinal carcinoma | 3.2 | 25 | CR + PR + <PR | 90% |
| Lung Carcinoma | 4.1 | 45 | CR + PR + <PR | 95% |
| Brain glioma/astrocytoma | 2.7 | 20 | CR + PR + <PR | 85% |
| Prostate Carcinoma | 5.3 | 35 | CR + PR + <PR | 85% |
| Lymphoma/Leukemia | 3.7 | 20 | CR + PR + <PR | 80% |
| Head and Neck Cancer | 5.9. | 30 | CR + PR + <PR | 80% |
| Renal and Bladder Cancer | 4.8 | 20 | CR + PR + <PR | 95% |
| Melanoma | 5.1 | 20 | CR + PR + <PR | 85% |
| Neuroblastoma | 2.1 | 25 | CR + PR + <PR | 85% |

Results: A total of 270 patients are patients treated. The number of patients for each tumor type and the results of treatment are summarized in Table 19. Positive tumor responses are observed in as high as 85-95% of the patients with breast, gastrointestinal, lung, prostate, renal and bladder tumors as well as melanoma and neuroblastoma as follows.

Two hundred and twenty seven of 270 entered with all tumors exhibit objective clinical responses for an overall response rate of 84%. Tumors generally start to diminish and objective remissions are evident after four weeks starting therapy. Responses endure for an median of 36 months.

Toxicity consists of mild short-lived fever, fatigue and anorexia not requiring treatment. The incidence of side effects (as % of total treatments) are as follows: chills—5%; fever—10%; pain—7; nausea—6; respiratory—1; headache—3; tachycardia—3; vomiting—2; hypertension—1; hypotension—1; joint pain—4; rash—2; flushing—3; diarrhea—1; itching/hives—2; bloody nose—2; dizziness—1. Fever and chills are the most common side effects observed. Toxic effects usually associated with systemically administered chemotherapeutic agents were not observed.

### Example 9

### Intravesical Administration of SEs and egc SEs

The main indication for institution of intravesicular SE or egc SE treatment include intermediate- and high-grade tumor, multiple neoplasms at presentation, a history of one or more tumor recurrences, advanced-stage (T1) superficial carcinoma, a large tumor (>3 cm in diameter), persistently positive urinary cytology following transurethral resection (TUR), and/or finding of concomitant CIS or severe dysplasia on random bladder biopsy. In these settings, TUR alone is insufficient in controlling the disease because of the unacceptable high rates of recurrence, progression and ultimate cystectomy. However, there are no uniformly rigid indications for the instillation of egc SE intravesical therapy and it may be used as preventative against recurrent tumor or for established tumors of the bladder at the discretion of the physician.

For the treatment of papillary transitional cell carcinoma of the bladder with egc SEs, patients are dehydrated for 8 to 12 hours prior to treatment and 1-1000 pg of each egc SE (in 25-50 mL of saline solution) is instilled into the bladder by catheter. The solution is retained for 2 hours. If the patient cannot retain 60 mL for 2 hours, the dose is given in a volume of 30 mL. The patient is positioned every 15 minutes for maximum area contact. The treatment is administered weekly for 4-8 weeks. The course is repeated if residual tumor remains. For local toxicity (chemical cystitis), a 50% dose reduction is used. For carcinoma-in-situ, depending on the individual tolerability, the dose is increased up to 80 mg. For prophylaxis of recurrences after transurethral resection of superficial tumours, 4 weekly administrations of 1-1000 pg of each egc SE followed by 11 monthly instillations at the same dosage is used. Generally, the instillate is retained in the bladder for one hour and during instillation, the pelvis of the patient is rotated to ensure the most extensive contact of the solution with the vesical mucosa. To avoid undue dilution with the urine, the patient is instructed not to drink any fluid in the twelve hours prior to instillation.

### Example 10

### Intrapericardial egc SEs

As indicated in Example 1, patient 3 (Table 8) had a recurrent pleural and pericardial effusion 15 months after his first treatment and was retreated twice with intrapleural and intrapericardial egc SAg. The patient was in pericardial tamponade showing distended neck veins, hypotension and muffled heart sounds. The pericardial effusion resolved after pericardiocentesis followed by a single instillation of egc SEs 400 pg. Pericardiocentesis was carried out via a subzyphoid approach using an 18-gauge short-bevel spinal needle attached to a 20-ml syringe. There was no further recurrence of the pericardial effusion.

The needle was aimed at the right shoulder or and was aspirated as it was advanced. Following the intrapericardial administration of egc SEs, the pericardial effusion did not recur. As described herein, chemotherapy may be administered locally before, together with the egc SEs or parenterally (preferably intravenously) at the same time or after (preferably up to 48 hours) intrapericardial egc SE instillation.

All the references cited above in this patent application are incorporated by reference in entirety, whether specifically incorporated or not. In addition, the following co-pending patent applications are incorporated by reference in their entirety:

| Inventor | Ser. No. | Filing Date | Title |
|---|---|---|---|
| Terman, D. S. | 60/665,654 | Mar. 23, 2005 | Enterotoxin Gene Cluster Superantigens (egc) to Treat |
| | | | Malignant Disease |
| Terman, D. S, | 60/626,159 | Nov. 6, 2004 | Enterotoxin Gene Cluster Superantigens (egc) to Treat |
| Etiene, J., | | | Malignant Disease |
| Vandenesch, F., | | | |
| Lina, G. | | | |
| Bohach, G. | | | |
| Terman, D. S. | 60/583,692 | Jun. 29, 2004 | Intrathecal and Intrapleural Superantigens to Treat |
| | | | Malignant Disease |
| Terman, D. S. | 60/550,926 | Mar. 5, 2004 | Intrathecal and Intrapleural Superantigens to Treat |
| | | | Malignant Disease |
| Terman, D. S. | 60/539,863 | Jan. 27, 2004 | Intrathecal and Intrapleural Superantigens to Treat |
| | | | Malignant Disease |
| Terman, D. S. | PCT/US03/ | May 8, 2003 | Intrathecal and Intrapleural Superantigens to Treat |
| | 14381 | | Malignant Disease |
| Terman, D. S. | Pending | May 5, 2003 | Composition and Methods for Treatment of Neoplastic |
| | | | Diseases |
| Terman, D. S. | 60/438,686 | Jan. 9, 2003 | Intrathecal and Intrapleural Superantigens to Treat |
| | | | Malignant Disease |
| Terman, D. S. | 60/415,310 | Oct. 1, 2002 | Intrathecal and Intratumoral Superantigens to Treat |
| | | | Malignant Disease. |
| Terman, D. S. | 60/406,750 | Aug. 29, 2002 | Intrathecal Superantigens to Treat Malignant Fluid |
| | | | Accumulation |
| Terman, D. S. | 60/415,400 | Oct. 2, 2002 | Composition and Methods for Treatment of Neoplastic |
| | | | Diseases |
| Terman, D. S. | 60/406,697 | Aug. 28, 2002 | Compositions and Methods for Treatment of |
| | | | Neoplastic Diseases |
| Terman, D. S. | 60/389,366 | Jun. 15, 2002 | Compositions and Methods for Treatment of |
| | | | Neoplastic Diseases |
| Terman, D. S. | 60/378,988 | May 8, 2002 | Compositions and Methods for Treatment of |
| | | | Neoplastic Diseases |
| Terman, D. S. | 09/870,759 | May 30, 2001 | Compositions and Methods for Treatment of |
| | | | Neoplastic Diseases |
| Terman, D. S. | 09/751,708 | Dec. 28, 2000 | Compositions and Methods for Treatment of |
| | | | Neoplastic Diseases |

Having now fully described this invention, it will be appreciated by those skilled in the art that the same can be performed within a wide range of equivalent parameters, concentrations, and conditions without departing from the spirit and scope of the invention and without undue experimentation.

What is claimed is:

1. A method for treating a subject with cancer who manifests intrathecal tumor with or without fluid accumulation, comprising administering intrathecally into an organ sheath or a body cavity of said subject an effective amount of a superantigen composition comprises one or a plurality of different superantigen molecules selected from the group consisting of:
(a) native enterotoxin G, I, M, N, O;
(b) biologically active fragments of the native enterotoxins G, I, M, N, O;
(c) biologically active homologues of the native enterotoxins of (a) or of said fragments of (b); and
(d) biologically active fusion protein comprising said native enterotoxins G, I, M, N, O of (a), said fragment of (b) or said homologue of (c), fused to a polypeptide, peptide or nucleic acid fusion partner

2. The method of claim 1 wherein the biologically active fragment, homologue of fusion protein has the biological activity of stimulating T cells via a T cell receptor Vβ or Vα region.

3. The method of claim 1 wherein said subject has a malignant pleural effusion and said intrathecal administration is by an intrapleural route.

4. The method of claim 1 wherein said subject has a malignant pericardial effusion and said intrathecal administration is by an intrapericardial route.

5. The method of claim 1 wherein said subject has malignant ascites and said intrathecal administration is by an intraperitoneal route.

6. The method of claim 1 wherein said subject has cerebral edema due to meningeal metastatic carcinomatosis and said administration is by intrathecal route into a spinal or meningeal space.

7. A method of treating a subject with a tumor of the lung and/or pleura and/or lung-associated lymphatic tissue, comprising administering to said subject (i) intrapleurally, (ii) intratumorally, (iii) intravenously, (iv) intralymphatically, or (v) by any one or more of routes (i)-(iv), an effective amount of a superantigen composition comprising one or a plurality native enterotoxins G, I, M, N, O molecules selected from the group consisting of:
(a) a native enterotoxin G, I, M, N, O protein;
(b) a biologically active fragment of the native enterotoxin G, I, M, N, O proteins;
(c) a biologically active homologue of the native enterotoxin G, I, M, N, O proteins of (a) or of said fragments of (b); and
(d) a biologically active fusion protein comprising said native enterotoxins G, I, M, N, O proteins of (a), said fragment of (b) or said homologue of (c), fused to a fusion partner polypeptide, peptide or nucleic acid.

8. The method of claim 7 wherein the biologically active fragment, homologue of fusion protein has the biological activity of stimulating T cells via a T cell receptor Vβ or Vα region.

9. A method for treating a subject with a tumor comprising administering intratumorally to said subject by injection, infusion or implantation an effective amount of a superantigen composition comprising one or more superantigen molecules selected from the group consisting of:
(a) a native enterotoxin G, I, M, N, O protein;
(b) a biologically active fragment of the native enterotoxin G, I, M, N, O proteins;
(c) a biologically active homologue of the native enterotoxins G, I, M, N, O protein of (a) or of said fragment of (b); and
(d) a biologically active fusion protein comprising said native enterotoxins G, I, M, N, O proteins of (a), said fragment of (b) or said homologue of (c), fused to a fusion partner polypeptide or peptide.

10. A method of treating a subject with a tumor comprising administering to said subject (i) intrapleurally, (ii) intratumorally, (iii) intravenously, (iv) intralymphatically, (v) intramuscularly (vi) intradermally (vii) subcutaneously (viii) intrathecally (ix) intravesicularly (x) intrapericardially or (xi) intraarticularly (xii) intraperitoneally by any one or more of routes (i)-(xi), by infusion, injection, instillation or implantation an effective amount of a superantigen composition comprising one or a plurality native enterotoxins G, I, M, N, O molecules selected from the group consisting of:
(a) a native enterotoxin G, I, M, N, O protein;
(b) a biologically active fragment of the native enterotoxin G, I, M, N, O proteins;
(c) a biologically active homologue of the native enterotoxin G, I, M, N, O proteins of (a) or of said fragments of (b); and
(d) a biologically active fusion protein comprising said native enterotoxins G, I, M, N, O proteins of (a), said fragment of (b) or said homologue of (c), fused to a fusion partner polypeptide, peptide or nucleic acid.

11. A method of claim 10 egc wherein the superantigens or superantigen homologue is administered as a preventative vaccine in a subject without evident tumor or with minimal tumor burden.

12. The method of claim 1-10 wherein the biologically active fragment or homologue of fusion protein has the biological activity of stimulating T cells via a T cell receptor Vβ or Vα region.

13. The method of claims 1-12 where the superantigen compositions are administered by injection, infusion, instillation or implantation.

14. The method of any of claims 1-13 wherein the superantigen composition comprises one or a plurality of said native superantigens.

15. The method of any of claims 1-14 wherein the superantigen composition comprises said one or a plurality of superantigen fragments.

16. The method of any of claims 1-15 wherein the superantigen composition comprises one or a plurality of said different superantigen homologues.

17. The method of any of claims 1-16 wherein the superantigen composition comprises said fusion proteins.

18. The method of claim 1-17 wherein said homologues have at least 20% amino acid sequence identity with said native superantigen as measured using a sequence comparison algorithm.

19. The method of claims 1-18 wherein, when said fusion protein comprises said homologue, said homologue has at least 20% amino acid sequence identity with said native superantigen as measured using a sequence comparison algorithm.

20. The method of claim 1-19 wherein said homologues have sequence homology to said native superantigen protein characterized by a z value exceeding 10 when the sequence of the homologue is compared to the sequence of the native superantigenic protein using an algorithm and Monte Carlo analysis according to W. R. Pearson and D. J. Lipman in the Proceedings of the National Academy of Science U.S.A., 85:2444-2448, 1988.

21. The method of claim 1-21 wherein, when said fusion protein comprises said homologue, said homologue has sequence homology to said native superantigen protein characterized by a z value exceeding 10 when the sequence of the homologue is compared to the sequence of the native superantigenic protein using an algorithm and Monte Carlo analysis according to W. R. Pearson and D. J. Lipman in the Proceedings of the National Academy of Science U.S.A., 85:2444-2448, 1988.

22. The method of any of claims 1-21 further comprising administering a chemotherapeutic drug before, together with or after administration of said superantigen composition.

23. The method of claim 1-22 wherein the chemotherapeutic drug(s) is administered between 1 week before to 1 week after administration of the superantigen composition.

24. The method of claims 1-23 wherein the chemotherapeutic drug is administered parenterally, intrathecally, intratumorally, intravenously, intramuscularly, subcutaneously, intrapleurally, intrapericardially, intravesicularly, intrathecally, intrapleurally, intrapericardially, intravesicularly, intraarticularly, intraperitoneally, intralymphatically, intradermally.

25. The method of claims 1-24 wherein the chemotherapeutic agent is administered by injection, infusion, instillation or implantation.

26. The method of claim 1-25 wherein the chemotherapeutic drug is administered intratumorally.

27. The method of claims 1-26 wherein the chemotherapeutic drug is administered in doses 10-95% below a therapeutically effective dose of said drug, which therapeutically effective dose is based on administration of said drug alone or in a combination therapy but without said superantigen composition.

28. The method of claim 1-27 wherein the chemotherapeutic drug is administered as a single agent or as a combination of more than one chemotherapeutic drugs.

29. The method of claim 1-28 wherein the chemotherapeutic drug is administered as a single agent or as a combination of more than one chemotherapeutic drugs.

30. The method of any of claims 1-29, wherein said superantigen composition is administered in a controlled release formulation.

31. The method of claims 1-30 wherein the superantigen composition, said chemotherapeutic drug, or both, are administered in a controlled release formulation by injection, infusion or implantation.

32. The method of claim 31 wherein said chemotherapeutic drug is administered before, together with or after said administration of superantigen composition.

33. The method of any of claims 1-10 wherein x-radiation is administered to the tumor before, at the same time, or after, said administration of said superantigen composition.

34. The method of claim 33 wherein x-radiation is administered to the tumor before, at the same time or after said administration of said superantigen composition and/or administration of said chemotherapeutic drug or drugs.

35. The method of claim 24 wherein x-radiation is administered to the tumor before, together with or after said administration of said superantigen composition and/or administration of said chemotherapeutic drug or drugs.

36. The method of claim 25 wherein x-radiation is administered to the tumor before, together with or after said administration of said superantigen composition and/or administration of said chemotherapeutic drug or drugs.

37. The methods of claims 1-10 said method comprising administering one or a plurality of cytokines by injection, infusion or implantation, intravenously, intrapleurally, intrathecally, intravesicularly, intraperitoneally, intralymphatically, subcutaneously, intradermally, intramuscularly, intraarticularly, intraarterially.

38. The method of claims 1-10 wherein said one or a plurality of cytokines are selected from the group consisting of hematopoietic growth factors, interleukins, interferons, immunoglobulin superfamily molecules, tumor necrosis factor family molecules and chemokines.

39. The method of claims 1-10 wherein one or a plurality of cytokines are selected from a group consisting of IL-2, IL-15, IL-7, IL-23 and most preferably IL-15.

40. The method of claims 1-10 where one or a plurality of cytokines is administered before, at the same time or after the superantigen composition.

42. The method of any of claims 1-10 further comprising administering an tumor angiostatic or angiolytic agent or drug(s) or a tumor growth factor inhibiting drug before, at the same time or after administration of said superantigen composition.

43. The method of treatment of a subject with cancer comprising administering one or a plurality of egc SE's wherein said egc SE's are produced by biochemical methodology.

44. The method of treatment of a subject with cancer comprising administering one or a plurality of egc SE's produced by recombinant methodology.

45. The method of claim 62 wherein the egc superantigens are prepared and administered in nucleic acid form.

46. A mixture comprising at least two of the staphylococcal enterotoxins G, I, M, N, O or homologues or fragments of said enterotoxins each with essentially the same biologic activity as an enterotoxin said mixture activating a human T cell populations expressing at least 5 different Vβ/α motifs and capable of inducing a tumoricidal response when adminstered parenterally intravenously, intrathecally, intradermally, subcutaneously, intrapleurally, intrapericardially, intravesicularly, intraperitoneally, intralymphatically, intraarticularly by injection, infusion or implantation.

47. The mixture of claim 37 wherein the Staphylococcal enterotoxins in said mixture are administered intrathecally by injection, infusion, instillation or implantation every 3-7 days for 1-5 weeks in doses of each enterotoxin ranging from 0.0001-1000 nanograms.

48. A method for inducing a tumoricidal reaction in vivo comprising:
(a) obtaining a sample comprising tumor-sensitized lymphocytes, wherein bodily fluids are substantially absent from said sample;
(b) contacting said sample with one or more staphylococcal enterotoxins G, I, M, N, O ex vivo with one or more cytokine(s) in a medium substantially free from tumor cells or other source of tumor antigen to produce stimulated cells; and
(c) infusing said stimulated cells into a tumor-bearing host with cytokine(s) so as to induce an in vivo therapeutic, tumoricidal reaction.

49. The method of claim 48 wherein the cytokines incubated with the egc SEs in vitro are selected from a group consisting of IL-2, IL-7, IL-15, IL-23.

50. The method of claim 48 wherein said sample is obtained from a source selected from the group consisting of spleen, lymph node, peripheral blood, and tumor tissue.

51. The method of claim 44 wherein said sample is from said tumor-bearing host.

52. The method of claim 44 wherein the one or more cytokines used for infusion are selected from a group consisting of IL-2, IL-7, IL15, IL-23.

53. The method of claim 44 wherein one or more cytokines used for infusion are administered several days before, at the same time or several days after each infusion.

54. The method of claim 42 wherein said tumor-sensitized lymphocytes are established as a cell line prior to contact with said one or more superantigens.

55. The method of claim 44 wherein said sample comprises tumor-sensitized T cells.

56. The method of claim 44 wherein said tumor-sensitized T cells comprise produce gamma interferon.

57. The method of claim 44 wherein said one or more superantigens comprise superantigen homologue, fragment, derivative, conjugate or fusion protein of a lymphocyte-stimulating toxin comprising one or a plurality from a group consisting of egc SE's SEG, SEI, SEM, SEN, SEQ with substantially the same stimulatory effects on lymphocytes as the selected toxin.

58. The method of claim 44 wherein said sample is obtained from a tumor-draining lymph node.

59. The method of claim 44 wherein said sample additionally comprises antigen presenting cells expressing MHC class II molecules.

60. The method of claim 44 wherein said sample additionally comprises antigen presenting cells expressing MHC class II molecules.

61. The method of claim 44 further comprising contacting said sample with an agent capable of enhancing T cell proliferation and secretion.

62. The method of claim 44 wherein said contacting comprises culturing said sample comprising tumor-sensitized lymphocytes in a culture medium containing said one or more superantigens.

63. The method of claim 44 further comprising the step of washing said stimulated cells prior to infusing said stimulated cells into said patient so as to essentially avoid introducing said one or more superantigens in vivo.

64. The method of claim 77 in which the egc SE's are conjugated to an immunotherapeutic antigen either biochemically or recombinantly.

65. The method of claims 77 and 78 wherein the egc SE's or conjugates of egc SE's with immunotherapeutic antigens are administered once or repeatedly by injection, infusion or implantation.

66. The method of claims 77-79 wherein the immunotherapeutic vaccine is administered parenterally, intramuscularly, intravenously, intrathecally, intrapleurally, intravesicularly, intraarticularly, intraperitoneally, intrapericardially, subcutaneously, intradermally, intralymphatically.
